(19) United States
(12) Patent Application Publication
(10) Pub. No.: US 2025/0095782 A1
(43) Pub. Date: Mar. 20, 2025

(54) SINGLE-LOCI AND MULTI-LOCI TARGETED SINGLE POINT AMPLICON FRAGMENT SEQUENCING

(71) Applicant: GUSTO GLOBAL, LLC, Charlotte, NC (US)

(72) Inventors: Daniel Van Der Lelie, Chapel Hill, NC (US); Lisa Ouellette, Huntersville, NC (US); Safiyh Taghavi, Chapel Hill, NC (US)

(21) Appl. No.: 18/780,156

(22) Filed: Jul. 22, 2024

Related U.S. Application Data
(63) Continuation of application No. PCT/US2023/011406, filed on Jan. 24, 2023.
(60) Provisional application No. 63/302,313, filed on Jan. 24, 2022.
(60) Provisional application No. 63/340,004, filed on May 10, 2022.

(51) Int. Cl. G16B 30/20; C12Q 1/6853; G16B 10/00; G16H 50/20

(52) U.S. Cl. CPC G16B 30/20; C12Q 1/6853; G16B 10/00; G16H 50/20

(57) ABSTRACT

The systems and methods described herein are directed to amplifying microbial cell free DNA (mcfDNA). In an aspect, described herein is a method of amplifying microbial cell free DNA (mcfDNA), comprising using one or more degenerate primers with complementarity to one or more conserved regions and a second primer comprising complementarity to a repaired version of an adaptor ligated to ends of the mcfDNA, wherein the one or more degenerate primers are oriented to prime polymerase extension of the hypervariable region to generate amplified mcfDNA fragments.

### CROSS REFERENCE

This application is a continuation application of International Application No. PCT/US2023/011406, filed Jan. 24, 2023, which claims the benefit of U.S. Provisional Application No. 63/302,313 filed Jan. 24, 2022, and U.S. Provisional Application No. 63/340,004 filed May 10, 2022, both of which are incorporated herein by reference in entirety.

### SEQUENCE LISTING

The instant application contains a Sequence Listing which has been submitted electronically in XML file format and is hereby incorporated by reference in its entirety. Said XML copy, created on Jul. 18, 2024, is named 63906-701_301_SL.xml and is 378,874 bytes in size.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### TECHNICAL FIELD

The presently disclosed subject matter relates to a high-throughput, high-resolution and low-cost method of next generation amplicon fragment sequencing of biological samples.

### BACKGROUND

Liquid biopsy based on circulating cell-free DNA (cfDNA) provides a new prospect for the diagnosis, monitoring and risk assessment of a range of diseases. cfDNA molecules circulating in peripheral blood originate from dying human cells as well as from viruses, parasites, and colonizing or invasive microbes that release their nucleic acids into the blood as they die and break down (Jahr et al, 2001). Human-derived cfDNA has evolved into an indispensable biomarker in clinical practice for rapid and noninvasive diagnosis in prenatal screening, organ transplantation, and oncology (Decker and Sholl, 2020; Liang et al, 2019; Sun and Yiang, 2019; Wu et al, 2020).

Although early studies did not focus on cfDNA of microbial origin (hereinafter referred to as mcfDNA), the development of circulating mcfDNA-based tests for infectious diseases has recently been gaining traction in clinical practice. An increasing number of studies have demonstrated that mcfDNA detection offers the potential to reliably identify a wide variety of infections, such as invasive fungal infection, tuberculosis, sepsis, cystic fibrosis (Rassoulian Barrett et al, 2020) and chorioamnionitis (Witt et al, 2020; for review see Han et al, 2020).

In addition to their role in infectious diseases, several studies have shown the presence of distinct cultivable bacteria in a range of cancers, including lung (Jin et al, 2019), prostate (Gorelick et al, 1988; Cohen et al, 2005), pancreas (Geller et al, 2017; Riquelme et al, 2019), and colon cancers (Bullman et al, 2019; Castellarin et al, 2012). It was only recently suggested that cancer types outside of the aerodigestive tract, such as breast (Urbaniak et al, 2016) or brain cancer (Venkataramani et al. 2019; Zeng et al, 2019), may also harbor microbiota with distinctive compositions (for review, see Sepich-Poore et al, 2021), including fungi (Narunsky-Haziza et al, 2022). Both Nejman et al. (2020) and Poore et al. (2020) suggested the existence of distinct intratumoral microbiomes among >30 cancer types; these microbiomes also vary in composition at different developmental stages of the tumor, thus providing biomarkers for disease progression and prognosis for patient outcomes. As for other bacteria that are colonizing or infecting the body, the tumor associated bacteria will release distinct mcfDNA in the blood stream, and this let Poore et al (2020) propose the analysis of mcfDNA from the peripheral blood as a tool to gain valuable information regarding the progression of various types of cancers.

Conventional amplicon-based sequencing approaches are routinely used to determine microbial community composition in a wide range of biological samples. The most used approach is amplicon sequencing of the 16S rRNA gene based on its variable regions, such as the V1-V2 and V3-V4 regions (Gupta et al, 2019). Shahir et al (2020) applied 16S rRNA gene sequencing to identify region-specific composition and aerotolerance profiles of mucosally adherent bacteria in biopsy samples taken from the colon and ileum of Crohn's disease and non-IBD patients. As an alternative to 16S rRNA gene sequencing, single copy proteins encoding housekeeping genes including the genes for the DNA gyrase subunit B (gyrB) (Poirier et al, 2018), RNA polymerase subunit B (rpoB) (Vos at al, 2012; Ogier et al, 2019), the heat shock protein 60 (hsp60), the superoxide dismutase A (sodA), the TU elongation factor (tuf) (Ghebremedhin et al, 2008) and the 60 kDa chaperonin protein (cpn60) (Links et al, 2012) have been proposed as phylogenetic marker genes.

Liquid biopsy samples, especially peripheral blood, represent unique challenges for the analysis of microbial signatures. The majority of mcfDNA fragments in blood was found to be approximately 40-100 bp in size (Bumham et al, 2016), as was confirmed by Rassoulian Barrett et al (2020). Due to the small size of mcfDNA fragments conventional amplicon-based sequencing approaches that target DNA fragments of several hundred nucleotides (>400) are not suitable for determining the composition of colonizing or invasive microorganisms using mcfDNA from liquid biopsy samples. For example, the V1-V2 and the V3-V4 regions of the 16S rRNA gene have an average length of 437 and 443 nucleotides, respectively. Furthermore, the concentrations of plasma cfDNA in healthy individuals varies greatly, generally within the range of 0-100 ng per milliliter of plasma, sometimes exceeding 1500 ng per milliliter. Human cfDNA accounts for the vast majority (>90% or even >99%), while mcfDNA accounts for only a small fraction with 0.08%-4.85% from bacteria, 0.00%-0.010% from fungi, and 0.00%-0.16% from viruses/phages. However, it should be noted that elevated levels of mcfDNA can sometimes be observed in certain pathological conditions, including infection, sepsis, trauma, and autoimmune diseases (Han et al, 2020). Because the analysis of mcfDNA requires deep next generation sequencing (NGS) of plasma cfDNA to overcome the limitations of small mcfDNA fragment size and low concentration, this approach is unsuitable for the testing of large patient cohorts or routine health screening.

For example, although a lot of progress has been made in reducing the cost and increasing the throughput of NGS sequencing, it remains very expensive to analyze the mcfDNA on a routine basis for community health screening and disease prognosis/diagnostics, as is routinely performed for many other health related parameters (blood cell panels, metabolic panels, etc.) or non-invasive early detection of diseases in at risk populations, such as the screening for colorectal cancer. Thus, there remains an unmet need for improved methods to accurately determine in a high throughput and cost-efficient way to detect the presence of colonizing and invasive microbes that contribute to mcfDNA present in peripheral blood as part of clinical diagnostics and community health screening. The presently disclosed subject matter provides such improved method for high resolution, high-throughput and low-cost detection of microorganisms.

### SUMMARY

In one embodiment, a method is provided for amplifying microbial cell free DNA (mcfDNA). The method includes performing, on a sample comprising microbial cell-free DNA (mcfDNA), an amplification reaction using (i) one or more degenerate primers comprising complementarity to one or more conserved regions, wherein the one or more conserved regions span at least 18 nucleotides of one or more phylogenetic marker genes designated for a set of reference microbes and (ii) a second primer comprising complementarity to a repaired version of an adaptor ligated to ends of the mcfDNA, wherein at least 25 adjacent nucleotides upstream or downstream of an end of the one or more conserved regions comprise a hypervariable region, and the one or more degenerate primers are oriented to prime polymerase extension of the hypervariable region to generate amplified mcfDNA fragments.

In another embodiment, a method is provided for amplifying microbial cell free DNA (mcfDNA), that includes performing an amplification reaction on a sample comprising microbial cell-free DNA (mcfDNA) to generate amplified mcfDNA fragments using: (i) one or more degenerate primers comprising complementarity to one or more conserved regions, wherein the one or more conserved regions span at least 18 nucleotides of one or more phylogenetic marker genes designated for a set of reference microbes, and (ii) a second amplification primer comprising complementarity to an end of the mcfDNA. In some cases, at least 25 adjacent nucleotides upstream or downstream of an end of the one or more conserved regions comprise a hypervariable region, and the one or more degenerate primers are oriented to prime polymerase extension of the hypervariable region. In some embodiments, the end of the mcfDNA can include an adaptor and the primer can include complementarity to a repaired version of the adaptor.

In some instances, the method described herein can further include sequencing the amplified mcfDNA fragments.

In some embodiments, the method can further include, using a computer: (a) aligning the mcfDNA fragment sequences on a sequence of the one or more degenerate primers and assigning matching sequences from the hypervariable region as representative of the same microbial species; (b) for each microbial species in part (a), searching a database of the one or more phylogenetic marker genes against the mcfDNA fragment sequences and assigning the microbial species based on the closest match; and; and (c) for the one or more phylogenetic marker genes, calculating a microbial community composition based on the relative abundance of the mcfDNA fragment sequences assigned to each microbial species. In the case of multicopy phylogenetic marker genes, such as the 16S rRNA gene, the method can further include correcting for copy number variation between each species. In the case where there are two or more phylogenetic marker genes, the method can further include determining a consolidated microbial community composition by calculating a mathematical mean of the relative abundance of each species for each of the two or more phylogenetic marker genes.

The methods described herein can be used to determine the presence of one or more microbial species and/or to determine a microbial community composition. In some cases, the microbial community composition comprises one or more members of Eukaryotes, bacteria, or fungi.

In other instances, a kit is provided that includes: (a) an adaptor for ligating to the ends of cfDNA; (b) one or more degenerate primers having complementarity to one or more conserved regions, and the one or more conserved regions span at least 18 nucleotides of one or more phylogenetic marker genes designated for a set of reference microbes, wherein at least 25 adjacent nucleotides upstream or downstream of an end of the one or more conserveds region comprise a hypervariable region on the one or more phylogenetic marker genes, and the degenerate primer is oriented to prime polymerase extension of the hypervariable region; (c) a primer complementary to a repaired version of the adaptor; and (d) instructions for performing an amplification reaction on mcfDNA having the adaptor-ligated ends with the one or more degenerate primers and the primer complementary to the repaired adaptor to generate amplified mcfDNA fragments. Like the methods described above, the amplified mcfDNA fragments generated in the amplification reaction using the kit can be sequenced. In addition, the mcfDNA fragments generated using the kit can be used to determine the presence of one or more microbial species and/or to determine the microbial community composition according to the methods provided herein.

In the cases where the microbial community composition is calculated as described above, the method can be utilized as a screening for: tuberculosis and other diseases caused by Mycobacterium species; pulmonary infection risks and causes in cystic fibrosis patients; the risk and onset of sepsis in patients with compromised immune systems; detection of opportunistic bacterial pathogens originating from the oral cavity that have been linked to Alzheimer's disease, pancreatic cancer and other conditions such as endocarditis; women's health issues including Chlamydia linked to mucopurulent cervicitis, pelvic inflammatory disease, tubal factor infertility, ectopic pregnancy and cervical cancer; detection and monitoring of progression in cancer; monitoring of minimal residual disease after oncology treatments; detection and monitoring of progression and minimal residual disease of breast cancer including triple negative breast cancer; detection of esophageal cancer, precancerous colonic polyps and early stage colorectal cancer, and detection and monitoring of progression and minimal residual disease of gastrointestinal cancers in general; detection and monitoring of progression and minimal residual disease in lung cancer; non-invasive analysis of the microbiome in pancreatic cancer patients to propose treatment protocols and prognostics for long-term survival; detection of Clostridium difficile infections; post-transplant bloodstream infections and Graft versus Host Disease (GvHD); detection of hospital acquired infections by emerging pathogens of clinical concern; detection of an infection in an immune compromised person; or detection of infection or inflammation of the gastrointestinal track in Irritable Bowel Disease (Crohn's disease, Ulcerative colitis); and combinations thereof.

In the methods and kits provided herein, the conserved region can have an average sequence variance score of greater than 0.175. In some cases, the hypervariable region can have an average sequence variance score of less than 0.075. In other instances, the hypervariable region can have an average sequence variance score of less than 0.15. In yet other cases, the hypervariable region can have an average sequence variance score of less than 0.1.

In the methods and kits, the one or more conserved regions can span 18 to 40 nucleotides, 20 to 30 nucleotides, or 22 to 28 nucleotides of the phylogenetic marker gene.

In some embodiments of the methods and kits, the at least 25 adjacent nucleotides upstream or downstream of an end of the conserved region that includes the hypervariable region is less than 150 adjacent nucleotides. The at least 25 adjacent nucleotides upstream or downstream of an end of the conserved region that includes the hypervariable region can be less than 75 adjacent nucleotides. In other embodiments, the at least 25 adjacent nucleotides upstream or downstream of an end of the conserved region that includes the hypervariable region is less than 50 adjacent nucleotides.

In the method and kit, the adaptor can be a double stranded asymmetric linker cassette comprising a 5′ asymmetrical end and a 3′ end where the two strands are complementary. The asymmetric linker cassette can be, for example, a Y-shaped linker cassette or a single arm linker cassette. In the case of the asymmetric linker cassette, the primer complementary to the adaptor is complementary to a repaired 5′ end of the asymmetric linker cassette and, in the PCR reaction, polymerase extension from the first degenerate primer results in repair of the asymmetric linker cassette.

The method can further include performing one or more reactions to repair the ends of the mcfDNA.

In the method, each of the primers in the amplification reaction can include one or more sequencing adapter sequences. In another embodiment, the method can further include adding one or more sequencing adapter sequences to the amplified mcfDNA fragments in a second PCR or amplification reaction.

In the methods and kits provided herein, the set of reference microbes can be eukaryotic, fungal, or bacterial, and combinations thereof. In one embodiment, the set of reference microbes are eubacterial microbes.

In the method and kit, the phylogenetic marker gene can include rpoB, cpn60, 16S rRNA, or combinations thereof.

In some embodiments, the one or more degenerate primers includes primers targeting the rpoB gene, the cpn60 gene, the 16S rRNA gene, or combinations thereof.

In the method and kit, the phylogenetic marker gene can include 16S rRNA and the conserved region can include a V3, V4, or V6 region of the 16S rRNA phylogenetic marker gene.

In the methods and kits provided herein, the phylogenetic marker gene can include rpoB and the conserved region can include nucleotide positions 1327-1355 based on the Escherichia coli rpoB gene sequence. Alternatively, the phylogenetic marker gene can include rpoB and the conserved region includes nucleotide positions 1627-1652 based on the Escherichia coli rpoB gene sequence. In another embodiment, the phylogenetic marker gene includes cpn60 and the conserved region includes nucleotide positions 571-596 based on the Escherichia coli cpn60 gene sequence. In other instances, the phylogenetic marker gene includes the 16S rRNA gene and the conserved region includes nucleotide positions 785-805 based on the Escherichia coli 16S rRNA gene sequence.

In some embodiments of the method and kit, the one or more degenerate primers includes RpoB1-R1327, RpoB6-R1630, RpoB-F1652, RpoB7-R2039, Cpn60-R571, 16S-V4-R, or combinations thereof.

In other instances, the one or more degenerate primers includes RpoB1-R1327, Cpn60-R571, or both RpoB1R1327 and Cpn60R571 degenerate primers.

In some embodiments of the method and kit, the set of reference microbes includes reference fungal microbes. In these instances, the method can be used to determine the presence of one or more fungi and/or to determine the fungal community composition. In this embodiment, the one or more phylogenetic marker genes comprise a human fungal phylogenetic marker gene designated for the set of reference fungal microbes, and the one or more degenerate primers comprises complementarity to a conserved region of a the human fungal phylogenetic marker gene. In some instances, the fungal phylogenetic marker gene can be nuclear ribosomal internal transcribed spacer region 1 (ITS1) or nuclear ribosomal internal transcribed spacer region 2 (ITS2). The microbial community composition that can be calculated based on the percent of the sequences assigned to each species is a fungal community composition. The amplified mcfDNA fragments can include mcfDNA from one or more members of the Ascomycota, Basidiomycota and Mucoromycota, including Alternaria species, Aspergillus species, Blastomyces species, Candida species, Capnodiales species, Cladosporium species, Malassezia species, Phaeosphaeria species, Pseudozyma species, Saccharomyces species, Sporobolomyces species, Vishniacozyma species, and Yarrowia species.

In the methods and kits, the one or more phylogenetic marker genes can be rpoB, chaperonin protein 60 (cpn60), 165 rRNA gene, ITS1, ITS2, DNA gyrase subunit B (gyrB), heat shock protein 60 (hsp60), superoxide dismutase A protein (sodA), TU elongation factor (tuf), DNA recombinase proteins (including recA, recE), trr1 gene that encodes for thioredoxin reductase; rim8 gene that encodes for a protein involved in the proteolytic activation of a transcriptional factor in response to alkaline pH; kre2 gene that encodes for α-1,2-mannosyltransferase; or erg6 gene that encodes for Δ(24)-sterol C-methyltransferase, and combinations thereof.

In one embodiment, the method or kit can further include adding in the amplification reaction a primer to determine the presence of a functional gene designated for the set of reference microbes. The functional gene primer has complementarity to a conserved region of the functional gene. In some cases, polymerase extension from the functional gene primer results in amplification of the mcfDNA only when the adaptor is ligated to a mcfDNA fragment of the mcfDNA that has the functional gene conserved region. The functional gene can be, for example, a pathogenicity factor, a PKS gene cluster essential for colibactin synthesis, or a choline trimethylaminelyase gene.

In another embodiment of the method and kit, a primer for a conserved viral gene is included in the amplification reaction, wherein the viral gene primer comprises complementarity to a conserved region of the viral gene to determine the presence of the virus. The viral gene can be a human DNA- or RNA-based oncovirus gene. The oncovirus can be one or a combination of Epstein-Barr Virus (EBV), Human Papillomavirus (HPV), Hepatitis B virus (HBV), Human Herpesvirus-8 (HHV-8), or Merkel Cell Polyomavirus (MCPyV). In other instances, the virus is SARS-CoV-2 and the conserved viral gene is SARS-CoV-2spike protein.

In the kit, the mcfDNA can be included in a sample. In the method and kit, the sample can be a bodily fluid, a tissue, or an extracellular bodily substance. The sample can be whole blood, a blood fraction, serum, plasma, or combinations thereof. In some instances, the sample is a biopsy sample from a solid tumor, a skin graft, a liquid biopsy samples other than blood, or combinations thereof. In one embodiment, the sample is a stool sample.

The mcfDNA can have an average fragment length of less than about 100 bp.

The percentage of the mcfDNA in the sample can be less than about 0.05%, less than about 0.1%, less than about 1%, less than about 5%, or less than about 15%.

In the cases of the method and kit where the microbial community composition is calculated, the community composition can include one or more members of Eukaryotes, bacteria, or fungi.

The amplified mcfDNA that is generated in the methods provided herein can include mcfDNA from one or more bacterial members of: Flavobacterium sp., Staphylococcus auricularis, Pseudomonas toyotomiensis, Rheinheimera sediminis, Finegoldia magna, Parvularcula sp., Pseudomonas stutzeri, Pseudomonas soyae, Pseudomonas saponiphila, Pseudomonas sp., Peptoniphilus harei, Quisquilii bacterium sp., Azoarcus sp., Sphingopyxis terrae, uncultured Clostridiales bacterium strain UMGS460, Staphylococcus schweitzeri, Flavobacterium erciyesense, Rhodococcus yananensis, Dietzia massiliensis, Cutibacterium acnes subsp. elongatum, Angustibacter aerolatus, Aerococcus urinae, Klebsiella quasivariicola, Comamonas fluminis, Mycobacterium tuberculosis, Mycobacterium abscessus, Mycobacterium avium, Mycobacterium chimaera, Mycobacterium leprae, Mycobacterium xenopi, Mycobacterium (para)intracellulare, Mycobacterium kansasii, Mycobacterium gilvum, Mycolicibacterium gen. nov. (“fortuitum-vaccae” clade), Mycobacterium gen. (“tuberculosis-simiae” clade), Staphylococcus aureus, Staphylococcus argenteus, Staphylococcus schweitzeri, Pseudomonas aeruginosa, Burkholderia cepacia complex, Burkholderia ubonensis, Burkholderia species Nov., Burkholderia multivorans, Burkholderia pseudomultivorans, Burkholderia pseudomallei, Burkholderia mallei, Trinickia species, Burkholderia thailandensis, Haemophilus influenzae, Haemophilus parainfluenzae, Streptococcus species at the various group and species levels, Streptococcus dysgalactiae, Streptococcus pyogenes, Streptococcus mutans, Streptococcus suis, Streptococcus mitis, Streptococcus pneumoniae, Streptococcus agalactiae, Streptococcus anginosus, Streptococcus intermedius, Streptococcus constellatus, Streptococcus equi subsp. zooepidemicus, Streptococcus oralis, Streptococcus gordonii, Streptococcus uberis, Streptococcus parasanguinis, Streptococcus sanguinis Streptococcus parauberis, Streptococcus infantarius, Streptococcus iniae, Streptococcus salivarius, Streptococcus thermophilus, Streptococcus vestibularis, Streptococcus bovis, Streptococcus gallolyticus subsp. gallolyticus, Streptococcus gallolyticus subsp. macedonicus, Streptococcus gallolyticus subsp. pasteurianus, Streptococcus equinus, Enterococcus faecalis, Enterococcus faecium, Porphyromonas gingivalis, Porphyromonas cangingivalis, Porphyromonas uenonis, Porphyromonas endodontalis, Propionibacterium acidifaciens, Porphyromonas asaccharolytica, Porphyromonas macacae, Prevotella pallens, Prevotella histicola, Prevotella melaninogenica, Prevotella copri, Prevotella intermedia, Prevotella oral, Prevotella nanceiensis, Prevotella salivae, Prevotella nigrescens, Prevotella denticola, Prevotella buccae, Prevotella stercorea, Prevotella oris, Prevotella disiens, Prevotella bryantii, Prevotella shahii, Tannerellaforsythia, Bacteroides fragilis, Helicobacter pylori, Chlamydia trachomatis, Neisseria meningitidis, Neisseria gonorrhoeae, Neisseria subflava, Neisseria perflava, Neisseria flavescens, Neisseria cinerea, Neisseria lactamica, Neisseria weaver, Neisseria zoodegmatis, Neisseria brasiliensis, Neisseria mucosa, Neisseria animaloris, Aggregatibacter actinomycetemcomitans, Aggregatibacter aphrophilus, Aggregatibacter segnis, Saccharopolyspora species, Bacillus clausii, members of the genera Pseudoxanthomonas and Streptomyces, Fusobacterium nucleatum subsp. polymorphum, Fusobacterium hwasookii, Fusobacterium canifelinum, Fusobacterium nucleatum subsp. animalis, Fusobacterium periodonticum, Fusobacterium necrophorum subsp. funduliforme, Fusobacterium mortiferum, Fusobacterium varium, Fusobacterium nucleatum subsp. nucleatum, Fusobacterium ulcerans, Fusobacterium nucleatum subsp. vincentii, Fusobacterium equinum, Fusobacterium gonidiaformans, Fusobacterium necrogenes, Fusobacterium naviforme, Peptostreptococcus stomatis, Pseudonocardia asaccharolytica, Parvimonas species including Parvimonas oral and Parvimonas micra, Gemella species including Gemella morbillorum, Gemella haemolysans, Gemella palaticanis and Gemella sanguinis, Clostridium difficile, Acinetobacter baumannii, Acinetobacter lactucae, Acinetobacter pittii, Acinetobacter calcoaceticus, Acinetobacter oleivorans, Acinetobacter nosocomialis, Acinetobacter radioresistens, Acinetobacter variabilis, Acinetobacter courvalinii, Acinetobacter ursingii, Enterobacteriaceae, Escherichia, or Klebsiella species.

In another embodiment, a system is provided for amplifying microbial cell free DNA (mcfDNA). The system includes a reaction vessel, a reagent dispensing module, and software to execute any of the methods for amplifying microbial mcfDNA described herein, where the method is executed robotically.

In one instance, a computer implemented method is provided for identifying a degenerate primer. The method includes using a computer and a database comprising more than one thousand DNA sequences of a phylogenetic marker gene from a set of microbes to perform the following steps: (i) identifying a highly conserved region within the DNA sequences of the phylogenetic marker gene, wherein the highly conserved region spans at least 18 nucleotides in length and has an average sequence variance score of greater than 0.175; (ii) calculating an average sequence variance score of 25-75 nucleotides upstream of the beginning of the highly conserved region and downstream of the end of the highly conserved region, wherein an average variance score of less than 0.15 is used to identify a hypervariable region; and (iii) designing a degenerate primer sequence complementary to the highly conserved DNA region based on the relative abundance of each nucleotide in the aligned phylogenetic marker gene sequences, wherein the degenerate primer sequence is oriented to prime polymerase extension of the hypervariable region. In the computer implemented method for identifying a degenerate primer, the conserved region can span 18 to 40 nucleotides, 20 to 30 nucleotides, or 22 to 28 nucleotides of the phylogenetic marker gene.

In the computer implemented method, the set of microbes can include one or more members of Proteobacteria (including representative α-, β-, γ-, δ- and ε-Proteobacteria), Firmicutes (including representatives for the classes Bacilli, Clostridia, Erysipelotrichia and Negativicutes), Acinetobacteria, and Fusobacteria. In another embodiment, the set of microbes can include one or more members of Ascomycota, Basidiomycota and Mucoromycota, including Alternaria species, Aspergillus species, Blastomyces species, Candida species, Capnodiales species, Cladosporium species, Malassezia species, Phaeosphaeria species, Pseudozyma species, Saccharomyces species, Sporobolomyces species, Vishniacozyma species, and Yarrowia species.

In one embodiment, a degenerate oligonucleotide primer RpoB1-R1327 is provided consisting of a mixture of oligonucleotides having the sequences 5′ to 3′: CGRTTDCCNARRTGRTCRATRTCRTC (SEQ ID NO: 1), wherein A=adenine, G=guanidine, C=cytosine, T=thymine, R=purine (A or G), D=not C (A, T or G), and N=any nucleotide (A, G, C or T).

In another embodiment, a degenerate oligonucleotide primer RpoB6-R1630 is provided consisting of a mixture of oligonucleotides having the sequences 5′ to 3′: TGHACRTCDCGNACYTCRWADCC (SEQ ID NO: 2), wherein A=adenine, G=guanidine, C=cytosine, T=thymine, R=purine (A or G), Y=pyrimidine (T or C), W=weak (A or T), H=not G (A, T or C), D=not C (A, T or G), and N=any nucleotide (A, G, C or T).

In another instance, a degenerate oligonucleotide primer Cpn60-R571 is provided consisting of a mixture of oligonucleotides having the sequences 5′ to 3′: CCNYKRTCRAABYGCATNCCYTC (SEQ ID NO: 3), wherein A=adenine, G=guanidine, C=cytosine, T=thymine, R=purine (A or G), Y=pyrimidine (T or C), K=amino (T or G), B=not A (T, G or C), and N=any nucleotide (A, G, C or T).

In other embodiments, degenerate oligonucleotide primers RpoB1-R1327, RpoB6-R1630, and Cpn60-R571 are provided in which one or more of the nucleotides at primer positions represented by B, D, or N are replaced by inosine.

### BRIEF DESCRIPTION OF THE DRAWINGS

FIG. 1 is a schematic of SPA fragment generation. The arrow indicates the position of the SPA primer (5′ to 3′). The SPA fragment refers to the mcfDNA fragment region that will be amplified.

FIG. 2 is a schematic overview of the protocol for generating single point amplification (SPA) fragments for sequencing. The various steps are numbered in order of their successive execution. Once single point amplicon fragments are generated, they are sequenced using the standard protocol for next generation paired-end Illumina sequencing.

FIG. 3A is a schematic overview of the protocol for the processing of single point amplicon sequencing data for the analysis of microbial community composition. The various steps are numbered in order of their successive execution. Blastn alignment of the longest bin fragment maximizes the accuracy of microbial species identification, while read-level normalization aims to achieve the best approximation of relative titers for microbial species identified.

FIG. 3B is a schematic overview of the protocol for the processing of SPA fragment sequencing data for the analysis of microbial community composition using multiple phylogenetic identifier genes.

FIG. 4 is a histogram of the lengths of the Amplicon Sequence Variants (ASVs) resulting from SPA fragment sequencing using the RpoB6-SPA-seq-F1652 primer.

FIG. 5 is a histogram of the lengths of the Amplicon Sequence Variants (ASVs) resulting from SPA fragment sequencing using the 16S-SPA-seq-V4-R primer.

FIG. 6 is an overview of an exemplary method used for SPA primer selection.

FIG. 7A shows nucleotide statistics for the rpoB gene region 1327-1352 and degenerate sequence (GAYGAYATYGAYCAYYTNGGHAAYCG (SEQ ID NO: 4)) which is the reverse complement sequence of degenerate primer RpoB1-R1327. The relative abundance of a nucleotide at a specific position was calculated using the nucleotide sequences of 47,505 aligned unique rpoB genes from the PATRIC database and used to design the degenerate sequence, which is provided from 5′ to 3′ using the following nucleotide codes: A: adenine; G: guanidine; C: cytosine; T: thymine; R: purine (A or G); Y: pyrimidine (T or C); H: not G (A, T or C); N: any nucleotide (A, G, C or T); *: presence of an ambiguous sequence at a specific rpoB gene position. The percentages of highly conserved nucleotide sequences used to determine the consensus sequence for the degenerate primer are highlighted. The position of the region is based on the nucleotide sequence of the Escherichia coli rpoB gene.

FIG. 7B shows nucleotide statistics for the cpn60 gene region 571-593 and degenerate sequence (GARGGNATGCRVTTYGAYMRNGG (SEQ ID NO: 5)) which is the reverse complement sequence of degenerate primer Cpn60-R571. The relative abundance of a nucleotide at a specific position was calculated using the nucleotide sequences of 40,989 aligned unique cpn60 genes from the PATRIC database and used to determine the degenerate sequence for this region, which is provided from 5′ to 3′ using the following nucleotide codes: A: adenine; G: guanidine; C: cytosine; T: thymine; R: purine (A or G); Y: pyrimidine (T or C); M: amino (A or C); V: not T (A, G or C); N: any nucleotide (A, G, C or T); *: presence of an ambiguous sequence at a specific cpn60 gene position. The percentages of highly conserved nucleotide sequences used to determine the consensus sequence for the degenerate primer are highlighted. The position of the region is based on the nucleotide sequence of the Escherichia coli cpn60 gene.

FIG. 8 shows nucleotide statistics for the rpoB gene region 1528-1550 and degenerate sequence (CARYTNTCNCARTTYATGGAYCA (SEQ ID NO: 6)). The relative abundance of a nucleotide at a specific position was calculated using the nucleotide sequences of 48,151 aligned unique rpoB genes from the PATRIC database and used to design the degenerate sequence, which is provided from 5′ to 3′ using the following nucleotide codes: A: adenine; G: guanidine; C: cytosine; T: thymine; R: purine (A or G); Y: pyrimidine (T or C); N: any nucleotide (A, G, C or T); *: presence of an ambiguous sequence at a specific rpoB gene position. The percentages of highly conserved nucleotide sequences used to determine the consensus sequence for the degenerate primer are highlighted. The position of the region is based on the nucleotide sequence of the Escherichia coli rpoB gene.

FIG. 9 shows nucleotide statistics for the rpoB gene region 1690-1709 and degenerate sequence (CCRATRTTNGGNCCYTCNGG (SEQ ID NO: 7)). The relative abundance of a nucleotide at a specific position was calculated using the nucleotide sequences of 47,505 aligned unique rpoB genes from the PATRIC database and used to design the degenerate sequence, which is provided from 5′ to 3′ using the following nucleotide codes: A: adenine; G: guanidine; C: cytosine; T: thymine; R: purine (A or G); Y: pyrimidine (T or C); N: any nucleotide (A, G, C or T); *: presence of an ambiguous sequence at a specific rpoB gene position. The percentages of highly conserved nucleotide sequences used to determine the consensus sequence for the degenerate primer are highlighted. The position of the region is based on the nucleotide sequence of the Escherichia coli rpoB gene.

FIG. 10A is a graph showing the variance of the 75 bp region located upstream (5′) of region recognized by the RpoB1-R1327 primer sequence. The variance score is calculated as the variance of the percentage of the nucleotide adenine, guanidine, cytosine and thymine at each position of the rpoB gene, calculated for the 47,505 rpoB genes which aligned on the RpoB1-R1327 primer. A lower number is indicative for more variance, while a higher number is indicative for less variance and a more conserved DNA sequence. The maximum theoretical variance score, plotted on the Y-axes, is 0.25 (100% conserved nucleotide at a position). The region recognized by the RpoB1-R1327 primer (nucleotide numbers 76-101 on the X-axes) is indicated by the arrow.

FIG. 10B is a graph showing the variance of the 75 bp region located downstream (3′) of region recognized by the RpoB1-F1352 primer sequence. The position of the region recognized by the RpoB1-F1352 primer (nucleotide numbers 1-26 on the X-axes) is indicated by the arrow.

FIG. 11 is a graph showing the number of unique SPA fragments with length of 25, 50, 75, 100 and 200 nucleotides for the regions located upstream or downstream of the annealing site for the RpoB1-R1327 and RpoB1-F1352 primer, respectively.

FIG. 12 is a schematic showing the whole genome-based Average Nucleotide Identity (Arahal, 2014) between representative Mycobacterium tuberculosis, Mycobacterium tuberculosis subsp. africanum, Mycobacterium canettii and Mycobacterium orygis strains identified by the presence of SPA fragments My1 and My2. The SPA fragments are 50 nucleotides in length and cover the region upstream of the RpoB1-R1327 primer annealing site.

FIG. 13 is a schematic showing the whole genome-based Average Nucleotide Identity (Arahal, 2014) between representative Mycobacterium avium strains identified by the presence of SPA fragments My8 and My9. The SPA fragments are 50 nucleotides in length and cover the region upstream of the RpoB1-R1327 primer annealing site.

FIG. 14 is a schematic showing the whole genome-based Average Nucleotide Identity (Arahal, 2014) between representative Mycobacterium strains identified by the presence of SPA fragments My17 and My18. The SPA fragments are 50 nucleotides in length and cover the region upstream of the RpoB1-R1327 primer annealing site.

FIG. 15 is a schematic showing the whole genome-based Average Nucleotide Identity (Arahal, 2014) between representative Staphylococcus strains identified by the presence of SPA fragments Sa1, Sa2, Sa3 and Sa4. The SPA fragments are 50 nucleotides in length and cover the region upstream of the RpoB1-R1327 primer annealing site.

FIG. 16 is a schematic showing the whole genome-based Average Nucleotide Identity (Arahal, 2014) between representative Pseudomonas strains identified by the presence of SPA fragments Pa1, Pa2, and Pa4. The SPA fragments are 50 nucleotides in length and cover the region upstream of the RpoB1-R1327 primer annealing site.

FIG. 17 is a schematic showing the whole genome-based Average Nucleotide Identity (Arahal, 2014) between representative Burkholderia pseudomallei group strains identified by the presence of SPA fragments Bpm1, Bpm2, Bpm3 and Bcc1. The SPA fragments are 50 nucleotides in length and cover the region upstream of the RpoB1-R1327 primer annealing site.

FIG. 18 is a schematic showing the whole genome-based Average Nucleotide Identity (Arahal, 2014) between representative Haemophilus influenzae and Haemophilus parainfluenzae strains identified by the presence of SPA fragments Hi1, H2, Hi6 and Hi7. The SPA fragments are 50 nucleotides in length and cover the region upstream of the RpoB1-R1327 primer annealing site.

FIG. 19 is a schematic showing the whole genome-based Average Nucleotide Identity (Arahal, 2014) between representative Streptococcus dysgalactiae and Streptococcus pyogenes strains identified by the presence of SPA fragments St2, St3 and St4. The SPA fragments are 50 nucleotides in length and cover the region upstream of the RpoB1-R1327 primer annealing site.

FIG. 20 is a schematic showing the whole genome-based Average Nucleotide Identity (Arahal, 2014) between representative Streptococcus gordonii, Streptococcus oligofermentans, Streptococcus mitis and Streptococcus oralis strains identified by their SPA fragments. The SPA fragments are 50 nucleotides in length and cover the region upstream of the RpoB1-R1327 primer annealing site.

FIG. 21 is a schematic showing the whole genome-based Average Nucleotide Identity (Arahal, 2014) between representative Streptococcus anginosus, Streptococcus constellatus and Streptococcus intermedius strains identified by the presence of SPA fragments St14 to St17. The SPA fragments are 50 nucleotides in length and cover the region upstream of the RpoB1-R1327 primer annealing site.

FIG. 22 is a schematic showing the whole genome-based Average Nucleotide Identity (Arahal, 2014) between representative Streptococcus thermophilus, Streptococcus vestibularis and Streptococcus salivarius strains identified by the presence of SPA fragments St30, St31 and St32. The SPA fragments are 50 nucleotides in length and cover the region upstream of the RpoB1-R1327 primer annealing site.

FIG. 23 is a schematic showing the whole genome-based Average Nucleotide Identity (Arahal, 2014) between representative Streptococcus gallolyticus subsp. gallolyticus, Streptococcus gallolyticus subsp. Macedonicus, Streptococcus gallolyticus subsp. pasteurianus and Streptococcus equinus strains identified by the presence of SPA fragments St33, St34 and St35. The SPA fragments are 50 nucleotides in length and cover the region upstream of the RpoB1-R1327 primer annealing site.

FIG. 24 is a schematic showing the whole genome-based Average Nucleotide Identity (Arahal, 2014) between representative Enterococcus faecalis and Enterococcus faecium strains identified by the presence of SPA fragments Ef1, Ef2, Ef3 and Ef4. The SPA fragments are 50 nucleotides in length and cover the region upstream of the RpoB1-R1327 primer annealing site.

FIG. 25 is a schematic showing the whole genome-based Average Nucleotide Identity (Arahal, 2014) between representative Porphyromonas strains identified by the presence of SPA fragments Pg1 to Pg9. The SPA fragments are 50 nucleotides in length and cover the region upstream of the RpoB1-R1327 primer annealing site.

FIG. 26 is a schematic showing the whole genome-based Average Nucleotide Identity (Arahal, 2014) between representative Bacteroides fragilis strains and related species identified by the presence of SPA fragments Bf1, Bf2 and Bf3. The SPA fragments are 50 nucleotides in length and cover the region upstream of the RpoB1-R1327 primer annealing site.

FIG. 27 is a schematic showing the whole genome-based Average Nucleotide Identity (Arahal, 2014) between representative Helicobacter pylori strains identified by the presence of SPA fragments Hp1, Hp2 and Hp3. The SPA fragments are 50 nucleotides in length and cover the region upstream of the RpoB1-R1327 primer annealing site.

FIG. 28 is a schematic showing the whole genome-based Average Nucleotide Identity (Arahal, 2014) between representative Aggregatibacter strains identified by the presence of unique SPA fragments. The SPA fragments are 50 nucleotides in length and cover the region upstream of the RpoB1-R1327 primer annealing site.

FIG. 29 is a schematic showing the whole genome-based Average Nucleotide Identity (Arahal, 2014) between representative Acinetobacter baumannii strains and related species identified by the presence of their unique SPA fragments. The SPA fragments are 50 nucleotides in length and cover the region upstream of the RpoB1-R1327 primer annealing site. SPA fragment ‘ref’ indicates a reference strain included.

FIG. 30 is a schematic showing the whole genome-based Average Nucleotide Identity (Arahal, 2014) between representative Acinetobacter baumannii strains and related species identified by the presence of their unique SPA fragments. The SPA fragments are 50 nucleotides in length and cover the region upstream of the RpoB1-R1327 primer annealing site. SPA fragment ‘ref’ indicates a reference strain included.

FIG. 31 is a schematic showing the whole genome-based Average Nucleotide Identity (Arahal, 2014) between representative Acinetobacter baumannii strains and related species identified by the presence of their unique SPA fragments. The SPA fragments are 50 nucleotides in length and cover the region upstream of the RpoB1-R1327 primer annealing site. SPA fragment ‘ref’ indicates a reference strain included.

FIG. 32 is a schematic showing the whole genome-based Average Nucleotide Identity (Arahal, 2014) between representative Klebsiella and related strains which share SPA fragment Ent2 (see Table 38). The 50 nucleotide SPA fragments upstream of the RpoB6-R1630 priming site are identified as SPA fragment “Ent” with a numerical identifier and with an asterisk symbol “*” indicating that the SPA fragment was generated from the region upstream of the RpoB1-R1630 priming site. SPA fragment ‘ref’ indicates a reference strain included.

FIG. 33A is a phylogenetic tree of Escherichia coli and related species based on the sequences of 50 nucleotide SPA fragments generated from the region upstream of the RpoB1-R1327 priming site. Clusters of Escherichia coli phylotype B2 sand D strains are indicated.

FIG. 33B is a phylogenetic tree of Escherichia coli and related species based on the sequences of 50 nucleotide SPA fragments generated from the region upstream of the RpoB6-R1630 priming site. Clusters of Escherichia coli phylotype B2 sand D strains are indicated.

FIG. 33C is a phylogenetic tree of Escherichia coli and related species based on the combination of 50 nucleotide SPA fragments sequences generated from the regions upstream of the RpoB1-R1327 and RpoB6-R1630 priming sites. Clusters of Escherichia coli phylotype B2 sand D strains are indicated.

FIG. 34A is a schematic showing the whole genome-based Average Nucleotide Identity (ANI) comparison for the Faecalibacterium species present in the consortium.

FIG. 34B is a schematic showing the whole genome-based Average Nucleotide Identity (ANI) comparison for the Bacteroides ovatus strains present in the consortium.

FIG. 35 is a graph showing the simulation of mcfDNA fragment length distribution. Average fragment lengths of 40, 60, 80 and 100 base pairs were used in the simulations, respectively. For each simulation, the size distribution of a million mcfDNA fragments around a truncated normal distribution was used.

### DETAILED DESCRIPTION

The presently disclosed subject matter now will be described more fully hereinafter. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the descriptions provided herein. Therefore, it is to be understood that the presently disclosed subject matter is not to be limited to the specific embodiments disclosed and that modifications and other embodiments are intended to be included within the scope of the appended claims.

Following long-standing patent law convention, the terms “a,” “an,” and “the” refer to “one or more” when used in this application, including the claims. Thus, for example, reference to “a sample” includes a plurality of samples, unless the context clearly is to the contrary, and so forth.

Throughout this specification and the claims, the terms “comprise,” “comprises,” and “comprising” are used in a non-exclusive sense, except where the context requires otherwise. Likewise, the terms “having” and “including” and their grammatical variants are intended to be non-limiting, such that recitation of items in a list is not to the exclusion of other like items that can be substituted or added to the listed items.

For the purposes of this specification and claims, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range. In addition, as used herein, the term “about”, when referring to a value can encompass variations of, in some embodiments +/−20%, in some embodiments +/−10%, in some embodiments +/−5%, in some embodiments +/−1%, in some embodiments +/−0.5%, and in some embodiments +/−0.100, from the specified amount, as such variations are appropriate in the disclosed compositions and methods. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

Throughout this specification and the claims, the term “subject” includes humans and animals and can be used interchangeably with the term “human” and the term “patient”.

The terms “SPA fragment” and “SPA fragment sequence” are herein used interchangeably.

The terms “PCR reaction” and “amplification reaction” are herein used interchangeably.

The term “phylogenetic marker gene” as used herein means any conserved gene from any organism, including but not limited to bacteria, fungi, parasites, and viruses, that is suitable for phylogenetic identification.

There are a wide range of diseases where microbial community analysis, especially of the gut microbiome, provides important information regarding the disease or its treatment options. This includes conditions such as IBD (Ananthakrishnan et al, 2017), metabolic diseases (Boulange et al, 2016), diseases of the central nervous system (Bhattacharjee and Lukiw, 2013) and cancer where the interaction of the gut microbiome can provide clues regarding the response to specific treatments including immune checkpoint inhibitors (Gopalakrishnan et al, 2020; Sepich-Poore et al, 2021). Deep microbial metagenome sequencing is the most informative approach when it comes to microbial community analysis, as it will provide detailed information regarding community composition as well as the key functions encoded by the community members. Unfortunately, despite major breakthroughs in metagenome sequencing technologies to reduce its costs, it is currently still too expensive for routine screening purposes of human associated microbial communities in large population screenings. Another disadvantage of deep microbial metagenome sequencing is the need for relatively large amounts of high-quality microbial DNA. This has hindered its application to study the microbial communities associated with liquid and solid biopsy samples, where only a small fraction of the total DNA is of microbial origin.

The amplification and subsequent sequencing of phylogenetic marker genes provides an alternative, cheaper high throughput method for microbial community analysis. For example, in tissue biopsy samples where there is sufficient concentration of DNA having average fragment length of about 5,000 bp or more, amplification-based sequencing approaches have been successfully applied to identify differences in microbial communities between healthy individuals and patients suffering from a wide range of diseases. Advantages of the amplification and subsequent sequencing method include that it requires significantly less DNA than metagenome sequencing, and because specific DNA primers are used to amplify phylogenetic target genes, there is little contamination with host DNA, making this method suitable to analyze the microbial communities associated with tissue biopsy samples, from which small amounts of high molecular weight DNA can be obtained. However, analysis of microbial signatures in liquid biopsy samples, especially peripheral blood samples, results in additional challenges as compared to tissue biopsy samples, due to the low concentration of mcfDNA having small fragment sizes.

For example, in plasma, human cfDNA accounts for the vast majority of cfDNA (>90% or even >99%), while mcfDNA accounts for only a small fraction with 0.08%-4.85% from bacteria, 0.00%-0.01% from fungi, and 0.00%-0.16% from viruses/phages (Han et al, 2020). However, the percentage of mcfDNA compared to cfDNA should be placed in the context of the human genome size and the size of an average microbial genome, with sizes of 6.4 billion and approximately 6 million nucleotides, respectively, therefore providing similar coverage. Thus, mcfDNA represents an important signal that is largely being ignored in liquid biopsy testing.

The intrinsic properties of cfDNA and mcfDNA, especially its small fragment sizes, make its analysis for disease detection and monitoring challenging. More than 70% of plasma cfDNA is smaller than 300 bp, with an average size of 170 bp (Fernández-Carballo et al, 2019). However, the size of mcfDNA fragments was found to be significantly smaller, approximately 40-100 bp (Burnham et al, 2016), as was confirmed by Rassoulian Barrett et al (2020). As a result of this size limitation, conventional amplicon-based sequencing approaches including 16S rRNA gene and rpoB gene amplicon sequencing that target DNA fragments of several hundred nucleotides, are not suitable for determining the composition of colonizing or invasive microorganisms using mcfDNA from peripheral blood and other liquid biopsy samples. The small size of mcfDNA makes it nearly impossible to use mcfDNA in amplicon-based sequencing protocols, such as 16S rRNA gene sequencing, leaving no other option than high-cost and low-throughput NGS sequencing.

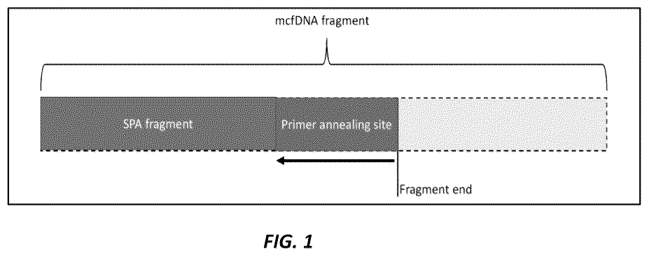

To overcome the above-mentioned limitations, the present inventors developed a single point amplification sequencing approach that exploits the combination of a degenerate primer for a conserved region of a marker gene located adjacent to a phylogenetic hypervariable region of the gene for a wide range of microbes. The method is based on the targeted amplification of high-resolution phylogenetic identifier fragments from mcfDNA, which comprises a fraction of the total cfDNA isolated from, for example, biopsy samples. To generate the phylogenetic identifier fragments, a hypervariable DNA region with high phylogenetic resolution is targeted. The hypervariable region located next to the highly conserved region that functions as a primer annealing site as is illustrated in FIG. 1. In the methods disclosed herein, the fragments resulting from specific amplification of the hypervariable DNA regions are referred to as SPA fragments.

In various embodiments, methods and kits are provided herein for generating the SPA fragments. The methods and kits provided herein can be used to determine the presence of one or more microbial species and/or to determine one or more microbial community compositions. In the methods and kits provided herein, the set of reference microbes can be eukaryotic, fungal, or bacterial, and combinations thereof. In one embodiment, the set of reference microbes are eubacterial microbes.

In the methods of the invention, the length of the SPA fragment is determined by the distance between the end of the mcfDNA fragment and the 3′-end of the primer annealing site. Only mcfDNA fragments that contain the primer annealing site will give SPA fragments, which can be subsequently sequenced and used for high resolution phylogenetic identification and analysis of community composition.

In one aspect of the invention, the degenerate primer is used in combination with an adaptor, such as, for example, an asymmetric linker cassette which is attached to the 3′ ends of all the cfDNA fragments in the sample. A PCR amplification reaction is performed using the degenerate primer and a primer complementary to the 5′ asymmetrical end of the linker cassette. The degenerate primer is designed to allow for DNA synthesis into the hypervariable region. However, successful PCR amplification of the hypervariable region occurs only when the asymmetric linker cassette is repaired. In a PCR reaction, the asymmetric linker cassette will be repaired only when located downstream from the degenerate primer annealing site, i.e, when the asymmetric linker cassette has been ligated to a mcfDNA fragment that contains the conserved region of the phylogenetic marker gene. In this manner, microbial DNA fragments that originate from the hypervariable region are selectively amplified.

In one example of the invention, to overcome the above-mentioned limitations for determining microbial profiles from mcfDNA, such as, for example, mcfDNA in liquid biopsy samples, the present inventors developed a unique approach that exploits the phylogenetic resolution of a hypervariable region of the rpoB gene. In another example of the invention, the present inventors developed a unique approach that exploits the phylogenetic resolution of V3-V4 hypervariable region of the 16S rRNA gene. In contrast to commonly used amplicon sequencing, in which regions between two conserved DNA sequences are targeted for PCR amplification, the methods provided herein use a single conserved DNA sequence as the primer annealing site to initiate PCR amplification. The amplification initiated from this single conserved DNA sequence allows for targeted amplification of the hypervariable region located adjacent to the primer annealing site, independent of the size of the fragment, followed by sequencing of the amplified fragment. In another example of the invention, the phylogenetic resolution of a hypervariable region of the chaperonin cpn60 gene is used in the presently disclosed methods. This method may be referred to herein as Single Point Amplification (SPA) fragment sequencing.

Alternative embodiments of the invention include use of a conserved DNA sequence as the primer annealing site for more than one site on a phylogenetic marker gene or for a site on two or more different phylogenetic marker genes in a single amplification reaction. In one instance, two degenerate primers targeting different regions of the rpoB gene are included in the presently disclosed methods. In another instance, a degenerate primer for both the cpn60 and the rpoB gene are included in the presently disclosed methods. The use of two or more degenerate primers for annealing to two or more conserved regions on a single or two different phylogenetic marker genes may be referred to herein as “multi-loci SPA fragment sequencing”.

In the specific examples provided herein the RNA polymerase subunit B (rpoB) gene and the chaperonin 60 (cpn60) gene were used, but it should be noted that the SPA fragment sequencing method is very broadly applicable to conserved housekeeping genes, including, but not limited to, the prokaryotic genes coding for the DNA gyrase subunit B (gyrB), the heat shock protein 60 (hsp60), the superoxide dismutase A protein (sodA), the TU elongation factor (tuf), and the DNA recombinase proteins (including recA, recE). The SPA fragment sequencing method can also be applied on the Prokaryotic 16S rRNA gene, for instance to amplify (part of) the V1-V2 or V3-V4 hypervariable region. The SPA fragment sequencing method can also be applied on the Eukaryotic internal transcribes spacer (ITS) regions ITS1, which is located between the 18S and 5.8S rRNA genes, and ITS2, which is located between the 5.8S and 28S rRNA genes. The SPA fragment sequencing method can also be applied to genes that are unique to pathogenic fungi including the trr1 gene that encodes for thioredoxin reductase; the rim8 gene that encodes for a protein involved in the proteolytic activation of a transcriptional factor in response to alkaline pH; the kre2 gene that encodes for α-1,2-mannosyltransferase; and the erg6 gene that encodes for Δ(24)-sterol C-methyltransferase (Abadio et al, 2011); or any conserved gene from any organism, including bacteria, fungi, parasites, and viruses that is suitable for phylogenetic identification. This includes conserved genes from the human DNA-based oncoviruses, more specifically the Epstein-Barr Virus (EBV), Human Papillomavirus (HPV), Hepatitis B virus (HBV), Human Herpesvirus-8 (HHV-8), and Merkel Cell Polyomavirus (MCPyV) (Mui et al, 2017). One or a combination of any conserved housekeeping gene can be used in the presently disclosed methods.

Advantages of the disclosed SPA fragment sequencing method include an increase in the diversity of hypervariable regions that can be targeted for amplicon analysis as the method only requires one neighboring conserved region to bind the primer (compared with the two required by dual primer approaches). As such, the SPA fragment sequencing method is more adaptable, flexible, and offers greatly improved resolution over current methods. In addition, the multi-loci SPA sequencing methods include the advantage of improving phylogenetic resolution for the identification of the community members on the species and subspecies level, as is highlighted in EXAMPLE 13. Further, the multi-loci SPA sequencing methods provide an internal control for improved error correction in the SPA fragment amplification and sequencing process, as similar results for community species abundances are expected independent of the phylogenetic identifier gene.

In addition to the degenerate primer for the conserved region, an adaptor such as, for example, an asymmetric linker cassette, can be used to introduce a DNA sequence that is targeted by a second primer in the PCR amplification reaction. In one embodiment, to avoid amplification of any DNA fragment flanked by two adaptors, the adaptors are “defective” or in other words “asymmetric”. This can be accomplished by designing an adaptor as an asymmetric linker cassette where the strand that serves as the template for primer annealing is missing. Typical asymmetric linker cassette configurations include, but are not limited to:
1. A “Y”-shaped linker cassette, where two single stranded DNA fragments that are only partially complementary are annealed. This results in an asymmetric linker cassette where one end is double stranded, allowing for ligation, but where the other end is comprised of two single stranded non-complementary DNA strands.
2. A “single arm” linker cassette, where a shorter single stranded DNA fragment is annealed to the complementary 3′-end of a longer single stranded DNA fragment. This results in an asymmetric linker cassette with a single stranded the 5′-end and a double stranded 3′-end.

Ideally, the single strands of the asymmetric linker cassette are complementary over a stretch of about at least 16 nucleotides with an annealing temperature of approximately 50° C. or higher, allowing for a linker cassette that is stable at room temperature. The single strand of the asymmetric linker can also contain 6 random nucleotides that constitute a Unique Molecular Identifier (UMI) to correct PCR induced errors and improve sequencing accuracy. To avoid self-ligation, in one example, the asymmetric linker cassette includes a 3′sticky end. The 3′sticky end can be formed by a single nucleotide, such as, for example, thymine. To avoid undesirable repair of the asymmetric linker cassette initiated from the shorter single stranded DNA fragment, the terminal 3′ nucleotide can be a dideoxy nucleotide that functions as a chain-elongating inhibitor of DNA polymerase.

In a PCR reaction, the asymmetric linker cassette will only be repaired when located downstream from the degenerate primer annealing site. For purposed of the specification and claims, the term “repaired” when used in the context of the asymmetric linker cassette, means that a new DNA strand is created in the PCR reaction that is complementary at the 5′ end of the asymmetric linker cassette. DNA synthesis initiated from the degenerate primer into the asymmetric linker cassette will restore the defective DNA strand complementary to the 5′-end of the linker and in this manner the asymmetric linker cassette is repaired. In subsequent PCR cycles this strand is used for primer annealing, allowing for the amplification of the hypervariable region. To allow for sample multiplexing and sequencing, the resulting amplicons can be further amplified in a second PCR reaction to introduce two Unique Dual Indexes (UDI), one at each end of the amplicons, and, for example, the Illumina sequencing anchors P5 and P7.

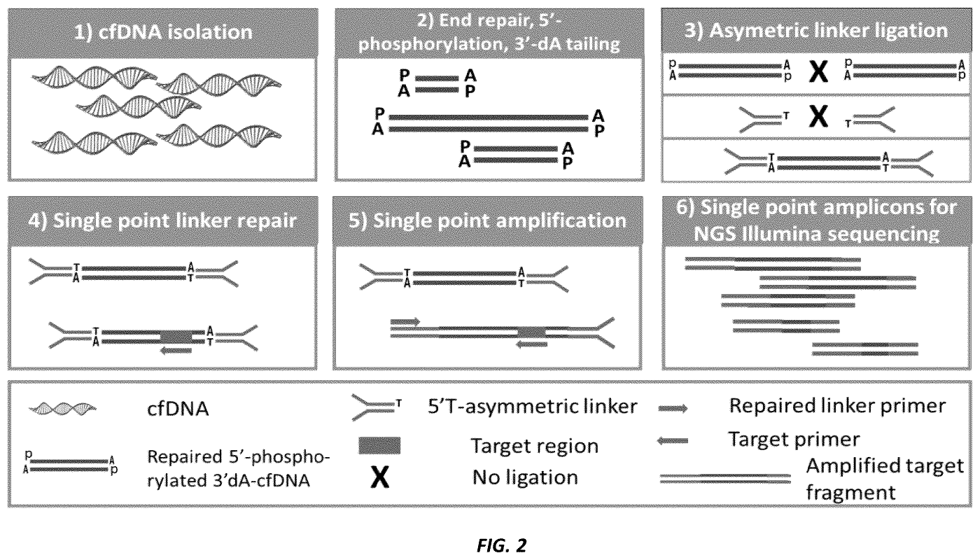

In one embodiment of the invention, the method includes one or more of the following steps as detailed in FIG. 2:
1. Isolation of cfDNA using standard protocols. Cell-free DNA can be extracted from 0.5 mL blood plasma using the typically yielding 0.1 ng to 10 ng to be used for sequencing. cfDNA can also be isolated from urine, saliva, stool and other biopsy samples.
2. End repair and 5′-phosphorylation of cfDNA fragments followed by the 3′ addition of a deoxy-adenine to create a 3′-sticky end formed by a single adenine nucleotide using standard protocols. A typical protocol to process cfDNA includes end repair (blunting and 5′ phosphorylation), 3′ A-tailing, followed by adaptor ligation. The fragment ends are repaired by blunting and 5′ phosphorylation with a mixture of enzymes, such as T4 polynucleotide kinase (PNK) and T4 DNA polymerase (T4 DNA pol). This end repair step is followed by 3′ A-tailing at 37° C. using a mesophilic polymerase such as Klenow Fragment 3′-5′ exonuclease minus (Head et al, 2014). Many commercial kits are available to perform this step.
3. Ligation of the adaptor, which in this case is an asymmetric linker cassette, using T4 DNA ligase. Many commercial kits are available to perform this step, including the NEB NEBNext® Ultra™ II Ligation kit or the IDT xGen™ DNA Lib Prep MC kit. The sequences of the oligonucleotides used for the design of the asymmetric linker cassette, referred to as SPA-cas1 and SPA-cas2, are provided in Table 1 along with other primer sequences that can be utilized in the methods and kits provided herein. Annealing of these two partially complementary single stranded DNA fragments results in a “Y”-shaped or single arm DNA linker cassette. On one end, the two strands of the linker cassette are not complementary. On other end, where the two strands are complementary, the linker cassette includes a 3′sticky end formed by a single thymine nucleotide. Due to the sticky ends, the only possible ligation is between cfDNA fragments and asymmetric linker cassettes, while self-ligation of linker cassettes and repaired cfDNA fragments is blocked.
4. Single point linker cassette repair. PCR is performed on the ligation product using the following primers: (a) the SPAT-amp primer that recognizes the repaired 5′ asymmetrical end of the linker cassette; (b) one or more primers that recognize the primer annealing site specific for the conserved region of the one or more phylogenetic marker genes. DNA amplification initiated from the gene-specific SPA primer will result in the repair of the asymmetric linker cassette but only when this cassette is bound to a cfDNA fragment that contains the primer annealing site on the conserved region. This will be limited to mcfDNA fragments that contain the targeted region of the phylogenetic marker gene such as, for example, positions 1630-1652 of the rpoB gene, which is absent in Eukaryotic DNA including human DNA. As such no human cfDNA fragments flanked by asymmetric linker cassettes will be repaired. In EXAMPLE 1 herein below, the RpoB6-SPA-seq-F1652 primer, which recognizes the rpoB gene sequence between positions 1630-1652, and the 16S-SPA-seq-V4-R primer which recognizes the 16S rRNA gene sequence between positions 785-805, were validated. In addition, due to the direction of the amplification reaction from the gene specific primer such as, for example, the forward RpoB6-SPA-seq-F1652 primer, only linker cassettes that are bound to the region upstream of the targeted region of the phylogenetic marker gene will be repaired (e.g., position 1630-1652 of the rpoB gene).
5. Once the asymmetric linker cassette has been repaired, the primer (SPA1-seq-F primer) that recognizes the repaired 5′ asymmetrical end of the linker cassette can anneal and PCR amplification is initiated. In the case of the RpoB6-SPA-seq-F1652 primer this will result in the amplification of DNA sequences located downstream of position 1652 of the rpoB gene. The forward (SPA1-seq-F) and reverse (e.g. RpoB6-SPA-seq-F1652) primers include a 5′ extension corresponding to the Illumina Read-1 and Read-2 sequences, respectively, to allow sequencing library preparation. After the amplification step has been completed, an optional enrichment step can be performed by annealing a 5′-biotinilated version of the one or more gene specific primers (e.g., RpoB6-SPA-seq-F1652 primer) followed by capturing the hybridized primer on magnetic streptavidin beads. Subsequently, the non-captured DNA fragments are washed away, and the targeted DNA fragments are eluted using a NaOH solution. After neutralization and precipitation, these fragments are ready for the construction of sequencing libraries.
As an alternative to the affinity-based enrichment step, an enrichment PCR protocol can be used to reduce background amplification of human DNA fragments resulting from nonspecific primer annealing. The enrichment PCR uses the SPA-amp primer in combination with one primer annealing to the conserved region extended by a few nucleotides (e.g. RpoB6-F1649) compared to the primer used in STEP 4 (e.g. RpoB6-SPA-seq-F1652). Neither primer used in the first step of the enrichment PCR contains the Illumina Read-1/2 extension.
6. In a second PCR reaction (PCR2), Unique Dual Indexes (UDI) and Illumina sequencing anchors (P5 and P7) are added to the amplified SPA fragments using P5-I5-Rd1 and P7-I7-Rd2 primers (see Table 1). The PCR2 is performed using unique sets of UDI for each sample, subsequently allowing the pooling of the libraries, after which fragments are paired-end sequenced using NGS Illumina sequencing, e.g. on the Illumina NEXTSEQ 1000 (Illumina, Inc., San Diego, CA). This approach will result in sequenced fragments that all share the sequence of the gene specific primer (e.g., RpoB6-SPA-seq-F1652 primer) followed by sequences that vary in length and nucleotide composition. Sequences derived from the same microorganisms will be identical except for the length of the sequenced fragment, which will vary as a function of the distance between the gene specific primer annealing site (e.g., RpoB6-SPA-seq-F1652 primer) and the end of the mcfDNA fragment.

TABLE 1

| Primer | | |
|---|---|---|
| Label | Sequence (5′→3′) | Utilization |
| | SPA fragment sequencing initiated from the rpoB gene region 1327-1355 | |
| RpoB1- | CGRTTDCCNARRTGRTCRATRTC | rpoB fragment single point |
| R1327 | RTC (SEQ ID NO: 1) | amplicon upstream of 1327 |
| RpoB1- | GCDCGRTTDCCNARRTGRTCRAT | Used for enrichment PCR with |
| R1330 | RTC (SEQ ID NO: 8) | SPA1-amp to amplify rpoB |
| | | fragment single point amplicons |
| | | upstream of 1330 |
| RpoB1- | 5′ | Used for PCR1 with SPA1-seq-F to |
| SPA- | GTCTCGTGGGCTCGGAGATGTGT | capture rpoB fragment single point |
| seq- | ATAAGAGACAG- | amplicons upstream of 1327 |
| R1327 | CGRTTDCCNARRTGRTCRATRTC | |
| | RTC (SEQ ID NO: 9) | |
| RpoB1- | GAYGAYATYGAYCAYYTNGGH | rpoB fragment single point |
| F1352 | AAYCG (SEQ ID NO: 4) | amplicon downstream of 1352 |
| | SPA fragment sequencing initiated from the rpoB gene region 1627-1652 | |
| RpoB6- | 5′ | rpoB fragment single point |
| F1652 | GGHTWYGARGTICGHGAYGTDC | amplicon downstream of 1652 |
| | A (SEQ ID NO: 10) | |
| RpoB6- | GCIGGHTWYGARGTICGHGAYG | Used for enrichment PCR with |
| F1649 | T (SEQ ID NO: 11) | SPA1-amp to amplify rpoB |
| | | fragment single point amplicons |
| | | downstream of 1649 |
| RpoB6- | 5′ | Used for PCR1 with SPA1-seq-F to |
| SPA- | GTCTCGTGGGCTCGGAGATGTGT | capture rpoB fragment single point |
| seq- | ATAAGAGACAG- | amplicons downstream of 1652 |
| F1652 | GGHTWYGARGTICGHGAYGTDC | |
| | A (SEQ ID NO: 12) | |
| RpoB6- | TGHACRTCDCGNACYTCRWADC | rpoB fragment single point |
| R1630 | C (SEQ ID NO: 2) | amplicon upstream of 1630 |
| RpoB6- | 5′ | Used for PCR1 with SPA1-seq-F to |
| SPA- | GTCTCGTGGGCTCGGAGATGTGT | capture rpoB fragment single point |
| seq- | ATAAGAGACAG- | amplicons upstream of 1630 |
| R1630 | TGHACRTCDCGNACYTCRWADC | |
| | C (SEQ ID NO: 13) | |
| | SPA fragment sequencing initiated from the rpoB gene region 2039-2063 | |
| RpoB7- | TGACGYTGCATGTTBGMRCCCA | rpoB fragment single point |
| R2039 | TMA | amplicon upstream of 2039 |
| RpoB7- | 5′ | Used for PCR1 with SPA1-seq-F to |
| SPA- | GTCTCGTGGGCTCGGAGATGTGT | capture rpoB fragment single point |
| seq- | ATAAGAGACAG- | amplicons upstream of 2039 |
| R2039 | TGACGYTGCATGTTBGMRCCCA | |
| | TMA (SEQ ID NO: 14) | |
| | SPA fragment sequencing initiated from the 16S rRNA gene | |
| 16S-V3- | CCTACGGGNGGCWGCAG (SEQ | 16S rRNA gene single point |
| F | ID NO: 15) | amplicon into V3 region |
| 16S- | 5′ | Used for PCR1 with SPA1-seq-F to |
| SPA-seq- | GTCTCGTGGGCTCGGAGATGTGT | capture 16S rRNA fragment single |
| V3-F | ATAAGAGACAG- | point amplicons for V3 region |
| | CCTACGGGNGGCWGCAG (SEQ | |
| | ID NO: 16) | |
| 16S-V4- | GACTACHVGGGTATCTAATCC | 16S rRNA gene single point |
| R | (SEQ ID NO: 17) | amplicon into V4 region |
| 16S- | 5′ | Used for PCR1 with SPA1-seq-F to |
| SPA-seq- | GTCTCGTGGGCTCGGAGATGTGT | capture 16S rRNA fragment single |
| V4-R | ATAAGAGACAG- | point amplicons for V4 region |
| | GACTACHVGGGTATCTAATCC | |
| | (SEQ ID NO: 18) | |
| | SPA fragment sequencing initiated from the cpn60 gene region 571-593 | |
| Cpn60- | CCNYKRTCRAABYGCATNCCYT | Cpn60 fragment single point |
| R571 | C (SEQ ID NO: 3) | amplicon upstream of pos. 571 |
| Cpn60- | 5′GTCTCGTGGGCTCGGAGATGTG | Used for PCR1 with SPA1-seq-F to |
| SPA- | TATAAGAGACAG- | capture cpn60 fragment single |
| seq- | CCNYKRTCRAABYGCATNCCYT | point amplicons upstream of 571 |
| R571 | C (SEQ ID NO: 19) | |
| | Asymmetric SPA linker cassette construction, SPA fragment amplification | |
| | and sequencing | |
| SPA- | GACAGGGATTTGCTGGTCGNNN | Forward strand of the asymmetric |
| cas1 | NNNAATTCAACTAGGCTTAATC | SPA linker cassette, including 6 |
| | CGACGT* (SEQ ID NO: 20) | random nucleotides (N6) to be used |
| | | as Unique Molecular Identifier |
| | | (UMI). |
| SPA- | /5Phos/CGTCGGATTAAGCCTAGT | Reverse strand of the asymmetric |
| cas2 | TGAGCA (SEQ ID NO: 21) | SPA linker cassette, |
| | | phosphorylated on the 5′ end. |
| | | The last 3 nucleotides at the 3′end |
| | | do not hybridize to APS-cas1 to |
| | | prevent repair of the asymmetric |
| | | linker. |
| SPA1- | GACAGGGATTTGCTGGTCG (SEQ | SPA repaired linker-initiated SPA |
| amp | ID NO: 22) | fragment amplification, used for |
| | | enrichment PCR of the SPA library |
| | | preparation |
| SPA1- | 5′ | Used for PCR1 of the SPA library |
| seq-F | TCGTCGGCAGCGTCAGATGTGTAT | preparation |
| | AAGAGACAG- | |
| | GGATTTGCTGGTCG (SEQ ID NO: | |
| | 23) | |
| | Illumina sequence library construction | |
| P5-15- | 5′ | Used for PCR2 with P7-17-Rd2 to |
| Rd1 | CAAGCAGAAGACGGCATACGAGA | add Illumina 15/17 indexes and |
| | T/Index5 (10 nt)/ | P5/P7 sequencing adapters to |
| | GTCTCGTGGGCTCGG (SEQ ID | RpoB-SPA amplicons from PCR1 |
| | NO: 24) | |
| P7-17- | 5′ | Used for PCR2 with P5-15-Rd1 to |
| Rd2 | AATGATACGGCGACCACCGAGAT | add Illumina 15/17 indexes and |
| | CTACAC/Index7 (10 nt)/ | P5/P7 sequencing adapters to |
| | TCGTCGGCAGCGTC (SEQ ID NO: | RpoB-SPA amplicons from PCR1 |
| | 25) | |

Overview of primer sequences. The following nucleotide codes were used: A: adenine; G: guanidine; C: cytosine; T: thymine; R: purine (A or G); Y: pyrimidine (T or C); W: weak (A or T); S: strong (G or C); M: amino (A or C); K: keto: (G or T); B: not A (T, C, or G); H: not G (A, T or C); D: not C (A, T or G); N: any nucleotide (A, G, C or T). The extended primer sequences used for multiplex Illumina sequencing are shown in italics. _*indicates a phosphorothioated DNA base to protect the linker from 3′ end degradation.

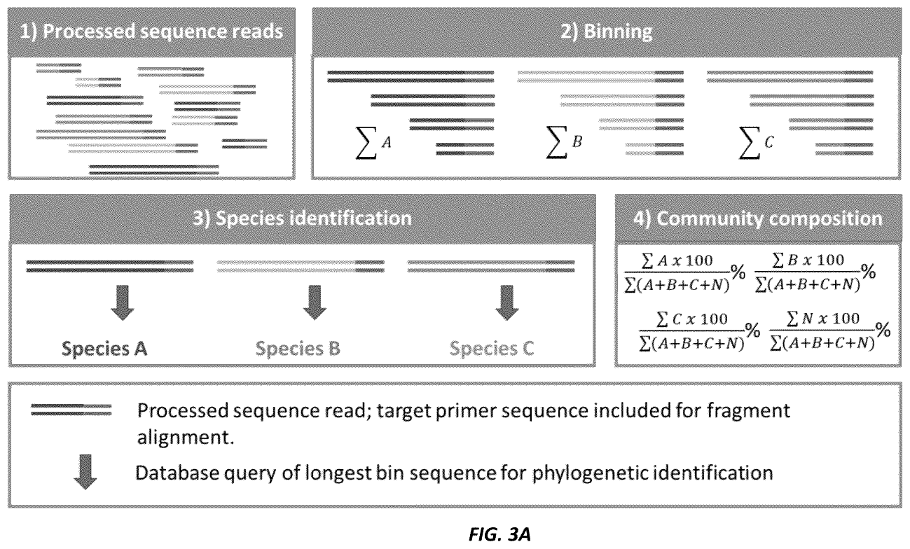

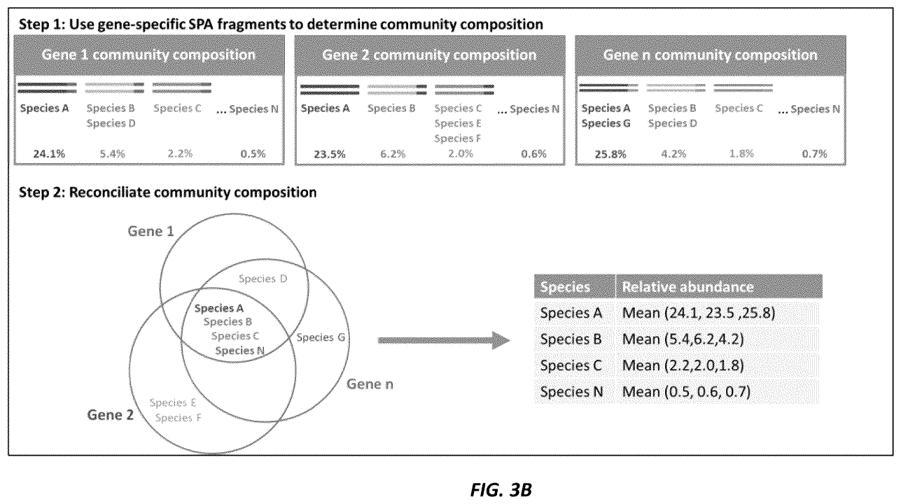

In some embodiments of the invention, the processing and analysis of the SPA fragment sequences includes one or more of the following steps as shown in FIG. 3A:
1. Reads are filtered based on read quality. Error correction is done using software such as DADA2 (Callahan et al, 2016), which makes use of a parametric error model. The remaining error-corrected reads of different lengths are deduplicated while recording the number of duplicates by sequence for calculating community composition.
2. Unique SPA fragments are aligned on the sequence of the RpoB6-SPA-seq-F1652 primer forming bins of matching sequences representative for the same species.
3. The database of bacterial rpoB genes is searched for the longest read in each bin of matching sequences for species identification. If a fragment does not match exactly to the database of bacterial rpoB genes, the closest match species is assigned, noting the likelihood of a false match.
4. Community composition is calculated based on the percent of reads assigned to each bin, taking into consideration the number of duplicate reads identified in step 1.

Additional primers besides those derived from the RpoB6-F1652 and the 16S-V4-R primers can be used for SPA fragment sequencing. EXAMPLE 2 describes the design of alternative rpoB gene specific primers. A RpoB1-R1327 primer, which recognizes the rpoB gene sequence between positions 1327-1352 (positions based on the Escherichia coli rpoB gene sequence) and allows for generation of SPA fragments upstream of this region, was validated in silico for the phylogenetic resolution of the sequences of 50 nucleotide Single Point Amplification (SPA) fragments as described in EXAMPLES 3 to 9. In EXAMPLE 7 a RpoB6-R1630 primer, which recognizes the rpoB gene sequence between positions 1630-1652 and allows for generation of SPA fragments upstream of this region, was validated, and EXAMPLE 10 describes the combined use of the RpoB1-R1327 primer and RpoB6-R1630 primer for improved identification of members of the Enterobacteriaceae. EXAMPLE 13 describes the Cpn60-R571 primer, which recognizes the cpn60 gene sequence between position 571-593, (position numbers based on the Escherichia coli cpn60 gene sequence). In another embodiment of the invention, a method is provided for multi loci SPA fragment sequencing. Use of two or more different gene-specific SPA primers in the same amplification reaction such as, for example, the RpoB1-R1327 and Cpn60-R571 primers is detailed in EXAMPLE 14. One example of a protocol for the method of amplifying mcfDNA provided herein is generally illustrated in FIG. 2 and is as follows:
1. Isolation of cfDNA using standard protocols.
2. End repair and 5′-phosphorylation of cfDNA fragments followed by the 3′ addition of a deoxy-adenine to create a 3′-sticky end formed by a single adenine nucleotide using standard protocols.
3. Ligation of an adaptor, which in this embodiment is an asymmetric linker cassette created by annealing the primers SPA-cas1 and SPA-cas2, using T4 DNA ligase.
4. Single point linker cassette repair. To generate multi loci SPA fragments, multiplexing PCR is performed on the ligation product using three primers: (a) the SPA1-amp primer that recognizes the repaired 5′ asymmetrical end of the linker cassette; (b) a primer that recognizes the primer annealing site specific for the conserved region of the first phylogenetic marker gene, such as the RpoB6-F1652 primer; and (c) a primer that recognizes the primer annealing site specific for the conserved region of the second phylogenetic marker gene, such as the 16S-V4-R primer. Alternatively, the RpoB1-1327R primer, the Cpn60-R571 primer, or combinations of these primers can be used. These primer sequences are provided in Table 1.
5. Once the asymmetric linker cassette has been repaired, the primer (SPA1-amp primer) that recognizes the repaired 5′ asymmetrical end of the linker cassette can anneal and PCR amplification is initiated. In the case of the reverse RpoB6-F1652 and Cpn60-R571 primers, this will result in the amplification of DNA sequences located downstream of position 1652 of the rpoB gene and upstream of position 571 of the cpn60 gene, respectively. An enrichment PCR protocol can be used to reduce background amplification of human DNA fragments resulting from nonspecific primer annealing.
6. In a follow up PCR step, adapter sequences are added to the amplified SPA fragments using the primers RpoB1-SPA-seq-R1327, Cpn60-SPA-seq-R571 and SPA1-seq-F (see Table 1). In a second PCR reaction (PCR2), UDI and sequencing anchors are added to the amplified SPA fragments using the primers P5-15-Rd1 and P7-I7-Rd2 (see Table 1). The PCR2 is performed using unique sets of UDI for each sample, subsequently allowing the pooling of the libraries, after which fragments are paired-end sequenced using NGS Illumina sequencing, e.g. on the Illumina NextSeq 1000 (Illumina, Inc., San Diego, CA). This approach will result in sequenced fragments that share the sequence of either the RpoB6-SPA-seq-F1652primer or the Cpn60-SPA-seq-R571 primer, followed by sequences that vary in length and nucleotide composition. Sequences derived from the same microorganisms and extended from the same primer will be identical except for the length of the sequenced fragment, which will vary as a function of the distance between the respective primer annealing site and the end of the mcfDNA fragment.

In one instance, the processing and analysis of the SPA fragment sequences includes the following steps:
1. Similar to single loci SPA fragment sequencing, the reads are filtered based on read quality. Error correction can be done using software such as DADA2 (Callahan et al, 2016), which makes use of a parametric error model. The remaining error-corrected reads of different lengths can be deduplicated while recording the number of duplicates by sequence for calculating community composition.
2. Multi loci SPA fragment sequencing can include a step to deconvolute the reads on the phylogenetic gene level. Unique SPA fragments are aligned on the sequences of the RpoB1-R1327 primer or the Cpn60-R571 primer and sorted in gene specific “buckets”. This is schematically shown in Step 1 of FIG. 3B. Subsequently, the sequences of each bucket are sorted into bins of matching sequences representative for the same species. In a next step, the rpoB and cpn60 gene databases are searched for the longest read in each bin of matching sequences for species identification. If a fragment does not match exactly to the database entries, the closest match species is assigned, noting the likelihood of a false match.
3. For each phylogenetic gene, the community composition is calculated based on the percent of reads assigned to each species, taking into consideration the number of duplicate reads identified in step 1.

To reconcile the outcomes obtained for the SPA fragments obtained from different phylogenetic identifier genes, their results are compared and consolidated into a consensus community description (species and their relative abundances), as is schematically shown in Step 2 of FIG. 3B.

In one embodiment of the invention, the reconciliation process of Step 2 in FIG. 3B works as follows:
1. To phylogenetically identify the community members, SPA fragments that provide the highest level of phylogenetic resolution are prioritized. Thus, SPA fragments that allow for species level identification have priority over SPA fragments that allow for identification at the genus level. For example, a subset of SPA fragments from gene 1 and gene 2 both specifically identify species A, confirming its presence as a community member. However, a second subset of SPA fragments from gene 1 identifies the closely related species B and D, while a second subset of SPA fragments from gene 2 is specific at the species level and indicates that only species B is present. It is therefore concluded that species B is present. Similar, a third subset of SPA fragments from gene 1 identifies the presence of species C, while a third subset of SPA fragments from gene 2 identifies the presence of the closely related species C, species E and species F. Therefore, it is concluded that species C is present.
2. To determine the abundances of the community members, the mean of the relative abundance for each species (as determined using the SPA fragments from each of the different phylogenetic identifier genes) is calculated.

The utility of the methods of the invention is exemplified in EXAMPLES 1-14 of the present disclosure. For example, in EXAMPLE 1 of the present disclosure, the inventors demonstrate that the primers RpoB6-SPA-seq-F1652 and 16S-SPA-seq-V4-R can be used to generate unique SPA fragments from mcfDNA present in blood that allowed for bacterial identification on the species level based on homology to the rpoB gene and the 16S rRNA gene, respectively. In EXAMPLE 2 of the present disclosure, the inventors demonstrate that a 50 nucleotide length cutoff enabled in silico generation of 20,919 unique SPA fragments covering the rpoB gene region upstream of the RpoB1-R1327 primer annealing site. The generated SPA fragments provided sufficient phylogenetic resolution to enable identification of many bacteria at the species level. These 50 nucleotide SPA fragments were generated from 50,569 unique rpoB gene sequences present in the PATRIC database (Wattam et al, 2014). Increasing this length to 75 nucleotides had only a marginal effect on the phylogenetic resolution of this method (22,603 unique fragments). The 50 nucleotide fragment size was selected based on the average length (40-100 nucleotides) of mcfDNA fragments. It should be noted that larger fragments will also be generated for each species, further improving the resolution for the phylogenetic identification.

EXAMPLES 3 to 9 demonstrate that, despite their relatively short size, the sequences of the 50 nucleotide long SPA fragments covering the rpoB gene region upstream of the RpoB1-R1327 primer annealing site allow for high resolution phylogenetic identification at the bacterial species level of many clinically relevant bacterial isolates.

EXAMPLE 10 describes a simulation showing that mcfDNA fragments with an average length of 60 base pairs can be reliably used to identify strains present at 0.5% or above in a known gut microbial community at the species and subspecies level. The species and subspecies are detectable in liquid biopsy samples, including peripheral blood. On average, strain abundances measured based on SPA fragments were within 1.4% of the actual abundance. For strains with less than 1% abundance, the average error was 1.8%, ranging from 0.1% to 7.2%; for strains with an abundance of 1% or higher, the average error was 1.2%, ranging from <0.1% to 4.5%.

EXAMPLE 11 describes an experiment to determine the phylogenetic accuracy of the SPA fragments generated using the RpoB1-R1327 primer in EXAMPLE 10. The results shows that the SPA fragments have very high phylogenetic specificity to reliably classify bacteria at both the taxonomic genus and species level.

EXAMPLE 12 is an experiment designed to access how the sensitivity and specificity of the SPA fragment sequencing methods compare to the current method of deep metagenome sequencing of cfDNA fragments followed by taxonomic classification using read-based metagenome analysis methods. The simulations described in EXAMPLE 12 using deep metagenome sequencing of cfDNA fragments followed by taxonomic classification of mcfDNA using read-based metagenome analysis methods show that current read-based tools are unsuitable for taxonomic classification of the short sequencing reads obtained from mcfDNA. As such the current approach lacks the sensitivity and specificity to provide meaningful insights for disease detection and progression monitoring. Overcoming this limitation would require very deep sequencing and assembly of short reads into larger fragments. In addition to higher sequencing costs, limitations in the assembly of short sequencing reads render the current approach unsuitable for scalable application to the routine analysis of microbial patterns in biopsy samples.

EXAMPLE 13 describes identification of a degenerate primer comprising complementarity to a conserved region spanning position 571 to 593 of the cpn60 gene (position numbers based on the Escherichia coli cpn60 gene, “Cpn60-R571 primer”) for SPA fragment sequencing. The results described in EXAMPLE 13 show that the simulated community compositions using rpoB gene-derived SPA fragments and cpn60 gene-derived SPA fragments are very similar. In addition, and unexpectedly, it was discovered that the Cpn60-R571 primer can be used in combination with the RpoB1-R1327 primer in the SPA fragment sequencing methods of the present disclosure to improve the phylogenetic resolution based solely on the rpoB gene. Based on this result a new method is provided, referred to as multi loci SPA fragment sequencing, which combines SPA fragments from multiple phylogenetic identifier genes to analyze the composition of microbial communities. The results of EXAMPLE 13 show that the multi loci SPA fragment sequencing method using two or more phylogenetic identifier genes, such as the rpoB and cpn60 genes, can have advantages over the SPA fragment sequencing method using a single locus. Such advantages include: (1) provision of an internal sample control for the SPA fragment amplification and sequencing process, as similar results for community species abundances are expected independent of the phylogenetic identifier gene; and (2) improvement in phylogenetic resolution for the identification of the community members on the species and subspecies level, as was highlighted in EXAMPLE 13.

The clinically relevant bacterial isolates that can be identified using the methods of the invention include, but are not limited to, Flavobacterium sp., Staphylococcus auricularis, Pseudomonas toyotomiensis, Rheinheimera sediminis, Finegoldia magna, Parvularcula sp., Pseudomonas stutzeri, Pseudomonas soyae, Pseudomonas saponiphila, Pseudomonas sp., Peptoniphilus harei, Quisquilii bacterium sp., Azoarcus sp., Sphingopyxis terrae, uncultured Clostridiales bacterium strain UMGS460, Staphylococcus schweitzeri, Flavobacterium erciyesense, Rhodococcus yananensis, Dietzia massiliensis, Cutibacterium acnes subsp. elongatum, Angustibacter aerolatus, Aerococcus urinae, Klebsiella quasivariicola, Comamonas fuminis, Mycobacterium tuberculosis, Mycobacterium abscessus, Mycobacterium avium, Mycobacterium chimaera, Mycobacterium leprae, Mycobacterium xenopi, Mycobacterium (para)intracellulare, Mycobacterium kansasii, Mycobacterium gilvum, Mycolicibacterium gen. nov. (“fortuitum-vaccae” clade), Mycobacterium gen. (“tuberculosis-simiae” clade), Staphylococcus aureus, Staphylococcus argenteus, Staphylococcus schweitzeri, Pseudomonas aeruginosa, Burkholderia cepacia complex, Burkholderia ubonensis, Burkholderia species Nov., Burkholderia multivorans, Burkholderia pseudomultivorans, Burkholderia pseudomallei, Burkholderia mallei, Trinickia species, Burkholderia thailandensis, Haemophilus influenzae, Haemophilus parainfluenzae, Streptococcus species at the various group and species levels, Streptococcus dysgalactiae, Streptococcus pyogenes, Streptococcus mutans, Streptococcus suis, Streptococcus mitis, Streptococcus pneumoniae, Streptococcus agalactiae, Streptococcus anginosus, Streptococcus intermedius, Streptococcus constellatus, Streptococcus equi subsp. zooepidemicus, Streptococcus oralis, Streptococcus gordonii, Streptococcus uberis, Streptococcus parasanguinis, Streptococcus sanguinis Streptococcus parauberis, Streptococcus infantarius, Streptococcus iniae, Streptococcus salivarius, Streptococcus thermophilus, Streptococcus vestibularis, Streptococcus bovis, Streptococcus gallolyticus subsp. gallolyticus, Streptococcus gallolyticus subsp. macedonicus, Streptococcus gallolyticus subsp. pasteurianus, Streptococcus equinus, Enterococcus faecalis, Enterococcus faecium, Porphyromonas gingivalis, Porphyromonas cangingivalis, Porphyromonas uenonis, Porphyromonas endodontalis, Propionibacterium acidifaciens, Porphyromonas asaccharolytica, Porphyromonas macacae, Prevotella pallens, Prevotella histicola, Prevotella melaninogenica, Prevotella copri, Prevotella intermedia, Prevotella oral, Prevotella nanceiensis, Prevotella salivae, Prevotella nigrescens, Prevotella denticola, Prevotella buccae, Prevotella stercorea, Prevotella oris, Prevotella disiens, Prevotella bryantii, Prevotella shahii, Tannerellaforsythia, Bacteroides fragilis, Helicobacter pylori, Chlamydia trachomatis, Neisseria meningitidis, Neisseria gonorrhoeae, Neisseria subflava, Neisseria perfiava, Neisseria flavescens, Neisseria cinerea, Neisseria lactamica, Neisseria weaver, Neisseria zoodegmatis, Neisseria brasiliensis, Neisseria mucosa, Neisseria animaloris, Aggregatibacter actinomycetemcomitans, Aggregatibacter aphrophilus, Aggregatibacter segnis, Saccharopolyspora species, Bacillus clausii, members of the genera Pseudoxanthomonas and Streptomyces, Fusobacterium nucleatum subsp. polymorphum, Fusobacterium hwasookii, Fusobacterium canifelinum, Fusobacterium nucleatum subsp. animalis, Fusobacterium periodonticum, Fusobacterium necrophorum subsp. funduliforme, Fusobacterium mortiferum, Fusobacterium varium, Fusobacterium nucleatum subsp. nucleatum, Fusobacterium ulcerans, Fusobacterium nucleatum subsp. vincentii, Fusobacterium equinum, Fusobacterium gonidiaformans, Fusobacterium necrogenes, Fusobacterium naviforme, Peptostreptococcus stomatis, Pseudonocardia asaccharolytica, Parvimonas species including Parvimonas oral and Parvimonas micra, Gemella species including Gemella morbillorum, Gemella haemolysans, Gemella palaticanis and Gemella sanguinis, Clostridium difficile, Acinetobacter baumannii, Acinetobacter lactucae, Acinetobacter pittii, Acinetobacter calcoaceticus, Acinetobacter oleivorans, Acinetobacter nosocomialis, Acinetobacter radioresistens, Acinetobacter variabilis, Acinetobacter courvalinii, Acinetobacter ursingii, and members of the Enterobacteriaceae, including Escherichia and Klebsiella species.

This phylogenetic identification of many clinically relevant bacterial isolates at the species level represents a significant improvement over methods such as Kaiju (Menzel et al, 2016) or Kraken (Wood and Salzberg, 2014), which are being used for sequence-read based identification of microorganisms represented by the mcfDNA at the genus level. As is well documented for many pathogenic bacteria, including Mycobacterium species, optimal patient treatment protocols including the use of antibiotics are species-level specific, showing the importance of the level of phylogenetic resolution that is uniquely obtained with the single point amplicon sequencing approach provided herein. Furthermore, by targeting genes that are absent or sufficiently different from the host genome, such as genes conserved in pathogenic fungi that are absent from the human genome (Abadio et al, 2011), the method provided herein can also be used to detect the presence of Eukaryotic infections, such as those caused by parasitic fungi and amoeba. Candidate fungal genes for SPA fragment sequencing include: trr1 that encodes for thioredoxin reductase; rim8 that encodes for a protein involved in the proteolytic activation of a transcriptional factor in response to alkaline pH; kre2 that encodes for α-1,2-mannosyltransferase; and erg6 that encodes for Δ(24)-sterol C-methyltransferase (Abadio et al, 2011).

In certain instances, disease phenotypes caused by bacteria will depend on the presence of virulence/pathogenicity factors located on mobile genetic elements, including conjugative and/or mobile plasmids, phages, and pathogenicity islands that can be horizontally transferred between bacteria, as is the case for Escherichia coli, Salmonella, Klebsiella, Listeria, Bacillus, pyogenic streptococci and Clostridium perfringens, among others (for review, see Gyles and Boerlin, 2014). As the result of horizontal gene transfer, in some instances phylogenetic information on species composition will be insufficient to predict disease pathology, and therefore needs to be complemented with information on community functionality. SPA fragment sequencing provides the flexibility to address both phylogenetic identification and community functionality: by selecting a degenerate primer that recognizes a conserved DNA region of a specific function, the same protocol outlined in FIG. 2 and FIGS. 3A and 3B is broadly applicable for SPA amplification and sequencing of functional genes.

For instance, the presence in Escherichia coli of the PKS pathogenicity island encoding, among other virulence factors, for genotoxic colibactin synthesis has been linked to increased risk for developing colorectal cancer (Pleguezuelos-Manzano et al, 2020). By designing a primer for SPA fragment amplification that specifically targets the PKS gene cluster essential for colibactin synthesis, the presence of genotoxic Escherichia coli strains (Pleguezuelos-Manzano et al, 2020) can be determined and combined with phylogenetic information for risk assessment of colorectal cancer.

Pan-cancer analyses recently revealed cancer-type-specific fungal ecologies and bacteriome interactions (Narunsky-Haziza et al, 2022). By designing a primer for SPA fragment amplification that specifically targets a human fungal phylogenetic marker such as the nuclear ribosomal internal transcribed spacer region 1 (ITS1) or region 2 (ITS2), the presence of human pathogenic fungi can be determined and combined with bacterial phylogenetic information to for risk assessment of cancer. The amplified mcfDNA that can be generated in the methods provided herein can include mcfDNA from fungal species including one or more members of the Ascomycota, Basidiomycota and Mucoromycota, including Alternaria species, Aspergillus species, Blastomyces species, Candida species, Capnodiales species, Cladosporium species, Malassezia species, Phaeosphaeria species, Pseudozyma species, Saccharomyces species, Sporobolomyces species, Vishniacozyma species, and Yarrowia species. The methods for amplifying mcfDNA provided herein can also be used for detecting viral DNA. For example, a primer for a conserved viral gene can be included in the amplification reaction, where the viral gene primer includes complementarity to a conserved region of the viral gene to determine the presence of the virus. The viral gene can be a human DNA- or RNA-based oncovirus gene. Assessing the risk and better understanding the cause of cancer can be improved by designing primers for SPA fragment amplification that specifically target conserved genes present in human oncoviruses. For example, the method can be used for determining the presence of human DNA-based oncoviruses such as, but not limited to, the Epstein-Barr Virus (EBV), Human Papillomavirus (HPV), Hepatitis B virus (HBV), Human Herpesvirus-8 (HHV-8), and Merkel Cell Polyomavirus (MCPyV).

In one aspect of the invention, phylogenetic and functional information can be obtained simultaneously by including both one or more degenerate primers that target the phylogenetic identifier gene(s) and a primer that targets a functional gene in the same reaction for the SPA fragment amplification step (FIG. 2, step 4). This approach may be referred to herein as multiplex SPA for the simultaneous detection of multiple targets in a single reaction. Thus, the method for amplifying mcfDNA provided herein can further include in the amplification reaction a primer for a functional gene designated for the set of reference microbes, wherein the functional gene primer comprises complementarity to a conserved region of the functional gene, to determine the presence of the functional gene. The functional gene can be, but is not limited to, a pathogenicity factor, a PKS gene cluster essential for colibactin synthesis, or a choline trimethylaminelyase gene.

Since 100,000 sequencing reads represent the standard depths for amplicon-based sequencing for complex microbial community analysis, the latest Illumina NEXTSEQ instruments allow for an unprecedented number of samples to be sequenced in parallel. For example, the Illumina NEXTSEQ 6000 allows to theoretically collect 20 billion reads with a single run, which would correspond to 100,000 paired-end sequenced samples.

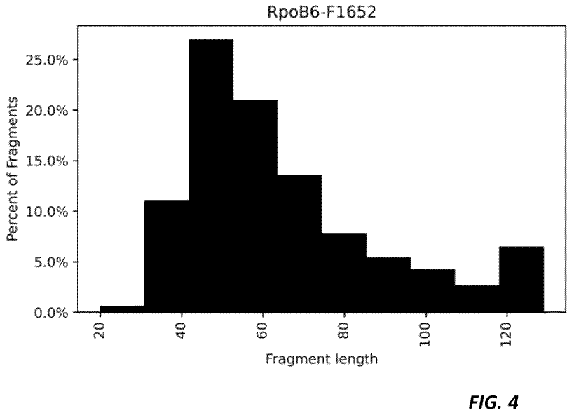

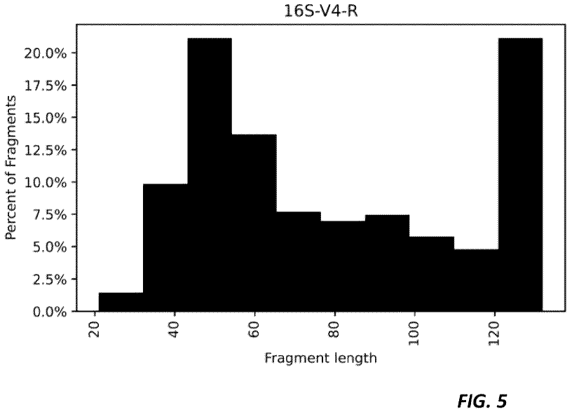

In addition to monitoring of specific diseases, SPA fragment sequencing can be useful as part of the general health screening. Unlike the stool microbiome, the microbiome of colonizing and infecting bacteria will be relatively stable, with changes occurring when the relation between host and microbes is changing. This includes situations of new invasions by infectious and colonizing microorganisms, such as the formation of stomach ulcers, the formation of intestinal polyps/adenomas and their progression into malignancies, gastrointestinal diseases including Irritable Bowel Disease (IBD), various tumors and their specific microbiomes including pancreatic cancer, lung cancer and cervical cancer, Central Nervous System (CNS) diseases including multiple sclerosis (MS) and Alzheimer's disease, minimal residual disease (MRD) monitoring, and other diseases characterized by dysbiotic and inflammatory microbiomes such as cystic fibrosis or tuberculosis, and general risk monitoring of infections in patient populations with a compromised immune system, positioning SPA fragment sequencing as an ideal tool for risk monitoring, early detection, prognostics and evaluation of disease progression. Contrary to PCR based detection methods that monitor for the presence of specific bacteria, SPA fragment sequencing provides an “open” diagnostics approach to detect any bacterium or fungus based on the presence of its mcfDNA in peripheral blood. FIGS. 4 and 5 show the distribution of SPA fragment lengths generated using primers targeting the rpoB gene and the 16S rRNA gene, respectively.

In one aspect of the invention, SPA fragment sequencing can provide an important non-invasive method for (early) detection and identification of infectious and colonizing bacteria using mcfDNA from peripheral blood samples, which can subsequently be linked to a broad range of diseases, including: screening for tuberculosis and other diseases caused by Mycobacterium species; determining pulmonary infection risks and causes in cystic fibrosis patients; determining the risk and onset of sepsis in patients with compromised immune systems; detection of opportunistic bacterial pathogens originating from the oral cavity that have been linked to Alzheimer's disease, pancreatic cancer and other serious conditions such as endocarditis; women's health issues including Chlamydia linked to mucopurulent cervicitis, pelvic inflammatory disease, tubal factor infertility, ectopic pregnancy and cervical cancer; detection and monitoring of progression of cancer; monitoring of minimal residual disease after oncology treatments; detection and monitoring of progression and minimal residual disease of breast cancer including triple negative breast cancer; detection of esophageal cancer, precancerous colonic polyps and early stage colorectal cancer, and detection and monitoring of progression and minimal residual disease of gastrointestinal cancers in general; detection and monitoring of progression and minimal residual disease in lung cancer; non-invasive analysis of the microbiome in pancreatic cancer patients to propose treatment protocols and prognostics for long-term survival; detection of Clostridium difficile infections; post-transplant bloodstream infections and Graft versus Host Disease (GvHD); detection of hospital acquired infections by emerging pathogens of clinical concern; detection of an infection in an immune compromised person; or detection of infection or inflammation of the gastrointestinal track in Irritable Bowel Disease (Crohn's disease, Ulcerative colitis); and combinations thereof. Therefore, SPA fragment sequencing represents a quantum leap forward to apply mcfDNA sequencing as a high-resolution, high-throughput and low-cost routine test in disease detection, patient monitoring, risk assessment and large-scale population screenings using mcfDNA informed biomarkers. For example the microbial footprint obtained with SPA fragment sequencing combined with the mutational footprint and methylation footprint that are currently being used as biomarkers for the detection, monitoring and prognostics of cancers, will provide a powerful tool for improved early detection and monitoring of progression of various types of cancer. It is expected that including the microbial footprint will increase the specificity and selectivity of screening tests, e.g. for the detection of early stage adenomas and carcinomas in colorectal cancer. Furthermore, once unique SPA fragments have been identified that correlate with the detection of specific diseases and monitoring of their progression, their sequences can be used to develop species-specific PCR-based screening assays as part of diagnostic platforms.

In addition to using mcfDNA from blood, the SPA fragment sequencing approach provided herein is applicable to analyze microbial DNA compositions in any sample type, especially when in samples having low amounts of small fragment microbial DNA. This includes biopsy samples from solid tumors, skin grafts, and other liquid biopsy samples besides peripheral blood, as well as mcfDNA present in stool samples.

In other instances, the methods and kits provided herein can be used for SPA fragment sequencing as a non-invasive method for (early) detection and identification of infectious and colonizing fungal microbes using mcfDNA from biological samples as described herein. For example, the set of reference microbes in this case includes reference fungal microbes. The method can be used to determine the presence of one or more fungi and/or to determine the fungal community composition. The one or more degenerate primers included in the amplification reaction in this embodiment includes complementarity to a conserved region of a human pathogenic fungal gene or DNA region designated for the set of reference fungal microbes. The conserved human pathogenic fungal gene or DNA region is herein referred to interchangeably for the purposes of the specification and claims as a “fungal phylogenetic marker gene”. In some instances, the fungal phylogenetic marker gene can be ITS1 or ITS2. The microbial community composition that can be calculated based on the percent of the sequences assigned to each species is a fungal community composition. The amplified mcfDNA fragments can include mcfDNA from one or more members of the Ascomycota, Basidiomycota and Mucoromycota, including Alternaria species, Aspergillus species, Blastomyces species, Candida species, Capnodiales species, Cladosporium species, Malassezia species, Phaeosphaeria species, Pseudozyma species, Saccharomyces species, Sporobolomyces species, Vishniacozyma species, and Yarrowia species.

In the SPA fragment sequencing method, a DNA region is identified in a suitable phylogenetic marker gene that has the following characteristics:
1. Presence of a highly conserved DNA region to design a degenerate primer for annealing to the phylogenetic marker gene.
2. Adjacent to the primer annealing site the presence of a highly variable DNA region with high phylogenetic resolution. This region will become part of the single point amplicon (SPA) fragment.

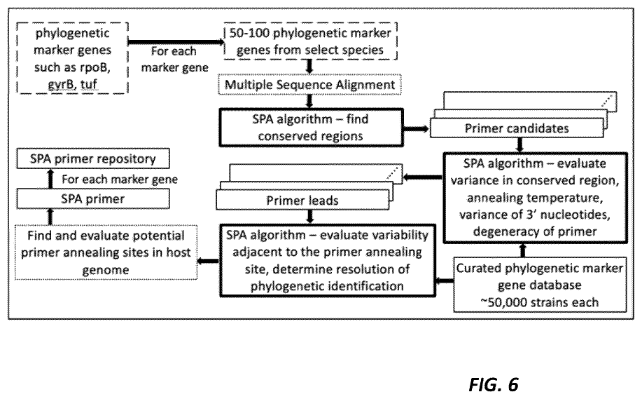

An overview of an exemplary SPA primer design method is shown in FIG. 6. For each phylogenetic marker gene, such as rpoB, cpn60, 16S rRNA, ITS1, ITS2, gyrB, tuf or other phylogenetic marker gene or conserved housekeeping gene including, but not limited to, those used by CheckM (Parks et al, 2015), 50-100 species are initially selected that cover the prokaryotic diversity, including members of the phylum Proteobacteria (including representative α-, β-, γ-, δ- and ε-Proteobacteria), the phylum Firmicutes (including representatives for the classes Bacilli, Clostridia, Erysipelotrichia and Negativicutes), and the phyla Acinetobacteria and Fusobacteria. Marker genes for these species are aligned using a multiple sequence alignment tool like ClustalW. The SPA algorithm is subsequently used to identify conserved regions as putative annealing sites for primer candidates by looking for the highest “average sequence variance” scores over 25 nucleotide-long DNA regions among this limited set of sequences. This is performed as follows:
Determine the percent of nucleotides for each nucleotide (GATC) at each position.
Calculate the variance of the percentages at each position.
Calculate region variance as the average of the variances of each position in the region.

A completely conserved nucleotide position will have 100% of one nucleotide and 0% for the other three nucleotides, and a variance of 0.25. A completely non-conserved region will have 25% of each nucleotide and a variance of 0. Primer candidates are prioritized based on their “average sequence variance” scores.

Primer candidates are evaluated for key properties including the level of primer degeneracy and annealing temperature (>50° C.). The sequences from the complete curated marker gene database are aligned to these conserved regions to determine their nucleotide compositions. The conservation of their 3′ nucleotide (must be >99% conserved among entries) and their “average sequence variance” scores are calculated (highly conserved regions have the highest score) and used to rank and select primer leads, prioritizing primers with the highest score.

In the next step, using a curated marker gene database, an algorithm (referred to as “SPA algorithm” in FIG. 6) is used to determine the “average sequence variance” for the regions adjacent to the primer annealing site. Primers with adjacent 25 nucleotide-long and 50 nucleotide-long regions with ideally an average sequence variance of <0.15 and <0.075, respectively, are prioritized based on the lowest score. The algorithm also identifies the resolution of phylogenetic identification for the regions adjacent to each primer lead by determining the number of unique SPA fragments. SPA primers with the highest phylogenetic resolution are added to the SPA primer repository.

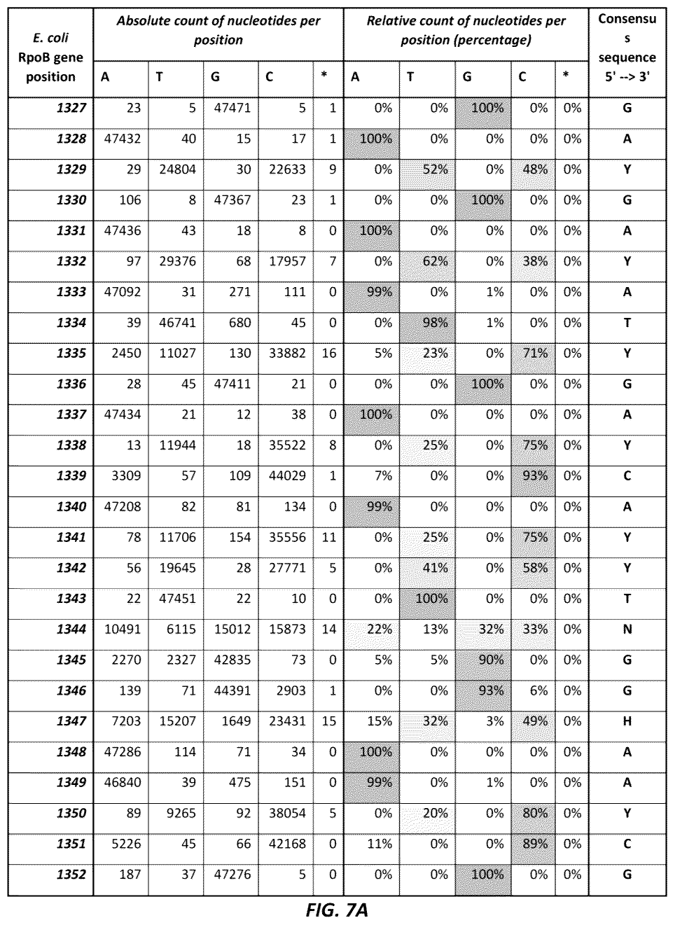

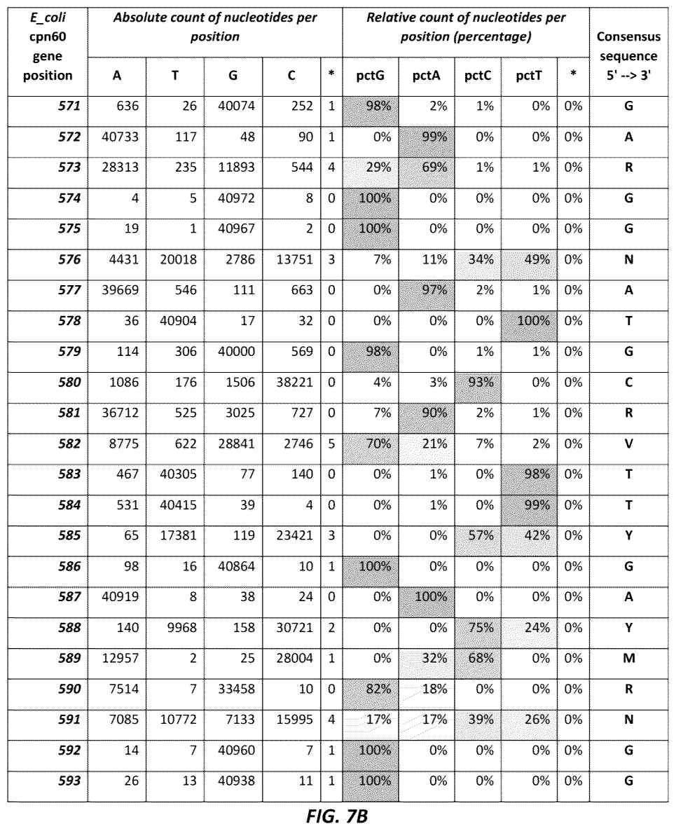

FIG. 7A shows nucleotide statistics for the rpoB gene region 1327-1352 and degenerate sequence (GAYGAYATYGAYCAYYTNGGHAAYCG (SEQ ID NO: 4)) which is the reverse complement sequence of degenerate primer RpoB1-R1327. In this specific example, the relative abundance of a nucleotide at a specific position was calculated using the nucleotide sequences of 47,505 aligned unique rpoB genes from the PATRIC database and used to design the degenerate sequence, which is provided from 5′ to 3′ using the following nucleotide codes: A: adenine; G: guanidine; C: cytosine; T: thymine; R: purine (A or G); Y: pyrimidine (T or C); H: not G (A, T or C); N: any nucleotide (A, G, C or T); *: presence of an ambiguous sequence at a specific rpoB gene position. The percentages of highly conserved nucleotide sequences used to determine the consensus sequence for the degenerate primer are highlighted. The position of the region is based on the nucleotide sequence of the Escherichia coli rpoB gene.

FIG. 7B shows nucleotide statistics for the cpn60 gene region 571-593 and degenerate sequence (GARGGNATGCRVTTYGAYMRNGG (SEQ ID NO: 5)) which is the reverse complement sequence of degenerate primer Cpn60-R517. The relative abundance of a nucleotide at a specific position was calculated using the nucleotide sequences of 40,989 aligned unique cpn60 genes from the PATRIC database and used to determine the degenerate sequence for this region, which is provided from 5′ to 3′ using the following nucleotide codes: A: adenine; G: guanidine; C: cytosine; T: thymine; R: purine (A or G); Y: pyrimidine (T or C); M: amino (A or C); V: not T (A, G or C); N: any nucleotide (A, G, C or T); *: presence of an ambiguous sequence at a specific cpn60 gene position. The percentages of highly conserved nucleotide sequences used to determine the consensus sequence for the degenerate primer are highlighted. The position of the region is based on the nucleotide sequence of the Escherichia coli cpn60 gene.

In the next step, the proposed degenerate primer sequences are matched to the human genome sequence and the number of hits with increased number of allowed mismatches is determined. To minimize annealing to human genomic DNA, a primer should ideally have two or more mismatches with the human genome.

Various modifications and variations of the disclosed methods, compositions, and uses of the invention will be apparent to the skilled person without departing from the scope and spirit of the invention. Although the invention has been disclosed in connection with specific preferred aspects or embodiments, the invention as claimed should not be unduly limited to such specific aspects or embodiments.

The present invention may be implemented using hardware, software, or a combination thereof and may be implemented in one or more computer systems or other processing systems. In one aspect, the invention is directed toward one or more computer systems capable of carrying out the functionality described herein.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter.

### Example 1

SPA Fragment Sequencing Using the 16S rRNA Gene and the rpoB Gene as Phylogenetic Markers

As representative examples, the SPA sequencing approach was successfully demonstrated for the rpoB gene and the 16S rRNA gene as an example of a single-copy and multi-copy phylogenetic marker, respectively.

To validate the RpoB6-F1652 primer and the 16S-V4-R primer for SPA fragment amplification from the rpoB gene and the 16S rRNA gene, the following protocol was followed. Following the steps outlined in FIG. 2, cfDNA isolation was performed using the Qiagen QIAamp ccfDNA/RNA Kit on 1.0 ml blood plasma from healthy volunteers.

To confirm the presence of mcfDNA in the blood samples, total cfDNA was isolated on 1.0 ml blood plasma and deep sequencing was used to determine the percentage of mcfDNA. In the case of these healthy donors, the percentage of mcfDNA was approximately 0.5% of the total cfDNA (data not shown). This is considerably lower than typically found in blood samples from e.g. cancer patients, where this ranged between approximately 1% to 4% (Poore et al, 2020).

Subsequently, following the supplier's instructions the xGen™ DNA Lib Prep MC kit (IDT) was used for end repair plus 5′-phosphorylation on 10 ng cfDNA fragments followed by the 3′ addition of a deoxy-adenine to create a 3′-sticky end of a single adenine nucleotide (Step 2), after which 20 ng of the asymmetric SPA-linker-UMI-Y was ligated to the repaired cfDNA fragments (Step 3) in a total volume of 16 μl.

The sequences of the two single stranded DNA fragments, SPA-cas1 and SPA-cas2, was used to create the asymmetric SPA-linker-UMI-Y linker cassette are listed in Table 1. The linker cassette was created by the following procedure. First, by annealing equal amounts (4 nmol) of SPA-cas1 and SPA-cas2. The mixture is first heated for 2 min. at 95° C., then for 10 min. at 65° C., 10 min. at 37° C., and finally 20 min. at room temperature. The mixture is kept on ice or stored at 4° C.

To repair the asymmetric linker cassette, a PCR reaction, referred to as PCR1, was performed on the ligation product using two primers: (a) the SPA1-seq-F primer that recognizes the repaired 5′ asymmetrical end of the linker cassette; (b) a primer that recognizes the primer annealing site specific for the conserved region of the phylogenetic marker gene, in this example the RpoB6-SPA-seq-F1652 primer. The forward (SPA1-seq-F) and reverse (e.g. RpoB6-SPA-seq-F1652) primers include a 5′ extension corresponding to the Illumina Read-1 and Read-2 sequences, respectively, to allow sequencing library preparation. The PCR1 was performed in 25 μl reaction containing 1×KAPA HiFi HotStart ReadyMix, 0.2 μM of each primer, and the Linker-cfDNA ligation products. The reaction was run in a thermocycler using the following program: 1 cycle at 95° C. for 10 min, 10 cycles at 98° C. for 20 sec, 65° C. to 50° C. for 30 sec and 72° C. for 15 sec, 35 cycles at 98° C. for 20 sec, 60° C. to 50° C. for 30 sec and 72° C. for 15 sec, and 1 cycle at 72° C. for 1 min. A similar protocol was followed for creating SPA fragments from the 16S rRNA gene using the 16S-seq-V4-R primer.

Once the asymmetric linker cassette was repaired, the SPA1-seq-F primer that recognizes the repaired 5′ asymmetrical end of the linker cassette can anneal and PCR1 amplification is initiated. In the case of the RpoB6-SPA-seq-F1652 primer this will result in the amplification of DNA sequences located downstream of position 1352 of the rpoB gene.

In a second PCR reaction (PCR2), Unique Dual Indexes (UDI) and Illumina sequencing anchors (P5 and P7) were added to the amplified SPA fragments using P5-15-Rd1 and P7-I7-Rd2 primers (see Table 1). The PCR2 was performed in 25 μl reaction containing 1×KAPA HiFi HotStart ReadyMix, 0.2 μM of each primer, and PCR1 bead cleaned products. The reaction was run in a thermocycler using the following program: 1 cycle at 95° C. for 3 min, 8 cycles at 95° C. for 30 sec, 55° C. for 30 sec and 72° C. for 30 sec, and 1 cycle at 72° C. for 5 min. The PCR2 was performed using unique sets of UDI for each sample, subsequently allowing the pooling of the libraries, after which fragments are paired-end sequenced using NGS Illumina sequencing, e.g. on the Illumina NEXTSEQ 1000 (Illumina, Inc., San Diego, CA). This approach resulted in sequenced fragments that all share the sequence of the gene specific primer (e.g., RpoB6-SPA-seq-F1652 primer) followed by sequences that vary in length and nucleotide composition. Sequences derived from the same microorganisms will be identical except for the length of the sequenced fragment, which will vary in function of the distance between the gene specific primer (e.g., RpoB6-SPA-seq-F1652 primer) annealing site and the end of the mcfDNA fragment. A similar protocol was followed for creating SPA fragments from the 16S rRNA gene using the 16S-seq-V4-R primer.

The analysis of the SPA fragment sequences included the following steps:
1. Adaptors and primers are trimmed from the sequences.
2. Using DADA2, an open-source software used for fast and accurate sample inference from amplicon data with single-nucleotide resolution (Callahan et al, 2016), the following steps are performed:
   a. Reads are filtered based on read quality.
   b. The remaining reads of different lengths are deduplicated.
   c. Reads are error-corrected using a parametric error model.
   d. Error-corrected reads are resolved to Amplicon Sequence Variants (ASVs).
3. ASVs of the RpoB6-F1652 primer or the 16S-V4-R primer are aligned to either the rpoB or 16s gene database using the basic local alignment search tool (BLAST, Altschul et al, 1990).

The database of bacterial rpoB genes was initially created by downloading their nucleotide sequences from the PATRIC database (Wattam et al, 2014) using the version available January 2021. If more than one (incomplete) rpoB gene was found for the same genome, we accepted the longest one, and rejected the shorter one(s). We confirmed for several instances our assumption that multiple rpoB genes in a single strain represented assembly errors, since each bacterium contains only one rpoB gene per genome. Genes were rejected if the genome had no taxonomy or if the gene was not annotated as “DNA-directed RNA polymerase beta subunit (EC 2.7.7.6)”. We evaluated all annotation rejections and found none that seemed to be rejected incorrectly. After January 2021, any new genome added to our genome database is searched for a rpoB gene by annotation, “DNA-directed RNA polymerase beta subunit (EC 2.7.7.6)”, and if found, its nucleotide sequence is added to the database of bacterial rpoB genes. These genomes come from PATRIC and NCBI (National Center for Biotechnology Information; https://www.ncbi.nlm.nih.gov/). Our curated database of bacterial rpoB genes contains 59,069 unique nucleotide sequences as of November 2021. For 16S sequences the 16S_ribosomal_RNA database was downloaded from NCBI.

The lengths of the ASV fragments for the RpoB6-F1652 primer and the 16S-V4-R primer are shown in FIG. 4 and FIG. 5, respectively. The SPA fragment length distributions are in line with the size distributions of mcfDNA. These fragments are slightly shorter than the lengths reported by Bumham et al (2016) as the primer annealing site was trimmed from the sequences.

Table 2 is a sample of alignment results for the RpoB6-F1652 primer-based SPA fragment sequences, while Table 3 provides a sample of alignment results for the 16S-V4-R primer-based SPA fragment sequences. The presented alignments were required to have an identity of at least 90% across 90% of the bases of the query. E-values represent the probability of the alignment occurring by chance. In the sample results for the 16S-V4-R primer, a SPA fragment as short as 40 nucleotides was aligned with confidence of an E-value of 1.94E-14 against the 16S rRNA gene of Comamonas fiuminis strain CJ34. Both 16S rRNA gene and rpoB gene derived SPA fragments were found for Flavobacterium, Staphylococcus, and Pseudomonas.

TABLE 2

| | | | | |
|---|---|---|---|---|
| Percent | Alignment | Fragment | | |
| Identity | Length | Length | E−value | Aligned rpoB Gene Genome Name |

Sequence:
CTACTCTCACTATGGTCGTATGTGTCCAATCGAAACACCAGAGGGTCCAA (SEQ
ID NO: 26)

| | | | | |
|---|---|---|---|---|
| 100 | 50 | 50 | 3.28E−19 | Staphylococcus auricularis strain |
| | | | | SNUC 993 |

Sequence:
CTATACTCACTACGGACGTTTATGTCCAATTGAAACTCCTGAGGGACCAAACAT
TGGTTTGATTTCATCTCTTGGGGTGTATGCTAAAGTGAATGGTA (SEQ ID NO: 27)

| | | | | |
|---|---|---|---|---|
| 99.0 | 98 | 98 | 8.67E−44 | Flavobacterium sp. strain UBA10157 |

Sequence:
CCCGACTCACTATGGTCGCGTGTGCCCGATCGAAACGCCGGAAGGTCCGAACA
TCGGTCTGATCAACTCGCTGGCTGCCTACGCCCGCACCAACCAGTACGGCTTCC
TGGAAAGCCCGTACCGCGTGG (SEQ ID NO: 28)

| | | | | |
|---|---|---|---|---|
| 100 | 128 | 128 | 5.51E−62 | Pseudomonas toyotomiensis strain |
| | | | | 718 |

Sequence:
CGACTCTCACTACGGTAGAATCTGTCCGATAGAAACACCAGAAGGACCAAACA
TCGGTCTTATAACTTCCATGACAACTTATTCTA (SEQ ID NO: 29)

| | | | | |
|---|---|---|---|---|
| 98.8 | 86 | 86 | 3.41E−37 | Finegoldia magna BVS033A4 |

Sequence:
CCCGACCCACTATGGCCGCATCTGCCCGATCGAGACGCCGGAAGGCCCGAATA
T (SEQ ID NO: 30)

| | | | | |
|---|---|---|---|---|
| 100 | 54 | 54 | 2.25E−21 | Parvularcula sp. strain NAT21 |

Sequence: CCCGACCCATTACGGTCGTGTGTGCCCGATCGAGACGCCGAAAGG
(SEQ ID NO: 31)

| | | | | |
|---|---|---|---|---|
| 95.3 | 43 | 45 | 1.69E−11 | Pseudomonas stutzeri ATCC 14405 = |
| | | | | CCUG 16156 |

Sequence:
CCCGACGCATTACGGTCGTGTATGCCCGATCGAAACGCCGGAAGGTCCGAACA
TCGGTCTGATCAACTCCCTGGCTGCCTATGCGCGCACCAACCAGTACGGCTTCC
TCGAAAGCCCATACCGTGTGG (SEQ ID NO: 32)

| | | | | |
|---|---|---|---|---|
| 100 | 128 | 128 | 5.51E−62 | Pseudomonas sp. strain NID84 |

Sequence: TAACTCACATTACGGAAGAATGTGTCCTATTGAGACACCAGAAGGT
(SEQ ID NO: 33)

| | | | | |
|---|---|---|---|---|
| 100 | 46 | 46 | 4.88E−17 | Peptoniphilus harei ACS-146-V- |
| | | | | Sch2b |

Sequence: TCCCACGCACTACGGCCGCGTCTGCCCGATCGAGACGCCTGAAGGCC
(SEQ ID NO: 34)

| | | | | |
|---|---|---|---|---|
| 97.9 | 47 | 47 | 6.58E−16 | Quisquiliibacterium sp. CC-CFT501 |

Sequence: TCCCACGCACTACGGCCGCGTCTGCCCGATCGAGACGCCTGAAGGCC
(SEQ ID NO: 35)

| | | | | |
|---|---|---|---|---|
| 100 | 44 | 44 | 5.79E−16 | Azoarcus sp. strain MCMED-G28 |

Sequence:
TCCGACGCACTATGGCCGTATCTGCCCGATCGAAACGCCGGAAGGCCCGAACA
TCGGTCTGATCAACAGACTCGC (SEQ ID NO: 36)

| | | | | |
|---|---|---|---|---|
| 98.7 | 75 | 75 | 3.72E−31 | Sphingopyxis terrae strain DE15.006 |
| | | | | strain JN15.010 |

Sequence:
TTGAAAGTGCCGCATGGTGAGAGCGGTATCGTCGTAGACGTAAAGAAATATTC
GCGTGCCAATGGCGACGATCTGGCACCGGGTCTTAACGAAGTCGTTCGCGTTT
ATATCGCGACAAAGCGCAAGA (SEQ ID NO: 37)

| | | | | |
|---|---|---|---|---|
| 99.213 | 127 | 127 | 9.14E−60 | uncultured Clostridiales bacterium |
| | | | | strain UMGS460 |

Sample alignment results for RpoB6-F1652 SPA fragments to the rpoB gene database. For each fragment, the percentage of identity, fragment length and alignment length to a reference genome are indicated. E−values represent the probability of the alignment occurring by chance.

TABLE 3

| | | | | |
|---|---|---|---|---|
| Percent | Alignment | Fragment | | |
| Identity | Length | Length | E−value | Aligned 16S rRNA Gene Genome Name |

Sequence:
TGTTTGATCCCCACGCTTTCGCACATCAGCGTCAGTTACAGACCAGAAAGTCGC
CTTCGCCACTGGTGTTCCTCCATATCTCTGCGCATTTCACCGCTACACATGGAA
TTCCACTTTCCTCTTCTGCACT (SEQ ID NO: 38)

| | | | | |
|---|---|---|---|---|
| 100 | 130 | 130 | 1.02E−63 | Staphylococcus schweitzeri strain |
| | | | | DSM 28300 |

Sequence:
TGTTCGCTACCCACGCTTTCGTCCATCAGCGTCAATCCATTAGTAGTAACC (SEQ
ID NO: 39)

| | | | | |
|---|---|---|---|---|
| 100 | 51 | 51 | 2.36E−20 | Flavobacterium erciyesense strain |
| | | | | F-328 |

Sequence:
TGTTTGCTCCCCACGCTTTCGCACCTGAGCGTCAGTGTTGTGCCAGGGGGCCGC
CTTCGCCACTGGTATTCCTCCAAATCTCTACGCATTTCACCGCTACACTTGGAA
TTCT (SEQ ID NO: 40)

| | | | | |
|---|---|---|---|---|
| 100 | 112 | 112 | 8.70E−54 | Rheinheimera sediminis strain |
| | | | | YQF-1 |

Sequence:
TGTTCGCTACCCACGCTTTCGCTCCTCAGCGTCAGTTACTGCCCAGAGACCCG
(SEQ ID NO: 41)

| | | | | |
|---|---|---|---|---|
| 100 | 53 | 53 | 1.94E−21 | Rhodococcus yananensis strain |
| | | | | FBM22-1 |

Sequence:
TGTTCGCTACCCATGCTTTCGCTCCTCAGCGTCAGTTACTACCCAGAGACCCGC
CTTCGCCACCGGTGTTCCTCCTGATATC (SEQ ID NO: 42)

| | | | | |
|---|---|---|---|---|
| 100 | 82 | 82 | 2.76E−37 | Dietzia massiliensis strain |
| | | | | Marseille-Q0999 |

Sequence:
TGTTCGCTCCCCACGCTTTCGCTCCTCAGCGTCAGGAAAGGCCCAGAGAACCG
CCTTCGCCACTGGTGTTCCTCCTGATATCTGCGCATTCCACCGCTCCACCAGGA
ATTCCATTCTCCCCTACCTTCCT (SEQ ID NO: 43)

| | | | | |
|---|---|---|---|---|
| 100 | 130 | 130 | 1.02E−63 | Cutibacterium acnes subsp. |
| | | | | elongatum strain K124 |

Sequence:
TGTTCGCTCCCCATGCTTTCGCTCCTCAGCGTCAGTTACGGCCCAGAGATCCG
(SEQ ID NO: 44)

| | | | | |
|---|---|---|---|---|
| 100 | 53 | 53 | 1.94E−21 | Angustibacter aerolatus strain |
| | | | | 7402J-48 |

Sequence:
TGTTTGCTACCCACGCTTTCGGGCCTCAGCGTCAGTGACAGACCAGAAAGTCG
CCTTCGCCACTGGTGTTCTTCCATATATCTACGCATTCCACCGCTACACATGGA
GTTCCACTTTCCTCTTCTGTACT (SEQ ID NO: 45)

| | | | | |
|---|---|---|---|---|
| 100 | 130 | 130 | 1.02E−63 | Aerococcus urinae strain NBRC |
| | | | | 15544 |

Sequence:
TGTTTGCTCCCCACGCTTTCGCACCTCAGTGTCAGTATCAGTCCAGGTGGTCGC
CTTCGCCACTGGTGTTCCTTCCTATATCTACGCATTT (SEQ ID NO: 46)

| | | | | |
|---|---|---|---|---|
| 100 | 91 | 91 | 3.15E−42 | Pseudomonas soyae strain JL117 |

Sequence:
TGTTTGCTCCCCACGCTTTCGCACCTCAGTGTCAGTATCAGTCCAGGTGGTCGC
CTTCGCCACTGGTGTTCCTTCCTATATCTACGTATTT (SEQ ID NO: 47)

| | | | | |
|---|---|---|---|---|
| 100 | 91 | 91 | 3.15E−42 | Pseudomonas saponiphila strain |
| | | | | DSM 9751 |

Sequence: TGTTTGCTCCCCACGCTTTCGCACCTGAGCGTCAGTCTTTGTCCAGG
(SEQ ID NO: 48)

| | | | | |
|---|---|---|---|---|
| 100 | 47 | 47 | 3.42E−18 | Klebsiella quasivariicola strain |
| | | | | KPN1705 |

Sequence: TGTTTGCTCCCCACGCTTTCGTGCATGAGCGTCAGTGCAG (SEQ ID
NO: 49)

| | | | | |
|---|---|---|---|---|
| 100 | 40 | 40 | 1.94E−14 | Comamonas fluminis strain CJ34 |

Sample alignment results of 16S-V4-R SPA fragments to the 16S rRNA gene database. For each fragment, the percentage of identity, fragment length and alignment length to a reference genome are indicated. E−values represent the probability of the alignment occurring by chance.

### Example 2

Primer Selection and SPA Protocol Based on the rpoB Gene as Phylogenetic Marker.

As a representative example, the SPA sequencing approach was successfully demonstrated for design of a rpoB gene specific SPA primer. A total of 50,569 unique rpoB gene sequences were downloaded from the PATRIC database (Wattam et al, 2014) using the version available in January 2021. RpoB gene sequences were identified based on their annotation as “DNA-directed RNA polymerase beta subunit (EC 2.7.7.6)”.

A subset of 50 rpoB gene sequences, representative for a broad range of phylogenetically distinct eubacterial reference microbes, were initially aligned by clustalW to identify conserved nucleotide regions of the rpoB gene, resulting in the identification of several conserved regions as primer candidates. This included the rpoB gene regions 1327-1352, 1528-1550, 1690-1709, 3766-3788 and 3808-3830, as well as the two regions identified by Ogier et al (2019), region 1630-1652 and region 2039-2063. The positions of the regions are based on the nucleotide sequence of the Escherichia coli rpoB gene.

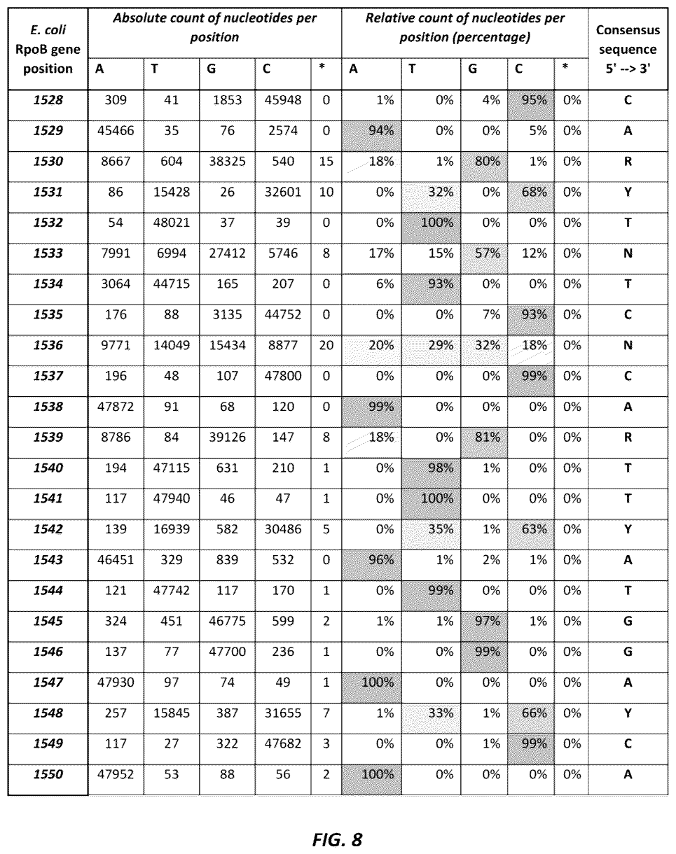

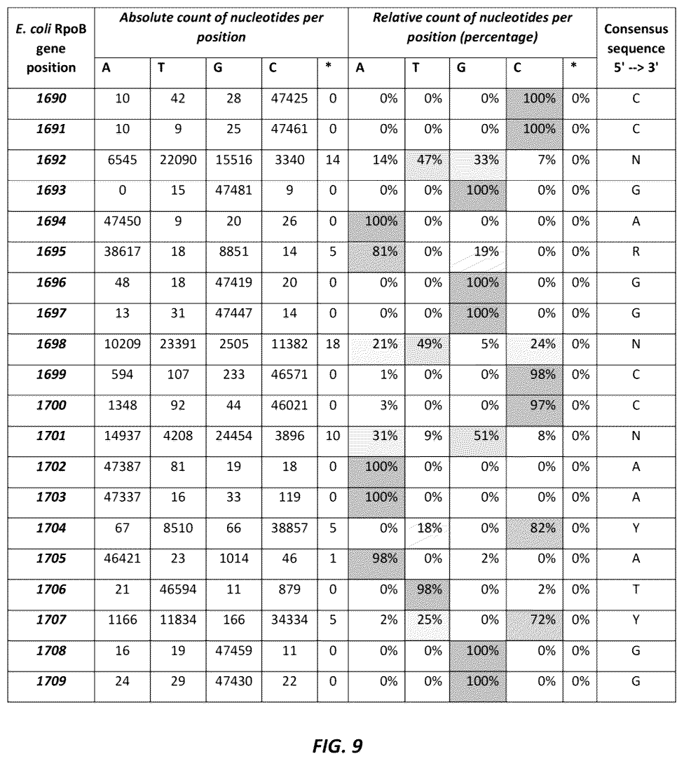

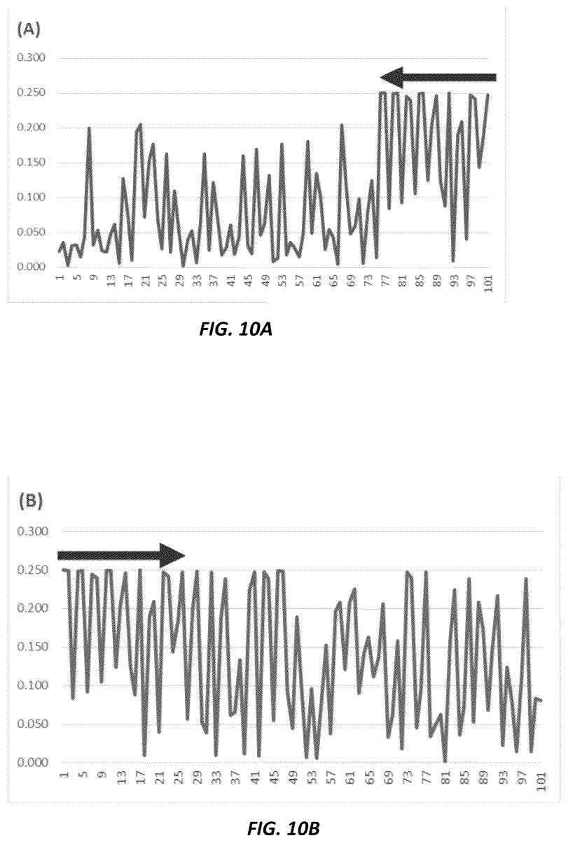

Using the SPA algorithm, the 50,569 unique rpoB genes sequences were aligned to these conserved regions to determine their nucleotide compositions. The conserved nucleotide sequences of the rpoB gene regions 1327-1352, 1528-1550 and 1690-1709 are provided in FIGS. 7A, 8 and 9 as representative examples. In Table 4, the average sequence variances for the primer candidates is shown, with all primer candidates having a similar score, making them all primer leads. Subsequently, the estimate of adjacent region conservation was calculated as described above. For each region, which represents a putative primer annealing site, the variance is shown for 25, 50, 75, 100 or 200 nucleotides (nt) upstream (5′) or downstream (3′) of the beginning or end of the sequence of the conserved region. The results are summarized in Table 4 and show that the nucleotide sequence upstream of the conserved region 1327-1352 is the most variable, as indicated by the lowest average variance scores of 0.0667 for both the 25 nucleotide-long and 50 nucleotide-long regions. This variability is also shown in FIGS. 10A and 10B, where the variance score for the 75 nucleotides upstream or downstream of the conserved region 1327-1352 has been plotted. FIGS. 10A and 10B also show the conservation of the nucleotides in the region 1327-1352, as well as the positions of the proposed degenerate primers RpoB1-R1327 and RpoB1-F1352, respectively. The sequences of the degenerate primers RpoB1-R1327 and RpoB1-F1352 are shown in Table 1. The identification of a hypervariable DNA region in the rpoB gene upstream of the conserved region 1327-1352 was unexpected, as it falls outside of the region that has previously been identified and used for RpoB gene amplicon sequencing (Ogier et al, 2019).

To select primers with the least risk for nonspecific annealing to human genomic DNA, the number of putative annealing sites of the proposed degenerate primer sequences to the human genome sequence (Reference: GCF_000001405.40_GRCh38.p14_genomic.fna) with increased number of allowed mismatches is determined. Results for the degenerate primers 16S-V3-F, 16S-V4-R, 16S-V6-R, RpoB6-F1652, RpoB7-R2039 and RpoB-R1327 are shown in Table 5. A primer should not have zero or one mismatch, and ideally no more than 10 instances of two mismatches with the human genome. Based on the results from this analysis, the primer 16S-V3-F showed an unexpectedly high number of putative annealing sites to the human genome, especially compared to the 16S-V4-R primer that also targets the V3-V4 region of the 16S rRNA gene and is, based on this result, considered unsuitable for SPA fragment sequencing.

TABLE 4
Average sequence variance for the primer regions and the regions upstream or downstream of candidate primer
annealing regions recognizing conserved rpoB gene sequences. For each region adjacent to the primer region,
the variance is shown for 25, 50, 75, 100 or 200 nucleotides (nt) upstream (5′) or downstream (3′) of
the beginning or end of the primer annealing sequence. The variance score is calculated as the average of the
variance of the percentage of the nucleotides adenine, guanidine, cytosine and thymine at each position of
the rpoB gene. A lower number is indicative for more variance, while a higher number is indicative for less variance
and a more conserved DNA sequence. The maximum theoretical variance score for a region is 0.25 (would represent
a 100% conserved DNA region). Regions with a variance score <0.1 are highlighted. The coordinates of the regions
recognized by the primers are based on the nucleotide sequence of the Escherichia coli rpoB gene.

| | Average of variance on RpoB gene | | | | | | | | | | |
|---|---|---|---|---|---|---|---|---|---|---|---|
| Primer name - | Region upstream of primer | | | | | | Region downstream of primer | | | | |
| recognized | 200 nt | 100 nt | 75 nt | 50 nt | 25 nt | Primer | 25 nt | 50 nt | 75 nt | 100 nt | 200 nt |
| RpoB gene | before | before | before | before | before | Primer | after | after | after | after | after |
| region | primer | primer | primer | primer | primer | region | primer | primer | primer | primer | primer |
| RpoB6 | 0.1356 | 0.1603 | 0.1548 | 0.1538 | 0.1546 | 0.1810 | 0.1492 | 0.1671 | 0.1633 | 0.1468 | 0.1199 |
| Forward - | | | | | | | | | | | |
| 1630-1652 | | | | | | | | | | | |
| RpoB7 | 0.1035 | 0.1393 | 0.1476 | 0.1576 | 0.1773 | 0.1964 | 0.1142 | 0.1312 | 0.1223 | 0.1077 | 0.0840 |
| Reverse - | | | | | | | | | | | |
| 2039-2063 | | | | | | | | | | | |
| RpoB1 - | 0.0495 | 0.0571 | 0.0675 | 0.0667 | 0.0667 | 0.1846 | 0.1390 | 0.1309 | 0.1240 | 0.1159 | 0.1170 |
| 1327-1352 | | | | | | | | | | | |
| RpoB2 - | 0.1123 | 0.1059 | 0.1139 | 0.1184 | 0.1266 | 0.1906 | 0.1368 | 0.1450 | 0.1491 | 0.1520 | 0.1477 |
| 1528-1550 | | | | | | | | | | | |
| RpoB3 - | 0.1525 | 0.1592 | 0.1616 | 0.1643 | 0.1632 | 0.1974 | 0.1160 | 0.1197 | 0.1247 | 0.1095 | 0.0836 |
| 1690-1709 | | | | | | | | | | | |
| RpoB4 - | 0.1277 | 0.1564 | 0.1665 | 0.1651 | 0.1418 | 0.1985 | 0.1846 | 0.1932 | 0.1808 | 0.1786 | 0.1584 |
| 3766-3788 | | | | | | | | | | | |
| RpoB5 - | 0.1513 | 0.1775 | 0.1748 | 0.1862 | 0.1879 | 0.2094 | 0.1614 | 0.1631 | 0.1620 | 0.1538 | 0.1384 |
| 3808-3830 | | | | | | | | | | | |

TABLE 5
Number of hits for primers to the human genome.
For each primer, the number of hits with zero, one or
two mismatches are presented. The number of hits was
determined based on homology to the nucleotide sequence
both DNA strands (+ and − strand) of the human chromosome
(Reference: GCF_000001405.40_GRCh38.p14_genomic.fna).

| | Number | Number | Number | Number |
|---|---|---|---|---|
| | of hits | of hits | of hits | of hits |
| | with zero | with one | with two | with three |
| | mismatch | mismatch | mismatches | mismatches |
| | (+strand; | (+strand; | (+strand; | (+strand; |
| Primer | −strand) | −strand) | −strand) | −strand) |
| 16S-V3-F | 1; 0 | 27; 36 | 1,049; 1,007 | 13,844; 13,496 |
| 16S-V4-R | 0; 0 | 0; 0 | 8; 2 | 67; 47 |
| RpoB6-F1652 | 0; 0 | 0; 0 | 1; 1 | 67; 61 |
| RpoB7-R2039 | 0; 0 | 0; 0 | 0; 0 | 2; 3 |
| RpoB1-R1327 | 0; 0 | 0; 0 | 1; 2 | 30; 26 |

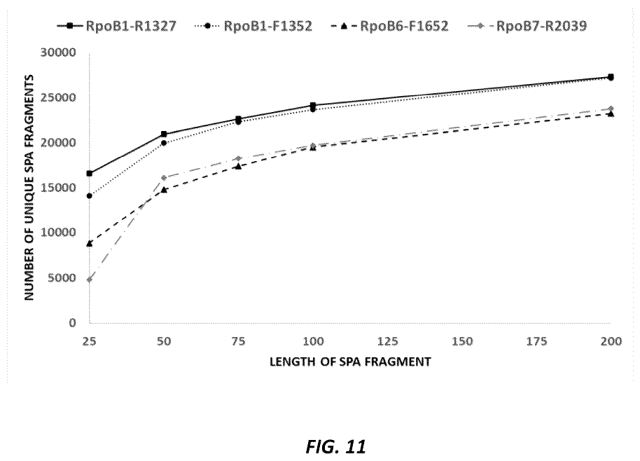

We subsequently analyzed the minimal length of the variable regions required to have sufficient sequence-based phylogenetic resolution for species level identification, while keeping in mind the size of mcfDNA fragments of approximately 40-100 bp as determined by Burnham et al (2016) and Rassoulian Barrett et al (2020). To do so we calculated the numbers of unique SPA fragments with length of 25, 50, 75, 100 and 200 nucleotides for the regions located downstream of the annealing sites for the RpoB1-R1327 and RpoB7-R2039 primers, and upstream of the RpoB1-F1352 and RpoB6-F1652 primers, respectively. The results are presented in FIG. 11 and show that the region upstream of the annealing site for primer RpoB1-R1327 consistently provided a higher number of unique SPA fragments compared to the other three primers, especially in the size range up to 75 nucleotides. For 50 nucleotide length, 20,919 unique SPA fragments could be generated for the upstream region. Based on the results presented in Table 4, FIGS. 10A and 10B, and FIG. 11 the degenerate RpoB-R1327 primer, which recognizes the conserved rpoB gene region 1327-1352 and allows for the generation of SPA fragments from the region upstream of the primer annealing site, was selected to validate in silico the Single Point Amplicon (SPA) fragment sequencing protocol for the rpoB gene and was added to our SPA primer repository.

The RpoB1-R1327 primer, which recognizes the rpoB gene sequence between positions 1327-1352 (positions based on the Escherichia coli rpoB gene sequence) and targets the region upstream of the primer annealing site, was validated in silico for the phylogenetic resolution of 50 nucleotide Single Point Amplification (SPA) fragments as described in EXAMPLES 3 to 9. In EXAMPLES 7 and 9 we also validated the RpoB6-R1630 primer, which recognizes the rpoB gene sequence between positions 1630-1652.

### Examples 3 to 9

To analyze their phylogenetic resolution, sequences of 50 nucleotide long SPA fragments located upstream of the RpoB1-R1327 priming site were generated in silico and analyzed on the genus or species level. Based on the size range of mcfDNA in blood of approximately 40-100 bp (Burnham et al, 2016) it very likely that SPA fragments of approximately 50 nucleotides can be obtained, this in addition to a small number of larger fragments. In EXAMPLES 3 to 9 we demonstrate that 50 nucleotide long SPA fragments provide sufficient phylogenetic resolution to distinguish a wide range of clinically relevant pathogenic bacteria at the species level. To further increase the resolution, we also validated in EXAMPLES 7 and 9 the RpoB6-R1630 primer, which recognizes the rpoB gene sequence between positions 1630-1652.

It should be noted that since we compare the SPA fragments for strain identification against a deduplicated database, the number of strains found for a SPA fragment represents the number of distinct rpoB gene sequences that share a common SPA fragment.

### Example 3

SPA fragment sequences for identification of Mycobacterium species.

Tuberculosis (TB) is an infectious disease for which cfDNA sequencing based diagnostics seems very promising. Clinical recognition of TB is hampered by its long latency and nonspecific presenting symptoms. In addition, people who have received the Bacillus Calmette-Guerin (BCG) vaccine cannot be tested for active TB using routine skin test screening (https://www.cdc.gov/tb/topic/testing/testingbcgvaccinated.htm). Of the estimated 10.4 million active TB cases occurring worldwide in 2016, it is estimated that 40% remained either undiagnosed or unreported, in large part due to inadequate diagnostics. Etiological diagnosis is typically delayed when reliant solely on the acid-fast bacillus (AFB) culture method, while invasive biopsies are often necessary to cultivate the pathogen from deep-seated infections. For an early diagnosis of tuberculosis, researchers have established several targeted Mycobacterium tuberculosis mcfDNA assays (PCR-based methods) to determine the presence of infection by detecting Mycobacterium tuberculosis mcfDNA in blood and urine specimens (Fernández-Carballo et al, 2019). More recently, the performance of deep plasma mcfDNA sequencing was evaluated in patients with tuberculosis infection, including the direct detection in a series of cases of invasive Mycobacterium chimaera infection (Nomura et al, 2019), providing accurate noninvasive microbiologic confirmation in approximately 4 days, which was more than one month faster than standard AFB culture method. Similarly, other successful applications in diseases such as opportunistic Mycobacterium avium or Mycobacterium tuberculosis infections in HIV/AIDS patients (Zhou et al, 2019) and aneurysms infected by Mycobacterium bovis due to Bacille Calmette-Guerin (BCG) instillation (Vudatha et al, 2019) demonstrate that mcfDNA analysis provides a promising, less-invasive diagnostic and monitoring tool for TB. Unfortunately, due to the need for costly deep NGS sequencing, mcfDNA sequencing is not feasible for routine and large-scale screening for TB. Therefore, there is an unmet need for high-resolution, high-throughput and low-cost detection of Mycobacterium tuberculosis and other disease-causing Mycobacterium strains, something SPA fragment sequencing can deliver. As such, TB and the detection of Mycobacterium species represents an important application for SPA fragment sequencing-based detection.

To evaluate its application for the reliable detection of TB and to analyze the discriminatory power of SPA fragment sequencing for high resolution phylogenetic identification of infecting Mycobacterium species, 50 nucleotide long SPA fragments located upstream of the RpoB1-R1327 priming site were generated in silico for Mycobacterium strains. The results are resented in Table 6.

TABLE 6

| Mycobacterium (My) specific SPA fragment | No. of |
|---|---|
| (50 nucleotides) sequence | strains |
| SPA fragment My1- | 291 |
| GTCAGACCACGATGACCGTTCCGGGCGGCGTCGAGGTGCCGGT | |
| GGAAACC (SEQ ID NO: 50) | |
| Mycobacterium tuberculosis | 286 |
| Mycobacterium tuberculosis subsp. africanum | 1 |
| Mycobacterium canettii | 3 |
| Mycobacterium orygis | 1 |
| SPA fragment My2- | 3 |
| GTCAGACCACGATGATCGTTCCGGGCGGCGTCGAGGTGCCGGT | |
| GGAAACC (SEQ ID NO: 51) | |
| Mycobacterium tuberculosis subsp. africanum | 1 |
| Mycobacterium tuberculosis | 2 |
| SPA fragment My3- | 42 |
| GCCAGACCACGATGACCGCCCCCGGTGGCGTCGAGGTGCCGGT | |
| GGATGTG (SEQ ID NO: 52) | |
| Mycobacterium abscessus | 42 |
| SPA fragment My4- | 37 |
| GCCAGACCACGATGACCGCCCCCGGCGGCGTCGAGGTGCCGGT | |
| GGACGTG (SEQ ID NO: 53) | |
| Mycobacterium abscessus | 34 |
| Mycobacterium abscessus subsp. massiliense | 3 |
| SPA fragment My5- | 9 |
| GCCAGACCACGATGACCGCCCCCGGCGGCGTCGAGGTGCCGGT | |
| GGATGTG (SEQ ID NO: 54) | |
| Mycobacterium abscessus | 9 |
| SPA fragment My6- | 5 |
| GCCAGACCACGATGACCGCCCCCGGGGGCGTCGAGGTGCCGGT | |
| GGATGTT (SEQ ID NO: 55) | |
| Mycobacterium abscessus | 5 |
| SPA fragment My7- | 4 |
| GCCAGACCACGATGACCGCCCCCGGGGGCGTCGAGGTGCCGGT | |
| GGATGTG (SEQ ID NO: 56) | |
| Mycobacterium abscessus | 4 |
| SPA fragment My8- | 3 |
| GTCAGCCCACGATGACCGTCCCGGGCGGCATCGAGGTGCCGGT | |
| GGAGACC (SEQ ID NO: 57) | |
| Mycobacterium avium | 3 |
| SPA fragment My9- | 6 |
| GTCAGCCCACGATGACCGTCCCCGGCGGCATCGAGGTGCCGGT | |
| GGAGACC (SEQ ID NO: 58) | |
| Mycobacterium avium | 4 |
| Mycobacterium MAC_011194 8550 | 1 |
| Mycobacterium MAC_080597_8934 | 1 |
| SPA fragment My10- | 2 |
| AGCCCGCTGTCATGACTGTCCCCGGCGGCATCGAGGTGCCGGT | |
| GGAGACC (SEQ ID NO: 59) | |
| Mycobacterium chimaera | 2 |
| SPA fragment My11- | 3 |
| GTCAGTCGACAATGACTGTCCCAGGTGGGGTAGAAGTGCCAGT | |
| GGAAACT (SEQ ID NO: 60) | |
| Mycobacterium leprae | 3 |
| SPA fragment My12- | 3 |
| GGCACGCCACGATGAAGGTCCCCGGTGGCGTCGAGGTGCCGGT | |
| GGAGACC (SEQ ID NO: 61) | |
| Mycobacterium xenopi | 3 |
| SPA fragment My13- | 12 |
| GCCAGCCCACGATGACCGTCCCCGGCGGCATCGAGGTGCCGGT | |
| GGAGACC (SEQ ID NO: 62) | |
| Mycobacterium intracellulare | 5 |
| Mycobacterium paraintracellulare | 7 |
| SPA fragment My14- | 4 |
| GCCAGGCCACGATGACCGTGCCGGGGGGGGTCGAGGTGCCGGT | |
| GGAAACC (SEQ ID NO: 63) | |
| Mycobacterium kansasii | 4 |
| SPA fragment My15- | 3 |
| AGCCCGCCGTCATGACTGTGCCCGGCGGGGTCGAGGTCCCGGT | |
| GGAAACC (SEQ ID NO: 64) | |
| Mycobacterium kansasii | 1 |
| Mycobacterium MK142 | 1 |
| Mycobacterium MK21 | 1 |
| SPA fragment My16- | 2 |
| GTGACCAGACGATGACCGCGCCCGGCGGCTCCGAGGTGCCCGT | |
| CGAGGTC (SEQ ID NO: 65) | |
| Mycobacterium gilvum | 2 |
| SPA fragment My17- | 8 |
| GCCAGACCACGATGACCGTCCCCGGCGGCGTCGAGGTCCCGGT | |
| CGAGGTG (SEQ ID NO: 66) | |
| Mycobacterium conceptionense | 1 |
| Mycobacterium neworleansense | 1 |
| Mycobacterium nonchromogenicum | 1 |
| Mycobacterium vulneris | 1 |
| Mycolicibacterium boenickei | 1 |
| Mycolicibacterium fortuitum | 2 |
| Mycolicibacterium senegalense | 1 |
| SPA fragment My18- | 18 |
| GCCAGACCGCGATGACCGCTCCGGGCGGTGTCGAGGTGCCGGT | |
| CGAGACC (SEQ ID NO: 67) | |
| Mycobacterium liflandii | 1 |
| Mycobacterium marinum | 12 |
| Mycobacterium pseudoshottsii | 1 |
| Mycobacterium shottsii | 1 |
| Mycobacterium ulcerans | 3 |
| SPA fragment My19- | 4 |
| GCCAGACCTCGATGACGGTGCCCGGCGGTGTCGAGGTGCCGGT | |
| CGAGGTG (SEQ ID NO: 68) | |
| Mycobacterium chlorophenolicum | 1 |
| Mycobacterium chubuense | 2 |
| Mycolicibacterium psychrotolerans | 1 |

Overview of the sequences of 50 nucleotide SPA fragments generated in silico for Mycobacterium species. For each SPA fragment, the Mycobacterium species and the number of strains is indicated. The SPA fragments representing 456 Mycobacterium strains are reported. Mycobacterium-specific (My) SPA fragments received a unique numerical identifier for reference in further analysis. Unique SPA fragments with a single Mycobacterium species hit were not reported.

The 50 nucleotide SPA fragments were found to be highly distinctive for clinically relevant Mycobacterium species, including Mycobacterium tuberculosis, Mycobacterium avium, Mycobacterium chimaera and Mycobacterium leprae. For instance, the dataset included 290 Mycobacterium tuberculosis plus Mycobacterium tuberculosis subsp. africanum strains that could be identified by two distinct SPA fragments, SPA fragments My1 and My2. SPA fragment My1 identified 291 strains. In addition to 286 Mycobacterium tuberculosis strains and one Mycobacterium tuberculosis subsp. africanum strain, this fragment was also present in three Mycobacterium canettii strains and one Mycobacterium orygis strain, both members of the Mycobacterium tuberculosis complex and very closely related to Mycobacterium tuberculosis.

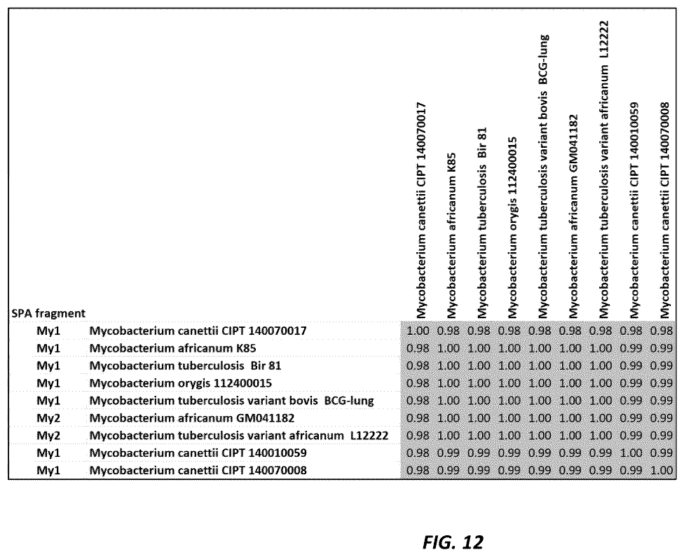

The similarities between the strains identified by SPA fragments My1 and My2 was analyzed using whole genome-based Average Nucleotide Identity (Arahal, 2014). The results are presented in FIG. 12 and show that representative strains of Mycobacterium tuberculosis, Mycobacterium tuberculosis subsp. africanum and Mycobacterium orygis shared ANI values of 100%, indicating that they represent identical species. The ANI values of these strains with the three Mycobacterium canettii strains ranged between 98% to 99%, similar to the ANI values shared between the three Mycobacterium canettii strains, indicating that all strains are very closely related and that Mycobacterium canettii is likely a Mycobacterium tuberculosis subspecies, as confirmed by the shared SPA fragment My1.

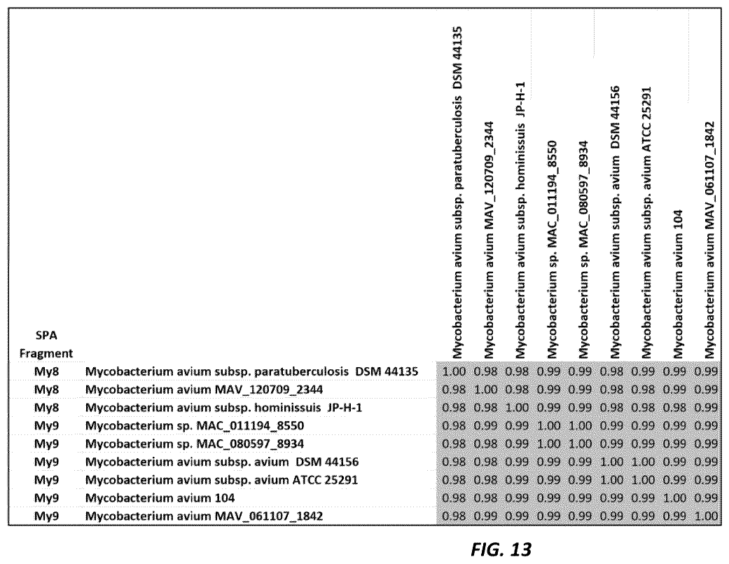

Mycobacterium avium strains, which can cause serious infection in immune compromised patients, such as HIV/AIDS patients, are identified by two distinct SPA fragments, My8 and My9. In addition to recognizing four Mycobacterium avium strains, SPA fragment My9 also identified two metagenome assembled genomes (MAG), Mycobacterium MAC_011194_8550 and Mycobacterium MAC_080597_8934. Based on the specificity of this fragment for Mycobacterium avium it is assumed that the two MAGs are representatives of Mycobacterium avium, as was confirmed by whole genome-based ANI analysis (FIG. 13).

The 97 strains belonging to Mycobacterium abscessus and Mycobacterium abscessus subsp. Massiliense could be identified by five distinct 50 nucleotide SPA fragments (My 3 to My7), with no other species being identified. Unique SPA fragments also identified the clinically relevant species Mycobacterium chimaera (My10) and Mycobacterium leprae (My11).

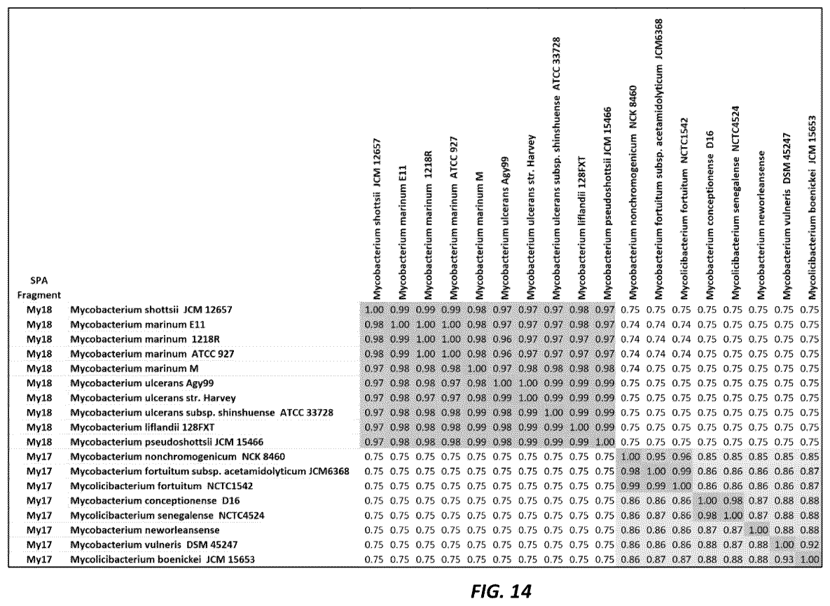

A few SPA fragments identified multiple distinct Mycobacterium species. For instance, eight strains of Mycobacterium conceptionense, Mycobacterium fortuitum (2 strains), Mycobacterium neworleansense, Mycobacterium nonchromogenicum, Mycobacterium vulneris, Mycolicibacterium boenickei, and Mycobacterium senegalense shared the common 50 nucleotide SPA fragment My17. Except for Mycobacterium nonchromogenicum, these strains all belong to the Mycolicibacterium gen. nov. (“fortuitum-vaccae” clade) and are very closely related (Gupto et al, 2018). It is generally accepted in the field that ANI values around 95% correspond to the 70% DNA-DNA hybridization cut-off value, which is widely used to delineate archaeal and bacterial species (Arahal, 2014). Whole genome-based ANI analysis (FIG. 14) showed that these strains indeed represent distinct species. Similar, closely related members of the emended genus Mycobacterium (“tuberculosis-simiae” clade) represented by Mycobacterium liflandii, Mycobacterium marinum, Mycobacterium pseudoshottsii, Mycobacterium shottsii and Mycobacterium ulcerans shared the common 50 nucleotide SPA fragment My18. In this specific case, the ANI values between the various strains ranged between 97% to 100%, confirming that they are closely related and part of the same genus Mycobacterium (“tuberculosis-simiae”) clade. This group (My18) is also highly distinct from the Mycobacterium strains identified by the SPA fragment My17, with ANI scores of 74% to 75% (FIG. 14). Increasing the length of the SPA fragments to 75 nucleotides did not significantly improve their phylogenetic resolution.

These results show that, unexpectedly, despite their relatively short size, sequences of 50 nucleotide long SPA fragments covering the region upstream of the RpoB1-R1327 primer annealing site allow for high resolution phylogenetic identification of Mycobacterium at the species or clade level (as summarized in Table 7), including the clinically relevant species. This shows the importance and potential of SPA fragment sequencing as a new approach for high-throughput TB screening, based on the (early) detection and identification of infectious Mycobacterium species using mcfDNA from peripheral blood and/or urine samples.

TABLE 7
Summary of the Mycobacterium (My) specific SPA fragments
as phylogenetic identifiers at the species or clade level. The SPA
fragments are 50 nucleotides in length and cover the
region upstream of the RpoB1-R1327 primer annealing site.

| Mycobacterium (My) | |
|---|---|
| specific SPA fragment | Species or Clade |
| SPA fragment My1, | Mycobacterium tuberculosis |
| SPA fragment My2 | |
| SPA fragment My3, | Mycobacterium abscessus |
| SPA fragment My4, | |
| SPA fragment My5, | |
| SPA fragment My6, | |
| SPA fragment My7 | |
| SPA fragment My8, | Mycobacterium avium |
| SPA fragment My9 | |
| SPA fragment My10 | Mycobacterium chimaera |
| SPA fragment My11 | Mycobacterium leprae |
| SPA fragment My12 | Mycobacterium xenopi |
| SPA fragment My13 | Mycobacterium (para)intracellulare |
| SPA fragment My14, | Mycobacterium kansasii |
| SPA fragment My15 | |
| SPA fragment My16 | Mycobacterium gilvum |
| SPA fragment My17 | Mycolicibacterium gen. nov. |
| | (“fortuitum-vaccae” clade) |
| SPA fragment My18 | Mycobacterium gen. |
| | (“tuberculosis-simiae” clade) |

### Example 4

SPA Fragment Sequences for the Detection of Bacterial Pathogens Associated with Pulmonary Infection Risks in Cystic Fibrosis Patients.

Cystic fibrosis (CF), the most common autosomal genetic disease in North America affecting 1:2000 Caucasian individuals, is characterized by chronic lung malfunction, pancreatic insufficiencies and high levels of chloride in sweat. Its high mortality index is evident when lung and spleen are affected, but other organs can also be affected. The persons affected die by progressive bronchiectasis and chronic respiratory insufficiency. CF patients will see a succession of lung inflammation by opportunistic pathogenic bacteria. During the first decade of life of CF patients, Staphylococcus aureus and Hemophilus influenzae are the most common bacteria, but in the second and third decade of life, Pseudomonas aeruginosa is the prevalent bacterium. Other important infectious bacterial pathogens associated with pulmonary infection risks in cystic fibrosis patients include Nontuberculous Mycobacteria (NTM) and Burkholderia cepacia (for review, see Coutinho et al, 2008). Therefore, there is an unmet need for high-resolution, high-throughput and low-cost detection of opportunistic pathogenic bacteria in CF patients, something SPA fragment sequencing can provide. The same is generally true for patients having a compromised immune system.

Mycobacterium species: The most common NTM infecting CF patients are Mycobacterium abscessus (identified by SPA fragments My3 to My7), Mycobacterium avium (identified by SPA fragments My8 and My9), and Mycobacterium (para)intracellulare (identified by SPA fragments My13), with Mycobacterium abscessus the NTM more likely associated with the disease, all of which can be identified by their unique SPA fragments (see Table 7).

Staphylococcus aureus: This is usually the first pathogen to infect and colonize the airways of CF patients. This microorganism is prevalent in children and may cause epithelial damage, opening the way to the adherence of other pathogens such as Pseudomonas aeruginosa. To evaluate its application for the reliable detection of chronic infection in CF patients by Staphylococcus aureus and related species, and to analyze the discriminatory power of SPA fragment sequencing for high resolution phylogenetic identification of infecting Staphylococcus species, 50 nucleotide long SPA fragments located upstream of the RpoB1-R1327 priming site were generated in silico for Staphylococcus strains. The results are presented in Table 8

TABLE 8

| Staphylococcus aureus (Sa) specific SPA fragment | No. of |
|---|---|
| (50 nucleotides) sequence | strains |
| SPA fragment Sa1- | 402 |
| TTGCTTCAATGAGTTACTTCTTTAACTTATTAAGCGGTATTGGAT | |
| ATACA (SEQ ID NO: 69) | |
| Staphylococcus aureus | 402 |
| SPA fragment Sa2- | 119 |
| TCGCTTCAATGAGTTACTTCTTTAACTTATTAAGTGGTATTGGAT | |
| ATACA (SEQ ID NO: 70) | |
| Staphylococcus aureus | 118 |
| Staphylococcus hyicus | 1 |
| SPA fragment Sa3- | 11 |
| TCGCTTCAATGAGTTATTTCTTTAACTTATTAAGTGGTATTGGAT | |
| ATACA (SEQ ID NO: 71) | |
| Staphylococcus argenteus | 8 |
| Staphylococcus aureus | 3 |
| SPA fragment Sa4- | 6 |
| TTGCTTCAATGAGTTATTTCTTTAACTTATTAAGTGGTATTGGAT | |
| ATACA (SEQ ID NO: 72) | |
| Staphylococcus aureus | 3 |
| Staphylococcus schweitzeri | 3 |
| SPA fragment Sa5- | 3 |
| TCGCTTCAATGAGTTACTTCTTTAACTTATTAAGCGGTATTGGAT | |
| ATACA (SEQ ID NO: 73) | |
| Staphylococcus aureus | 3 |
| SPA fragment Sa6- | 2 |
| TCGCTTCAATGAGTTACTTCTTTAATTTATTAAGTGGTATTGGAT | |
| ATACA (SEQ ID NO: 74) | |
| Staphylococcus aureus | 2 |
| SPA fragment Sa7- | 2 |
| GTTGAAACTTGCGCACATGGTTGATGATAAATTACATGCGCGTT | |
| CAACAG (SEQ ID NO: 75) | |
| Staphylococcus aureus | 2 |

Overview of the sequences of 50 nucleotide SPA fragments generated in silico for Staphylococcus aureus species. For each SPA fragment, the Staphylococcus species and the number of strains is indicated. The SPA fragments representing 545 Staphylococcus aureus and strains that shared their SPA fragment are reported. Staphylococcus aureus-specific (Sa) SPA fragments received a unique numerical identifier for reference in further analysis. Unique SPA fragments with a single Staphylococcus aureus species hit were not reported.

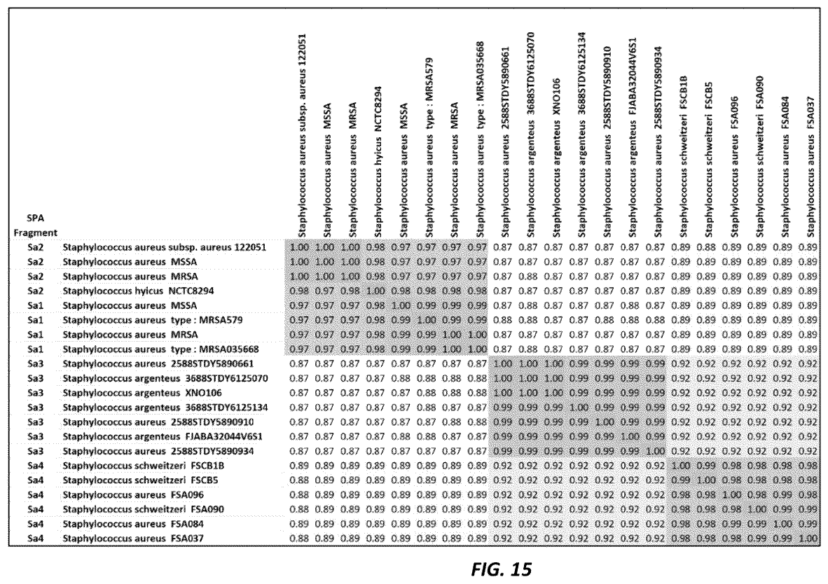

Based on the SPA fragment sequences, four mixed clusters were identified, each with their unique 50 nucleotide fragment (Table 8), that contained Staphylococcus aureus. Whole genome-based ANI analysis on representative members of these four clusters revealed that they grouped in three highly distinct species (FIG. 15).

ANI group I, comprised of strains identified by SPA fragments Sa1 and Sa2. With the exception of a single Staphylococcus hyicus strain, the 521 strains identified by Sa1 and Sa2 were all Staphylococcus aureus. Since the Staphylococcus hyicus strain had a 98% ANI score with the Staphylococcus aureus strains, similar to the score between Staphylococcus aureus strains, it also belongs to this species (Arahal, 2014). This confirms that SPA fragments Sa1 and Sa2 are specific for the identification of Staphylococcus aureus strains.

ANI group II, comprised of strains identified by SPA fragment Sa3. These strains had been previously identified as Staphylococcus argenteus and Staphylococcus aureus. Since these strains had ANI scores of 87% to 88% with the ANI group I Staphylococcus aureus strains, they represent a different species (Arahal, 2014), most likely Staphylococcus argenteus. Thus, SPA fragment Sa3 seems to be specific for the identification of Staphylococcus argenteus strains.

ANI group III, comprised of strains identified by SPA fragment Sa4. These strains had been previously identified as Staphylococcus schweitzeri and Staphylococcus aureus. Since these strains had ANI scores of 88% to 89% with the ANI group I Staphylococcus aureus strains and 92% with the ANI group II Staphylococcus argenteus strains, they represent a different species (Arahal, 2014), most likely Staphylococcus schweitzeri. Thus, SPA fragment Sa4 seems to be specific for the identification of Staphylococcus schweitzeri strains.

Despite their relatively short size, 50 nucleotide long SPA sequencing fragments covering the region upstream of the RpoB1-R1327 primer annealing site allow for high resolution phylogenetic identification of Staphylococcus at the species level (as summarized in Table 9), including the clinically relevant species Staphylococcus aureus and Staphylococcus argenteus.

TABLE 9
Summary of the Staphylococcus aureus (Sa) specific SPA
fragments as phylogenetic identifiers at the species level.
The SPA fragments are 50 nucleotides in length and
cover the region upstream of the RpoB1-R1327
primer annealing site.

| Staphylococcus (Sa) | |
|---|---|
| specific SPA fragment | Species |
| SPA fragment Sa1, | Staphylococcus aureus |
| SPA fragment Sa2, | |
| SPA fragment Sa5, | |
| SPA fragment Sa6, | |
| SPA fragment Sa7 | |
| SPA fragment Sa3 | Staphylococcus argenteus |
| SPA fragment Sa4 | Staphylococcus schweitzeri |

Pseudomonas aeruginosa This species is part of the normal microbial population of the respiratory tract, where it is an opportunistic pathogen in CF patients. Pseudomonas aeruginosa causes infections in more than 50% of CF patients, especially in adult CF patients, as infection has been shown in 20% CF patients 0-2 years old while in 81% in adult groups (>18 years old). To evaluate its application for the reliable detection of chronic infection in CF patients by Pseudomonas aeruginosa and related species, and to analyze the discriminatory power of SPA fragment sequencing for high resolution phylogenetic identification of infecting Pseudomonas aeruginosa species, 50 nucleotide long SPA fragments located upstream of the RpoB11-R1327 priming site were generated in silico for Pseudomonas aeruginosa strains. The results are presented in Table 10.

TABLE 10

| Pseudomonas aeruginosa (Pa) specific SPA fragment | No. of |
|---|---|
| (50 nucleotides) sequence | strains |
| SPA fragment Pal- | 543 |
| TCGATGTGCTCAAGACCCTCGTCGACATCCGTAACGGCAAGGGC | |
| ATCGTC (SEQ ID NO: 76) | |
| Pseudomonas aeruginosa | 532 |
| Pseudomonas FDAARGOS_761 | 1 |
| Pseudomonas fluorescens | 1 |
| Pseudomonas HMSC063H08 | 1 |
| Pseudomonas HMSC066A08 | 1 |
| Pseudomonas HMSC066B03 | 1 |
| Pseudomonas HMSC066B11 | 1 |
| Pseudomonas HMSC067F09 | 1 |
| Pseudomonas HMSC070B12 | 1 |
| Pseudomonas HMSC075A08 | 1 |
| Pseudomonas RW410 | 1 |
| Acinetobacter baumannii | 1 |
| SPA fragment Pa2- | 15 |
| TCGATGTGCTCAAGACCCTGGTCGACATCCGTAACGGCAAGGGC | |
| ATCGTC (SEQ ID NO: 77) | |
| Pseudomonas aeruginosa | 13 |
| Pseudomonas psychrotolerans | 1 |
| Pseudomonas SL25 | 1 |
| SPA fragment Pa3- | 3 |
| TCGATGTGCTCAAGACCCTCGTCGATATCCGTAACGGCAAGGGC | |
| ATCGTC (SEQ ID NO: 78) | |
| Pseudomonas aeruginosa | 3 |
| SPA fragment Pa4- | 3 |
| TCGAGGTCCTTAAGACCCTGGTCGATATCCGTAACGGCAAAGGC | |
| ATTGTC (SEQ ID NO: 79) | |
| Pseudomonas aeruginosa | 1 |
| Pseudomonas p99-361 | 1 |
| Pseudomonas putida | 1 |

Overview of the sequences of 50 nucleotide SPA fragments generated in silico for Pseudomonas aeruginosa species. For each SPA fragment, the Pseudomonas species and the number of strains is indicated. The SPA fragments representing 564 Pseudomonas aeruginosa and strains that shared their SPA fragment are reported. Pseudomonas aeruginosa-specific (Pa) SPA fragments received a unique numerical identifier for reference in further analysis. Unique SPA fragments with a single Pseudomonas aeruginosa species hit were not reported.

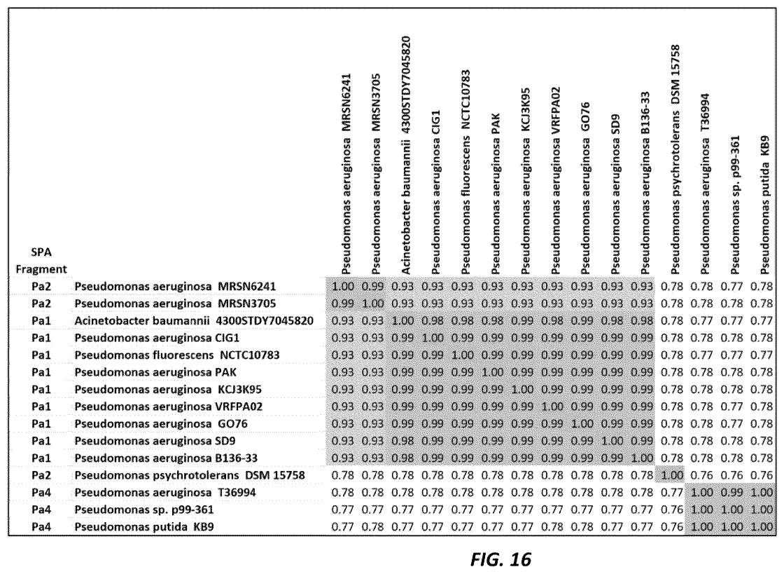

Based on the SPA fragment sequences, four clusters were identified, each with their unique 50 nucleotide fragment (Table 10), that contained Pseudomonas aeruginosa. ANI analysis on representative members of these four clusters revealed that they grouped in three highly distinct species (FIG. 16). Based on the results presented in FIG. 16, two major ANI groups can be distinguished for the Pseudomonas strains identified by the SPA fragments Pa1, Pa2 and Pa4.

ANI group I, which is comprised of strains identified by SPA fragments Pa1 and Pa2, represents Pseudomonas aeruginosa. Based on their ANI scores of 98% to 99%, the Pseudomonas fluorescens strain NCTC10783 and the Acinetobacter baumannii strain 4300STDY7045820 were previously misclassified and represent Pseudomonas aeruginosa strains. The only strain identified by SPA fragment Pa2 that fell outside of ANI group I was Pseudomonas psychrotolerans strain DSM 15758. This should cause no problem as this species, which grows at lower temperature than P. aeruginosa, is not clinically relevant.

ANI group III, which is comprised of strains identified by SPA fragments Pa4. This group, which includes three Pseudomonas strains, is based on its ANI score (76% to 78%) distinct from the Pseudomonas aeruginosa strains identified by SPA fragments Pa1 and Pa2.

Thus, despite their relatively short size, sequences of 50 nucleotide long SPA fragments covering the region upstream of the RpoB1-R1327 primer annealing site allow for high resolution phylogenetic identification of Pseudomonas aeruginosa at the species level (as summarized in Table 11).

TABLE 11
Summary of the Pseudomonas aeruginosa (Pa)
specific SPA fragments as phylogenetic identifiers at the
species level. The SPA fragments are 50 nucleotides
in length and cover the region upstream of
the RpoB1-R1327 primer annealing site.

| Pseudomonas aeruginosa | |
|---|---|
| (Pa) specific | |
| SPA fragment | Species |
| SPA fragment Pa1, | Pseudomonas |
| SPA fragment Pa2, | aeruginosa |
| SPA fragment Pa3 | |
| SPA fragment Pa4 | Pseudomonas species |

Burkholderia cepacia complex (BCC): A bacterial complex with twenty genomic species (genomovars): genomovar I (B. cepacia), II (B. multivorans), III (B. cenocepacia), IV (B. stabilis), V (B. vietnamiensis), VI (B. dolosa), VII (B. ambifaria), VIII (B. anthina), IX (B. pyrrocinia), and more recently B. stagnalis, B. territorii, B. ubonensis, B. contaminans, B, seminalis, B. metallica, B. arboris, B. lata, B. latens, B. pseudomultivorans, and B. diffusa was reported by Depoorter et al (2016). Infected CF patients show high levels of BCC in the salivary fluid, with transmission rates, prognosis and mortality being distinctly characteristic for each genomovar, as are the treatment strategies. Of the over 20 formally named species within the complex, Burkholderia multivorans (genomovar II) and Burkholderia cenocepacia (genomovar III) together account for approximately 85-97% of all BCC infections in CF (Savi et al, 2019). To evaluate its application for the reliable detection of chronic infection in CF patients by Bulkholderia cepacia and related BCC complex species, and to analyze the discriminatory power of SPA fragment sequencing for high resolution phylogenetic identification of infecting Burkholderia species, 50 nucleotide long SPA fragments located upstream of the RpoB1-R1327 priming site were generated in silico for Burkholderia strains. The results are presented in Table 12.

TABLE 12

| Burkholderia cepacia complex (Bcc) specific SPA fragment | No. of |
|---|---|
| (50 nucleotides) sequence | strains |
| SPA fragment Bcc1- | 486 |
| TCGCGACGATCAAGATCCTCGTCGAGCTGCGCAACGGCAAGGGC | |
| GAAGTG (SEQ ID NO: 80) | |
| Burkholderia strains | 51 |
| Burkholderia ambifaria-(VII) | 5 |
| Burkholderia anthina-(VIII) | 1 |
| Burkholderia cenocepacia-(III) | 50 |
| Burkholderia cepacia-(I) | 46 |
| Burkholderia contaminans-(XIII) | 12 |
| Burkholderia diffusa | 1 |
| Burkholderia lata | 1 |
| Burkholderia latens | 3 |
| Burkholderia metallica | 2 |
| Burkholderia multivorans-(II) | 68 |
| Burkholderia pseudomultivorans | 9 |
| Burkholderia pyrrocinia-(IX) | 5 |
| Burkholderia seminalis | 6 |
| Burkholderia stabilis-(IV) | 2 |
| Burkholderia stagnalis | 19 |
| Burkholderia territorii | 25 |
| Burkholderia thailandensis | 1 |
| Burkholderia ubonensis | 141 |
| Burkholderia vietnamiensis-(V) | 28 |
| Paraburkholderia bannensis | 1 |
| Paraburkholderia caryophylli | 1 |
| Paraburkholderia tropica | 2 |
| Paraburkholderia strains | 5 |
| Trinickia 7GSK02 | 1 |
| SPA fragment Bcc2- | 40 |
| TCGCGACGATCAAGATCCTCGTCGAACTGCGCAACGGCAAGGGC | |
| GAAGTG (SEQ ID NO: 81) | |
| Burkholderia ambifaria-(VII) | 1 |
| Burkholderia cepacia-(I) | 11 |
| Burkholderia diffusa | 9 |
| Burkholderia pyrrocinia-(IX) | 1 |
| Burkholderia ubonensis | 6 |
| Burkholderia strain | 10 |
| Paraburkholderia strain | 2 |
| SPA fragment Bcc3- | 9 |
| TCGCGACGATCAAGATCCTCGTCGAGTTGCGCAACGGCAAGGGC | |
| GAAGTG (SEQ ID NO: 82) | |
| Burkholderia cenocepacia-(III) | 4 |
| Burkholderia cepacia-(I) | 3 |
| Burkholderia dabaoshanensis* | 1 |
| Burkholderia LK4 | 1 |
| SPA fragment Bcc4- | 5 |
| TCGCGACGATCAAGATCCTCGTCGAGCTGCGCAACGGCAAGGGC | |
| GAAGTA (SEQ ID NO: 83) | |
| Burkholderia cepacia-(I) | 3 |
| Burkholderia territorii | 2 |
| SPA fragment Bcc5- | 4 |
| TCGCGACGATCAAGATCCTCGTCGAGCTGCGCAATGGCAAGGGC | |
| GAAGTG (SEQ ID NO: 84) | |
| Burkholderia lata | 1 |
| Burkholderia multivorans-(II) | 2 |
| Burkholderia ubonensis | 1 |
| SPA fragment Bcc6- | 14 |
| TCGCGACGATCAAGATCCTGGTCGAGCTGCGCAACGGCAAGGGC | |
| GAAGTG (SEQ ID NO: 85) | |
| Burkholderia strains | 2 |
| Burkholderia ubonensis | 4 |
| Burkholderia vietnamiensis-(V) | 6 |
| Paraburkholderia strains | 1 |
| SPA fragment Bcc7- | 9 |
| TCGCGACGATCAAGATTCTCGTCGAGCTGCGCAACGGCAAGGGC | |
| GAAGTG (SEQ ID NO: 86) | |
| Burkholderia strains | 4 |
| Burkholderia ubonensis | 5 |

Overview of the sequences of 50 nucleotide SPA fragments generated in silico for members of the Burkholderia cepacia complex. For each SPA fragment, the Burkholderia species and the number of strains is indicated. The SPA fragments representing 567 Burkholderia cepacia complex members (marked in bold) and related strains that shared their SPA fragment are reported. Burkholderia cepacia complex-specific (Bcc) SPA fragments received a unique numerical identifier for reference in further analysis. Unique SPA fragments with a single Burkholderia cepacia complex species hit were not reported.
*Indicates species whose name and has not been officially accepted.

Based on the SPA fragment sequences, seven clusters were identified, each with their unique 50 nucleotide fragment (Table 12), that contained Burkholderia cepacia. ANI analysis on representative members of various clusters defined by the SPA fragments Bcc1 (, Bcc1 and Bcc2, Bcc1 and Bcc3, and Bcc1, Bcc6 and Bcc7, revealed that 50 nucleotide SPA fragments fail to phylogenetically distinguish between the Burkholderia cepacia complex strains. In addition, a very limited number of strains that fall outside the Burkholderia cepacia complex were found to have similar SPA fragments. ANI analysis confirmed that these strains, such as Parabacteroides strains found to have SPA fragment Bcc1, were not misclassifie

To address the lack of phylogenetic resolution of 50 nucleotide SPA fragments for Burkholderia cepacia complex strains, larger SPA fragments were analyzed. Increasing the SPA fragment length to 75 nucleotides had only a minor effect on the phylogenetic resolution. For instance, the extended 75 nucleotide version of SPA fragment Bcc1 identified 479 strains, with the major difference being the removal of five Paraburkholderia strains. However, increasing the SPA fragment length to 100 nucleotides resulted in the breakup of the SPA fragment Bcc1 group with increased phylogenetic resolution that allowed for differentiation between several species belonging to the Burkholderia cepacia cluster. Since we expect to get for each species a limited number of SPA fragments with sizes around 100 nucleotides, as we showed in EXAMPLE 1, SPA fragment sequencing should allow for classification of Burkholderia cepacia cluster species with sufficient phylogenetic resolution. This is shown in Table 13 and Table 14 for the strains initially identified by the 50 nucleotide SPA fragment Bcc1.

TABLE 13

| Burkholderia cepacia complex (Bcc) specific SPA fragment | No. of |
|---|---|
| (100 nucleotides) | strains |
| SPA fragment Bcc8*- | 82 |
| CAGCCGTGACGAGATCACCGGCCCGATGACGCTGCAGGACGACGA | |
| CATCCTCGCGACGATCAAGATCCTCGTCGAGCTGCGCAACGGCAAG | |
| GGCGAAGTG (SEQ ID NO: 87) | |
| Burkholderia cepacia-(I) | 41 |
| Burkholderia contaminans-(XIII) | 11 |
| Burkholderia ambifaria-(VII) | 4 |
| Burkholderia pyrrocinia-(IX) | 4 |
| Burkholderia stabilis-(IV) | 2 |
| Burkholderia anthina-(VIII) | 1 |
| Burkholderia species | 19 |
| SPA fragment Bcc9*- | 3 |
| CGGCCGCGACGAGATCACCGGCCCGATGACGCTGCAGGACGACGA | |
| CATCCTCGCGACGATCAAGATCCTCGTCGAGCTGCGCAACGGCAAG | |
| GGCGAAGTG (SEQ ID NO: 88) | |
| Burkholderia ubonensis | 3 |
| SPA fragment Bcc10*- | 3 |
| CGGCCGTGACGAAATCACCGGCCCGATGACGCTGCAGGACGACGA | |
| CATCCTCGCGACGATCAAGATCCTCGTCGAGCTGCGCAACGGCAAG | |
| GGCGAAGTG (SEQ ID NO: 89) | |
| Burkholderia Bp5365 | 1 |
| Burkholderia thailandensis ($) | 1 |
| Burkholderia MSMB1588 | 1 |
| SPA fragment Bcc11*- | 68 |
| CGGCCGTGACGAAATCACGGGCCCGATGACGCTGCAGGACGACGA | |
| CATCCTCGCGACGATCAAGATCCTCGTCGAGCTGCGCAACGGCAAG | |
| GGCGAAGTG (SEQ ID NO: 90) | |
| Burkholderia multivorans-(II) | 67 |
| Paraburkholderia caryophylli | 1 |
| SPA fragment Bcc12*- | 34 |
| CGGCCGTGACGAAATCGTCGGCCCGATGACGCTGCAGGACGACGA | |
| CATCCTCGCGACGATCAAGATCCTCGTCGAGCTGCGCAACGGCAAG | |
| GGCGAAGTG (SEQ ID NO: 91) | |
| Burkholderia vietnamiensis-(V) | 27 |
| Burkholderia ubonensis | 3 |
| Paraburkholderia Cy-641 | 1 |
| Paraburkholderia CNPSo | 1 |
| Burkholderia species | 2 |
| SPA fragment Bcc13*- | 156 |
| CGGCCGTGACGAGATCACCGGCCCGATGACGCTGCAGGACGACGA | |
| CATCCTCGCGACGATCAAGATCCTCGTCGAGCTGCGCAACGGCAAG | |
| GGCGAAGTG (SEQ ID NO: 92) | |
| Burkholderia ubonensis | 131 |
| Burkholderia stagnalis | 19 |
| Burkholderia pyrrocinia-(IX) | 1 |
| Burkholderia multivorans-(II) | 1 |
| Burkholderia ambifaria-(VII) | 1 |
| Burkholderia species | 3 |
| SPA fragment Bcc14*- | 6 |
| CGGCCGTGACGAGATCATCGGCCCGATGACGCTGCAGGACGACGA | |
| CATCCTCGCGACGATCAAGATCCTCGTCGAGCTGCGCAACGGCAAG | |
| GGCGAAGTG (SEQ ID NO: 93) | |
| Burkholderia MSMB1498 | 1 |
| Burkholderia MSMB617WGS | 1 |
| Burkholderia MSMB2042 | 1 |
| Burkholderia BDU19 | 1 |
| Burkholderia BDU18 | 1 |
| Burkholderia MSMB0852 | 1 |
| SPA fragment Bcc15*- | 97 |
| CGGCCGTGACGAGATCGTCGGCCCGATGACGCTGCAGGACGACGA | |
| CATCCTCGCGACGATCAAGATCCTCGTCGAGCTGCGCAACGGCAAG | |
| GGCGAAGTG (SEQ ID NO: 94) | |
| Burkholderia cenocepacia-(III) | 47 |
| Burkholderia territorii | 25 |
| Burkholderia seminalis | 6 |
| Burkholderia cepacia-(I) | 4 |
| Burkholderia metallica | 1 |
| Burkholderia latens | 1 |
| Burkholderia species | 13 |
| SPA fragment Bcc16*- | 3 |
| CGGCCGTGATGAAATCGTCGGTCCGATGACGCTGCAGGACGACGA | |
| CATTCTCGCGACGATCAAGATCCTCGTCGAGCTGCGCAACGGCAAG | |
| GGCGAAGTG (SEQ ID NO: 95) | |
| Paraburkholderia species | 3 |
| SPA fragment Bcc17*- | 2 |
| CGGTCGCGACGAGATCGTCGGCCCGATGACGCTGCAGGACGACGA | |
| CATCCTCGCGACGATCAAGATCCTCGTCGAGCTGCGCAACGGCAAG | |
| GGCGAAGTG (SEQ ID NO: 96) | |
| Burkholderia ubonensis | 2 |
| SPA fragment Bcc18*- | 3 |
| CGGTCGTGACGAAATCGTCGGCCCGATGACGCTGCAGGACGACGA | |
| CATCCTCGCGACGATCAAGATCCTCGTCGAGCTGCGCAACGGCAAG | |
| GGCGAAGTG (SEQ ID NO: 97) | |
| Burkholderia latens | 2 |
| Burkholderia cenocepacia-(III) | 1 |
| SPA fragment Bcc19*- | 6 |
| GGGCCGTGACGAAATCACCGGCCCGATGACGCTGCAGGACGACGA | |
| CATCCTCGCGACGATCAAGATCCTCGTCGAGCTGCGCAACGGCAAG | |
| GGCGAAGTG (SEQ ID NO: 98) | |
| Burkholderia pseudomultivorans | 5 |
| Burkholderia TJI49 | 1 |
| SPA fragment Bcc20*- | 4 |
| GGGCCGTGACGAAATCGTCGGCCCGATGACGCTGCAGGACGACGA | |
| CATCCTCGCGACGATCAAGATCCTCGTCGAGCTGCGCAACGGCAAG | |
| GGCGAAGTG (SEQ ID NO: 99) | |
| Paraburkholderia tropica | 2 |
| Burkholderia vietnamiensis-(V) | 1 |
| Paraburkholderia bannensis | 1 |
| SPA fragment Bcc21*- | 3 |
| GGGTCGTGACGAAATCACCGGCCCGATGACGCTGCAGGACGACGA | |
| CATCCTCGCGACGATCAAGATCCTCGTCGAGCTGCGCAACGGCAAG | |
| GGCGAAGTG (SEQ ID NO: 100) | |
| Burkholderia pseudomultivorans | 2 |
| Burkholderia cenocepacia-(III) | 1 |
| SPA fragment Bcc22*- | 2 |
| CGGCCGCGACGAGATCGTCGGCCCGATGACGCTGCAGGACGACGA | |
| CATCCTCGCGACGATCAAGATCCTCGTCGAGCTGCGCAACGGCAAG | |
| GGCGAAGTG (SEQ ID NO: 101) | |
| Burkholderia USM | 1 |
| Burkholderia AU16741 | 1 |
| SPA fragment Bcc23*- | 2 |
| CGGCCGCGATGAAATCGTCGGCCCGATGACGCTGCAGGACGACGA | |
| CATCCTCGCGACGATCAAGATCCTCGTCGAGCTGCGCAACGGCAAG | |
| GGCGAAGTG (SEQ ID NO: 102) | |
| Trinickia 7GSK02 | 1 |
| Burkholderia DHOD12 | 1 |

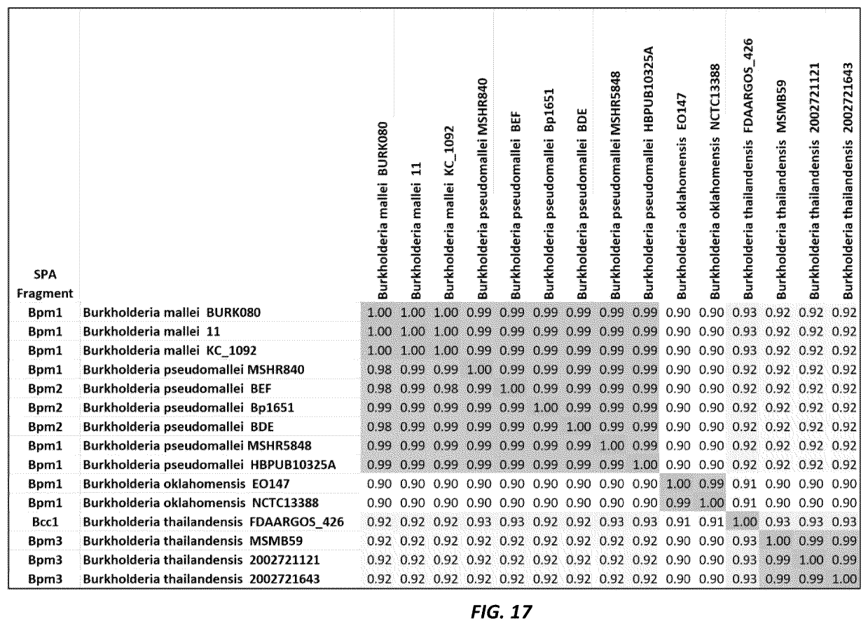

Overview of the sequences of 100 nucleotide SPA fragments generated in silico for members of the Burkholderia cepacia complex that share the SPA fragment Bcc1. For each SPA fragment, the Burkholderia species and the number of strains is indicated. The SPA fragments representing 471 Burkholderia cepacia complex members (marked in bold) and related strains that shared their SPA fragment are reported. Burkholderia cepacia complex- specific (Bcc) SPA fragments received a unique numerical identifier for reference in further analysis.
*Indicates 100 nucleotide SPA fragments. Unique SPA fragments with a single Burkholderia cepacia complex species hit were not reported. ($) indicates that Burkholderia thailandensis was incorrectly identified as this species, and as shown in FIG. 17 represents a new Burkholderia species.

Using 100 nucleotide long SPA sequencing fragments covering the region upstream of the RpoB1-R1327 primer annealing site significantly increased the resolution for phylogenetic identification of Burkholderia cepacia complex species, as is summarized in Table 14.

TABLE 14
Summary of the Burkholderia cepacia complex (Bcc) specific
SPA fragments and their phylogenetic resolution for strains that that
share the SPA fragment Bcc1. The SPA fragments are 100
nucleotides in length and cover the region upstream
of the RpoB1-R1327 primer annealing site.

| Burkholderia cepacia | |
|---|---|
| complex (Bcc) | |
| specific SPA fragment | Phylogenetic resolution |
| SPA fragment Bcc8* | Burkholderia cepacia complex |
| SPA fragment Bcc9* | Burkholderia ubonensis |
| SPA fragment Bcc10* | Burkholderia species Nov. |
| SPA fragment Bcc11* | Burkholderia multivorans-(II) |
| SPA fragment Bcc12* | Burkholderia cepacia complex ($) |
| SPA fragment Bcc13* | Burkholderia cepacia complex |
| SPA fragment Bcc14* | Burkholderia species Nov. |
| SPA fragment Bcc15* | Burkholderia cepacia complex |
| SPA fragment Bcc16* | Paraburkholderia species |
| SPA fragment Bcc16* | Burkholderia ubonensis |
| SPA fragment Bcc18* | Burkholderia cepacia complex |
| SPA fragment Bcc19* | Burkholderia pseudomultivorans |
| SPA fragment Bcc20* | Paraburkholderia species ($) |
| SPA fragment Bcc21* | Burkholderia cepacia complex |
| SPA fragment Bcc22* | Burkholderia species Nov. |
| SPA fragment Bcc23* | Trinickia species |

($) indicates the presence of species from outside the Burkholderia cepacia complex.

Burkholderia pseudomallei group: Most members of the Burkholderia pseudomallei group including Burkholderia mallei, Burkholderia oklahomensis and

Burkholderia pseudomallei are considered pathogenic. Table 15 shows that two unique SPA fragments, Bpm1 and Bpm2, reliably identified these clinically relevant species. Burkholderia thailandensis, also a member of the Burkholderia pseudomallei complex, is generally considered nonpathogenic. Burkholderia thailandensis could be identified by its own unique SPA fragment, Bpm3. This result, which was also confirmed by the ANI analysis of FIG. 16, further demonstrates the clinical relevance of the SPA as an important method for (early) detection and identification of Burkholderia species at the level of their major pathogenic complexes using mcfDNA from peripheral blood samples. The results form FIG. 17 also show that the Burkholderia thailandensis strain, previously shown to have SPA fragment Bcc1, was incorrectly identified as this species, but instead represents a new Burkholderia species.

TABLE 15
Overview of the sequences of 50 nucleotide SPA fragments generated in silico for
members of the Burkholderia pseudomallei group. For each SPA fragment, the Burkholderia
pseudomallei group species and the number of strains is indicated. The SPA fragments
representing 137 Burkholderia pseudomallei group members (marked in bold) and related
strains that shared their SPA fragment are reported. Burkholderia pseudomallei group-specific
(Bpm) SPA fragments received a unique numerical identifier for reference in further analysis.
Unique SPA fragments with a single Burkholderia pseudomallei group species hit were not
reported.

| Burkholderia pseudomallei (Bpm) specific SPA fragment (50 nucleotides) | No. of |
|---|---|
| sequence | strains |
| SPA fragment Bpm1 - | 119 |
| TCGCGACGATCAAGATCCTCGTCGAGCTGCGCAACGGCAAGGGC | |
| GAAGTC (SEQ ID NO: 103) | |
| Burkholderia 117 | 1 |
| Burkholderia ABCPW-14 | 1 |
| Burkholderia BDU8 | 1 |
| Burkholderia mallei | 8 |
| Burkholderia oklahomensis | 2 |
| Burkholderia pseudomallei | 105 |
| Paraburkholderia 7Q-K02 | 1 |
| SPA fragment Bpm2 - | 6 |
| TCGCGACGATCAAGATCCTCGTCGAGTTGCGCAACGGCAAGGGC | |
| GAAGTC (SEQ ID NO: 104) | |
| Burkholderia pseudomallei | 6 |
| SPA fragment Bpm3 - | 12 |
| TCGCGACGATCAAGATTCTCGTCGAGCTGCGCAACGGCAAGGGC | |
| GAAGTC (SEQ ID NO: 105) | |
| Burkholderia thailandensis | 12 |

Haemophilus influenzae: This species usually infects younger CF patients. For example, in Brazil, 20.4% of CF children between 6 and 12 years old are infected by Haemophilus influenzae. This bacterium hyper-mutates, which can be related to its resistance to antibiotics, making treatment more difficult. To evaluate its application for the reliable detection of chronic infection in CF patients by Haemophilus influenzae and related species, and to analyze the discriminatory power of SPA fragment sequencing for high resolution phylogenetic identification of infecting Haemophilus influenzae species, 50 nucleotide long SPA fragments located upstream of the RpoB1-R1327 priming site were generated in silico for Haemophilus influenzae strains. The results are presented in Table 16.

TABLE 16
Overview of the sequences of 50 nucleotide SPA fragments generated in silico for
Haemophilus influenzae species. For each SPA fragment, the Haemophilus influenzae species
and the number of strains is indicated. The SPA fragments representing 136 Haemophilus
influenzae strains and Haemophilus strains that shared their SPA fragment are reported.
Haemophilus influenzae -specific (Hi) SPA fragments received a unique numerical identifier
for reference in further analysis. Unique SPA fragments with a single Haemophilus influenzae
species hit were not reported.

| Haemophilus influenza (Hi) specific SPA fragment (50 nucleotides) | No. of |
|---|---|
| sequence | strains |
| SPA fragment Hi1 - | 79 |
| TTGCGGTAATGCGTAAATTGATCGACATCCGTAATGGTCGTGGC | |
| GAAGTA (SEQ ID NO: 106) | |
| Haemophilus aegyptius | 1 |
| Haemophilus HMSC066A11 | 1 |
| Haemophilus influenzae | 77 |
| SPA fragment Hi2 - | 14 |
| TTCGTGTGATGAAAAAACTCATCGATATCCGTAATGGTCGTGGT | |
| GAAGTG (SEQ ID NO: 107) | |
| Haemophilus HMSC068C11 | 1 |
| Haemophilus influenzae | 1 |
| Haemophilus parainfluenzae | 11 |
| Pasteurellaceae HGM20799 | 1 |
| SPA fragment Hi3 - | 12 |
| TTGCGGTAATGCGTAAATTGATTGACATCCGTAATGGTCGTGGC | |
| GAAGTA (SEQ ID NO: 108) | |
| Haemophilus influenzae | 12 |
| SPA fragment Hi4 - | 12 |
| TTCGTGTGATGAAAAAACTCATCGACATCCGTAATGGTCGTGGT | |
| GAAGTG (SEQ ID NO: 109) | |
| Haemophilus HMSC61B11 | 1 |
| Haemophilus parainfluenzae | 11 |
| SPA fragment Hi5 - | 7 |
| TTGCGGTAATGCGTAAATTGATTGACATCCGTAATGGTCGCGGC | |
| GAAGTA (SEQ ID NO: 110) | |
| Haemophilus influenzae | 7 |
| SPA fragment Hi6 - | 4 |
| TTCGTGTGATGAAAAAACTCATCGACATCCGTAATGGTCGTGGT | |
| GAAGTA (SEQ ID NO: 111) | |
| Haemophilus influenzae | 1 |
| Haemophilus parainfluenzae | 3 |
| SPA fragment Hi7 - | 3 |
| TTGCGGTAATGCGTAAATTAATCGACATCCGTAATGGTCGTGGC | |
| GAAGTA (SEQ ID NO: 112) | |
| Haemophilus haemolyticus | 1 |
| Haemophilus influenzae | 2 |
| SPA fragment Hi8 - | 3 |
| TCGCGGTAATGCGTAAATTGATTGACATCCGTAATGGTCGTGGC | |
| GAAGTA (SEQ ID NO: 113) | |
| Haemophilus influenzae | 3 |
| SPA fragment Hi9 - | 2 |
| TTGCGGTAATGCGTAAATTAATTGACATCCGTAATGGTCGTGGC | |
| GAAGTA (SEQ ID NO: 114) | |
| Haemophilus influenzae | 2 |

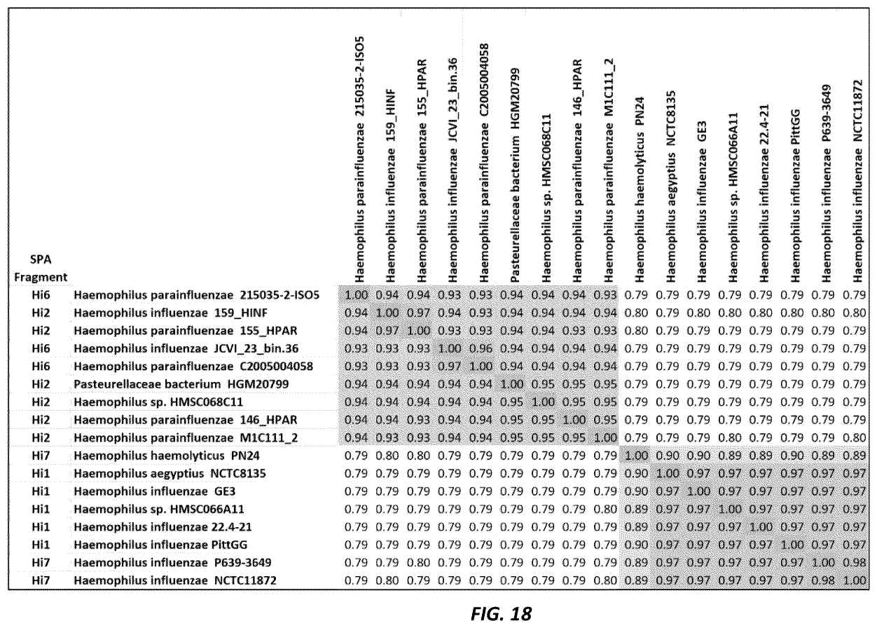

The species identified by the SPA fragments Hi1, H2, Hi6 and Hi7 were further analyzed by ANI, which resulted in the identification of two distinct ANI groups (FIG. 18):

ANI group I, comprised of strains identified by SPA fragments Hi2 and Hi6, represents the Haemophilus parainfluenzae strains. It also shows that Pasteurellaceae HGM20799, which has an ANI score of 94% to 95% with the other strains in this cluster, should be reclassifies as Haemophilus parainfluenzae.

ANI group II, comprised of strains identified by SPA fragments Hi1 and Hi7, represents the Haemophilus influenzae strains. It also shows that the Haemophilus aegyptius strain, which has ANI scores of 97% with the other strains in this cluster, should be reclassifies as Haemophilus influenzae. The Haemophilus haemolyticus strain, which was identified by SPA fragment Hi7, seems to be an outlier in this group with an ANI score of 89% with the other strains in this cluster.

Compared to other species, the ANI scores between members of the same ANI group are relatively low, around 95% instead of 98% to 99%. This might reflect the hyper-mutation phenotype of members of the genus Haemophilus. Overall, sequences of 50 nucleotide long SPA fragments covering the region upstream of the RpoB1-R1327 primer annealing site allow for high resolution phylogenetic identification of Haemophilus influenzae and Haemophilus parainfluenzae at the species level (as summarized in Table 17).

TABLE 17
Summary of the Haemophilus (para)influenzae (Hi)
specific SPA fragments as phylogenetic identifiers at the
species level. The SPA fragments are 50 nucleotides
in length and cover the region upstream of the
RpoB1-R1327 primer annealing site.

| Haemophilus influenza | |
|---|---|
| (Hi) specific | |
| SPA fragment | Species |
| SPA fragment Hi1, | Haemophilus |
| SPA fragment Hi3, | influenzae |
| SPA fragment Hi5, | |
| SPA fragment Hi7, | |
| SPA fragment Hi8, | |
| SPA fragment Hi9 | |
| SPA fragment Hi2, | Haemophilus |
| SPA fragment Hi4, | parainfluenzae |
| SPA fragment Hi6 | |

Overall, SPA fragments are capable of high resolution phylogenetic identification of opportunistic pathogenic bacteria frequently found to cause infections in CF patients. As such, SPA fragment sequencing represents a powerful tool to evaluate infections in CF patients as their treatment, including the selection of antibiotics, depends on the correct identification of the infectious species.

### Example 5

### SPA Fragment Sequences to Identify Opportunistic Bacterial Pathogens Linked to Sepsis.

Opportunistic pathogens of clinical relevance, including Pseudomonas aeruginosa, Mycobacterium abcessus, and Staphylococcus aureus, have been found as the cause of sepsis in patients with compromised immune systems. The successful use of SPA fragments for the high-resolution phylogenetic identification of these species has been described in EXAMPLES 3 and 4.

Streptococcus species, including S. pneumonia, S. pyogenes and S. intermedius are also frequently found as opportunistic pathogens in patients with compromised immune systems, such as HIV/AIDS patients, organ transplant patients or cancer patients undergoing chemotherapy. In addition, other clinically relevant Streptococcus species such as Streptococcus gallolyticus, Streptococcus macedonicus, Streptococcus pasteurianus and Streptococcus equinus, have been linked to cancer. Therefore, there is an unmet need for high-resolution, high-throughput and low-cost detection of opportunistic pathogenic Streptococcus species, something SPA fragment sequencing can provide. To evaluate its application for the reliable detection in peripheral blood of opportunistic pathogenic bacteria leading to sepsis by Streptococcus species, and to analyze the discriminatory power of SPA fragment sequencing for high resolution phylogenetic identification of infecting Streptococcus species, 50 nucleotide long SPA fragments located upstream of the RpoB1-R1327 priming site were generated in silico for Streptococcus strains. The results are presented in Table 18.

TABLE 18
Overview of the sequences of 50 nucleotide SPA fragments generated in silico for
Streptococcus species. For each SPA fragment, the Streptococcus species and the number of
strains is indicated. The SPA fragments representing 1,712 Streptococcus species and strains
that shared their SPA fragment are reported. Streptococcus-specific (St) SPA fragments
received a unique numerical identifier for reference in further analysis. Unique SPA fragments
with at least seven Streptococcus strain hit were reported, with the exception of Streptococcus
intermedius and Streptococcus gallolyticus subsp. gallolyticus

| | No. of |
|---|---|
| Streptococcus species (St) specific SPA fragment (50 nucleotides) sequence | strains |
| SPA fragment St1 - | 782 |
| TTGCTGAGATGAGCTACTTCCTCAACTTGGCTGAAGGACTTGGC | |
| CGTGTA (SEQ ID NO: 115) | |
| Streptococcus pneumoniae | 744 |
| Streptococcus pseudopneumoniae | 22 |
| Streptococcus mitis | 14 |
| Streptococcus D19 | 1 |
| Streptococcus OH4692_COT-348 | 1 |
| SPA fragment St2 - | 219 |
| TGGCAGAAATGTCTTACTTCTTGAACCTTGCTGAAGGTCTTGGAA | |
| AAGTT (SEQ ID NO: 116) | |
| Streptococcus dysgalactiae | 27 |
| Streptococcus NCTC | 1 |
| Streptococcus pyogenes | 189 |
| SPA fragment St3 - | 87 |
| TGGCAGAAATGTCTTACTTCTTGAACCTTGCAGAAGGTCTTGGA | |
| AAAGTT (SEQ ID NO: 117) | |
| Streptococcus pyogenes | 87 |
| SPA fragment St4 - | 19 |
| TGGCAGAAATGTCATACTTCTTGAACCTTGCTGAAGGTCTTGGA | |
| AAAGTT (SEQ ID NO: 118) | |
| Streptococcus pyogenes | 19 |
| SPA fragment St5 - | 75 |
| TAGCTGAAATGTCTTATTTCCTTAACTTGGCTGAGGGTCTAGGTA | |
| AAGTT (SEQ ID NO: 119) | |
| Streptococcus mutans | 75 |
| SPA fragment St6 - | 66 |
| TGGCTGAAATGAGCTACTTCCTCAACTTGGCTGAGGGTCTTGGT | |
| CGTGTA (SEQ ID NO: 120) | |
| Streptococcus suis | 66 |
| SPA fragment St7 - | 24 |
| TGGCTGAAATGAGCTACTTCCTCAACTTGGCTGAAGGACTTGGT | |
| CGCGTA (SEQ ID NO: 121) | |
| Streptococcus suis | 24 |
| SPA fragment St8 - | 47 |
| TTGCCGAGATGAGCTACTTCCTCAACTTGGCTGAAGGACTTGGC | |
| CGTGTA (SEQ ID NO: 122) | |
| Streptococcus mitis | 1 |
| Streptococcus pneumoniae | 46 |
| SPA fragment St9 - | 9 |
| TTGCTGAGATGAGCTACTTCCTCAACTTGGCTGAAGGCCTTGGC | |
| CGTGTA (SEQ ID NO: 123) | |
| Streptococcus mitis | 1 |
| Streptococcus pneumoniae | 3 |
| Streptococcus pseudopneumoniae | 5 |
| SPA fragment St10 - | 9 |
| TTGCTGAGATGAGTTACTTCCTCAACTTGGCTGAAGGACTTGGC | |
| CGTGTA (SEQ ID NO: 124) | |
| Streptococcus mitis | 4 |
| Streptococcus pneumoniae | 5 |
| SPA fragment St11 - | 9 |
| TTGCTGAAATGAGCTACTTCCTCAACTTGGCTGAAGGACTTGGC | |
| CGTGTA (SEQ ID NO: 125) | |
| Streptococcus mitis | 2 |
| Streptococcus pneumoniae | 5 |
| Streptococcus pseudopneumoniae | 1 |
| Streptococcus UMB0029 | 1 |
| SPA fragment St12- | 7 |
| TTGCTGAGATGAGCTACTTCCTCAACTTGGCTGAAGGGCTTGGC | |
| CGTGTA (SEQ ID NO: 126) | |
| Streptococcus mitis | 4 |
| Streptococcus pneumoniae | 3 |
| SPA fragment St13 - | 43 |
| TAGCAGAGATGTCATACTTCTTAAACCTTGCAGAGGGTATCGGT | |
| AAGGTA (SEQ ID NO: 127) | |
| Streptococcus agalactiae | 43 |
| SPA fragment St14 - | 27 |
| TGGCTGAGATGAGCTACTTCCTCAACTTAGCAGAAGGCATCGGC | |
| CGTGTG (SEQ ID NO: 128) | |
| Streptococcus anginosus | 2 |
| Streptococcus AS20 | 1 |
| Streptococcus constellatus | 8 |
| Streptococcus FDAARGOS_146 | 1 |
| Streptococcus HMSC067A03 | 1 |
| Streptococcus intermedius | 14 |
| SPA fragment St15 - | 3 |
| TGGCTGAGATGAGCTACTTCCTCAACTTAGCAGAGGGCATCGGC | |
| CGTGTG (SEQ ID NO: 129) | |
| Streptococcus intermedius | 3 |
| SPA fragment St16 - | 2 |
| TGGCTGAGATGAGCTACTTCCTCAACTTAGCAGAAGGCATCGGC | |
| CGTGTA (SEQ ID NO: 130) | |
| Streptococcus intermedius | 2 |
| SPA fragment St17 - | 11 |
| TGGCTGAGATGAATTACTTCTTGAACCTCGCTGAAGGACTTGGT | |
| CGTGTG (SEQ ID NO: 131) | |
| Streptococcus anginosus | 7 |
| Streptococcus constellatus | 1 |
| Streptococcus HF-100 | 1 |
| Streptococcus HF-2466 | 1 |
| Streptococcus KCOM | 1 |
| SPA fragment St18 - | 26 |
| TGGCTGAGATGTCTTATTTCCTTAACCTTGCTGAAGGTCTTGGAA | |
| AGGTC (SEQ ID NO: 132) | |
| Streptococcus equi | 26 |
| SPA fragment St19 - | 24 |
| TTGCAGAGATGAGCTACTTCCTTAACTTGGCAGAAGGTATCGGA | |
| CGTGTG (SEQ ID NO: 133) | |
| Streptococcus FDAARGOS 256 | 1 |
| Streptococcus GMDIS | 1 |
| Streptococcus GMD3S | 1 |
| Streptococcus mitis | 3 |
| Streptococcus oralis | 16 |
| Streptococcus pneumoniae | 1 |
| Streptococcus UMGS867 | 1 |
| SPA fragment St20 - | 9 |
| TTGCAGAGATGAGCTACTTCCTCAACTTGGCTGAAGGTATCGGA | |
| CGTGTG (SEQ ID NO: 134) | |
| Streptococcus GMD5S | 1 |
| Streptococcus HMSC066F01 | 1 |
| Streptococcus mitis | 1 |
| Streptococcus oralis | 6 |
| SPA fragment St21 - | 22 |
| TGGCTGAGATGAGCTACTTCCTCAACTTGGCAGAAGGTATCGGT | |
| CGTGTG (SEQ ID NO: 135) | |
| Streptococcus gordonii | 19 |
| Streptococcus mitis | 1 |
| Streptococcus oligofermentans | 2 |
| SPA fragment St22 - | 15 |
| TTGCAGAGATGAGCTACTTCCTCAACTTGGCGGAAGGTATCGGA | |
| CGTGTG (SEQ ID NO: 136) | |
| Streptococcus CM6 | 1 |
| Streptococcus mitis | 3 |
| Streptococcus NPS | 1 |
| Streptococcus oralis | 10 |
| SPA fragment St23 - | 16 |
| TGGCTGAAATGTCATACTTCTTAAATCTTTCTGAAGGGATTGGAA | |
| AAGTT (SEQ ID NO: 137) | |
| Streptococcus uberis | 16 |
| SPA fragment St24 - | 13 |
| TGGCAGAAATGAGCTATTTCTTGAACCTTGCAGAAGGTATTGGC | |
| CGCGTG (SEQ ID NO: 138) | |
| Streptococcus HMSC061E03 | 1 |
| Streptococcus HMSC072C09 | 1 |
| Streptococcus HMSC072G04 | 1 |
| Streptococcus JCVI_31A_bin.20 | 1 |
| Streptococcus parasanguinis | 9 |
| SPA fragment St25 - | 8 |
| TGGCAGAAATGAGCTATTTCTTGAACCTTGCAGAAGGCCTTGGC | |
| CGTGTA (SEQ ID NO: 139) | |
| Streptococcus parasanguinis | 8 |
| SPA fragment St26 - | 8 |
| TGGCTGAGATGAGCTACTTCCTCAACTTGGCTGAAGGCATTGGT | |
| CGCGTG (SEQ ID NO: 140) | |
| Streptococcus sanguinis | 8 |
| SPA fragment St27 - | 9 |
| TTGCAGAAATGTCTTATTTCTTAAACCTTTCTGAAGGTATTGGTA | |
| AAGTA (SEQ ID NO: 141) | |
| Streptococcus parauberis | 9 |
| SPA fragment St28 - | 9 |
| TGGCTGAAATGTCATACTTCCTTAACCTTGCTGAAGGTCTAGGTA | |
| AAGTT (SEQ ID NO: 142) | |
| Streptococcus CNU | 2 |
| Streptococcus infantarius | 3 |
| Streptococcus KCJ4932 | 1 |
| Streptococcus KCJ4950 | 1 |
| Streptococcus SL1232 | 1 |
| Streptococcus UBA11297 | 1 |
| SPA fragment St29 - | 7 |
| TTGCAGAAATGTCATATTTCTTGAACCTTGCAGAGGGTCTTGGAA | |
| AAGTT (SEQ ID NO: 143) | |
| Streptococcus iniae | 7 |
| SPA fragment St30 - | 61 |
| TGGCTGAAATGAGCTACTTCCTCAACCTTGCTGAAGGTATCGGT | |
| AAAGTA (SEQ ID NO: 144) | |
| Streptococcus 1004_SSPC | 1 |
| Streptococcus equinus | 1 |
| Streptococcus FDAARGOS_192 | 1 |
| Streptococcus HMSC068F04 | 1 |
| Streptococcus HMSC072D03 | 1 |
| Streptococcus salivarius | 13 |
| Streptococcus thermophilus | 39 |
| Streptococcus vestibularis | 4 |
| SPA fragment St31 - | 14 |
| TGGCTGAAATGAGTTACTTCCTCAACCTTGCTGAAGGTATCGGT | |
| AAAGTA (SEQ ID NO: 145) | |
| Streptococcus CCH5-D3 | 1 |
| Streptococcus HMSC10E12 | 1 |
| Streptococcus MGYG-HGUT-02550 | 1 |
| Streptococcus salivarius | 11 |
| SPA fragment St32 - | 12 |
| TGGCTGAAATGAGCTACTTCCTCAACCTTGCTGAAGGTATCGGT | |
| AAAGTT (SEQ ID NO: 146) | |
| Streptococcus CCH8-H5 | 1 |
| Streptococcus HMSC064H09 | 1 |
| Streptococcus JCVI_32_bin.27 | 1 |
| Streptococcus salivarius | 9 |
| SPA fragment St33 - | 14 |
| TGGCTGAAATGTCATACTTCCTTAATCTTGCTGAAGGTCTTGGTA | |
| AAGTT (SEQ ID NO: 147) | |
| Streptococcus bovis | 1 |
| Streptococcus gallolyticus subsp. gallolyticus | 3 |
| Streptococcus gallolyticus subsp. macedonicus | 4 |
| Streptococcus gallolyticus subsp. pasteurianus | 6 |
| SPA fragment St34 - | 10 |
| TGGCAGAAATGTCTTACTTCCTTAACCTTGCTGAAGGTCTAGGTA | |
| AAGTT (SEQ ID NO: 148) | |
| Streptococcus AS08sgBPME_176 | 1 |
| Streptococcus equinus | 8 |
| Streptococcus gallolyticus | 1 |
| SPA fragment St35 - | 3 |
| TGGCTGAAATGTCATACTTCCTTAACCTTGCTGAAGGTCTTGGTA | |
| AAGTT (SEQ ID NO: 149) | |
| Streptococcus gallolyticus subsp. gallolyticus | 3 |

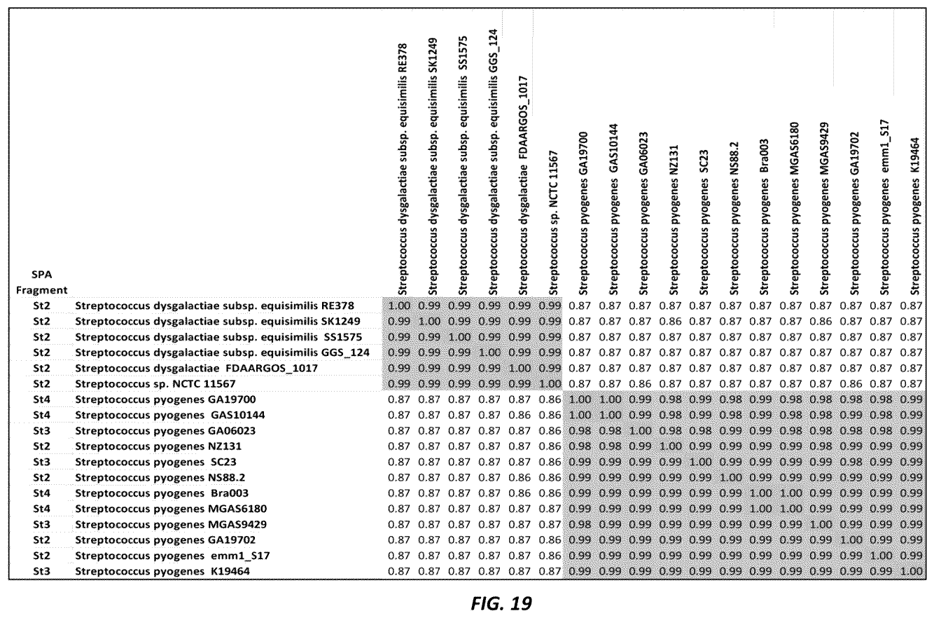

Overall, 50 nucleotide long SPA sequencing fragments covering the region upstream of the RpoB1-R1327 primer annealing site allow for high resolution phylogenetic identification of Streptococcus at the cluster or species level (as summarized in Table 20). For instance, unique SPA fragments were able to phylogenetically identify Streptococcus mutans, the cause of dental cavities; Streptococcus suis, a pathogen in pigs that can cause severe systemic infection in humans; Streptococcus agalactiae and Streptococcus equi, the causative agent of strangles which is the most frequently diagnosed infectious disease of horses; and Streptococcus parauberis, an important fish pathogen. When multiple species were identified by the same 50 nucleotide SPA fragment, whole genome-based ANI analysis on representative members was used to confirm the results based on the SPA fragments. Representative examples are shown in FIGS. 19 to 23, where ANI analysis was used to confirm the phylogenetic specificity of the Streptococcus SPA fragments.

The ANI results shown in FIG. 19 confirm that Streptococcus dysgalactiae is only identified by SPA fragment St2, while Streptococcus pyogenes is identified by SPA fragments St2, St3 and St4. Both species are very closely related and belong to the Lancefield Group A Streptococci.

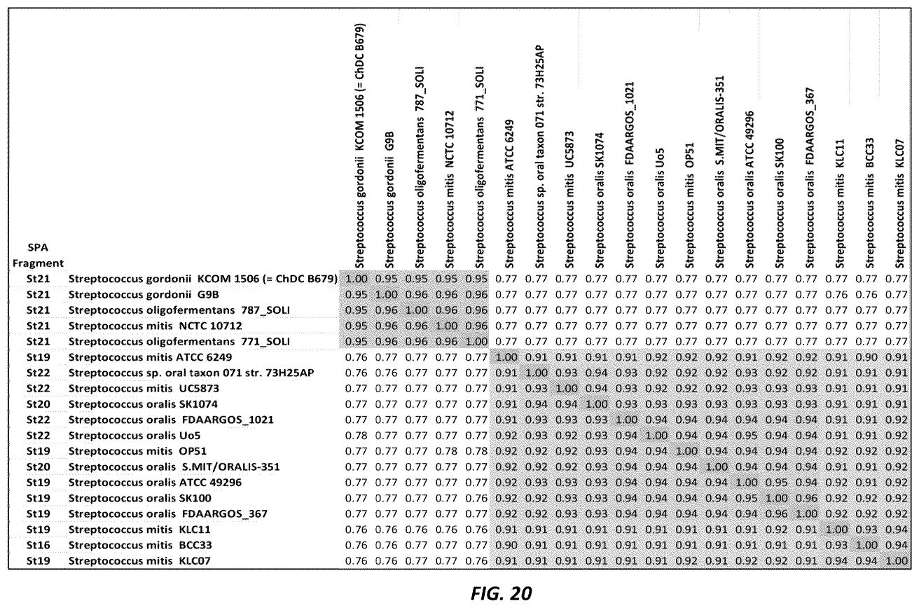

Similarly, based on ANI results, the SPA fragments St1, St8, St9, St10, St11 and St12 can be used to identify bacterial strains belonging to the Streptococcus mitis, Streptococcus pneumoniae and Streptococcus pseudopneumoniae cluster. Members of this cluster have previously been referred to as the viridans group streptococci (VGS), Streptococcus mitis group, and based on their ANI analysis, group together. A second group of strains, identified by the SPA fragments St19, St20 and St22, represents bacterial strains previously identified as Streptococcus mitis and Streptococcus oralis (FIG. 20). Based on their ANI score, these strains belong to a different group than those identified by the SPA fragments St1, St8, St9, St10, St11 and St12. As most of the strains identified by SPA fragments St19, St20 and St22 were identified as Streptococcus oralis, with ANI scores between the Streptococcus mitis and Streptococcus oralis strains of this ANI group being similar (91% to 94%) and significantly different from the ANI scores of the Streptococcus mitis/Streptococcus pneumoniae/Streptococcus pseudopneumoniae group members (86%), it is concluded that these strains are Streptococcus oralis. The results shown in FIG. 20 also confirm that the strains identified by SPA fragment St21 are Streptococcus gordonii and Streptococcus oligofermentans. Based on their ANI scores of 95% to 96% these two oral Streptococcus species are very closely related.

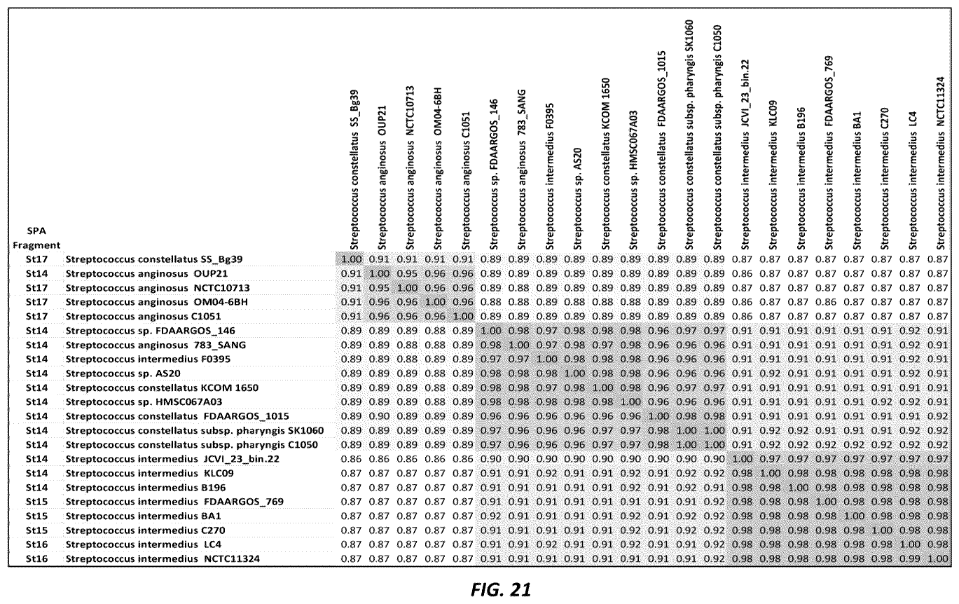

As shown in FIG. 21, Streptococcus anginosus, Streptococcus constellatus and Streptococcus intermedius form a cluster of tightly related strains. Based on ANI analysis, three ANI groups can be distinguished: ANI group I, comprised of Streptococcus anginosus strains identified by SPA fragments St14 and St17; ANI group III, comprised of Streptococcus intermedius strains identified by SPA fragments St14, St15 and St16; and ANI group II, comprised of Streptococcus anginosus, Streptococcus constellatus and Streptococcus intermedius strains all identified by SPA fragment St14. Based on their whole genome-based ANI scores, the ANI group II strains belong to the same species and are distinct from the Streptococcus anginosus, and Streptococcus intermedius strains of ANI groups I and II, and most likely represent Streptococcus constellatus.

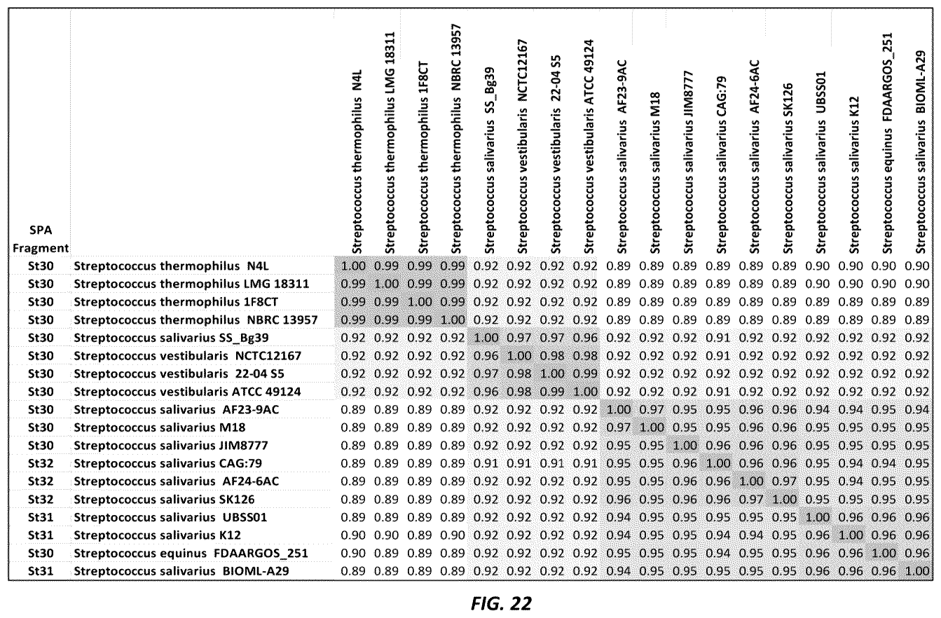

The whole genome-based ANI analysis for the Streptococcus equinus, Streptococcus salivarius, Streptococcus thermophilus and Streptococcus vestibularis strains identified by SPA fragments St30, St31 and St32 is shown in FIG. 22 and identifies three distinct ANI groups: ANI group I and II representing Streptococcus thermophilus strains and Streptococcus vestibularis strains, respectively, identified by SPA fragment St30; and ANI group III representing Streptococcus salivarius strains identified by SPA fragments St30, St31 and St32. Based on the ANI score it can also be concluded that Streptococcus equinus strain FDAARGOS_251, identified by SPA fragment St30, was misidentified and represents a Streptococcus salivarius strain.

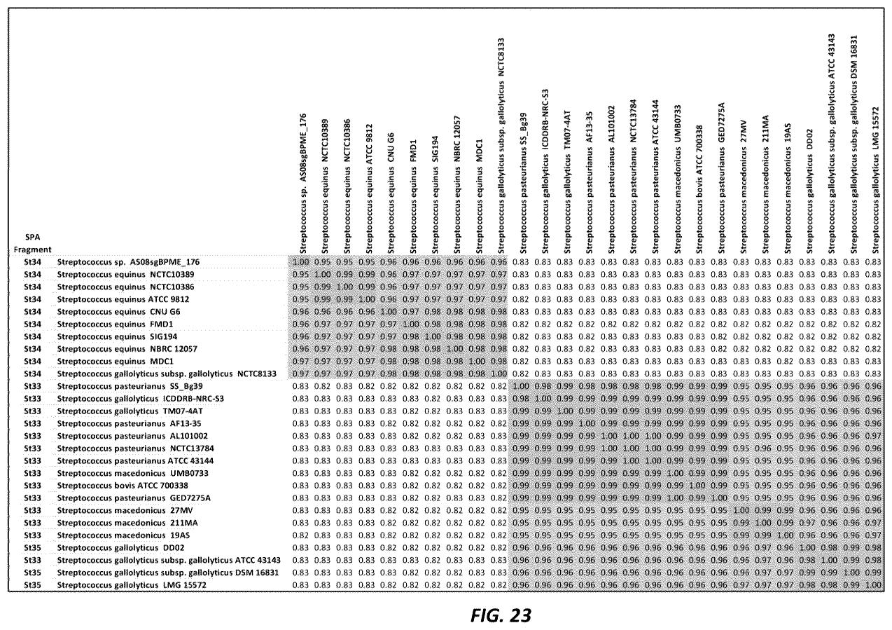

Streptococcus gallolyticus subsp. gallolyticus (formerly known as Streptococcus bovis type I) has recently been recognized as the main causative agent of septicemia and infective endocarditis in elderly and immunocompromised persons. It also has been strongly associated to colorectal cancer (CRC; defined as carcinomas and premalignant adenomas) (Boleij et al, 2011; Pasquereau-Kotula et al, 2018). Several previous studies failed to clearly attribute an association between Streptococcus bovis and CRC; this can, however, be explained by the lack of a proper distinction between Streptococcus bovis type I (Streptococcus gallolyticus strains), type 11.1 (Streptococcus infantarius) and type I1.2 (Streptococcus gallolyticus subsp. macedonicus and Streptococcus gallolyticus subsp. pasteurianus), with Streptococcus bovis type I being prevalently associated to CRC, and to a lesser extend Streptococcus bovis type I1.2 (Abdulamir et al, 2011). The phylogenetic resolution of 50 nucleotide SPA fragments allowed to discriminate between Streptococcus infantarius (SPA fragment St28) and Streptococcus gallolyticus (SPA fragments St33 and St35) strains. Therefore, SPA fragment sequencing represents a promising approach for CRC screening based on the presence of Streptococcus gallolyticus strains (Streptococcus bovis type I and I1.2) in peripheral blood. The whole genome-based ANI analysis presented in FIG. 23 show that the three subspecies Streptococcus gallolyticus subsp. gallolyticus, Streptococcus gallolyticus subsp. Macedonicus and Streptococcus gallolyticus subsp. pasteurianus are very closely related. It also shows that Streptococcus gallolyticus subsp. gallolyticus NCTC8133 should be classified as Streptococcus equinus.

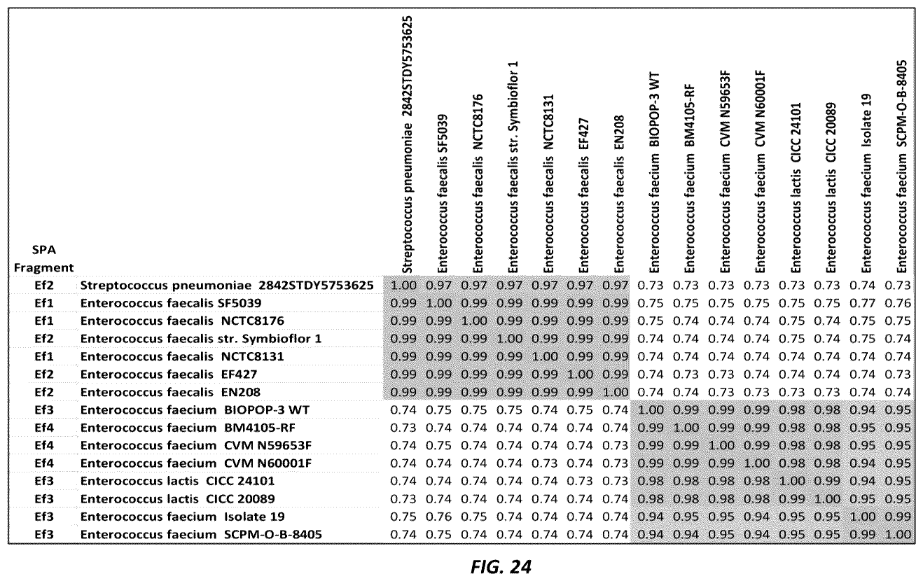

Since Enterococcus faecalis and Enterococcus faecium also belong to the Lancefield group D “Streptococci” (Table 20), the phylogenetic resolution of their 50 nucleotide SPA fragments was determined (Table 19). SPA fragments Ef1 and Ef2 were found to be specific for Enterococcus faecalis, while SPA fragments Ef3 and Ef4 were found to be specific for Enterococcus faecium. The results of the whole genome-based ANI analysis, shown in FIG. 24, confirmed the separate clustering of these two species. It also confirmed the misclassification of the Streptococcus pneumoniae and the Enterococcus lactis strains listed in Table 18 among the Enterococcus faecalis and Enterococcus faecium strains identified by SPA fragments Ef2 and Ef3. Based on their ANI scores, these strains should be reclassified as Enterococcus faecalis and Enterococcus faecium strains, respectively.

TABLE 19
Overview of the sequences of 50 nucleotide SPA fragments generated in silico for
Enterococcus faecalis and Enterococcus faecium strains. For each SPA fragment, the
Enterococcus faecalis and Enterococcus faecium species and the number of strains is indicated.
The SPA fragments representing 266 Enterococcus species and strains that shared their SPA
fragment are reported. Enterococcus faecalis and Enterococcus faecium-specific (Ef) SPA
fragments received a unique numerical identifier for reference in further analysis. Unique SPA
fragments with a single Enterococcus faecalis or Enterococcus faecium species hit were not
reported.

| Enterococcus faecalis and Enterococcus faecium (Ef) specific SPA fragment | No. of |
|---|---|
| (50 nucleotides) sequence | strains |
| SPA fragment Ef1 - | 125 |
| TTGCTTCAATGAGCTACTTCTTCAACTTAATGGAAGATATCGGCA | |
| ATGTC (SEQ ID NO: 150) | |
| Enterococcus faecalis | 125 |
| SPA fragment Ef2 - | 17 |
| TTGCTTCAATGAGCTACTTCTTCAACTTAATGGAAGATATCGGTA | |
| ATGTC (SEQ ID NO: 151) | |
| Enterococcus faecalis | 16 |
| Streptococcus pneumoniae | 1 |
| SPA fragment Ef3 - | 111 |
| TTGCTTCAATGAGCTATTTCTTGAACTTGATGGAAGGTATCGGCA | |
| ATGTC (SEQ ID NO: 152) | |
| Enterococcus faecium | 108 |
| Enterococcus lactis | 2 |
| Enterococcus N4D85 | 1 |
| SPA fragment Ef4 - | 13 |
| TTGCTTCAATGAGCTATTTCTTGAACTTGATGGAAGGTATCGGCA | |
| ATGTT (SEQ ID NO: 153) | |
| Enterococcus faecium | 12 |
| Enterococcus FM11-1 | 1 |

TABLE 20
Summary of the phylogenetic specificity of 50 nucleotide SPA
fragments generated upstream of the RpoB1-R1327 primer
annealing site for clinically relevant Streptococcus species
(SPA fragments St1 to St35) and Enterococcus species
(SPA fragments Ef1 to Ef4). Where applicable, the Lancefield
group (Lancefield, 1933) or the viridans group streptococci
(VGS) subgroup are indicated, as well as the standard of care
antibiotic treatment for infections caused by specific
Streptococcus species.

| Streptococcus | | | Preferred |
|---|---|---|---|
| (St) specific | | | antibiotic |
| SPA fragment | Species | Group | treatment |
| SPA fragment St1, | Streptococcus mitis, | Viridans | Amoxicillin |
| SPA fragment St9, | Streptococcus | group | alone or |
| SPA fragment St11 | pneumoniae, | streptococci | amoxicillin/ |
| | Streptococcus | (VGS), | clavulanic |
| | pseudopneumoniae | S. mitis | acid, a |
| | | group | fluoroquinolone |
| | | | or ceftriaxone. |
| SPA fragment St2 | Streptococcus | Lancefield | Penicillin; |
| | dysgalactiae or | group | Erythromycin, |
| | Streptococcus | | clindamycin |
| | pyogenes | | (resistance |
| | | | increasing in |
| | | | the US). |
| SPA fragment St3, | Streptococcus | Lancefield | Penicillin; |
| SPA fragment St4 | pyogenes | group A | Erythromycin, |
| | | | clindamycin |
| | | | (resistance |
| | | | increasing in |
| | | | the US). |
| SPA fragment St5 | Streptococcus | Viridans | Ampicillin, |
| | mutans | group | ceftotaxime |
| | | streptococci | cefazolin, |
| | | (VGS), | methicillin and |
| | | S. mutans | clindamycin as |
| | | group | most common |
| | | | treatments. |
| SPA fragment St6, | Streptococcus suis | Lancefield | Common pathogen |
| SPA fragment St7 | | group R & S | in pigs. Beta-lactam |
| | | | antibiotics (penicillin, |
| | | | ceftriaxone and |
| | | | ceftiofur) and |
| | | | fluoroquinalone |
| | | | antibiotics such as |
| | | | enrofloxacin. |
| SPA fragment St8, | Streptococcus mitis, | Viridans | Amoxicillin alone or |
| SPA fragment St10, | Streptococcus | group | amoxicillin/clavulanic |
| SPA fragment St12 | pneumoniae | streptococci | acid, a |
| | | (VGS), | fluoroquinolone |
| | | S. mitis | or ceftriaxone. |
| | | group | |
| SPA fragment St13 | Streptococcus | Lancefield | Penicillin, ampicillin, |
| | agalactiae | group B | and other β-lactams; |
| | | | cephalosporins, |
| | | | vanomycin. |
| SPA fragment St14 | Streptococcus | S. anginosus | Penicillin, ampicillin, |
| | anginosus, | group; | and other β-lactams. |
| | Streptococcus | Group F, | |
| | intermedius, | G & L | |
| | Streptococcus | | |
| | constellatus | | |
| SPA fragment St15, | Streptococcus | S. anginosus | Penicillin, ampicillin, |
| SPA fragment St16 | intermedius | group | and other β-lactams. |
| SPA fragment St17 | Streptococcus | S. anginosus | Penicillin, ampicillin, |
| | anginosus, | group; | and other β-lactams. |
| | Streptococcus | Lancefield | |
| | constellatus | group F, | |
| | | G & L | |
| SPA fragment St18 | Streptococcus | Lancefield | Major horse pathogen. |
| | equi. subsp. | group C | Penicillin, ceftiofur, |
| | zoopidemicus | | or ampicillin. |
| SPA fragment St19 | Streptococcus oralis, | Viridans | Amoxicillin alone or |
| | Streptococcus | group | amoxicillin/clavulanic |
| | pneumoniae | streptococci | acid, a |
| | | (VGS), | fluoroquinolone |
| | | S. mitis | or ceftriaxone. |
| | | group | |
| SPA fragment St20, | Streptococcus oralis | viridans | Amoxicillin alone or |
| SPA fragment St22 | | group | amoxicillin/clavulanic |
| | | (VGS), | acid, a |
| | | S. mitis | fluoroquinolone |
| | | group | or ceftriaxone. |
| SPA fragment St21 | Streptococcus gordonii | Viridans | Combined treatment |
| | | group | with vancomycin- |
| | | (VGS), | gentamicin, imipenem- |
| | | S. sanguinis | gentamicin and |
| | | group | teicoplanin-gentamicin |
| | | | in patients with |
| | | | infective |
| | | | endocarditis caused by |
| | | | penicillin-resistant |
| | | | Streptococcus sanguinis |
| | | | group bacteria. |
| SPA fragment St23 | Streptococcus uberis | Some strains | Responsible for a high |
| | | are reported | percentage of |
| | | to belong to | mastitis in |
| | | Lancefield | dairy cattle and it is |
| | | group E, G, | rarely associated with |
| | | P, or U | human infections. |
| SPA fragment St24, | Streptococcus | Viridans | Combined treatment |
| SPA fragment St25 | parasanguinis | group | with vancomycin- |
| | | (VGS), | gentamicin, imipenem- |
| | | S. sanguinis | gentamicin and |
| | | group | teicoplanin-gentamicin |
| | | | in patients with |
| | | | infective |
| | | | endocarditis caused by |
| | | | penicillin-resistant |
| | | | Streptococcus sanguinis |
| | | | group bacteria. |
| SPA fragment St26 | Streptococcus sanguinis | Viridans | Combined treatment |
| | | group | with vancomycin- |
| | | (VGS), | gentamicin, imipenem- |
| | | S. sanguinis | gentamicin and |
| | | group | teicoplanin-gentamicin |
| | | | in patients with |
| | | | infective |
| | | | endocarditis caused by |
| | | | penicillin-resistant |
| | | | Streptococcus sanguinis |
| | | | group bacteria. |
| SPA fragment St27 | Streptococcus | Non- | Amoxicillin, |
| | parauberis | Lancefield | erythromycin, |
| | | Streptococcus | vancomycin. |
| SPA fragment St28 | Streptococcus | Streptococcus | Pencillin, ampicillin, |
| | infantarius | bovis/ | vancomycin (plus an |
| | | Streptococcus | aminoglycoside for |
| | | equinus | serious infection). |
| | | complex | |
| | | (SBSEC); | |
| | | Lancefield | |
| | | group D; | |
| | | Streptococcus | |
| | | bovis biotype | |
| | | II | |
| SPA fragment St29 | Streptococcus iniae | Non- | β-lactam antibiotics; |
| | | Lancefield | penicillin, ampicillin. |
| | | Streptococcus | |
| SPA fragment St30 | Streptococcus | Viridans | Uncommon cause of |
| | salivarius, | group | invasive infections. |
| | Streptococcus | streptococci | |
| | thermophilus, | (VGS), | |
| | Streptococcus | Streptococcus | |
| | vestibularis | salivarius | |
| | | group | |
| SPA fragment St31, | Streptococcus | Viridans | Uncommon cause of |
| SPA fragment St32 | salivarius | group | invasive infections. |
| | | streptococci | |
| | | (VGS), | |
| | | Streptococcus | |
| | | salivarius | |
| | | group | |
| SPA fragment St33 | Streptococcus bovis | Streptococcus | Penicillin, ampicillin, |
| | Streptococcus | bovis/ | vancomycin (plus an |
| | gallolyticus subsp. | Streptococcus | aminoglycoside for |
| | gallolyticus | equinus | serious infection). |
| | Streptococcus | complex | |
| | gallolytics subsp. | (SBSEC); | |
| | macedonicus | Lancefield | |
| | Streptococcus | group D; | |
| | gallolytics subsp. | Streptococcus | |
| | pasteurianus | bovis | |
| | | biotype I | |
| SPA fragment St34 | Streptococcus equinus | Streptococcus | |
| | | bovis/ | |
| | | Streptococcus | |
| | | equinus | |
| | | complex | |
| | | (SBSEC); | |
| | | Lancefield | |
| | | group D | |
| SPA fragment St35 | Streptococcus | Streptococcus | |
| | Gallolytics subsp. | bovis/ | |
| | gallolyticus | Streptococcus | |
| | | equinus | |
| | | complex | |
| | | (SBSEC); | |
| | | Lancefield | |
| | | group D | |
| SPA fragment Ef1 | Enterococcus faecalis | Lancefield | Penicillin, ampicillin, |
| | | group D | vancomycin (plus an |
| SPA fragment Ef2 | Enterococcus faecalis | Lancefield | aminoglycoside for |
| | | group D | serious infection). |
| SPA fragment Ef3 | Enterococcus faecium | Lancefield | Vancomycin-resistant |
| | | group D | entercocci: |
| SPA fragment Ef4 | Enterococcus faecium | Lancefield | Streptogramins |
| | | group D | (quinupritsin/ |
| | | | dalfopristin), |
| | | | oxazolidinones |
| | | | (linezolid), |
| | | | lipopeptide |
| | | | (daptomycin). |

These results show that, unexpectedly, despite their relatively short size, 50 nucleotide long SPA fragment sequences covering the region upstream of the RpoB1-R1327 primer annealing site allow for high resolution phylogenetic identification of clinically relevant Streptococcus and Ernterococcus at the species or group level (as summarized in Table 20), thus providing an important method for (early) detection and identification of these infectious species using mcfDNA from peripheral blood samples. The high phylogenetic resolution of the SPA fragments can be directly linked to the standard of care for the most appropriate antibiotic regime to treat infections by Streptococcus and Enterococcus species, further demonstrating the clinical relevance of SPA fragment sequencing.

In addition, clinically relevant Streptococcus species including Streptococcus gallolyticus, Streptococcus macedonicus, Streptococcus pasteurianus and Streptococcus equinus have been linked to cancer. Therefore, the detection of Streptococcus species in peripheral blood is important for detection and prognostics of various types of cancer, as will also be discussed in EXAMPLE 7.

Furthermore, the analysis of EXAMPLE 5 shows the promise of SPA fragment sequencing as a new approach for assessing the risk of sepsis in immune compromised individuals, based on the (early) detection and identification of infectious and opportunistic pathogenic bacterial species using mcfDNA from peripheral blood samples.

### Example 6

SPA Fragment Sequences to Identify Opportunistic Bacterial Pathogens Originating from the Oral Cavity.

The oral cavity represents a source of opportunistic pathogenic bacteria that can have significant health implications when entering the body. Porphyromonas gingivalis is an example of an oral pathogen that has received a lot of attention. Not only is this bacterium the cause of gingivitis (Socransky et al, 1998; Chen et al, 2018), but several studies have implicated this bacterium in Alzheimer's disease (Dominy et al, 2019; Kanagasingam et al, 2020). Therefore, in the fight against Alzheimer's disease there is an unmet need for high-resolution, high-throughput and low-cost early detection of this bacterium in peripheral blood, something SPA fragment sequencing can provide. To evaluate its application for high-resolution detection of Porphyromonas gingivalis in peripheral blood, saliva or stool to complement risk screening for developing Alzheimer's disease, and to analyze the discriminatory power of SPA fragment sequencing for high resolution phylogenetic identification of infecting Porphyromonas gingivalis strains, 50 nucleotide long SPA fragments located upstream of the RpoB1-R1327 priming site were generated in silico for Porphyromonas strains. The results are presented in Table 21.

TABLE 21
Overview of the sequences of 50 nucleotide SPA fragments generated in silico for
Porphyromonas gingivalis strains and related species. For each SPA fragment, the
Porphyromonas species and the number of strains is indicated. The SPA fragments
representing 63 Porphyromonas species and related strains are reported. Porphyromonas
(gingivalis) -specific (Pg) SPA fragments received a unique numerical identifier (for reference
in further analysis.

| | No. of |
|---|---|
| Porphyromonas (Pg) specific SPA fragment (50 nucleotides) sequence | strains |
| SPA fragment Pg1 - | 27 |
| TTGAGATCATCAAGTATCTTATTGAGTTAGTAAATTCCAAGGCAT | |
| CAGTA (SEQ ID NO: 154) | |
| Porphyromonas gingivalis | 27 |
| SPA fragment Pg2 - | 2 |
| CTGCGATCATTGCTCATCTCGTAGAGTTGAAGAACAGCAAGCAG | |
| GTCGTC (SEQ ID NO: 155) | |
| Porphyromonas cangingivalis | 2 |
| SPA fragment Pg3 - | 17 |
| TGGCCATCATCAAGTACCTCATCGGGCTTGTCAACTCTAAGGAG | |
| GTCGTC (SEQ ID NO: 156) | |
| Porphyromonadaceae | 17 |
| SPA fragment Pg4 - | 4 |
| TGGCCATCATCAAGTACCTCATCGGGCTTGTCAACTCTAAGGAA | |
| GTCGTC (SEQ ID NO: 157) | |
| Porphyromonadaceae | 4 |
| SPA fragment Pg5 - | 3 |
| TTGCTATCATACGCCACCTGATCAAGCTCGTCAATGGTAAGGCA | |
| CCTGTC (SEQ ID NO: 158) | |
| Porphyromonas uenonis | 3 |
| SPA fragment Pg6 - | 3 |
| TTGCGATCATACGTCATCTGATCAAGCTCGTCAATGGTAAGGCT | |
| CCTGTC (SEQ ID NO: 159) | |
| Porphyromonadaceae | 3 |
| SPA fragment Pg7 - | 2 |
| TTTCCATTGTTAACCACCTTCTATTGTTAGCAACAACGGGTGCTA | |
| ACGTT (SEQ ID NO: 160) | |
| Porphyromonas endodontalis | 1 |
| Propionibacterium acidifaciens | 1 |
| SPA fragment Pg8 - | 2 |
| TTGCGATCATACGTCACTTGATCAAGCTCGTCAATGGTAAGGCT | |
| CCAGTC (SEQ ID NO: 161) | |
| Porphyromonas asaccharolytica | 2 |
| SPA fragment Pg9 - | 2 |
| TGGCCATCATCAAGTACCTCATCGGTCTTGTCAACTCTAAGGAG | |
| GTCGTC (SEQ ID NO: 162) | |
| Porphyromonadaceae JCVI 49 bin. 7 | 2 |
| SPA fragment Pg10 - | 1 |
| TTGAAATTATTAAATATCTGATTCAATTAGTTAACTCCAAAGCGG | |
| TGGTG (SEQ ID NO: 163) | |
| Porphyromonas macacae | 1 |

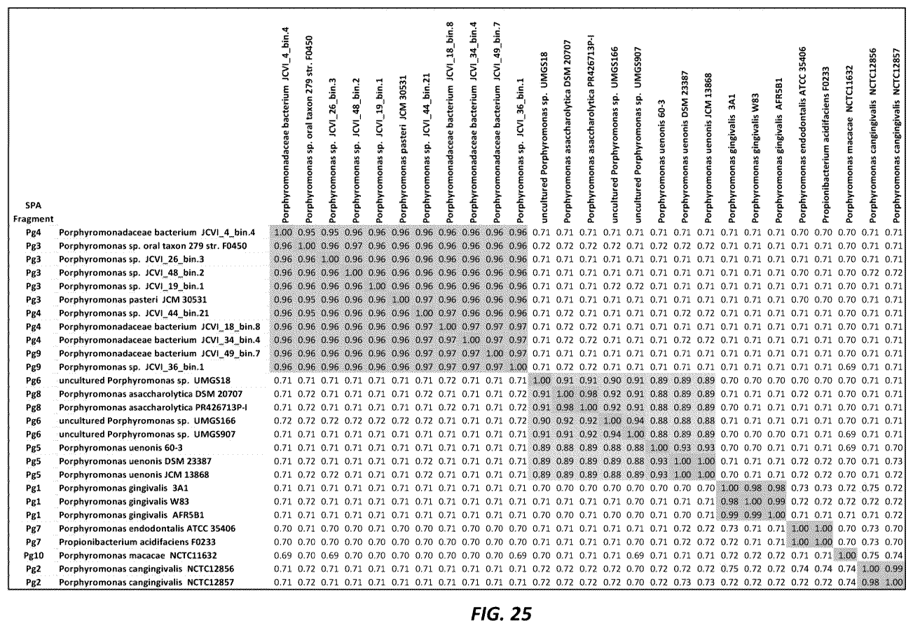

As shown in Table 21, the 50 nucleotide SPA fragments generated in silico for Porphyromonas gingivalis strains and related species distinguish Porphyromonas at the species level, as was also confirmed by whole genome-based ANI analysis (FIG. 25). The ANI analysis shows that the Porphyromonadaceae identified by the SPA fragments Pg3, Pg4 and Pg9 form a new ANI group. ANI analysis also confirms that the Porphyromonas endodontalis and Propionibacterium acidifaciens strains, identified by SPA fragment Pg7, are very closely related (100% ANI score) and therefore represent the same species. These results show that unexpectedly, despite their relatively short size, 50 nucleotide long SPA fragment sequences covering the region upstream of the RpoB1-R1327 primer annealing site allow for high resolution phylogenetic identification of clinically relevant Porphyromonas at the species level (Table 21), including Porphyromonas gingivalis, thus providing an important method for its (early) detection using mcfDNA from peripheral blood, saliva and stool samples. This shows the importance of SPA fragment sequencing as a new approach as part of risk screening for Alzheimer's disease based on the (early) detection and identification of Porphyromonas gingivalis species.

Prevotella are bacteria that inhabit many parts of the body. Although common in the gut microbiome, if found elsewhere, they may be a sign of infection. Prevotella oris represents an example of an opportunistic pathogenic bacterium that has been associated with several serious oral and systemic infections. Prevotella oris can been identified in clinical specimens by bacterial culture and biochemical tests, which are generally unreliable (Riggo and Lennon, 2007). Therefore, there is an unmet need for high-resolution, high-throughput and low-cost early detection of this bacterium in peripheral blood, something SPA fragment sequencing can provide. To evaluate its application for the reliable detection in peripheral blood of Prevotella species, and to analyze the discriminatory power of SPA fragment sequencing for high resolution phylogenetic identification of infecting Prevotella strains, 50 nucleotide long SPA fragments located upstream of the RpoB1-R1327 priming site were generated in silico for Prevotella strains. The results are presented in Table 22.

TABLE 22
Overview of the sequences of 50 nucleotide SPA fragments generated in silico for
Prevotella species. For each SPA fragment, the Prevotella species and the number of strains is
indicated. The SPA fragments representing 63 Prevotella species strains are reported.
Prevotella-specific (Pr) SPA fragments received a unique numerical identifier for reference in
further analysis.

| | No. of |
|---|---|
| Prevotella (Pr) specific SPA fragment (50 nucleotides) sequence | strains |
| SPA fragment Pr1 - | 19 |
| TCGCTATCATTAAGTATTTGATAAATCTTGTAAATTCAAATGCAA | |
| CAGTT (SEQ ID NO: 164) | |
| Prevotella pallens | 19 |
| SPA fragment Pr2 - | 14 |
| TTGAAATTATCAAGTACCTTATAAGTCTTGTAAATTCAAATGCTA | |
| CAGTC (SEQ ID NO: 165) | |
| Prevotella histicola | 14 |
| SPA fragment Pr3 - | 12 |
| TTGAGATTATTAAGTATCTTATCAGCCTTATCAATTCAAATGCTA | |
| CGGTT (SEQ ID NO: 166) | |
| Prevotella melaninogenica | 12 |
| SPA fragment Pr4 - | 11 |
| TTGAGATCATTAAATATCTTATTCAGCTGATCAACTCTAGTGCAA | |
| CAGTT (SEQ ID NO: 167) | |
| Prevotella copri | 11 |
| SPA fragment Pr5 - | 7 |
| TTGAGATTATTAAATATCTTATTCAGCTGATTAACTCTAGTGCAA | |
| CAGTT (SEQ ID NO: 168) | |
| Prevotella copri | 7 |
| SPA fragment Pr6 - | 7 |
| TCGAGATTATCAAGTATTTGATAAACCTCGTAAATTCGAATGCAA | |
| CAGTT (SEQ ID NO: 169) | |
| Prevotella intermedia | 7 |
| SPA fragment Pr7 - | 5 |
| TCGAGATTATCAAGTATTTGATTAACCTCGTAAATTCGAATGCAA | |
| CAGTT (SEQ ID NO: 170) | |
| Prevotella intermedia | 5 |
| SPA fragment Pr8 - | 6 |
| TTGAGATTATCAAGTACCTCATTAGCTTAGTCAATTCAAATGCAA | |
| CCGTT (SEQ ID NO: 171) | |
| Prevotella oral | 6 |
| SPA fragment Pr9 - | 6 |
| TCGCAATTATACGATACTTGATTCAGCTTATCAATTCGAATGCAA | |
| CAGTC (SEQ ID NO: 172) | |
| Prevotella nanceiensis | 6 |
| SPA fragment Pr10 - | 5 |
| TTGCGATTATCAAATATCTCATTCAGCTTGTCAATTCTAATGTTA | |
| CAGTT (SEQ ID NO: 173) | |
| Prevotella salivae | 5 |
| SPA fragment Pr11 - | 2 |
| TTGCGATTATCAAATACCTTATTCAGCTTGTCAATTCTAATGTTA | |
| CAGTT (SEQ ID NO: 174) | |
| Prevotella salivae | 2 |
| SPA fragment Pr12 - | 5 |
| TCGCGATTATAAAATATTTGATAAACCTTGTGAATTCAAATGCCA | |
| CTGTT (SEQ ID NO: 175) | |
| Prevotella nigrescens | 5 |
| SPA fragment Pr13 - | 4 |
| TTGAAATCATCAAATATCTCATCAGCCTGATCAACTCAAATGCCA | |
| CGGTT (SEQ ID NO: 176) | |
| Prevotella denticola | 4 |
| SPA fragment Pr14 - | 3 |
| TTGAGATTATCAAATATCTGATTCAGCTGATTAACTCCAATGCTA | |
| CTGTA (SEQ ID NO: 177) | |
| Prevotella buccae | 3 |
| SPA fragment Pr15 - | 3 |
| TTGCCATCATCCGCTATCTCATCCAGTTGGTTAACTCTAACGCAA | |
| CTGTT (SEQ ID NO: 178) | |
| Prevotella stercorea | 3 |
| SPA fragment Pr16 - | 3 |
| TTGAAATCATAAAATATCTCATCCAGTTGGTTAATTCCAATGCCA | |
| CTGTT (SEQ ID NO: 179) | |
| Prevotella oris | 3 |
| SPA fragment Pr17 - | 3 |
| TTGAGATTATCAAATATTTGATAAACCTCATCAATTCTAACGCAA | |
| CTGTT (SEQ ID NO: 180) | |
| Prevotella disiens | 3 |
| SPA fragment Pr18 - | 2 |
| TTGCTATTATCAAGTACTTGATTAAGCTTGTTAATTCTCAGGCTA | |
| CTGTT (SEQ ID NO: 181) | |
| Prevotella bryantii | 2 |
| SPA fragment Pr19 - | 2 |
| TTGAAATTATCAAATATCTCATTCAGCTGGTTAACTCTAATGCAA | |
| CCGTG (SEQ ID NO: 182) | |
| Prevotella shahii | 2 |

As shown in Table 22, the 50 nucleotide SPA fragments generated in silico for Prevotella strains distinguish Prevotella at the species level. These results show that unexpectedly, despite their relatively short size, 50 nucleotide long SPA fragment sequences covering the region upstream of the RpoB1-R1327 primer annealing site allow for high resolution phylogenetic identification of clinically relevant Prevotella at the species level (Table 22), including Prevotella oris, thus providing an important method for its (early) detection as an infecting pathogen using mcfDNA from peripheral blood samples.

### Example 7

SPA Fragment Sequences of Bacteria Linked to Tumor Microbiomes and their Use as Biomarkers for Cancer Detection and Progression Monitoring.

Several clinically relevant bacteria have been identified as playing a key role in the onset and progression of cancer, such as Streptococcus bovis type I (Streptococcus gallolyticus strains) which has been associated with CRC (see Example 5). Therefore, the use of SPA fragment sequencing for screening of peripheral blood or stool of cancer patients for the presence of bacteria as biomarkers for the detection, monitoring of disease progression, prognostics for survival and minimal residual disease, will provide important information complementary to customary blood biopsy- and stool-based detection and monitoring approaches that use cfDNA and focus on the methylation and mutation footprints in specific genetic loci as tumor biomarkers.

Contrary to PCR-based detection methods that monitor for the presence of specific bacteria, SPA fragment sequencing provides an “open” diagnostics approach to detect any bacterium based on the presence of its mcfDNA in peripheral blood. Due to its high phylogenetic resolution, SPA fragment sequencing can be used to identify novel microbiome signatures in blood and stool as biomarkers for the (early) detection of cancer. Once these signatures have been identified and validated as cancer-relevant biomarkers, SPA fragment sequencing is ideally positioned as a novel high-resolution, high-throughput and low-cost approach for population screening, e.g. adults between the ages 45 to 85, with a focus on (early) detection. In what follows, examples are provided for SPA fragments as biomarkers to detect and monitor the progression of cancer based on the presence of microbial signatures characterized by bacteria that have been associated with specific cancers and their developmental stage.

Risk screening for esophageal cancer: Esophageal cancer is the eighth most common cause of cancer deaths worldwide. Tannerella forsythia and Porphyromonas gingivalis, both of which have been implicated in periodontal diseases as part of red complex of periodontal pathogens, have been found to be associated with an increased risk of esophageal cancer (Malinowski et al, 2019). As shown in Table 21 of EXAMPLE 6, Porphyromonas gingivalis strains can be specifically identified by SPA fragment Pg1. To evaluate its application for the reliable detection in peripheral blood, saliva and stool of Tannerella forsythia to complement risk screening for developing esophageal cancer, and to analyze the discriminatory power of SPA fragment sequencing for high resolution phylogenetic identification of infecting Tannerella forsythia strains, 50 nucleotide long SPA fragments located upstream of the RpoB1-R1327 priming site were generated in silico for Tannerella forsythia strains. The results are presented in Table 23.

TABLE 23
Overview of the sequences of 50 nucleotide SPA fragments generated in silico for
Tannerella forsythia and the related species Tannerella oral. For each SPA fragment, the
Tannerella species and the number of strains is indicated. The SPA fragments representing 10
Tannerella strains are reported. Tannerella (forsythia)-specific (Tf) SPA fragments received a
unique numerical identifier for reference in further analysis.

| | No. of |
|---|---|
| Tannerella forsythia (Tf) specific SPA fragment (50 nucleotides) sequence | strains |
| SPA fragment Tf1 - | 7 |
| TTGAGATTATCAAATATCTGATTGAATTGATCAACTCGAAGGCGG | |
| TGGTA (SEQ ID NO: 183) | |
| Tannerella forsythia | 7 |
| SPA fragment Tf2 - | 2 |
| TTGAGATTATCAAATATCTGATTGAACTGATTAATTCGAAGGCAG | |
| TTGTA (SEQ ID NO: 184) | |
| Tannerella forsythia | 2 |
| SPA fragment Tf3 - | 1 |
| TCGAAATCATCAAATACCTCATCGAGCTGATCAACTCCAAGGCG | |
| GTTGTT (SEQ ID NO: 185) | |
| Tannerella oral | 1 |

As shown in Table 23, the 50 nucleotide SPA fragments generated in silico for Tannerella strains distinguish between Tannerella forsythia and the related species Tannerella oral. These results show that unexpectedly, despite their relatively short size, 50 nucleotide long SPA fragment sequences covering the region upstream of the RpoB1-R1327 primer annealing site allow for high resolution phylogenetic identification of the clinically relevant species Tannerella forsythia (Table 23). Therefore, SPA fragments for Tannerella forsythia and Porphyromonas gingivalis can be used as biomarkers using mcfDNA from peripheral blood, saliva and stool samples for the risk profiling and (early) detection of esophageal cancer.

Risk screening for precancerous colonic polyps: The common commensal bacterium, nontoxigenic Bacteroides fragilis (NTBF), is enriched in patients with precancerous colonic polyps. NTBF isolated from polyps is enriched in genes involved in LPS biosynthesis, which may allow for its increased ability to activate the immune system and cause inflammation (Kordahi et al, 2021). Therefore, there is an unmet need for high-resolution, high-throughput and low-cost early detection of this bacterium in peripheral blood and stool samples, something SPA fragment sequencing can provide. To analyze the discriminatory power of SPA fragment sequencing for the early detection of Bacteroides fragilis as an indicator species for the presence of precancerous colonic polyps, 50 nucleotide long fragments located upstream of the RpoB1-R1327 priming site were generated in silico for Bacteroides fragilis strains. The results are presented in Table 24.

TABLE 24
Overview of the sequences of 50 nucleotide SPA fragments generated in silico for
Bacteroides fragilis and related species. For each SPA fragment, the Bacteroides species and
the number of strains is indicated. The SPA fragments representing 80 Bacteroides fragilis
strains and related species are reported. Bacteroides fragilis-specific (Bf) SPA fragments
received a unique numerical identifier reference in further analysis.

| | No. of |
|---|---|
| Bacteroides fragilis (Bf) specific SPA fragment (50 nucleotides) sequence | strains |
| SPA fragment Bf1 - | 17 |
| TTGAGATCATTAAATATCTGATTGAGTTGATTAACTCTAAAGCAG | |
| ATGTG (SEQ ID NO: 186) | |
| Bacteroides fragilis | 14 |
| Bacteroides NSJ-2 | 1 |
| Bacteroides PHL | 1 |
| Bacteroides UW | 1 |
| SPA fragment Bf2 - | 2 |
| TCGAGATCATCAAATATCTGATTGAGCTGATTAATTCAAAAGCAG | |
| ATGTA (SEQ ID NO: 187) | |
| Bacteroides fragilis | 2 |
| SPA fragment Bf3 - | 61 |
| TCGAGATCATCAAATATCTGATTGAGCTGATTAACTCAAAAGCAG | |
| ATGTA (SEQ ID NO: 188) | |
| Bacteroides 2_1_16 | 1 |
| Bacteroides 3_2_5 | 1 |
| Bacteroides cellulosilyticus | 1 |
| Bacteroides fragilis | 58 |

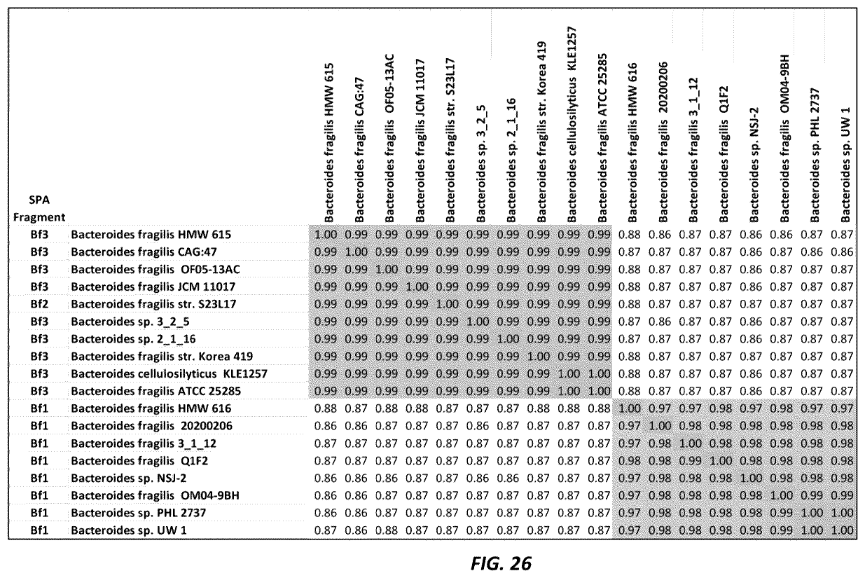

As shown in Table 24, the 50 nucleotide SPA fragments generated in silico for Bacteroides fragilis strains and related species distinguish Bacteroides fragilis at the species level, as was also confirmed by whole genome-based ANI analysis presented in FIG. 26. Whole genome-based ANI analysis shows that the Bacteroides fragilis strains identified by the SPA fragments Bf2 and Bf3 form an ANI group distinct from the Bacteroides fragilis identified by the SPA fragment Bf1 and might represent a different species or subspecies. ANI analysis also confirms that the Bacteroides cellulyticus strain, identified by SPA fragment Bf3, is nearly identical (100% ANI score) to Bacteroides fragilis strains and therefore represent the same species. Overall, despite their relatively short size, 50 nucleotide long SPA fragment sequences covering the region upstream of the RpoB1-R1327 primer annealing site allow for high resolution phylogenetic identification of clinically relevant Bacteroides fragilis at the species and likely subspecies level (Table 24; FIG. 26), thus providing an important method for its (early) detection using mcfDNA from peripheral blood samples. This shows the importance of SPA fragment sequencing as a new approach for the detection of precancerous colonic polyps based on the (early) detection and identification of Bacteroides fragilis species.

Risk screening for precancerous stomach ulcers: Stomach ulcers, caused by Helicobacter pylori, are a cause for stomach cancer when left untreated. Therefore, there is an unmet need for high-resolution, high-throughput and low-cost early detection of this bacterium in peripheral blood and stool, something SPA fragment sequencing can provide. To analyze the discriminatory power of SPA fragment sequencing for the early detection of Helicobacter pylori as an indicator species for the presence of stomach ulcers and potentially early-stage stomach cancer, 50 nucleotide long fragments located upstream of the RpoB1-R1327 priming site were generated in silico for Helicobacter pylori strains. The results are presented in Table 25.

TABLE 25
Overview of the sequences of 50 nucleotide SPA fragments generated in silico for
Helicobacter pylori. For each SPA fragment the number of Helicobacter pylori strains is
indicated. The SPA fragments representing 6 Helicobacter pylori strains are reported.
Helicobacter pylori-specific (Hp) SPA fragments received a unique numerical identifier for
reference in further analysis.

| | No. of |
|---|---|
| Helicobacter pylori (Hp) specific SPA fragment (50 nucleotides) sequence | strains |
| SPA fragment Hp1 - | 3 |
| TCACCACCGTTAAATACCTCATGAAAATCAAAAACAATCAGGGC | |
| AAGATT (SEQ ID NO: 189) | |
| Helicobacter pylori | 3 |
| SPA fragment Hp2 - | 2 |
| TCACCACCGTTAAATACCTCATGAAGATCAAAAACAATCAAGGC | |
| AAGATT (SEQ ID NO: 190) | |
| Helicobacter pylori | 2 |
| SPA fragment Hp3 - | 1 |
| TCACCACCGTTAAATACCTCATGAAGATCAAAAACAATCAGGGC | |
| AAGATT (SEQ ID NO: 191) | |
| Helicobacter pylori | 1 |

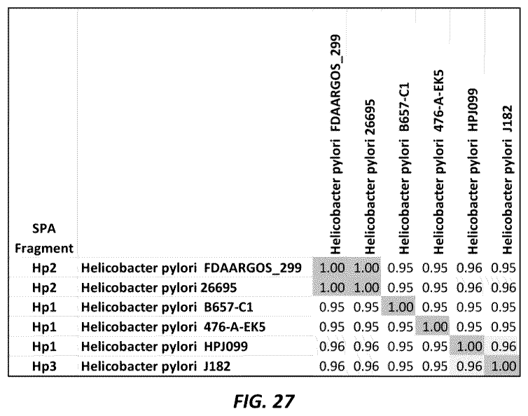

As shown in FIG. 27, whole genome-based ANI analysis reveals the presence of at least five select subspecies of Helicobacter pylori, with the strains identified by SPA fragment Hp1 breaking up in three ANI groups. Overall, despite their relatively short size, 50 nucleotide long SPA fragment sequences covering the region upstream of the RpoB1-R1327 primer annealing site allow for high resolution phylogenetic identification of the clinically relevant species Helicobacter pylori (Table 25). Therefore, SPA fragments for Helicobacter pylori can be used as biomarkers using mcfDNA from peripheral blood and stool samples for the risk profiling and (early) detection of precancerous stomach ulcers. The blood antibody test, a blood test to evaluate whether your body has made antibodies to Helicobacter pylori bacteria, is commonly used to determine if a patient is either currently infected or has been infected in the past with this bacterium. The advantage of SPA fragment sequencing is that it will only detect an active infection by Helicobacter pylori.

Women's health risk screening: Chlamydia trachomatis, a bacterium which is commonly transmitted sexually, is the major cause of mucopurulent cervicitis, pelvic inflammatory disease, tubal factor infertility, and ectopic pregnancy. Thus, the healthcare costs due to complications caused by Chlamydia trachomatis are enormous.

Cervical cancer is the most common cancer in women worldwide. Infection with Chlamydia trachomatis greatly increases the risk of cervical cancer (Anttila et al, 2001). Although infections with oncogenic strains of human papillomavirus remain the prime cause of cervical cancer, coinfections with some strains of Chlamydia trachomatis and Neisseria gonorrhoeae seem to contribute to that risk and the severity of the disease, especially high-grade squamous intraepithelial cervical lesions (De Abreu et al, 2016). This finding is important because chlamydia, though frequently asymptomatic, is one of the most common sexually transmitted diseases and can be treated with appropriate antibiotics. In the United States, between four million and eight million new cases of chlamydia are reported yearly.

Neisseria gonorrhoeae is a bacterial pathogen responsible for gonorrhea and various sequelae that tend to occur when asymptomatic infection ascends within the genital tract or disseminates to distal tissues. Like Chlamydia trachomatis, Neisseria gonorrhoeae is an important sexually transmitted pathogen and a major cofactor in HIV-1 infection. Global rates of gonorrhea continue to rise, facilitated by the emergence of broad-spectrum antibiotic resistance that has recently afforded the bacteria ‘superbug’ status. Therefore, there is an unmet need for high-resolution, high-throughput and low-cost early detection of these bacteria in peripheral blood and vaginal smear samples, something SPA fragment sequencing can provide. To analyze the discriminatory power of SPA fragment sequencing for the early detection of Chlamydia trachomatis and Neisseria gonorrhoeae as indicator species for women's health issues including the risk to develop cervical cancer, 50 nucleotide long fragments located upstream of the RpoB1-R1327 priming site were generated in silico for Chlamydia trachomatis and Neisseria gonorrhoeae strains. The results are presented in Table 26 and Table 27.

TABLE 26

| Chlamydia trachomatis (Ct) specific SPA fragment | No. of |
|---|---|
| (50 nucleotides) sequence | strains |
| SPA fragment Ct1- | 25 |
| GACAAACCCTGTCGCAGAATTGACGCACAAGCGTCGTCTGTCAG | |
| CATTAG (SEQ ID NO: 192) | |
| Chlamydia trachomatis | 25 |
| SPA fragment Ct2- | 2 |
| TAAGATCCACGCTCGTTCTATAGGACCTTACTCTCTCGTTACGCA | |
| GCAAC (SEQ ID NO: 193) | |
| Chlamydia trachomatis | 2 |

Overview of the sequences of 50 nucleotide SPA fragments generated in silico for Helicobacter pylori. For each SPA fragment the number of Chlamydia trachomatis strains is indicated. The SPA fragments representing 27 Chlamydia trachomatis strains are reported. Chlamydia trachomatis-specific (Ct) SPA fragments received a unique numerical identifier for reference in further analysis.

These results indicate that unexpectedly, despite their relatively short size, 50 nucleotide long SPA fragment sequences covering the region upstream of the RpoB1-R1327 primer annealing site allow for high resolution phylogenetic identification of the clinically relevant species Chlamydia trachomatis (Table 26)

TABLE 27

| Neisseria species (Ne) specific SPA fragment | No. of |
|---|---|
| (50 nucleotides) sequence | strains |
| SPA fragment Ne1- | 113 |
| TCGCCTCGATTGCGACTTTGGTCGAGTTGCGTAACGGCCATGGC | |
| GAAGTG (SEQ ID NO: 194) | |
| Neisseria gonorrhoeae | 41 |
| Neisseria meningitidis | 72 |
| SPA fragment Ne2- | 33 |
| TCGCCTCGATTGCGACTTTGGTCGAGTTGCGTAACGGCCATGGT | |
| GAAGTT (SEQ ID NO: 195) | |
| Neisseria meningitidis | 33 |
| SPA fragment Ne3- | 4 |
| TCGCCTCGATTGCGACTTTGGTCGAGCTGCGTAACGGTCACGGC | |
| GAAGTG (SEQ ID NO: 196) | |
| Neisseria lactamica | 4 |
| SPA fragment Ne4- | 3 |
| TTGCTTCTATTGCGACATTGGTTGAACTGCGTAACGGTCATGGC | |
| GAAGTA (SEQ ID NO: 197) | |
| Neisseria flavescens | 1 |
| Neisseria perflava | 1 |
| Neisseria subflava | 1 |
| SPA fragment Ne5- | 3 |
| TCGCCTCGATTGCGACTTTGGTCGAGTTGCGTAACTACCATGGC | |
| GAAGTG (SEQ ID NO: 198) | |
| Neisseria gonorrhoeae | 3 |
| SPA fragment Ne6- | 2 |
| TTGTTTCAATTGCTACCTTAGTTGAATTACGTAATCATAATGATG | |
| GTGTT (SEQ ID NO: 199) | |
| Neisseria weaver | 2 |
| SPA fragment Ne7- | 2 |
| TTGCATCAATTGCTACTTTAGTTGAATTGCGAAACGGTCATGGCG | |
| AAGTG (SEQ ID NO: 200) | |
| Neisseria mucosa | 2 |
| SPA fragment Ne8- | 2 |
| TGGCTTCGATTGCAACGTTGGTTGAGTTGCGTAACGGTCACGGT | |
| GAAGTG (SEQ ID NO: 201) | |
| Neisseria 10009 | 1 |
| Neisseria 10022 | 1 |
| SPA fragment Ne9- | 2 |
| TGGCTTCCATCGCCACTTTGGTGGAGTTGCGCAACGGGCATGGC | |
| GAAGTG (SEQ ID NO: 202) | |
| Neisseria shayeganii | 2 |
| SPA fragment Ne10- | 2 |
| TCGCTTCGATTGCCACTTTGGTTGAATTGCGTAACGGTCACGGC | |
| GAAGTG (SEQ ID NO: 203) | |
| Neisseria brasiliensis | 1 |
| Neisseria N95_16 | 1 |
| SPA fragment Ne11- | 1 |
| TTGTTTCTATTGCCACTTTAGTTGAGCTGCGTAATGGACATGGTG | |
| AAGTA (SEQ ID NO: 204) | |
| Neisseria zalophi | 1 |

Overview of the sequences of 50 nucleotide SPA fragments generated in silico for Neisseria species. For each SPA fragment, the Neisseria species and the number of strains is indicated. The SPA fragments representing 167 Neisseria strains and related species are reported. Neisseria-specific (Ne) SPA fragments received a unique numerical identifier for reference in further analysis.

Except for SPA fragments Ne1 and Ne4, the Neisseria-specific (Ne) SPA fragments were found to be species specific. The major combined group, identified by SPA fragment Ne1, was formed by Neisseria gonorrhoeae and Neisseria meningitidis strains. Neisseria meningitidis (meningococcus) causes significant morbidity and mortality in children and young adults worldwide through epidemic or sporadic meningitis and/or septicemia.

To improve the phylogenetic resolution of SPA fragment sequencing for Neisseria species, 50 nucleotide long fragments located upstream of the RpoB6-R1630 priming site were generated in silico for Neisseria strains. As shown in Table 4, the region upstream of the RpoB6-R1630 priming site has less sequence variance than the region upstream of the RpoB1-R1327 priming site. However, we found that this region provided a high degree of phylogenetic resolution of several c112linically important bacteria, including strains belonging to the genus Neisseria. An overview of the phylogenetic resolution of RpoB6-R1630-based SPA fragment sequencing for Neisseria species is provided in Table 28.

TABLE 28

| Neisseria species (Ne) specific SPA fragment | No. of |
|---|---|
| (50 nucleotides) sequence | strains |
| SPA fragment Ne12- | 48 |
| AACGCCGCGTATCCGCATTGGGTCCGGGCGGTTTGACCCGCGAA | |
| CGTGCA (SEQ ID NO: 205) | |
| Neisseria meningitidis | 48 |
| SPA fragment Ne13- | 36 |
| AACGCCGTGTATCTGCATTGGGCCCGGGCGGTTTGACCCGCGAA | |
| CGTGCC (SEQ ID NO: 206) | |
| Neisseria gonorrhoeae | 36 |
| SPA fragment Ne14- | 20 |
| AACGCCGCGTATCTGCATTGGGTCCGGGCGGTTTGACCCGCGAA | |
| CGTGCC (SEQ ID NO: 207) | |
| Neisseria meningitidis | 20 |
| SPA fragment Ne15- | 18 |
| AACGCCGCGTATCCGCATTGGGTCCGGGCGGTTTGACCCGCGAA | |
| CGTGCC (SEQ ID NO: 208) | |
| Neisseria meningitidis | 18 |
| SPA fragment Ne16- | 15 |
| AACGCCGTGTATCTGCATTGGGTCCGGGCGGTTTGACCCGCGAA | |
| CGTGCA (SEQ ID NO: 209) | |
| Neisseria meningitidis | 15 |
| SPA fragment Ne17- | 7 |
| AACGCCGTGTATCTGCATTGGGCCCGGGCGGTTTGACTCGCGAA | |
| CGTGCA (SEQ ID NO: 210) | |
| Neisseria meningitidis | 3 |
| Neisseria subflava | 1 |
| Neisseria perflava | 1 |
| Neisseria flavescens | 1 |
| Neisseria cinerea | 1 |
| SPA fragment Ne18- | 6 |
| AACGCCGTGTATCTGCATTGGGTCCGGGCGGTTTGACCCGCGAA | |
| CGTGCC (SEQ ID NO: 211) | |
| Neisseria gonorrhoeae | 6 |
| SPA fragment Ne19- | 4 |
| AACGCCGTGTATCTGCGTTGGGTCCGGGCGGTTTGACCCGCGAA | |
| CGTGCA (SEQ ID NO: 212) | |
| Neisseria lactamica | 4 |
| SPA fragment Ne20- | 2 |
| AGCGTCGTGTGTCTGCTTTAGGTCCAGGTGGTTTGACACGTGAA | |
| CGTGCA (SEQ ID NO: 213) | |
| Neisseria weaveri | 2 |
| SPA fragment Ne21- | 2 |
| AGCGTCGTGTGTCTGCTTTAGGTCCGGGTGGTTTGACACGTGAA | |
| CGTGCA (SEQ ID NO: 214) | |
| Neisseria zoodegmatis | 2 |
| SPA fragment Ne22- | 2 |
| AACGTCGTGTATCTGCATTGGGTCCGGGCGGTTTGACCCGCGAA | |
| CGTGCA (SEQ ID NO: 215) | |
| Neisseria meningitidis | 2 |
| SPA fragment Ne23- | 2 |
| AACGTCGTGTTTCTGCCTTGGGCCCGGGTGGTTTGACCCGTGAG | |
| CGTGCC (SEQ ID NO: 216) | |
| Neisseria 10022 | 1 |
| Neisseria 10009 | 1 |
| SPA fragment Ne24- | 2 |
| AACGTCGTGTTTCTGCTTTGGGTCCAGGCGGTTTGACCCGTGAA | |
| CGTGCT (SEQ ID NO: 217) | |
| Neisseria N95_16 | 1 |
| Neisseria brasiliensis | 1 |
| SPA fragment Ne25- | 2 |
| AACGCCGTGTATCCGCATTGGGTCCGGGCGGCTTGACCCGCGAA | |
| CGTGCA (SEQ ID NO: 218) | |
| Neisseria meningitidis | 2 |
| SPA fragment Ne26- | 2 |
| AACGCCGTGTATCTGCATTGGGCCCTGGTGGTTTGACTCGCGAA | |
| CGTGCA (SEQ ID NO: 219) | |
| Neisseria mucosa | 1 |
| Neisseria JCVI_22A_bin.7 | 1 |
| SPA fragment Ne27- | 1 |
| AGCGTCGTGTGTCTGCTTTAGGTCCGGGCGGTTTGACACGTGAA | |
| CGTGCG (SEQ ID NO: 220) | |
| Neisseria animaloris | 1 |

Overview of the sequences of 50 nucleotide SPA fragments generated in silico for Neisseria species from the region upstream of the RpoB6-R1630 priming site. For each SPA fragment, the Neisseria species and the number of strains is indicated. The SPA fragments representing 169 Neisseria strains and related species are reported. Neisseria-specific (Ne) SPA fragments received a unique numerical identifier or reference in further analysis.

As shown in Table 28, SPA fragments generated in silico for Neisseria species from the region upstream of the RpoB6-R1630 priming site allowed to distinguish with high phylogenetic resolution between Neisseria gonorrhoeae and Neisseria meningitidis strains. The practical implications of using an alternative primer annealing site or a combination of two primers that target different phylogenetic identifier regions are discussed in EXAMPLE 9.

Overall, despite their relatively short size, 50 nucleotide long SPA fragment sequences covering the region upstream of the RpoB6-R1630 primer annealing site allow for high resolution phylogenetic identification of the clinically relevant species Neisseria gonorrhoeae (Table 28). Therefore, SPA fragments for Chlamydia trachomatis and Neisseria gonorrhoeae can be used as biomarkers using mcfDNA from peripheral blood and/or vaginal smear samples for the risk profiling and (early) detection of women's health issues related to these bacteria including the risk to develop cervical cancer.

Prognostic correlations with the microbiome of breast cancer subtypes: There are four subtypes of breast cancer (BC) that are based on the status of the estrogen receptor, progesterone receptor, and human epidermal growth (Her2) expression in cancerous breast cells. As shown by Banerjee et al (2021), the subtypes of BC have specific viromes and microbiomes, with estrogen receptor positive (ER+) and triple negative (TN) tumors showing the most and least diverse microbiomes, respectively. These specific microbial signatures allowed successful discrimination between the different BC subtypes. Furthermore, Banerjee et al (2021) demonstrated correlations between the presence and absence of specific microbes in BC subtypes with the clinical outcomes. Therefore, there is an unmet need for high-resolution, high-throughput and low-cost early detection of bacteria associated with breast cancer subtypes in peripheral blood, something SPA fragment sequencing can provide.

TN BC (15-20% of BC patients) is the most aggressive of all the BCs, is non-responsive to treatment, is highly angiogenic, highly proliferative, and has the lowest survival rate. TN breast cancer showed decreased microbial diversity and increased levels of Aggregatibacter species; significant levels of this species were not detected in other BC types. Therefore, there is an unmet need for high-resolution, high-throughput and low-cost early detection of this bacterium in peripheral blood, something SPA fragment sequencing can provide. To analyze the discriminatory power of SPA fragment sequencing for the detection of Aggregatibacter species as indicator and prognostics species for TN breast cancer, 50 nucleotide long fragments located upstream of the RpoB1-R1327 priming site were generated in silico for Aggregatibacter strains. The results are presented in Table 29.

TABLE 29

| Aggregatibacter species (Ag) specific SPA fragment | No. of |
|---|---|
| sequence(50 nucleotides) | strains |
| SPA fragment Ag1- | 27 |
| TCAGTGTGATGAAAAAATTGATTGATATCCGTAATGGCCGTGGT | |
| GAAGTG (SEQ ID NO: 221) | |
| Aggregatibacter actinomycetemcomitans | 27 |
| SPA fragment Ag2- | 4 |
| TCAGTGTGATGAAGAAACTGATTGATATTCGTAATGGTCGCGGT | |
| GAAGTG (SEQ ID NO: 222) | |
| Aggregatibacter aphrophilus | 4 |
| SPA fragment Ag3- | 3 |
| TCAGTGTGATGAAGAAATTGATTGATATCCGTAATGGCCGTGGT | |
| GAAGTG (SEQ ID NO: 223) | |
| Aggregatibacter actinomycetemcomitans | 3 |
| SPA fragment Ag4- | 2 |
| TCAGTGTGATGAAAAAACTGATTGATATTCGTAATGGTCGCGGA | |
| GAAGTG (SEQ ID NO: 224) | |
| Aggregatibacter aphrophilus | 2 |
| SPA fragment Ag5- | 1 |
| TAAGTGTCATGAAGAAATTGATCGAAATTCGTAACGGTCGTGGT | |
| GAAGTG (SEQ ID NO: 225) | |
| Aggregatibacter segnis | 1 |

Overview of the sequences of 50 nucleotide SPA fragments generated in silico for Aggregatibacter species. For each SPA fragment, the Aggregatibacter species and the number of strains is indicated. The SPA fragments representing 37 Aggregatibacter strains and related species are reported. Aggregatibacter-specific (Ag) SPA fragments received a unique numerical identifier for reference in further analysis.

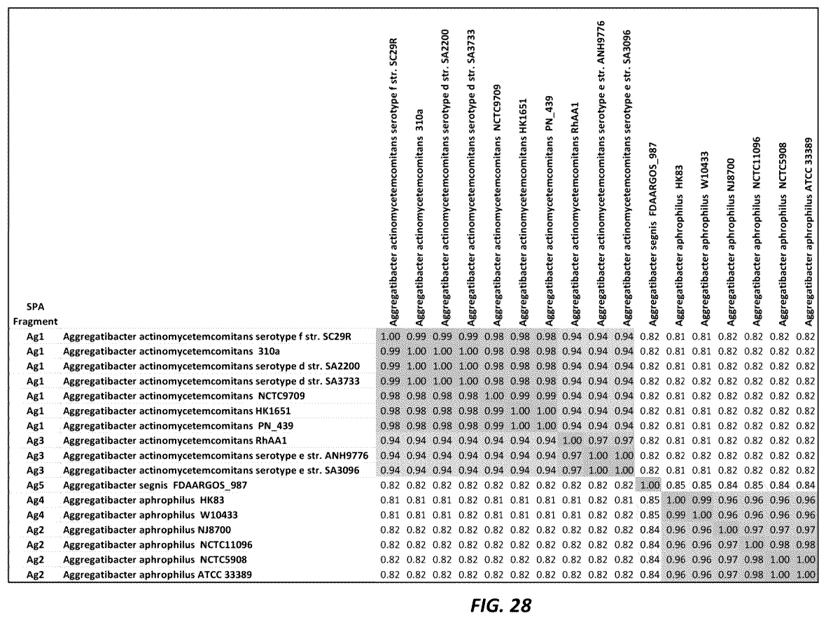

The results presented in Table 29 showed that the Aggregatibacter species could be identified by their unique SPA fragments. This was further confirmed by whole genome ANI analysis (FIG. 28).

The whole genome-based ANI results in FIG. 28 confirmed that Aggregatibacter actinomycetemcomitans could be identified by SPA fragments Ag1 and Ag3; that Aggregatibacter aphrophilus could be identified by SPA fragments Ag2 and Ag4; and that Aggregatibacter segnis could be identified by SPA fragment Ag5 (see also Table 29). Overall, these results show that unexpectedly, despite their relatively short size, 50 nucleotide long SPA fragment sequences covering the region upstream of the RpoB1-R1327 primer annealing site allow for high resolution phylogenetic identification of clinically relevant Aggregatibacter species. Therefore, SPA fragments for Aggregatibacter can be used as biomarkers using mcfDNA from peripheral blood and/or saliva samples for the risk profiling and (early) detection of TN breast cancer, as well as other cancers. For instance, a prospective population-based nested case-control study demonstrated that the presence of Porphyromonas gingivalis or Aggregatibacter actinomycetemcomitans in the oral cavity was indicative of increasing the risk of pancreatic cancer (Chandra and McAllister, 2021).

Prognostic correlations with the microbiome of pancreatic cancer: Pancreatic cancer, particularly pancreatic ductal adenocarcinoma (PDAC), is an aggressive disease with a poor prognosis. Chandra and McAllister (2021) pointed out the importance of microbial biomarkers for risk prognosis for pancreatic cancer. Risk factors for pancreatic cancer included periodontal disease and oral microbial dysbiosis, with abundances of Porphyromonas gingivalis, Aggregatibacter actinomycetemcomitans, Neisseria elongate and Streptococcus mitis as indicator species. As discussed previously, 50 nucleotide SPA fragments covering the region upstream of the RpoB1-R1327 primer annealing site can be used to successfully identify these species.

Of specific interest is the tumor microbiome composition of PDAC patients, as it holds clues for their treatment options and long-term survival. Geller et al (2017) reported the presence of bacteria in human PDACs and demonstrated that intra-tumoral Gamma-proteobacteria, among the most common bacteria detected in human pancreatic tumors, reduce the efficacy of chemotherapeutic drugs like gemcitabine, which these bacteria can metabolize into its inactive form via their cytidine deaminase. Thus, one application of SPA fragment sequencing would be to link phylogenetic identification to metabolic strain models, thereby predicting the impact of the tumor microbiome on drug metabolism and efficacy.

Riquelme et al (2019) profiled intra-tumoral bacteria from patients with resected PDAC and compared short-term and long-term survivors. Long-term survivors had greater intra-tumoral microbial α-diversity than did those who died of the disease within 5 years after resection. Overall tumor microbial characterization revealed a microbial composition similar to the one in human PDAC previously described by Geller et al (2017), but unique enrichment in the following microbes was found in tumors from long-term survivors: Pseudoxanthomonas, Streptomyces, Saccharopolyspora and Bacillus clausii, the last two species have documented immunomodulatory functions that might play a role in slowing down the disease progression. Therefore, there is an unmet need for high-resolution, high-throughput and low-cost early detection of these bacteria in peripheral blood, something SPA fragment sequencing can provide. To analyze the discriminatory power of SPA fragment sequencing for the detection of Pseudoxanthomonas, Streptomyces, Saccharopolyspora and Bacillus clausii species as indicator and prognostics species as prognostics for long term survival of PDAC patients, 50 nucleotide long fragments located upstream of the RpoB1-R1327 priming site were generated in silico for Pseudoxanthomonas, Streptomyces, Saccharopolyspora and Bacillus clausii strains. Unique SPA fragments were found able to identify Pseudoxanthomonas and Streptomyces at the genes level, and Saccharopolyspora and Bacillus clausii at the species level. The results for Bacillus clausii are presented in Table 30.

TABLE 30

| Bacillus clausii (Bcl) specific SPA fragment | No. of |
|---|---|
| (50 nucleotides) sequence | strains |
| SPA fragment Bcl1- | 14 |
| TCGCTTCCATCAGCTATTTCTTCAACTTGCTGCATGGTGTCGGCG | |
| ATACA (SEQ ID NO: 226) | |
| Bacillus clausii | 13 |
| Bacillus 7520-S | 1 |
| SPA fragment Bcl2- | 1 |
| TCGCTTCCATCAGCTATTTCTTCAACTTGTTGCATGGTGTCGGCG | |
| ATACA (SEQ ID NO: 227) | |
| Bacillus clausii | 1 |

Overview of the sequences of 50 nucleotide SPA fragments generated in silico for Bacillus clausii strains. For each SPA fragment, the Bacillus clausii species and the number of strains is indicated. The SPA fragments representing 14 Bacillus clausii strains and related species are reported. Bacillus clausii-specific (Bcl) SPA fragments received a unique numerical identifier for reference in further analysis.

These results show that overall, unexpectedly, despite their relatively short size, 50 nucleotide long SPA fragment sequences covering the region upstream of the RpoB1-R1327 primer annealing site allow for high resolution phylogenetic identification of clinically relevant Pseudoxanthomonas, Streptomyces, Saccharopolyspora and Bacillus clausii strains. Therefore, SPA fragments can be used as biomarkers using mcfDNA from peripheral blood samples for the risk profiling and prognostics for long-term survival of PDAC patients.

Prognostic correlations with the microbiome of lung cancer: Lung cancer is the most common cancer, excluding nonmelanoma skin cancer, and the most common cause of cancer-related death in the world, with approximately 1.8 million diagnoses and 1.6 million deaths per year. Peters et al (2019) pointed out the importance of microbial biomarkers for risk prognosis for lung cancer, observing that greater abundance of family Koribacteraceae in normal long tissue was associated with increased recurrence-free survival (RFS) and long-term disease-free survival (DFS), whereas greater abundance of family Lachnospiraceae, and genera Faecalibacterium and Ruminococcus (from Ruminococcaceae family), and Roseburia and Ruminococcus (from Lachnospiraceae family) were associated with reduced RFS and DFS. Taxa associated only with RFS (P<0.05) included family S24-7 (increased RFS), and family Bacteroidaceae and genus Bacteroides (reduced RFS). Taxa associated only with DFS (P<0.05) included family Sphingomonadaceae and genus Sphingomonas (increased DFS), and family Ruminococcaceae (reduced DFS). However, this study was performed using 16S rRNA gene sequencing and lacked the phylogenetic resolution to identify biomarker species at the species level. The 50 nucleotide long SPA fragment sequences covering the region upstream of the RpoB1-R1327 primer annealing site allow for the high resolution phylogenetic identification at the species level of the clinically relevant bacteria associated with the prognosis for recurrence-free survival (RFS) and long-term disease-free survival (DFS) of lung cancer patients. SPA sequencing is therefore well positioned to monitor disease progression and prognosis for lung cancer patients.

Risk screening for gastrointestinal tumors: Fusobacterium spp. is important in the development and progression of gastrointestinal tumors. In line with this, Poore et al (2020) showed that the Fusobacterium genus was overabundant in primary tumors compared to normal solid-tissue. Furthermore, pan-cancer analyses also showed an overabundance of Fusobacterium when comparing all broadly-defined gastrointestinal (GI) cancers against non-GI cancers in both primary tumor tissue and adjacent normal solid-tissue, pointing to Fusobacterium species as a biomarker for GI cancer. Therefore, there is an unmet need for high-resolution, high-throughput and low-cost early detection of this bacterium in peripheral blood and stool samples, something SPA fragment sequencing can provide. To analyze the discriminatory power of SPA fragment sequencing for the early detection of Fusobacterium species as biomarker for the risk to develop gastrointestinal cancer. 50) nucleotide long fragments located upstream of the RpoB1-R1327 priming site were generated in silico for Fusobacterium species. The results are presented in Table 31.

TABLE 31

| Fusarium species (Fs) specific SPA fragment | No. of |
|---|---|
| (50 nucleotides) sequence | strains |
| SPA fragment Fs1- | 12 |
| CTATTAAATATGTTATAGAGCTTAATAATGGTGATCAAAATGTTC | |
| ATACT (SEQ ID NO: 228) | |
| Fusobacterium canifelinum | 1 |
| Fusobacterium nucleatum | 10 |
| Fusobacterium OBRC1 | 1 |
| SPA fragment Fs2- | 8 |
| CTATTAAATATGTTATAGATCTTAATAATGGCGATCAAAATGTTC | |
| ATACT (SEQ ID NO: 229) | |
| Fusobacterium HMSC065F01 | 1 |
| Fusobacterium nucleatum | 7 |
| SPA fragment Fs3- | 8 |
| CAATGAAATATGTTACTGACCTTTATAATGGTGACCAAAATGTTC | |
| ATACA (SEQ ID NO: 230) | |
| Fusobacterium periodonticum | 8 |
| SPA fragment Fs4- | 7 |
| CGATACAATATGTCATTGATTTAAATAATGGGGAATCTCATGTCC | |
| ATACC (SEQ ID NO: 231) | |
| Fusobacterium necrophorum | 7 |
| SPA fragment Fs5- | 6 |
| CAATGAAATATGTTACTGACCTTTATAATGGTGATCAAAATGTTC | |
| ATACA (SEQ ID NO: 232) | |
| Fusobacterium periodonticum | 6 |
| SPA fragment Fs6- | 4 |
| TAGCTACAATGAAGTATGTAATTAACTTAAATAATGGAAATGGAC | |
| ATACT (SEQ ID NO: 233) | |
| Fusobacterium FSA-380-WT-2B | 1 |
| Fusobacterium mortiferum | 3 |
| SPA fragment Fs7- | 4 |
| CTATTAAGTATGTTATAGAGCTAAATAATGGTGACCAAAATGTTC | |
| ATACT (SEQ ID NO: 234) | |
| Fusobacterium hwasookii | 2 |
| Fusobacterium nucleatum | 2 |
| SPA fragment Fs8- | 4 |
| CTATTAGATATGTTATAGATCTTAATAATGGCGATCAAAATGTTC | |
| ATACT (SEQ ID NO: 235) | |
| Fusobacterium nucleatum | 4 |
| SPA fragment Fs9- | 4 |
| TAGGAACAATGAAATATGTAATTAATCTAAATAATGGAAATGGAC | |
| ACACT (SEQ ID NO: 236) | |
| Fusobacterium UBA10773 | 1 |
| Fusobacterium varium | 3 |
| SPA fragment Fs10- | 3 |
| CTATTAAGTATGTTATAGAACTTAATAATGGTGAACAAAATGTTC | |
| ATACT (SEQ ID NO: 237) | |
| Fusobacterium nucleatum | 3 |
| SPA fragment Fs11- | 2 |
| TTGGAACAATGAAATATGTAATTAATCTAAATAATGGAAATGGAC | |
| ATACT (SEQ ID NO: 238) | |
| Fusobacterium ulcerans | 2 |
| SPA fragment Fs12- | 2 |
| CTATTAAATATGTTATAGAACTTAATAATGGTGATCAAAATGTTC | |
| ATACT (SEQ ID NO: 239) | |
| Fusobacterium nucleatum | 2 |
| SPA fragment Fs13- | 2 |
| CTATTAAATATGTTATAGATCTTAATAATGGTGATCAAAATGTTC | |
| ATACT (SEQ ID NO: 240) | |
| Fusobacterium CM1 | 1 |
| Fusobacterium nucleatum | 1 |
| SPA fragment Fs14- | 2 |
| CTATTAAATATGTAATAGAGCTTAATAATGGTGATCAAAATGTTC | |
| ATACT (SEQ ID NO: 241) | |
| Fusobacterium nucleatum | 2 |
| SPA fragment Fs15- | 2 |
| CGATTCAATATGTCATTGATTTAAATAATGGAGAATCCCATGTAC | |
| ATACA (SEQ ID NO: 242) | |
| Fusobacterium equinum | 1 |
| Fusobacterium gonidiaformans | 1 |
| SPA fragment Fs16- | 1 |
| TTGGAACAATGAAATATGTAATTAATTTGAATAATGGAAATGGGC | |
| ATACT (SEQ ID NO: 243) | |
| Fusobacterium varium | 1 |
| SPA fragment Fs17- | 1 |
| TTGCAACTATGAAGTATGTAATTAATTTAAACAATGGAAATGGAC | |
| ATACT (SEQ ID NO: 244) | |
| Fusobacterium necrogenes | 1 |
| SPA fragment Fs18- | 1 |
| TCGCCTCCATCAATTACAACATGCATATCGAGGAGGGCATCGGC | |
| AGCAAC (SEQ ID NO: 245) | |
| Fusobacterium naviforme | 1 |

Overview of the sequences of 50 nucleotide SPA fragments generated in silico for Fusobacterium species. For each SPA fragment, the Fusobacterium species and the number of strains is indicated. The SPA fragments representing 73 Fusobacterium strains and related species are reported. Fusobacterium-specific (Fs) SPA fragments received a unique numerical identifier for reference in further analysis.

As shown in Table 31, the 50 nucleotide SPA fragments generated in silico for Fusobacterium strains mostly allowed to distinguish Fusobacterium at the (sub) species level, as was also confirmed by whole genome-based ANI analysis. The following exceptions were observed: In addition to identifying Fusobacterium nucleatum subsp. polymorphum, SPA fragment Fs1 also identified the closely related Fusobacterium canifelinum. Whole genome-based ANI analysis confirmed the similarity between these two species. In addition to identifying Fusobacterium hwasookii, SPA fragment Fs7 also identified the closely related Fusobacterium nucleatum subsp. polymorphum. Whole genome-based ANI analysis confirmed the similarity between these two species; it also showed that Fusobacterium nucleatum ChDC F128 strain should be reclassified as Fusobacterium hwasookii. Whole genome-based ANI analysis also showed that Fusobacterium equinum and Fusobacterium gonidiaformans, both identified by SPA fragment Fs15, represent the same species. A summary of the Fusobacterium species (Fs) specific SPA fragments as phylogenetic identifiers at the (sub)species level is provided in Table 32.

These results show that unexpectedly, despite their relatively short size, 50 nucleotide long SPA fragment sequences covering the region upstream of the RpoB-R1327 primer annealing site allow for high resolution phylogenetic identification of clinically relevant Fusobacterium at the (sub)species level (Table 32), thus providing an important method for its (early) detection using mcfDNA from peripheral blood and stool samples. This shows the importance of SPA fragment sequencing as a new approach as part of risk screening for broadly-defined gastrointestinal (GI) cancers based on the (early) detection and identification of Fusobacterium species.

TABLE 32
Summary of the Fusobacterium species (Fs) specific SPA fragments as
phylogenetic identifiers at the species level. The SPA fragments
are 50 nucleotides in length and cover the region upstream
of the RpoB1-R1327 primer annealing site.

| Fusobacterium species | |
|---|---|
| (Fs) specific | |
| SPA fragment | (Sub)species |
| SPA fragment Fs1, | Fusobacterium nucleatum subsp. |
| SPA fragment Sf7 | polymorphum, Fusobacterium hwasookii, |
| | Fusobacterium canifelinum |
| SPA fragment Fs2, | Fusobacterium nucleatum subsp. animalis |
| SPA fragment Sf8, | |
| SPA fragment Sf13 | |
| SPA fragment Fs3, | Fusobacterium periodonticum |
| SPA fragment Sf5 | |
| SPA fragment Sf4 | Fusobacterium necrophorum subsp. |
| | funduliforme |
| SPA fragment Sf6 | Fusobacterium mortiferum |
| SPA fragment Sf9, | Fusobacterium varium |
| SPA fragment Sf16 | |
| SPA fragment Sf10 | Fusobacterium nucleatum subsp. nucleatum |
| SPA fragment Sf11 | Fusobacterium ulcerans |
| SPA fragment Sf12 | Fusobacterium nucleatum subsp. |
| | polymorphum |
| SPA fragment Sf14 | Fusobacterium nucleatum subsp. vincentii |
| SPA fragment Sf15 | Fusobacterium equinum*, Fusobacterium |
| | gonidiaformans* |
| SPA fragment Sf17 | Fusobacterium necrogenes |
| SPA fragment Sf18 | Fusobacterium naviforme |

*Whole genome- based ANI analysis indicates that these species are nearly identical.

Several studies successfully demonstrated that including the microbial footprint increases the specificity and sensitivity of screening tests for the detection of early-stage adenomas and carcinomas in colorectal cancer. For example, a metagenomics-based classification model, using abundance changes of Fusobacterium nucleatum ssp. vincentii and Fusobacterium nucleatum ssp. animalis, Peptostreptococcus stomatis and Pseudonocardia asaccharolytica in CRC patients versus healthy controls combined with standard CRC diagnostics improved CRC-detection sensitivity for the guaiac-based fecal occult blood test (gFOBT) by >45% (Zeller et al, 2014). A microbiota-based random forest model using abundance changes of Fusobacterium, Peptostreptococcus, Porphyromonas, Prevotella, Parvimonas, Bacteroides and Gemella species complemented the fecal immunochemical test (FIT) (Baxter et al, 2016). The microbiota-based random forest model detected 91.7% of cancers and 45.5% of adenomas while FIT alone detected 75.0% and 15.7%, respectively. Of the colonic lesions missed by FIT, the model detected 70.0% of cancers and 37.7% of adenomas.

The present inventors confirmed that Peptostreptococcus stomatis and Pseudonocardia asaccharolytica can be identified by their single unique SPA fragments; that Parvimonas species, including Parvimonas oral and Parvimonas micra could be identified by a single SPA fragment; and that Gemella species, including Gemella morbillorum, Gemella haemolysans, Gemella palaticanis and Gemella sanguinis each had their unique SPA fragment. Therefore, combining tumor-specific biomarkers (including mutational footprint, methylation footprint, and blood detection in stool) with the quantitative detection of biomarker microorganisms using SPA fragment sequencing at the species and subspecies level will significantly increase the sensitivity and specificity of colorectal cancer screening. In addition, a further application of the SPA sequencing method is that once unique SPA fragments have been identified that correlate with the detection of specific diseases and monitoring of their progression, the unique SPA fragment sequences can be used to develop species-specific screening assays as part of PCR-based diagnostic platforms.

In certain instances, disease phenotypes caused by bacteria will depend on specific metabolic properties; as a result, accurate disease detection, monitoring and prognostics will require additional functional insights besides phylogenetic identification and community composition. For example, a random forest-based model using abundance changes of Fusobacterium nucleatum, Peptostreptococcus stomatis, Pseudonocardia asaccharolytica, Prevotella species, Parvimonas species, Gemella morbillorum and other bacteria, combined with gFOBT, improved the sensitivity/specificity of CRC detection (Thomas et al, 2019). This study also found that the choline trimethylaminelyase gene, which encodes Trimethylamine (TMA) synthesis from dietary quaternary amines (mainly choline and camitine), was overabundant in the microbiomes of CRC patients (P=0.001), identifying a relationship between gut microbiome choline metabolism and CRC. Trimethylamine (TMA) has previously been associated with atherosclerosis and severe cardiovascular disease. Importantly, SPA fragment sequencing provides the flexibility to address both phylogenetic identification and community functionality. For example, this is performed by selecting a degenerate primer that recognizes a conserved DNA region of a specific function, the same protocol outlined in FIGS. 2 and 3A is broadly applicable for SPA amplification and sequencing of functional genes. Furthermore, phylogenetic and functional information can be obtained simultaneously by including both a degenerate primer that targets the phylogenetic identifier gene and a degenerate primer that targets the functional gene in the same reaction for the SPA fragment amplification step (FIG. 2, step 4). We refer to this approach as multiplex SPA for the simultaneous detection of multiple targets in a single PCR reaction. In the specific case of colorectal cancer, a primer targeting the choline trimethylaminelyase gene can be combined with the RpoB1-R1327 primer for improved detection, monitoring and progression of adenomas and carcinomas.

### Example 8

### SPA Fragment Sequences for the Detection of Infections Caused by Emerging Pathogenic Bacteria of Clinical Concern.

Risk screening for developing Clostridium difficile infection: Clostridium difficile is the leading cause of health-care-associated infective diarrhea. Due to increased use of antibiotics that disrupt the healthy gut microbiome, creating a niche for Clostridium difficile to thrive, the incidence of Clostridium difficile infection (CDI) has been rising worldwide with subsequent increases in morbidity, mortality, and health care costs. Asymptomatic colonization with Clostridium difficile is common and a high prevalence has been found in specific cohorts, e.g., hospitalized patients, adults in nursing homes and in infants. Therefore, there is an unmet need for high-resolution, high-throughput and low-cost early detection of this bacterium in peripheral blood stool samples, something SPA fragment sequencing can provide. To analyze the discriminatory power of SPA fragment sequencing for the early detection of Clostridium difficile as biomarker for the risk to develop CDI, 50 nucleotide long fragments located upstream of the RpoB1-R1327 priming site were generated in silico for Clostridium difficile strains. The results are presented in Table 33.

TABLE 33

| Clostridium difficile strain (Cd) specific SPA fragment | No. of |
|---|---|
| (50 nucleotides) sequence | strains |
| SPA fragment Cd1- | 60 |
| TAGCTTCAATAAGTTATGAGTTCAATATATTCTATAATATAGGA | |
| AATATT (SEQ ID NO: 246) | |
| Clostridium difficile | 59 |
| Clostridium UMGS188 | 1 |

Overview of the sequences of 50 nucleotide SPA fragments generated in silico for Clostridium difficile strains. For each SPA fragment, the number of Clostridium difficile strains is indicated. The unique SPA fragment representing 60 Clostridium difficile strains is reported. The Clostridium difficile-specific (Cd) SPA fragment received a unique numerical identifier for reference in further analysis.

The results in Table 33 show that Clostridium difficile strains can be identified by the highly specific SPA fragment Cd1, thus providing an important method for its (early) detection using mcfDNA from peripheral blood samples. This shows the importance of SPA fragment sequencing as a novel approach as part of risk screening, e.g. after surgery or prolonged treatment with broad spectrum antibiotics, for developing CDI based on the (early) detection and identification of Clostridium difficile in peripheral blood and/or stool samples.

Risk screening for developing hospital-acquired infections: Acinetobacter baumannii is an opportunistic bacterial pathogen primarily associated with hospital-acquired infections. The recent increase in incidence, coupled with a dramatic increase in the incidence of multidrug-resistant (MDR) strains, has significantly raised the profile of this emerging opportunistic pathogen. Therefore, there is an unmet need for high-resolution, high-throughput and low-cost early detection of this bacterium in peripheral blood, something SPA fragment sequencing can provide. To analyze the discriminatory power of SPA fragment sequencing for the early detection of Acinetobacter baumannii as biomarker for the risk to developing a hospital-acquired infection from this pathogen, 50 nucleotide long fragments located upstream of the RpoB1-R1327 priming site were generated in silico for Acinetobacter baumannii strains. The results are presented in Table 34.

TABLE 34

| Acinetobacter baumannii species (Ab) specific SPA fragment | No. of |
|---|---|
| (50 nucleotides) sequence | strains |
| SPA fragment Ab1- | 352 |
| TCGATGTATTACGTACATTGGTTGAAATCCGTAACGGTAAAGGT | |
| GAAGTC (SEQ ID NO: 247) | |
| Acinetobacter baumannii | 346 |
| Klebsiella pneumoniae | 3 |
| Acinetobacter calcoaceticus | 1 |
| Acinetobacter pittii | 1 |
| Acinetobacter Tr-809 | 1 |
| SPA fragment Ab2- | 58 |
| TTGATGTATTACGTACATTAGTTGAAATCCGTAACGGTAAAGGTG | |
| AAGTC (SEQ ID NO: 248) | |
| Acinetobacter baumannii | 18 |
| Acinetobacter BS1 | 1 |
| Acinetobacter cl | 1 |
| Acinetobacter calcoaceticus | 1 |
| Acinetobacter KU | 1 |
| Acinetobacter lactucae | 5 |
| Acinetobacter NRRL | 1 |
| Acinetobacter pittii | 30 |
| SPA fragment Ab3- | 33 |
| TCGATGTATTACGTACGTTGGTTGAAATCCGTAACGGTAAAGGC | |
| GAAGTA (SEQ ID NO: 249) | |
| Acinetobacter baumannii | 18 |
| Acinetobacter nosocomialis | 15 |
| SPA fragment Ab4- | 22 |
| TCGATGTATTACGTACATTAGTTGAAATCCGTAACGGTAAAGGTG | |
| AAGTC (SEQ ID NO: 250) | |
| Acinetobacter AC1-2 | 1 |
| Acinetobacter ACIN00229 | 1 |
| Acinetobacter baumannii | 1 |
| Acinetobacter calcoaceticus | 5 |
| Acinetobacter oleivorans | 12 |
| Acinetobacter UBA11343 | 1 |
| Acinetobacter V2 | 1 |
| SPA fragment Ab5- | 8 |
| TCGATGTATTACGTACTTTAGTTGAAATTCGTAACGGTAAGGGTG | |
| AGGTC (SEQ ID NO: 251) | |
| Acinetobacter baumannii | 4 |
| Acinetobacter radioresistens | 4 |
| SPA fragment Ab6- | 7 |
| TTGATGTATTACGTACATTGGTTGAAATCCGTAACGGTAAAGGTG | |
| AAGTC (SEQ ID NO: 252) | |
| Acinetobacter baumannii | 2 |
| Acinetobacter NRRL | 1 |
| Acinetobacter pittii | 2 |
| Acinetobacter vivianii | 2 |
| SPA fragment Ab7- | 5 |
| TCGATGTGTTACGTACTTTAGTTGAAATTCGTAACGGTAAGGGTG | |
| AGGTC (SEQ ID NO: 253) | |
| Acinetobacter baumannii | 2 |
| Acinetobacter radioresistens | 3 |
| SPA fragment Ab8- | 5 |
| TAGATGTATTACGTACGTTGGTTGAAATCCGTAACGGTAAAGGC | |
| GAAGTA (SEQ ID NO: 254) | |
| Acinetobacter baumannii | 2 |
| Acinetobacter FDAARGOS 541 | 1 |
| Acinetobacter nosocomialis | 1 |
| Acinetobacter RQ Bin 15 | 1 |
| SPA fragment Ab9- | 5 |
| CTGATGTATTAAAAACATTAGTAGAAATCCGTAACGGTAAAGGT | |
| GAAGTC (SEQ ID NO: 255) | |
| Acinetobacter ACNIH1 | 1 |
| Acinetobacter baumannii | 1 |
| Acinetobacter GFQ9D192M | 1 |
| Acinetobacter variabilis | 2 |
| SPA fragment Ab10- | 4 |
| TTGATGTACTGCGTACATTGGTAGAAATCCGTAACGGTAAAGGT | |
| GAAGTC (SEQ ID NO: 256) | |
| Acinetobacter baumannii | 3 |
| Acinetobacter courvalinii | 1 |
| SPA fragment Ab11- | 3 |
| TTGATGTACTGCGTACATTGGTTGAAATCCGTAACGGTAAAGGT | |
| GAAGTC (SEQ ID NO: 257) | |
| Acinetobacter baumannii | 2 |
| Acinetobacter C16S1 | 1 |
| SPA fragment Ab12- | 2 |
| TCGATGTATTACGTACATTGGTTGAAATCCGTAATGGTAAAGGTG | |
| AAGTC (SEQ ID NO: 258) | |
| Acinetobacter baumannii | 2 |
| SPA fragment Ab13- | 2 |
| CTGATGTACTACGTACATTGGTTGAGATTCGTAACGGTAAAGGT | |
| GAAGTT (SEQ ID NO: 259) | |
| Acinetobacter baumannii | 1 |
| Acinetobacter ursingii | 1 |

Overview of the sequences of 50 nucleotide SPA fragments generated in silico for Acinetobacter baumannii strains and related species. For each SPA fragment, the Acinetobacter species and the number of strains is indicated. The SPA fragments representing 506 Acinetobacter baumannii strains and related species are reported. Acinetobacter baumannii-specific (Ab) SPA fragments received a unique numerical identifier for reference in further analysis.

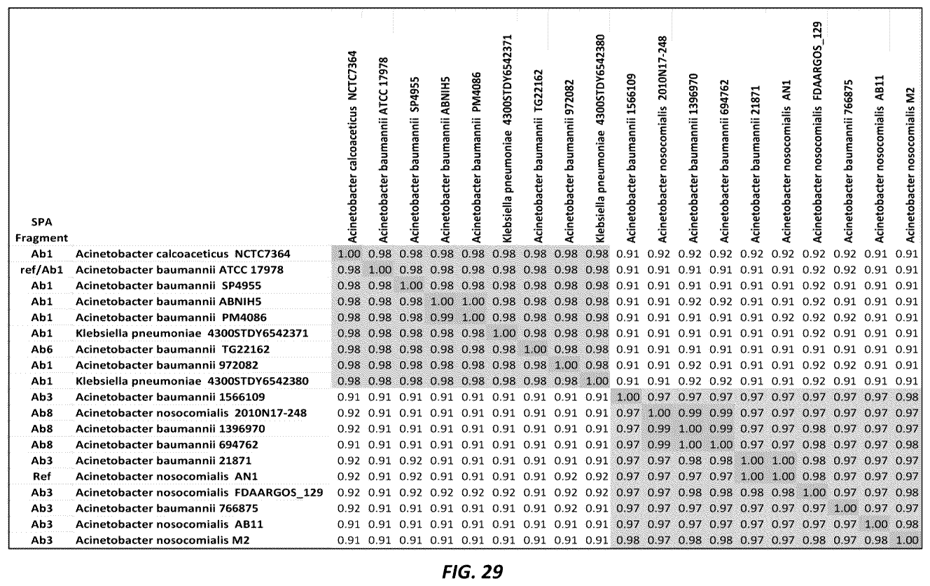

As shown in Table 34, the 50 nucleotide SPA fragments generated in silico for Acinetobacter baumannii strains, especially SPA fragment Ab1, largely allowed to distinguish Acinetobacter baumannii at the species level. However, several SPA fragments identified both Acinetobacter baumannii and related species, as well as some unexpected strains including Klebsiella pneumonia strains identified by SPA fragment Ab1. To clarify this result, whole genome-based ANI analysis was performed on selected Acinetobacter baumannii strains and representatives from related species that were identified by the same SPA fragments. Where available, the genomes sequences of the Acinetobacter species type strains were included in this analysis, of which the results are shown in FIGS. 29, 30 and 31. A total of eight ANI groups were identified:

ANI group I, which contains the strains identified by SPA fragment Ab1 (FIG. 29). This group included representatives of the 346 Acinetobacter baumannii strains as well as three Klebsiella pneumoniae strains and an Acinetobacter calcoaceticus strain. Based on their ANI scores with Acinetobacter baumannii strains, including the type strain ATCC 17978, it was concluded that the Klebsiella pneumoniae strains and a Acinetobacter calcoaceticus strain had been misidentified and should be reclassified as Acinetobacter baumannii.

ANI group II, which contains Acinetobacter baumannii and Acinetobacter nosocomialis strains identified by SPA fragments Ab3 and Ab8 (FIG. 29). Strains of ANI group II share very high ANI scores (>97%), indicating that they are the same species. Based on their low ANI scores with the ANI group I strains (91% to 92%), they represent a species closely related but distinct from Acinetobacter baumannii. Since the Acinetobacter nosocomialis type strain ANI was part of this group, the members of ANI group II should all be classified as Acinetobacter nosocomialis.

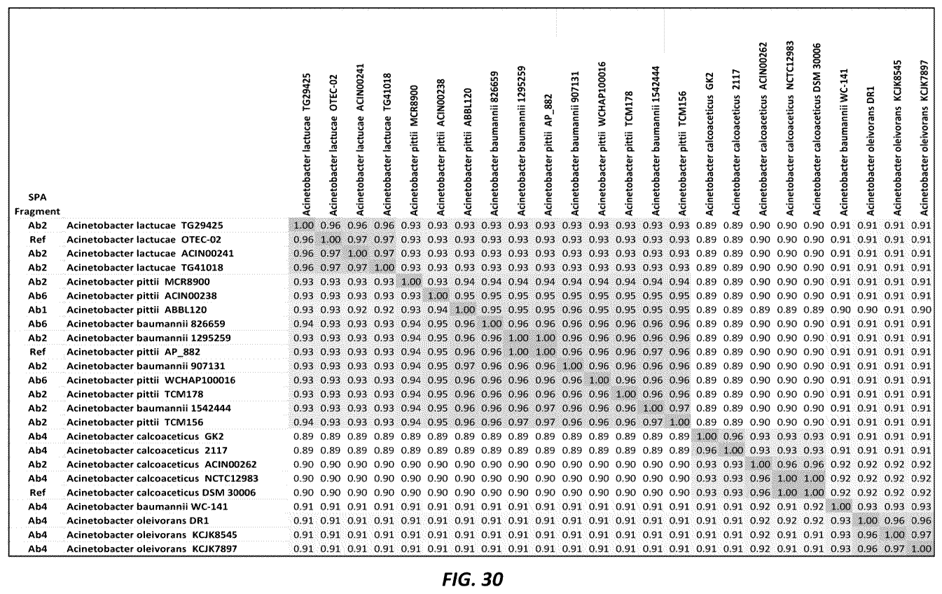

ANI group III, which contains Acinetobacter lactucae and Acinetobacter pittii strains identified by SPA fragment Ab2 (FIG. 30). The group also contains an Acinetobacter pittii strain identified by SPA fragment Ab1. Further analysis of the genome of this strain, which represents a metagenome assembled genome (MAG) of poor quality sequence, indicated that this MAG was highly contaminated and represented a chimeric assembly between Acinetobacter baumannii and Acinetobacter pittii. As such this MAG should be eliminated from the reference database. The group also contains Acinetobacter pittii strains identified by SPA fragment Ab6, as well as Acinetobacter baumannii strains identified by SPA fragments Ab1 and Ab6. Based on their whole genome-based ANI scores these strains are very similar to Acinetobacter pittii strains and should be reclassified as such.

ANI group IV, which contains closely related Acinetobacter calcoaceticus and Acinetobacter oleivorans strains identified by SPA fragments Ab2 and Ab4, as well as a strain identified by SPA fragment Ab4 that was misclassified as Acinetobacter baumannii (FIG. 30).

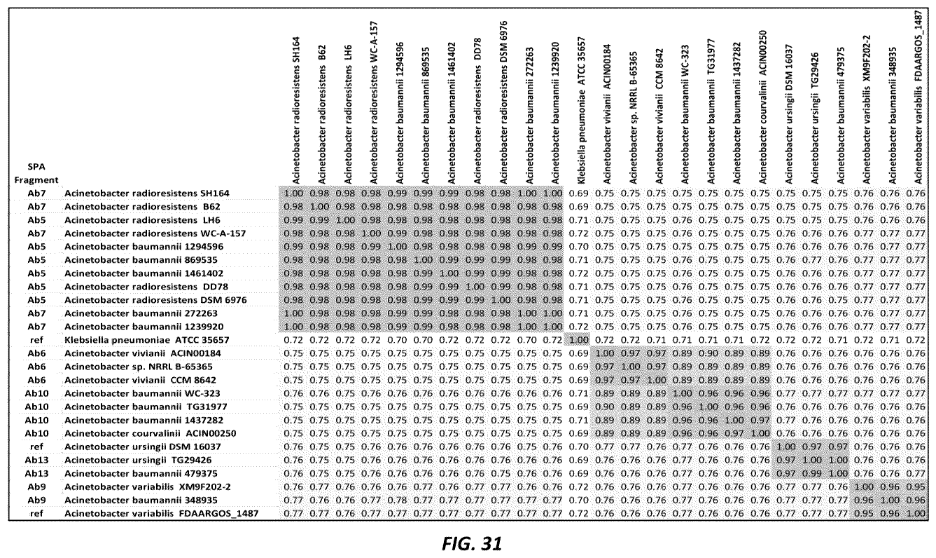

ANI group V, which contains Acinetobacter baumannii and Acinetobacter radioresistens strains identified by SPA fragments Ab5 and Ab7 (FIG. 31). Strains of ANI group V share very high ANI scores (>98%), indicating that they are the same species. Based on their low ANI scores with the ANI group I strains (75%), they represent a species different from Acinetobacter baumannii. Since the Acinetobacter radioresistens type strain DSM 6976 was part of this group, the members of ANI group V should all be classified as Acinetobacter radioresistens.

ANI group VI, which contains Acinetobacter baumannii and Acinetobacter courvalinii strains identified by SPA fragment Ab10 (FIG. 31). Based on their low ANI scores with the ANI group I strains (77%), they represent a species distinct from Acinetobacter baumannii, and therefore, the Acinetobacter baumannii strains in this group should all be reclassified as Acinetobacter courvalinii. In addition, ANI group VI includes the Acinetobacter vivianii strains identified by SPA fragment Ab6.

ANI group VII, which contains Acinetobacter baumannii and Acinetobacter ursingii strains, including the Acinetobacter ursingii type strain DSM 16037, identified by SPA fragment Ab13 (FIG. 31). Based on their low ANI scores with the ANI group I strains (76%), they represent a species distinct from Acinetobacter baumannii, and therefore, the members of this group should all be reclassified as Acinetobacter ursingii.

ANI group VIII, which contains Acinetobacter baumannii and Acinetobacter variabilis strains identified by SPA fragment Ab9 (FIG. 31). Based on their low ANI scores with the ANI group I strains (76%), they represent a species distinct from Acinetobacter baumannii, and therefore, the members of this group should all be reclassified as Acinetobacter variabilis.

Overall, these results confirm the phylogenetic resolution of 50 nucleotide SPA fragments to not only correctly identify Acinetobacter baumannii, but also point out strains that have been previously misclassified. A summary of the Acinetobacter baumannii strains and related species (Ab) specific SPA fragments as phylogenetic identifiers at the species level is provided in Table 35. These results show that unexpectedly, despite their relatively short size, 50 nucleotide long SPA fragment sequences covering the region upstream of the RpoB1-R1327 primer annealing site allow for high resolution phylogenetic identification of clinically relevant Acinetobacter strains at the species level, thus providing an important method for their (early) detection using mcfDNA from peripheral blood samples. This shows the importance of SPA fragment sequencing as a new approach as part of risk screening for hospital acquired infections based on the (early) detection and identification of Acinetobacter species.

TABLE 35
Summary of the Acinetobacter baumannii strains and related
species (Ab) specific SPA fragments as phylogenetic identifiers
at the species level. The SPA fragments are 50
nucleotides in length and cover the region upstream of
the RpoB1-R1327 primer annealing site.

| Acinetobacter baumannii | |
|---|---|
| species (Ab) | |
| specific SPA fragment | Species |
| SPA fragment Ab1, | Acinetobacter baumannii |
| SPA fragment Ab11, | |
| SPA fragment Ab12 | |
| SPA fragment Ab2 | Acinetobacter lactucae, |
| | Acinetobacter pittii |
| SPA fragment Ab2, | Acinetobacter calcoaceticus, |
| SPA fragment Ab4 | Acinetobacter oleivorans |
| SPA fragment Ab3, | Acinetobacter nosocomialis |
| SPA fragment Ab8 | |
| SPA fragment Ab5, | Acinetobacter radioresistens |
| SPA fragment Ab7 | |
| SPA fragment Ab6 | Acinetobacter vivianii, |
| | Acinetobacter pittii |
| SPA fragment Ab9 | Acinetobacter variabilis |
| SPA fragment Ab10 | Acinetobacter courvalinii |
| SPA fragment Ab13 | Acinetobacter ursingii |

### Example 9

### Phylogenetic Identification Based on the Combination of Multiple SPA Fragment Sequences.

In addition to the previous examples, the example presented below demonstrates how the SPA fragment sequencing method is generalizable and adaptable to improve phylogenetic resolution in a targetable fashion, which is informed by the existing knowledgebase of sequence variation at the species and subspecies level. Just as a lens can be refocused, resolution can be redirected to identify new taxa and subspecies of interest.

To address a limited number of cases where 50 nucleotide long fragments located upstream of the RpoB1-R1327 priming site fail to identify bacteria at the genus or species level, the combination of two SPA fragments can be used to improve the phylogenetic resolution. In the example provided for the Enterobacteriaceae, this is done by generating SPA fragments from two distinct regions of the rpoB gene and combining this information. However, the same can be achieved by combining the information of SPA fragments generated from two or more separate conserved housekeeping genes, including the prokaryotic genes coding for the DNA gyrase subunit B (gyrB), the chaperone protein (GroEL), the heat shock protein 60 (hsp60), the superoxide dismutase A protein (sodA), the TU elongation factor (tuf), the 60 kDa chaperonin protein (cpn60), and DNA recombinase proteins (including recA, recE). Practically, the same protocol as outlined in FIG. 2 would be used, except that two SPA primers would be included in the PCR reaction of Steps 4 and 5, resulting in the simultaneous generation of SPA fragments representing two regions for phylogenetic identification.

Screening for Enterobacteriaceae: The Enterobacteriaceae represents a group of often closely related bacteria, many of clinical importance. Key genera involve Escherichia, Shigella, Klebsiella, Salmonella and Serratia, many of which have been linked to sometimes life threatening and lethal infections, especially in immune compromised patients, including transplant patients where these bacteria are linked to post-transplant bloodstream infections, Graft versus Host Disease (GvHD), and increased mortality. Therefore, there is an unmet need for high-resolution, high-throughput and low-cost early detection of these bacteria in peripheral blood and other biopsy samples, something SPA fragment sequencing can provide. To analyze the discriminatory power of SPA fragment sequencing for the detection of Enterobacteriaceae, 50 nucleotide long fragments located upstream of the RpoB1-R1327 priming site were initially generated in silico for members of the Enterobacteriaceae. The results for the two SPA fragments able to identify the largest number of strains are presented in Table 36.

TABLE 36

| Enterobacteriaceae (Ent) specific SPA fragment | No. of |
|---|---|
| (50 nucleotides) sequence | strains |
| SPA fragment Ent1- | 1155 |
| TTGATGTTATGAAAAAGCTCATCGATATCCGTAACGGTAAAGGC | |
| GAAGTC (SEQ ID NO: 260) | |
| Escherichia coli | 1006 |
| Shigella flexneri | 40 |
| Shigella sonnei | 32 |
| Escherichia fergusonii | 14 |
| Escherichia albertii | 12 |
| Shigella dysenteriae | 9 |
| Shigella boydii | 9 |
| Enterobacteriaceae strains | 33 |
| SPA fragment Ent2- | 834 |
| TCGAAGTGATGAAGAAGCTCATCGATATCCGTAACGGTAAAGGC | |
| GAAGTG (SEQ ID NO: 261) | |
| Klebsiella pneumoniae | 535 |
| Enterobacter cloacae | 90 |
| Enterobacter asburiae | 38 |
| Klebsiella quasipneumoniae | 33 |
| Leclercia adecarboxylata | 20 |
| Serratia fonticola | 17 |
| Enterobacter kobei | 14 |
| Enterobacter mori | 5 |
| Enterobacter bugandensis | 4 |
| Klebsiella aerogenes | 3 |
| Enterobacter roggenkampii | 3 |
| Yokenella regensburgei | 3 |
| Escherichia coli | 2 |
| Lelliottia nimipressuralis | 2 |
| Enterobacteriaceae strains | 65 |

Overview of the sequences of 50 nucleotide SPA fragments generated in silico for Enterobacteriaceae. For each SPA fragment, the Enterobacteriaceae species and the number of strains is indicated. The SPA fragments representing 1,989 Enterobacteriaceae strains. Enterobacteriaceae-specific (Ent) SPA fragments received a unique numerical identifier for reference in further analysis.

As shown in Table 36, the 50 nucleotide SPA fragments generated in silico for strains belonging to the Enterobacteriaceae from the region upstream of the RpoB1-R1327 priming site failed to phylogenetically distinguish between strains on the genus level. This prompted us to evaluate if a combination of SPA fragments generated from two distinct regions of the rpoB gene would improve the phylogenetic identification of Enterobacteriaceae at the genus and species level. The results are presented in Table 37 and Table 38 for strains initially identified by SPA fragments Ent1 and Ent2, respectively.

TABLE 37

| Enterobacteriaceae (Ent) specific SPA fragment | No. of |
|---|---|
| (50 nucleotides) sequence | strains |
| SPA fragment Ent1- | 1155 |
| TTGATGTTATGAAAAAGCTCATCGATATCCGTAACGGTAAAGGCG | |
| AAGTC (SEQ ID NO: 260) | |
| SPA fragment Ent3*- | 851 |
| AACGTCGTATCTCCGCACTCGGCCCAGGCGGTCTGACCCGTGAA | |
| CGTGCA (SEQ ID NO: 262) | |
| Escherichia coli | 766 |
| Shigella flexneri | 40 |
| Shigella dysenteriae | 9 |
| Shigella boydii | 8 |
| Shigella sonnei | 6 |
| Escherichia strains | 22 |
| SPA fragment Ent4*- | 70 |
| AACGTCGTATCTCGGCACTCGGCCCAGGCGGTCTGACCCGTGAA | |
| CGTGCA (SEQ ID NO: 263) | |
| Escherichia coli | 69 |
| Shigella boydii | 1 |
| SPA fragment Ent5*- | 57 |
| AACGTCGTATCTCCGCACTCGGCCCGGGTGGTCTGACCCGTGAA | |
| CGTGCA (SEQ ID NO: 264) | |
| Escherichia coli | 34 |
| Shigella sonnei | 23 |
| SPA fragment Ent6*- | 52 |
| AACGTCGTATCTCCGCACTCGGCCCAGGTGGTCTGACCCGTGAA | |
| CGTGCA (SEQ ID NO: 265) | |
| Escherichia coli | 52 |
| SPA fragment Ent7*- | 24 |
| AGCGTCGTATCTCCGCACTCGGCCCAGGCGGTCTGACCCGTGAA | |
| CGTGCA (SEQ ID NO: 266) | |
| Escherichia fergusonii | 13 |
| Escherichia coli | 8 |
| Escherichia 0.2392 | 1 |
| Escherichia HH41S | 1 |
| Escherichia 94.0001 | 1 |
| SPA fragment Ent8*- | 17 |
| AACGTCGTATCTCGGCACTTGGCCCAGGCGGTCTGACCCGTGAA | |
| CGTGCA (SEQ ID NO: 267) | |
| Escherichia coli | 17 |
| SPA fragment Ent9*- | 13 |
| AACGTCGTATCTCCGCACTCGGCCCTGGCGGTCTGACTCGTGAA | |
| CGCGCG (SEQ ID NO: 268) | |
| Escherichia albertii | 13 |
| SPA fragment Ent10*- | 12 |
| AACGTCGTATCTCGGCCCTTGGCCCAGGCGGTCTGACCCGTGAA | |
| CGTGCA (SEQ ID NO: 269) | |
| Escherichia coli | 12 |
| SPA fragment Ent11*- | 7 |
| AACGTCGTATCTCAGCACTCGGCCCAGGTGGTCTGACCCGTGAA | |
| CGTGCA (SEQ ID NO: 270) | |
| Escherichia coli | 7 |
| SPA fragment Ent12*- | 5 |
| AACGTCGTATCTCCGCACTCGGCCCGGGCGGTCTGACCCGTGAA | |
| CGTGCA (SEQ ID NO: 271) | |
| Escherichia coli | 5 |
| SPA fragment Ent13*- | 5 |
| AACGTCGTATTTCCGCACTCGGCCCAGGCGGTCTGACCCGTGAA | |
| CGTGCA (SEQ ID NO: 272) | |
| Escherichia coli | 5 |
| SPA fragment Ent14*- | 5 |
| AACGTCGTATCTCTGCACTCGGCCCAGGCGGTCTGACCCGTGAA | |
| CGTGCA (SEQ ID NO: 273) | |
| Escherichia coli | 2 |
| Escherichia MOD1-EC6475 | 1 |
| Escherichia 4726-5 | 1 |
| Escherichia 93.0816 | 1 |
| SPA fragment Ent15*- | 4 |
| AACGTCGTATCTTCGCACTCGGCCCAGGCGGTCTGACCCGTGAA | |
| CGTGCA (SEQ ID NO: 274) | |
| Escherichia coli | 4 |
| SPA fragment Ent16*- | 3 |
| AACGTCGTATCTCCGCACTCGGTCCAGGCGGTCTGACCCGTGAA | |
| CGTGCA (SEQ ID NO: 275) | |
| Escherichia coli | 2 |
| Shigella sonnei | 1 |
| SPA fragment Ent17*- | 2 |
| AACGTCGTATCTCTGCACTCGGTCCAGGCGGTCTGACCCGTGAA | |
| CGTGCA (SEQ ID NO: 276) | |
| Escherichia MR | 1 |
| Escherichia coli | 1 |

Overview of the sequences of 50 nucleotide SPA fragments generated in silico for Enterobacteriaceae. Strains were initially selected based on the presence of the 50 nucleotide SPA fragment Ent1 (see table 36), generated upstream of the RpoB1-R1327 priming site. Subsequently, 50 nucleotide SPA fragments were generated upstream of the RpoB6-R1630 priming site. The sequences of these SPA fragments are presented and for each of these SPA fragments, the Enterobacteriaceae species and the number of strains is indicated. SPA fragments identifying a single strain were left out. Enterobacteriaceae-specific (Ent) SPA fragments received a unique numerical identifier for reference in further analysis, with an asterisk symbol “*” indicating that the SPA fragment was generated from the region upstream of the RpoB1-R1630 priming site.

TABLE 38

| Enterobacteriaceae (Ent) specific SPA fragment | No. of |
|---|---|
| (50 nucleotides) sequence | strains |
| SPA fragment Ent2- | 834 |
| TCGAAGTGATGAAGAAGCTCATCGATATCCGTAACGGTAAAGGC | |
| GAAGTG (SEQ ID NO: 261) | |
| SPA fragment Ent18*- | 557 |
| AACGTCGTATCTCCGCACTCGGCCCAGGCGGTCTGACCCGTGAG | |
| CGCGCA (SEQ ID NO: 277) | |
| Klebsiella pneumoniae | 517 |
| Klebsiella quasipneumoniae | 33 |
| Klebsiella aerogenes | 3 |
| Serratia liquefaciens | 1 |
| Klebsiella 18A069 | 1 |
| Enterobacteriaceae S05 | 1 |
| Klebsiella 01A030 | 1 |
| SPA fragment Ent19*- | 75 |
| AACGTCGTATCTCTGCACTCGGCCCAGGCGGTCTGACCCGTGAA | |
| CGTGCA (SEQ ID NO: 278) | |
| Enterobacter cloacae | 26 |
| Enterobacter kobei | 12 |
| Enterobacter bugandensis | 4 |
| Enterobacter asburiae | 4 |
| Enterobacter roggenkampii | 3 |
| Lelliottia nimipressuralis | 2 |
| Enterobacter 725m/11 | 1 |
| Enterobacter ODB01 | 1 |
| Enterobacter AM17-18 | 1 |
| Enterobacter mori | 1 |
| Enterobacter 35730 | 1 |
| Leclercia adecarboxylata | 1 |
| Enterobacter 44593 | 1 |
| Enterobacter GN02366 | 1 |
| Enterobacter 50588862 | 1 |
| Enterobacter M4-VN | 1 |
| Enterobacter RHBSTW-00901 | 1 |
| Enterobacter N18-03635 | 1 |
| Enterobacter T2 | 1 |
| Enterobacter Acro-832 | 1 |
| Enterobacter WCHEn090040 | 1 |
| Enterobacter DC1 | 1 |
| Enterobacter Tr-810 | 1 |
| Enterobacter E12 | 1 |
| Enterobacter WCHEs120002 | 1 |
| Enterobacter GN02186 | 1 |
| Leclercia LK8 | 1 |
| Enterobacter GN02266 | 1 |
| Enterobacter 35669 | 1 |
| Enterobacter GN02283 | 1 |
| SPA fragment Ent20*- | 75 |
| AACGTCGTATCTCTGCACTCGGCCCAGGCGGTCTGACCCGTGAA | |
| CGCGCA (SEQ ID NO: 279) | |
| Enterobacter cloacae | 39 |
| Enterobacter asburiae | 26 |
| Enterobacter SECR19-1250 | 1 |
| Klebsiella pneumoniae | 1 |
| Enterobacter kobei | 1 |
| Enterobacter mori | 1 |
| Enterobacter RHBSTW-01064 | 1 |
| Enterobacter DC3 | 1 |
| Enterobacter WCHECI1597 | 1 |
| Enterobacter GN02174 | 1 |
| Enterobacter 35699 | 1 |
| Enterobacter JMULE2 | 1 |
| SPA fragment Ent21*- | 19 |
| AGCGTCGTATCTCTGCACTCGGCCCAGGCGGTCTGACCCGTGAG | |
| CGCGCA (SEQ ID NO: 280) | |
| Leclercia adecarboxylata | 15 |
| Enterobacteriaceae w17 | 1 |
| Leclercia LSNIH1 | 1 |
| Enterobacteriaceae w6 | 1 |
| Leclercia 1106151 | 1 |
| SPA fragment Ent22*- | 15 |
| AACGTCGTATCTCTGCATTGGGCCCAGGCGGTCTGACCCGTGAA | |
| CGTGCC (SEQ ID NO: 281) | |
| Serratia fonticola | 14 |
| Serratia 3ACOL1 | 1 |
| SPA fragment Ent23*- | 13 |
| AACGTCGTATCTCTGCACTCGGCCCAGGCGGTCTGACTCGTGAA | |
| CGCGCA (SEQ ID NO: 282) | |
| Enterobacter cloacae | 11 |
| Enterobacter WCHEn045836 | 1 |
| Enterobacter GN02534 | 1 |
| SPA fragment Ent24*- | 8 |
| AGCGTCGTATCTCTGCACTCGGCCCAGGCGGTCTGACCCGTGAA | |
| CGCGCA (SEQ ID NO: 283) | |
| Enterobacter cloacae | 5 |
| Enterobacteriaceae ATCC | 1 |
| Enterobacter A11 | 1 |
| Enterobacter BIDMC92 | 1 |
| SPA fragment Ent25*- | 7 |
| AACGTCGTATCTCTGCACTCGGCCCAGGCGGTCTGACCCGTGAG | |
| CGCGCA (SEQ ID NO: 284) | |
| Enterobacter asburiae | 2 |
| Leclercia LSNIH6 | 1 |
| Enterobacter SES19 | 1 |
| Enterobacter mori | 1 |
| Leclercia LSNIH7 | 1 |
| Enterobacter NFIX59 | 1 |
| SPA fragment Ent26*- | 6 |
| AACGTCGTATTTCTGCACTCGGCCCAGGCGGTCTGACCCGTGAA | |
| CGTGCA (SEQ ID NO: 285) | |
| Enterobacter cloacae | 2 |
| Enterobacter GN02225 | 1 |
| Enterobacter GN02204 | 1 |
| Enterobacter asburiae | 1 |
| Enterobacter 42202 | 1 |
| SPA fragment Ent27*- | 5 |
| AGCGTCGTATCTCTGCACTCGGCCCAGGCGGTCTGACCCGTGAA | |
| CGTGCA (SEQ ID NO: 286) | |
| Yokenella regensburgei | 3 |
| Enterobacter asburiae | 1 |
| Enterobacter cloacae | 1 |
| SPA fragment Ent28*- | 5 |
| AACGTCGTATCTCTGCACTCGGCCCGGGCGGTCTGACCCGTGAG | |
| CGCGCA (SEQ ID NO: 287) | |
| Enterobacter mori | 2 |
| Enterobacter cloacae | 1 |
| Escherichia coli | 1 |
| Enterobacter tabaci | 1 |
| SPA fragment Ent29*- | 4 |
| AGCGTCGTATCTCTGCACTCGGCCCGGGCGGTCTGACCCGTGAG | |
| CGCGCA (SEQ ID NO: 288) | |
| Leclercia UBA9585 | 1 |
| Leclercia adecarboxylata | 1 |
| Enterobacter UMGS201 | 1 |
| Leclercia 119287 | 1 |
| SPA fragment Ent30*- | 3 |
| AACGTCGTATCTCCGCACTCGGCCCGGGCGGTCTGACCCGTGAA | |
| CGTGCA (SEQ ID NO: 289) | |
| Enterobacter asburiae | 1 |
| Kluyvera SCKS090646 | 1 |
| Enterobacter cloacae | 1 |
| SPA fragment Ent31*- | 3 |
| AGCGTCGTATCTCTGCATTGGGCCCAGGCGGTCTGACCCGTGAA | |
| CGTGCC (SEQ ID NO: 290) | |
| Serratia fonticola | 3 |

Overview of the sequences of 50 nucleotide SPA fragments generated in silico for Enterobacteriaceae. Strains were initially selected based on the presence of the 50 nucleotide SPA fragment Ent2 (see table 36), generated upstream of the RpoB1-R1327 priming site. Subsequently, 50 nucleotide SPA fragments were generated upstream of the RpoB6-R1630 priming site. The sequences of these SPA fragments are presented and for each of these SPA fragments, the Enterobacteriaceae species and the number of strains is indicated. SPA fragments identifying a single strain were left out. Enterobacteriaceae-specific (Ent) SPA fragments received a unique numerical identifier for reference in further analysis with an asterisk symbol “*” indicating that the SPA fragment was generated from the region upstream of the RpoB1-R1630 priming site.

Comparing the results from Table 36 and Table 37 shows the improved phylogenetic classification of species that clustered together for SPA fragment Ent1 after they were further classified using 50 nucleotide SPA fragments generated from the region upstream of the position of the RpoB6-R1630 priming site. For instance, the 1006 Escherichia coli strains previously identified by SPA fragment Ent1 broke into several subgroups, most of the 32 Shigella sonnei strains ended up in different groups, as did the 14 Escherichia fergusonii and the 13 Escherichia albertii strains. The results from whole genome-based ANI show that strains identified by SPA fragments Ent3*, Ent4*, Ent5* and Ent16*, despite representing different species, are very closely related with ANI scores of >0.97. Members of the genus Shigella have high genomic similarity to Escherichia coli and are often considered to be atypical members of this species. In line with the observation that many Shigella and Escherichia coli strains were identified by the same SPA fragment, Shigella species were reclassified as Escherichia species in the Genome Taxonomy Database (GTDB) using an operational average nucleotide identity (ANI)-based approach nucleated around type strains (Parks et al, 2021).

SPA fragment Ent7* identified Escherichia coli and Escherichia fergusonii strains, and SPA fragment Ent9* identified Escherichia albertii strains. Based on whole genome-based ANI it can also be concluded that Shigella boydii strain 60_SBOY (Ent4) should be assigned as Escherichia coli, that Escherichia coli strain 102606_aEPEC (Ent9) should be reassigned as Escherichia albertii, and that Escherichia coli strain JL_F4_1 (Ent16) and Shigella sonnei strain ECSW+02 (Ent16) represent the same species with an ANI score of 1.00.

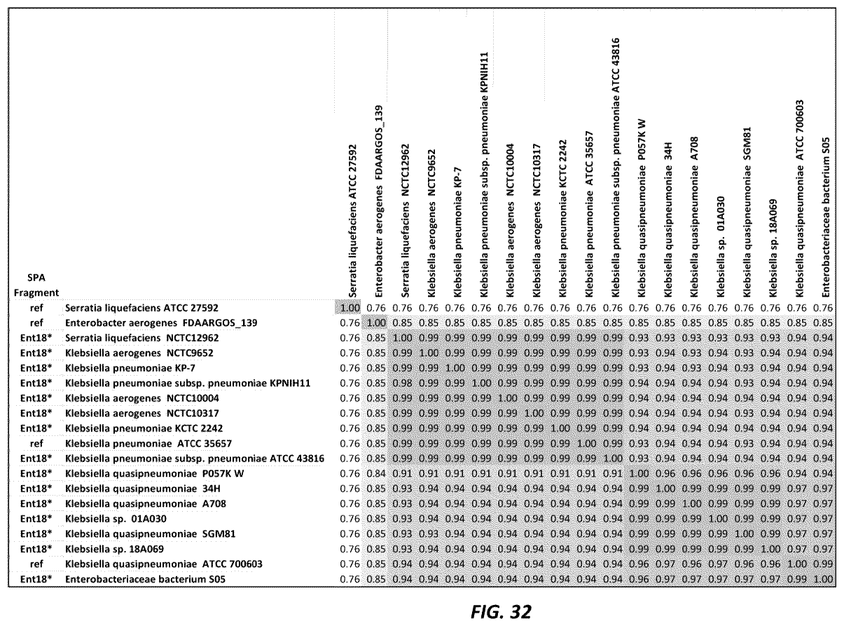

Similarly, comparing the results from Table 36 and Table 38 shows the improved phylogenetic classification of species that clustered together for SPA fragment Ent2 after they were further classified using 50 nucleotide SPA fragments generated from the region upstream of the position of the RpoB6-R1630 priming site. For instance, SPA fragment Ent18* specifically grouped closely related Klebsiella pneumoniae, Klebsiella quasipneumoniae and Klebsiella aerogenes strains. This was confirmed by whole genome-based ANI as shown in FIG. 32, where two major groups could be distinguished. Strains of ANI group I share very high ANI scores (>99%), indicating that the Klebsiella pneumoniae, Klebsiella quasipneumoniae and Klebsiella aerogenes strains of this group represent members of the same species. Since this group includes the Klebsiella pneumoniae ATCC 43816 type-strain, members of this group should be identified as Klebsiella pneumoniae. Similarly, members of the ANI group II, which include the Klebsiella quasipneumoniae ATCC 700603 type-strain, should be identified as Klebsiella quasipneumoniae.

In addition to Klebsiella pneumoniae and Klebsiella quasipneumoniae strains, SPA fragment Ent2 identified closely related Enterobacter sp. strains that could be further classified using 50 nucleotide SPA fragments generated from the region upstream of the position of the RpoB6-R1630 priming site, as was confirmed by whole genome-based ANI. Based on the ANI results it can be concluded that many strains that were previously identified as Enterobacter cloacae represent in fact different but closely related species. However, the strains designated as Enterobacter cloacae identified by SPA fragments Ent20* and Ent23* represent true Enterobacter cloacae; this also includes the Enterobacter cloacae ATCC 13047 type-strain. SPA fragment Ent20* also identifies Enterobacter asburiae strains. However, based on their ANI score of 0.88 with Enterobacter cloacae ATCC 13047, the strains identified by SPA fragment Ent24* represent a different species, which is confirmed by their unique SPA fragment.

SPA fragment Ent19* grouped closely related Enterobacter sp. strains, including Enterobacter kobei strains, Enterobacter roggenkampii strains, Enterobacter bugandensis strains, and Enterobacter asburiae strains. Based on whole genome ANI, Leclercia adecarboxylata UMB0660 identified by SPA fragment Ent19* represents an Enterobacter bugandensis strain. In addition to SPA fragment Ent19*, Enterobacter asburiae strains were identified by SPA fragment Ent20*, Ent25*, Ent26*, Ent30*, and Ent27*, which also identified the reference strain Enterobacter asburiae 35734 and the type-strain Yokenella regensburgei ATCC 49455

SPA fragment Ent20* identified strains from the closely related species Enterobacter cloacae and Enterobacter asburiae. Serratia fonticola strains were specifically identified by SPA fragments Ent22* and Ent31*. SPA fragment Ent28* was found to be specific for Enterobacter mori, while SPA fragments Ent21* and Ent29* were found to be specific for Leclercia adecarboxylata and a closely related Leclercia species; this species was also identified by SPA fragment Ent25*. The results also show that Leclercia adecarboxylata strain UMB0660, identified by SPA fragment Ent19*, should be reassigned to Enterobacter bugandensis. The results for the Enterobacteriaceae specific SPA fragments are summarized in Table 39.

TABLE 39
Summary of Enterobacteriaceae species (Ent) specific SPA fragments as
phylogenetic identifiers at the species level. The 50 nucleotide as
SPA fragment “Ent” and a numerical identifier, with an asterisk
symbol “*” indicating that the SPA fragment was generated from
the region upstream of the RpoB1-R1630 priming site.
SPA fragments are identified

| Enterobacteriaceae species | |
|---|---|
| (Ent) specific | |
| SPA fragment | Species |
| SPA fragment Ent3* | Escherichia coli, Shigella flexneri, |
| | Shigella dysenteriae, |
| | Shigella boydii, Shigella sonnei |
| SPA fragment Ent4*, | Escherichia coli |
| SPA fragment Ent6*, | |
| SPA fragment Ent8*, | |
| SPA fragment Ent10* | |
| SPA fragment Ent11*, | |
| SPA fragment Ent12*, | |
| SPA fragment Ent13*, | |
| SPA fragment Ent14*, | |
| SPA fragment Ent15*, | |
| SPA fragment Ent17* | |
| SPA fragment Ent5*, | Escherichia coli, Shigella sonnei |
| SPA fragment Ent16* | |
| SPA fragment Ent7* | Escherichia coli, |
| | Escherichia fergusonii |
| SPA fragment Ent9* | Escherichia albertii |
| SPA fragment Ent18* | Klebsiella pneumoniae, |
| | Klebsiella quasipneumoniae |
| SPA fragment Ent19* | Enterobacter kobei, Enterobacter |
| | bugandensis, Enterobacter asburiae, |
| | Enterobacter roggenkampii |
| SPA fragment Ent20* | Enterobacter cloacae, |
| | Enterobacter asburiae |
| SPA fragment Ent21*, | Leclercia adecarboxylata, |
| SPA fragment Ent25* | Leclercia sp. Nov. |
| SPA fragment Ent22*, | Serratia fonticola |
| SPA fragment Ent31* | |
| SPA fragment Ent23* | Enterobacter cloacae |
| SPA fragment Ent24* | Enterobacter sp. Nov. |
| SPA fragment Ent25* | Leclercia sp. Nov., Enterobacter asburiae |
| SPA fragment Ent26* | Enterobacter asburiae, Enterobacter kobei |
| SPA fragment Ent27* | Yokenella regensburgei, Enterobacter |
| | asburiae |
| SPA fragment Ent28* | Enterobacter mori |
| SPA fragment Ent29* | Leclercia adecarboxylata |
| SPA fragment Ent30* | Enterobacter asburiae |

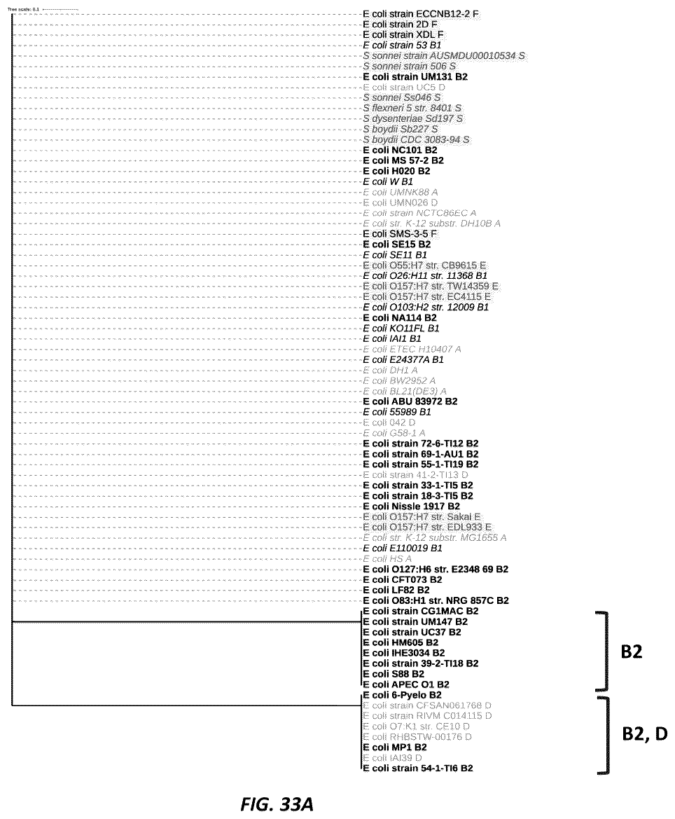

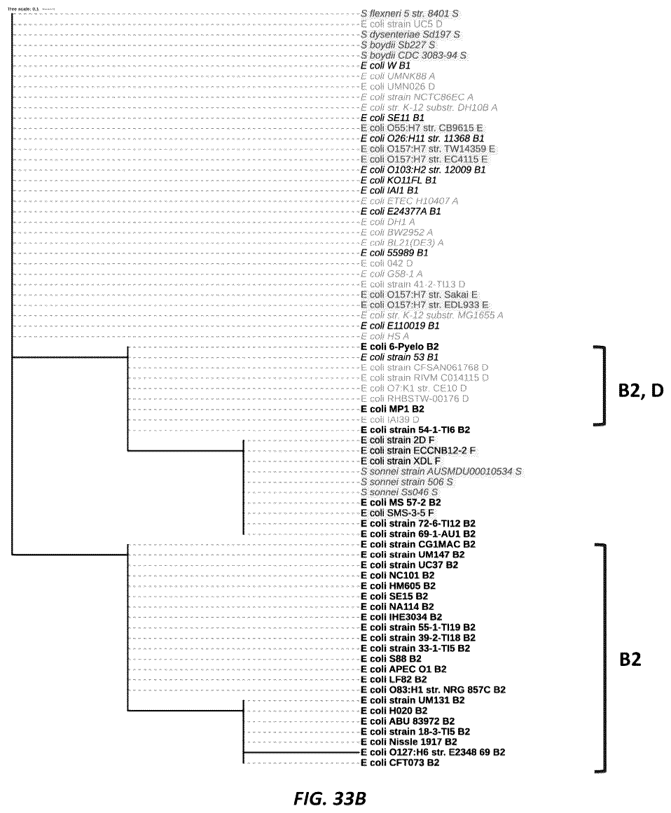

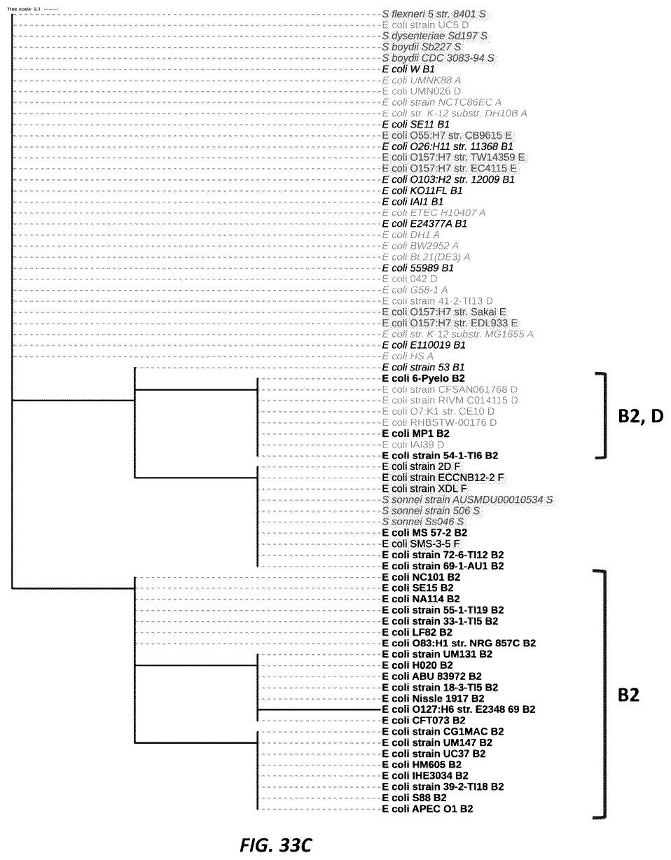

To show the synergy of using two SPA fragments generated from two distinct regions of the rpoB gene for phylogenetic identification of closely related bacteria we compared the phylogenetic classification of 121 Escherichia coli strains and related species belonging to different phylotypes as described by Fang et al (2018). This includes Escherichia coli phylotype B2 strains, which are prevalent in IBD patients and have distinct metabolic capabilities that allow them to colonize mucosa. The results are presented in FIGS. 33A, 33B and 33C. FIG. 33A shows the phylogenetic tree of the strains when the sequences of 50 nucleotide SPA fragments generated from the region upstream of the RpoB1-R1327 priming site are used. Except for a subset of Escherichia coli phylotype B2 strains and a small group of Escherichia coli phylotype B2 and D strains, all strains clustered together, including the Shigella species that are closely related to Escherichia coli phylotype A and B1 strains. FIG. 33B shows the phylogenetic tree of the strains when the sequences of 50 nucleotide SPA fragments generated from the region upstream of the RpoB1-R1630 priming site are used. This resulted in a significant improvement of the phylogenetic clustering, especially for the Escherichia coli phylotype B2 strains. FIG. 33C shows the phylogenetic tree of the strains when the combination of sequences of 50 nucleotide SPA fragments generated from the regions upstream of the RpoB1-R1327 and RpoB6-R1630 priming sites are used. The combined use of SPA fragments that represents different gene regions with phylogenetic information refines the phylogenetic clustering of the Escherichia coli strains, including the phylotype B2 strains, to a resolution that is not obtained when any of the two fragments are individually used. Therefore, for the identification of closely related species, the SPA fragment method (FIG. 2) can include one or more additional primers to simultaneously target different regions for phylogenetic identification. These regions can be located on the same gene, as demonstrated for the rpoB gene, or on different phylogenetic genes, especially conserved housekeeping genes. Subsequently, data from the individual primers are processed for community composition and species identification. In case of inconclusive identification, the information from both SPA fragment sets is combined to enhance the phylogenetic resolution. In addition, having more than one primer serves as an internal control for community composition. Overall, the results demonstrate how the disclosed SPA fragment sequencing method is generalizable and adaptable to improve phylogenetic resolution in a targetable fashion for the identification of closely related species of clinical importance, including members of the Enterobacteriaceae.

In certain instances, disease phenotypes caused by bacteria will depend on the presence of virulence/pathogenicity factors located on mobile genetic elements, including conjugative and/or mobile plasmids, phages, and pathogenicity islands that can be horizontally transferred between bacteria, as is the case for Escherichia coli, Salmonella, Klebsiella, Listeria, Bacillus, pyogenic streptococci and Clostridium perfringens, among others (for review, see Gyles and Boerlin, 2014). As the result of horizontal gene transfer, phylogenetic information on species composition will be insufficient to predict disease pathology, and therefore needs to be complemented with information on community functionality. For instance, the presence in Escherichia coli of the PKS pathogenicity island encoding, among other virulence factors, for genotoxic colibactin synthesis has been linked to increased risk for developing colorectal cancer (Pleguezuelos-Manzano et al, 2020). As discussed for colorectal cancer in Example 7, multiplex SPA fragment sequencing provides the flexibility to address both phylogenetic identification and community functional in a same amplification step. By designing a primer for SPA fragment amplification that specifically targets the PKS gene cluster essential for colibactin synthesis, the presence of genotoxic Escherichia coli strains (Pleguezuelos-Manzano et al, 2020) can be determined, and combined with phylogenetic information be used for improved risk assessment and detection of colorectal cancer.

### Example 10: Sensitivity Analysis of Spa Fragment Sequencing

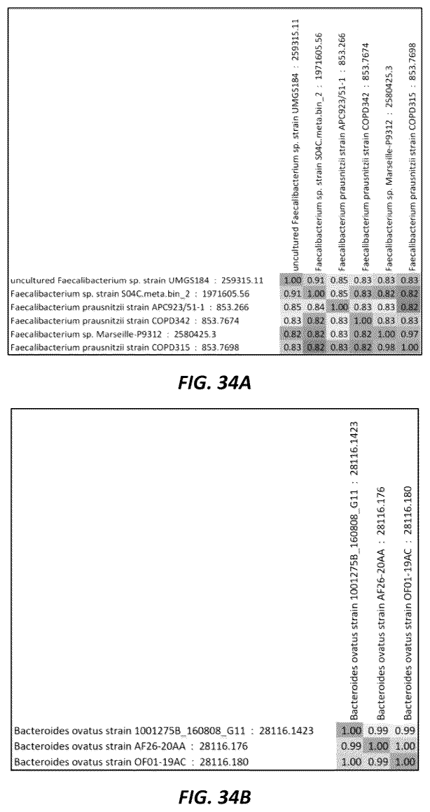

vDescription of the community used for the simulations: To understand the sensitivity of the SPA fragment sequencing method, the gut microbiome community of a person suffering from intestinal complications was used for in silico simulations. The assumption was that this microbiome would leave a similar signature in the mcfDNA. This consortium (see Table 40), whose composition was determined using long-read PacBio sequencing, is interesting as it includes six Metagenome Assembled Genomes (MAGs) representing closely related Faecalibacterium strains that based on their Average Nucleotide Identity (ANI) represent five different species/subspecies (FIG. 34A). Therefore, one of the questions is whether the SPA fragment sequencing method can provide the level of phylogenetic resolution to discriminate between these strains, and if this would be at the 25 base pair or 50 base pair SPA fragment length. This consortium also includes three MAGs representing Bacteroides ovatis, which were found to be very similar based on their ANI score of 0.99 (FIG. 34B), and that their assignment to different MAGs was most likely the result of binning errors. As such it is expected that these strains would share the same SPA fragment. Since the PacBio sequencing did not result in complete MAGs for all strains, especially for strains with lower abundances, whole genome sequences from the closest related strains as identified with ANI were used in the simulations.

TABLE 40
Composition (species name and genome ID) and relative species
abundances of the gut microbiome community used for the simulations.
Strains with identical SPA fragments of 25 base pairs
(see Table 41) are indicated by the same *number.
Relative Abundance % = (number of genome copies of each
species/sum of genome copies of all species) × 100%.

| | | Relative |
|---|---|---|
| | PATRIC | Abundance |
| Microbial species | Genome ID | % |
| Alistipes onderdonkii strain D10-10 | 328813.45 | 0.54 |
| Clostridia bacterium strain | 2044939.1074 | 0.58 |
| Blautia sp. AF19-10LB | 2292961.3 | 0.58 |
| Roseburia intestinalis ERR321618-bin.7 | 166486.952 | 0.59 |
| Dorea longicatena strain MSK.11.4 | 88431.960 | 0.63 |
| Lachnospiraceae bacterium strain | 1898203.1773 | 0.64 |
| MGYG-HGUT-00193 | | |
| Roseburia inulinivorans strain | 360807.1171 | 0.71 |
| SRR5519173-bin.6 *1 | | |
| Roseburia inulinivorans strain | 360807.64 | 0.71 |
| AF28-15 *1 | | |
| Faecalibacterium sp. strain | 1971605.56 | 0.72 |
| S04C.meta.bin_2 | | |
| Bacteroidaceae bacterium strain | 2212467.8 | 0.72 |
| MGYG-HGUT-00144 | | |
| Bacteroides caccae strain BIOML-A1 *2 | 47678.882 | 0.73 |
| Parabacteroides merdae strain | 46503.2088 | 0.83 |
| 1001136B_160425_B1 | | |
| Parabacteroides distasonis strain LMAG:27 | 823.3168 | 0.86 |
| Bacteroides caccae strain BIOML-A2 *2 | 47678.881 | 0.87 |
| uncultured Dialister sp. strain | 278064.91 | 0.88 |
| ERR414242-bin.5 | | |
| Coprococcus comes strain MSK.16.14 | 410072.533 | 0.88 |
| uncultured Eubacterium sp. strain UMGS39 | 165185.165 | 0.94 |
| Ruminococcaceae bacterium | 1898205.22 | 0.96 |
| strain UBA9091 | | |
| uncultured Clostridiales bacterium strin | 172733.1407 | 0.99 |
| UMGS84 | | |
| Alistipes finegoldii DSM 17242 | 679935.3 | 1.00 |
| uncultured Faecalibacterium sp. strain | 259315.11 | 1.03 |
| UMGS184 | | |
| Agathobaculum butyriciproducens strain | 1628085.84 | 1.04 |
| COPD228 | | |
| Eubacterium sp. 38_16 | 1897002.3 | 1.07 |
| Subdoligranulum sp. strain S08B.meta.bin_8 | 2053618.24 | 1.07 |
| Anaerostipes hadrus strain S01C.meta.bin_9 | 649756.2503 | 1.1 |
| [Ruminococcus] lactaris strain | 46228.446 | 1.15 |
| SRR7721875-bin.26 | | |
| Ruminococcus sp. D40t1_170626_H2 *3 | 2787081.3 | 1.2 |
| Blautia faecis strain MSK.11.45 *3 | 871665.25 | 1.26 |
| Bifidobacterium longum subsp. | 1679.11 | 1.37 |
| longum strain 9 | | |
| Acetatifactor sp. strain COPD172 | 1872090.5 | 1.44 |
| Firmicutes bacterium AM31-12AC | 2292892.3 | 1.46 |
| Faecalibacterium prausnitzii strain | 853.266 | 1.47 |
| APC923/51-1 | | |
| Ruminococcus sp. strain UBA10663 | 41978.12 | 1.5 |
| Bacteroides ovatus strain OF01-19AC *4 | 28116.180 | 1.6 |
| Bacteroides sp. AM30-16 | 2292949.3 | 1.73 |
| Bifidobacterium pseudocatenulatum strain | 28026.777 | 1.76 |
| Alistipes obesi MGYG-HGUT-01415 | 1118064.514 | 1.93 |
| Faecalibacterium sp. Marseille-P9312 *5 | 2580425.3 | 2.01 |
| Faecalibacterium prausnitzii strain | 853.7698 | 2.04 |
| COPD315 *5 | | |
| Ruminococcus sp. AM40-10AC | 2293212.3 | 2.07 |
| Blautia wexlerae strain | 418240.389 | 2.11 |
| 1001270J_160509_E6 | | |
| [Eubacterium] rectale strain BIOML-A1 | 39491.2479 | 2.2 |
| Paraprevotella clara CAG:116 strain | 1263095.48 | 2.23 |
| MGS:116 | | |
| Ruminococcus sp. CAG:9 | 1262967.3 | 2.36 |
| Bacteroides ovatus AF26-20AA *4 | 28116.176 | 2.45 |
| Faecalibacterium prausnitzii strain | 853.7674 | 2.73 |
| COPD342 | | |
| Alistipes putredinis DSM 17216 | 445970.5 | 2.92 |
| Blautia massiliensis strain MSK.13.24 | 1737424.64 | 3.14 |
| Bacteroides ovatus strain | 28116.1423 | 3.69 |
| 1001275B_160808_G11 *4 | | |
| Agathobacter sp. strain COPD130 | 2021311.24 | 4.26 |
| Bacteroides vulgatus strain VPI-5710 | 821.3904 | 5.65 |
| strain not applicable | | |
| Bacteroides stercoris strain AM51-2BH | 46506.122 | 21.61 |

Faecalibacterium species are marked in bold.

In silico generation of the SPA fragments for the individual community members: To demonstrate the discriminatory power of SPA fragment sequencing targeting the RpoB gene, 25 base pair and 50 base pair long SPA fragments located 3′ of the RpoB1-R1327 primer annealing site were generated in silico for each of the community members. The results for the 25 base pair long SPA fragments that identified more than one bacterial strain present in the community are presented in Table 41. Identical results were obtained for the 50 base pair SPA fragments. It should be noted that for the simulations, we still consider that all strains can be identified by their individual SPA fragments.

Using the sequences of either the 25 or 50 base pair long SPA fragments, 50 of the 52 strains in the community could be identified on the species level by their unique SPA fragments. Four SPA fragments obtained in silico with the RpoB1-R1327 primer identified multiple but very closely related strains (Table 41), as was confirmed by their identical genome taxonomy. Based on genome taxonomy and ANI it was concluded that each recognized strains belonging to the same species, and that their assignment to different MAGs was most likely the result of binning errors.

The six Faecalibacterium strains, classified on whole genome-based ANI as belonging to five different (sub)species (FIG. 34A), were each identifiable by their unique SPA fragment sequence of 25 base pairs or longer, except for two strains that both belonged to Faecalibacterium prausnitzii subgroup G, and that shared ANI scores of 97%, indicating that they represent the same species, as confirmed by these two strains sharing the same 50 base pair long SPA fragment. As such whole genome-based ANI and SPA fragment sequences provided the same phylogenetic resolution to discriminate these strains at the (sub)species level. The Bacteroides ovatus strains, that based on genome taxonomy and whole genome ANI were closely related and represented the same species (FIG. 34B), shared the same 25 base pair and 50 base pair SPA fragment sequence, also pointing to similar phylogenetic resolution of the two methods. The only exception was for the two closely related Roseburia species that shared common 25 and 50 base pair long SPA fragments, but that according to their genome taxonomy based on the Genome Taxonomy Database (Parks et al, 2018) represented two different species. Overall, these results confirm the specificity of SPA fragment sequences obtained 3′ of the RpoB1-R1327 primer annealing site for the high-resolution identification of bacterial strains at the (sub)species level.

TABLE 41
25 base pair SPA Fragment/Strain Name/Genome Taxonomy

SPA fragment 1: TTGAAATCATCAAATATCTGATTGA (SEQ ID NO: 291)
Bacteroides ovatus strain 1001275B_160808_G11
d_Bacteria; p_Bacteroidota; c_Bacteroidia; o_Bacteroidales; f_Bacteroidaceae;
g_Bacteroides; s_Bacteroides ovatus
Bacteroides ovatus strain AF26-20AA
d_Bacteria; p_Bacteroidota; c_Bacteroidia; o_Bacteroidales; f_Bacteroidaceae;
g_Bacteroides;s_Bacteroides ovatus
Bacteroides ovatus strain OF01-19AC
d_Bacteria; pBacteroidota; c_Bacteroidia; o_Bacteroidales;f_Bacteroidaceae;
g_Bacteroides; s_Bacteroides ovatus
SPA fragment 2: TTGCTTCTATTAATTACAATATGCA (SEQ ID NO: 292)
Blautia faecis strain MSK.11.45
d_Bacteria; p_Firmicutes_A; c_Clostridia; o_Lachnospirales; f_Lachnospiraceae;
g_Blautia A; s_Blautia_A faecis
Ruminococcus sp. D40t1_170626_H2
d_Bacteria; p_Firmicutes A; c_Clostridia; o__Lachnospirales; f_Lachnospiraceae;
g_Blautia_A; s_Blautia_A faecis
SPA fragment 3: TCGCATCCATCAATTACAATATGCA (SEQ ID NO: 293)
Roseburia inulinivorans strain AF28-15
d_Bacteria; p_Firmicutes A; c_Clostridia; o_Lachnospirales; f_Lachnospiraceae;
g_Roseburia; s_Roseburia inulinivorans
Roseburia inulinivorans strain SRR5519173-bin.6
d_Bacteria; p_Firmicutes_A; c_Clostridia; o_Lachnospirales; f_Lachnospiraceae;
g_Roseburia; s_Roseburia sp900552665
SPA fragment 4: TTGAAATCATTAAATATCTGATTGA (SEO ID NO: 294)
Bacteroides caccae strain BIOML-A1
d_Bacteria; p_Bacteroidota; c_Bacteroidia; o_Bacteroidales; f_Bacteroidaceae;
g_Bacteroides; s_Bacteroides caccae
Bacteroides caccae strain BIOML-A2
d_Bacteria; p_Bacteroidota; c_Bacteroidia; o_Bacteroidales; f_Bacteroidaceae;
g_Bacteroides; s_Bacteroides caccae
SPA fragment 5: TGTCTTCCATCAACTATCTGAACGG (SEQ ID NO: 295)
Faecalibacterium prausnitzii strain COPD315
d_Bacteria; p_Firmicutes A; c_Clostridia; o_Oscillospirales; f_Ruminococcaceae;
g_Faecalibacterium; s_Faecalibacterium prausnitzii_G
Faecalibacterium sp. Marseille-P9312
d_Bacteria; p_Firmicutes A; c_Clostridia; o_Oscillospirales; f_Ruminococcaceae;
g_Faecalibacterium; s_Faecalibacterium prausnitzii_G
Overview of 25 base pair long SPA fragments with more than one identified bacterial strain in the consortium. The detailed genome taxonomy is based on the Genome Taxonomy Database (Parks et al, 2018). The nucleotide sequences of the 25 base pair long SPA fragments are included. d_: domain; p_: phylum; c_: class; o_: order; f _: family; g_: genus; s_: species.

Description of the parameters to simulate the effect of SPA fragment length on community composition: Four simulations, each having 30 trials, were run with varying average length of mcfDNA fragments (40, 60, 80 and 100 base pairs). For the simulations we used of 1 ml liquid biopsy sample containing 100 ng/ml cfDNA and assumed that 1% of the total cfDNA represents mcfDNA (1 ng/ml). These estimates are considered realistic; for instance, in patients with metastatic breast cancer, the median plasma cfDNA concentration was found to be 112 ng/ml (Fernandez-Garcia et al, 2019). To be very conservative, we also estimate that due to technical limitations only 10% of the mcfDNA is effectively processed. As such, the simulations assume that fragments are only generated from 0.1 ng mcfDNA.

For each genome in the microbial community, length weighted relative abundance of total sample fragments was determined to account for the larger number of mcfDNA fragments generated from larger bacterial genomes. This abundance was subsequently used to determine the number of mcfDNA fragments per genome. The mcfDNA fragment sizes are randomly selected using a truncated normal distribution with fragment sizes between 1 and 200 base pairs. The fragment ends (start and end positions) were randomly selected from the genome. If a fragment contains the SPA primer annealing site, an in silico SPA fragment is generated from the 3′-end of the SPA primer to the end of the fragment (FIG. 1).

As described herein above, SPA fragments of 50 base pairs or longer, obtained using the RpoB1-R1327 primer, provide high resolution phylogenetic identification for most bacteria at the species and subspecies level. Therefore, the “number of SPA fragments generated with length 50 base pairs or greater” is used as one of the criteria to determine the sensitivity of the method for species identification in function of the various parameters. It should also be noted that many more SPA fragments with smaller length will be generated.

As previously shown herein above SPA fragments with length 25 base pairs or greater, obtained using the RpoB1-R1327 primer, show good resolution at the genus level. Therefore, the “relative abundance numbers of SPA fragment with length 25 base pairs or greater” will be used to calculate the community composition.

The parameters used in the four simulations are presented in Table 42. The following formula is used to calculate the “total number of cfDNA molecules”, based on X ng cfDNA with an average length of Y bp for the mcfDNA: (X ng×[6.022×10²³] molecules/mol)/(Y bp×[1×10⁹]ng/g×618 g/mol).

TABLE 42
Overview of the conditions used for the simulations to determine
the sensitivity of the SPA fragment sequencing method.
The estimate of generated mcfDNA
fragments being 0.1% of the cfDNA is based on the conservative
assumption that 1% of cfDNA represents mcfDNA, and that
due to technical limitations and losses during processing steps,
approximately 10% of mcfDNA fragments will be correctly
processed and contribute to SPA fragments.

| | | Average | Total | mcfDNA |
|---|---|---|---|---|
| | Amount of | mcfDNA | number of | fragments |
| | cfDNA | fragment length | cfDNA | (0.1% |
| Simulation | (X ng) | (Y bp) | molecules | cfDNA) |
| 40-100 ng | 100 | 40 | 2.436E+12 | 2.436E+09 |
| 60-100 ng | 100 | 60 | 1.624E+12 | 1.624E+09 |
| 80-100 ng | 100 | 80 | 1.218E+12 | 1.218E+09 |
| 100-100 ng | 100 | 100 | 9.744E+11 | 9.744E+08 |

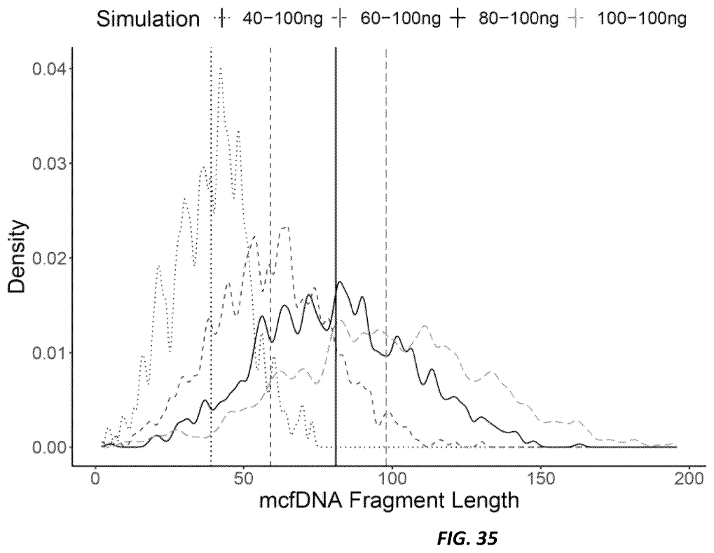

Simulation of fragment size distributions: We first evaluated the distribution of fragment sizes. To do so, we simulated the size distribution of a million mcfDNA fragments based on a truncated normal distribution with averages of 40, 60, 80 and 100 nucleotides in length, respectively. The results are presented in FIG. 35. Of the four simulations, the size distribution obtained for the simulation around an average fragment length of 60 base pairs came closest to the reported size distribution for mcfDNA (Burnham et al, 2016). We therefore consider this simulation the most relevant. The simulation for fragments with an average length of 40 base pairs missed nearly all fragments larger than 70 base pairs, while the simulations for fragments with average lengths of 80 base pairs and 100 base pairs underrepresented the smaller fragments and overrepresented fragments larger than 100 base pairs.

Simulation of SPA fragment generation for species identification and community composition analysis: For each simulation, the trial was repeated 30 times. The Wilcoxon rank sum test was performed on each of the simulations, by genome, with the two null hypotheses being: “the count of SPA fragments of 50 base pairs or greater was less than 3” (key criterium for species identification); or “the count of SPA fragments of 25 base pairs or greater was less than 10” (key criterium for species abundance). The results for the simulations using mcfDNA fragments with an average length of 40 base pairs or 60 base pairs are presented in Table 43 and Table 44, respectively; the RpoB1-R1327 was used to create the SPA fragments targeting the rpoB gene for phylogenetic identification.

Based on the results presented in Table 43, the null hypotheses “the count of 3 SPA fragments of 50 base pairs or greater was less than 3” gets accepted for the simulation using mcfDNA fragments with an average length of 40 base pairs. This indicates that for the conditions used in this simulation no reliable strain identification can be obtained at the species and subspecies level based on the presence of SPA fragments of 50 base pairs or greater. However, the null hypothesis “the count of 10 SPA fragments of 25 base pairs or greater was less than 10” gets rejected for strains that are present at approximately 1.25% or above with a p-value <0.05. This indicates that under the simulated conditions, using mcfDNA with an average fragment length of 40 base pairs, species present at approximately 1.25% in the community can be reliably identified by their 25 base pairs or greater SPA fragments at the genus level, and in many cases at the species level. In addition, the relative abundances of these species can be calculated.

Based on the results presented in Table 44, the null hypotheses “the count of SPA fragments of 50 base pairs or greater was less than 3” (key criterium for species identification) and “the count of SPA fragments of 25 base pairs or greater was less than 10” (key criterium for species abundance) both get rejected with a p-value <0.0001. This indicates that mcfDNA fragments with an average length of 60 base pairs can be reliably used for the identification of strains at the species and subspecies level, when the strains represent approximately 0.5% of the microbial community composition. In addition, mcfDNA fragments with an average length of 60 base pairs can be used to determine the community composition for species present at approximately 0.5%. Very similar results were obtained for the simulations using average mcfDNA fragment lengths of 80 base pairs and 100 base pairs.

On average, approximately 14,500 mcfDNA fragments that contain the RpoB1-R1327 primer annealing site were generated per trial for the simulation using mcfDNA fragments with an average length of 60 base pairs, of which approximately 5650 fragments would generate SPA fragments of 25 base pairs or greater. This should provide ample targets for the amplification step in the SPA fragment protocol, and subsequent sequencing.

Conclusions: Overall, the simulations show that mcfDNA fragments with an average length of 60 base pairs can be reliably used for the identification of strains at the species and subspecies level when they are present at 0.5% or above in the microbial community detectable in liquid biopsy samples, including peripheral blood. On average, strain abundances measured based on SPA fragments were within 1.4% of the actual abundance. For strains with less than 1% abundance, the average error was 1.8%, ranging from 0.1% to 7.2%; for strains with an abundance of 1% or higher, the average error was 1.2%, ranging from <0.1% to 4.5%.

TABLE 43
Summary of Simulation 40-100 ng (average generated mcfDNA length of 40, 100 ng of
cfDNA) using the RpoB1-R1327 primer. Bacterial species, represented by their genome ID,
whose presence and abundance were considered as significant (p-value < 0.05) are
highlighted in grey. Total mcfDNA Fragments per Genome with Conserved Region for Primer
indicates the total number of fragments generated for the 30 trials of the simulation. SPA
Fragments >24 bp long refers to SPA fragments of 25 base pairs or greater; SPA
Fragments >49 bp long refers to SPA fragments of 50 base pairs or greater.

| | Total | Average | | | | |
|---|---|---|---|---|---|---|
| | mcfDNA | mcfDNA | Average | | | |
| | Fragments | Fragments | mcfDND | | Average | Average |
| | per Genome | per Genome | Fragment | Average | Maximum | Count |
| | with Conserved | with Conserved | Length | SPA | SPA | of SPA |
| | Region | Region | with | Fragment | Fragment | Fragments >24 |
| Genome | for Primer | for Primer | [symbol] | Length | Length | bp long |
| 328813.45 | 1459 | 49 | 50 | 12 | 37 | 5 |
| 2044939.1074 | 1602 | 53 | 50 | 12 | 38 | 6 |
| 2292961.3 | 1602 | 53 | 50 | 13 | 38 | 6 |
| 166486.952 | 1667 | 56 | 50 | 12 | 38 | 6 |
| 88431.960 | 1702 | 57 | 50 | 12 | 40 | 6 |
| 1898203.1773 | 1728 | 58 | 50 | 12 | 40 | 6 |
| 360807.1171 | 1970 | 66 | 50 | 12 | 37 | 7 |
| 360807.64 | 1894 | 63 | 50 | 13 | 40 | 7 |
| 1971605.56 | 1944 | 65 | 50 | 12 | 39 | 7 |
| 2212467.8 | 1943 | 65 | 50 | 12 | 38 | 7 |
| 47678.882 | 2015 | 67 | 50 | 12 | 38 | 7 |
| 46503.2088 | 2193 | 73 | 50 | 13 | 41 | 9 |
| 823.3168 | 2406 | 80 | 50 | 12 | 40 | 9 |
| 47678.881 | 2312 | 77 | 50 | 12 | 39 | 8 |
| 278064.91 | 2413 | 80 | 50 | 12 | 40 | 9 |
| 410072.533 | 2344 | 78 | 50 | 12 | 40 | 9 |
| 165185.165 | 2524 | 84 | 50 | 12 | 39 | 8 |
| 1898205.22 | 2644 | 88 | 50 | 12 | 41 | 10 |
| 172733.1407 | 2726 | 91 | 50 | 12 | 41 | 10 |
| 679935.3 | 2739 | 91 | 51 | 12 | 41 | 11 |
| 259315.11 | 2858 | 95 | 50 | 12 | 42 | 10 |
| 1628085.84 | 2841 | 95 | 50 | 12 | 42 | 9 |
| 1897002.3 | 2951 | 98 | 50 | 12 | 41 | 11 |
| 2053618.24 | 2904 | 97 | 50 | 12 | 40 | 10 |
| 649756.2503 | 3028 | 101 | 50 | 12 | 40 | 11 |
| 46228.446 | 3179 | 106 | 50 | 12 | 44 | 12 |
| 2787081.3 | 3174 | 106 | 50 | 12 | 41 | 11 |
| 871665.25 | 3435 | 115 | 50 | 12 | 42 | 13 |
| 1679.11 | 3721 | 124 | 50 | 12 | 42 | 13 |
| 1872090.5 | 3857 | 129 | 50 | 12 | 42 | 13 |
| 2292892.3 | 4019 | 134 | 50 | 12 | 44 | 14 |
| 853.266 | 3993 | 133 | 50 | 12 | 43 | 14 |
| 41978.12 | 4018 | 134 | 50 | 12 | 43 | 14 |
| 28116.180 | 4399 | 147 | 50 | 12 | 43 | 16 |
| 2292949.3 | 4630 | 154 | 50 | 12 | 44 | 18 |
| 28026.777 | 4753 | 158 | 50 | 12 | 43 | 17 |
| 1118061.514 | 5292 | 176 | 50 | 12 | 43 | 19 |
| 2580425.3 | 5405 | 180 | 50 | 12 | 44 | 20 |
| 853.7698 | 5487 | 183 | 50 | 12 | 43 | 18 |
| 2293212.3 | 5564 | 185 | 50 | 12 | 43 | 19 |
| 418240.389 | 5679 | 189 | 50 | 12 | 44 | 21 |
| 39491.2479 | 5902 | 197 | 50 | 12 | 44 | 20 |
| 1263095.48 | 6085 | 203 | 50 | 12 | 44 | 22 |
| 1262967.3 | 6445 | 215 | 50 | 12 | 45 | 23 |
| 28116.176 | 6630 | 221 | 50 | 12 | 45 | 24 |
| 853.7674 | 7444 | 248 | 50 | 12 | 45 | 27 |
| 445970.5 | 8110 | 270 | 50 | 12 | 44 | 27 |
| 1737424.64 | 8456 | 282 | 50 | 12 | 46 | 29 |
| 28116.1423 | 9942 | 331 | 50 | 12 | 46 | 34 |
| 2021311.24 | 11494 | 383 | 50 | 12 | 46 | 41 |
| 821.3904 | 15224 | 507 | 50 | 12 | 48 | 54 |
| 46506.122 | 59073 | 1969 | 50 | 12 | 51 | 209 |

| | | | | p-value | p-value |
|---|---|---|---|---|---|
| | | Calculated % | | Wilcoxon | Wilcoxon |
| | | Relative | | test | test |
| | Average | Abundance | | H₀: Count | H₀: Count |
| | Count | Based | Theoretical | of SPA | of SPA |
| | of SPA | on SPA | Relative | fragments | fragments |
| | Fragments >49 | Fragments >24 | Abundance | longer than | longer than |
| Genome | bp long | bp long | % Input | 49 bp < [symbol] | 24 bp <10 |
| 328813.45 | 0 | 0.52 | 0.54 | 1.000 | 1.000 |
| 2044939.1074 | 0 | 0.58 | 0.58 | 1.000 | 1.000 |
| 2292961.3 | 0 | 0.66 | 0.58 | 1.000 | 1.000 |
| 166486.952 | 0 | 0.62 | 0.59 | 1.000 | 1.000 |
| 88431.960 | 0 | 0.63 | 0.63 | 1.000 | 1.000 |
| 1898203.1773 | 0 | 0.58 | 0.64 | 1.000 | 1.000 |
| 360807.1171 | 0 | 0.70 | 0.71 | 1.000 | 1.000 |
| 360807.64 | 0 | 0.79 | 0.71 | 1.000 | 1.000 |
| 1971605.56 | 0 | 0.72 | 0.72 | 1.000 | 1.000 |
| 2212467.8 | 0 | 0.71 | 0.72 | 1.000 | 1.000 |
| 47678.882 | 0 | 0.73 | 0.73 | 1.000 | 1.000 |
| 46503.2088 | 0 | 0.91 | 0.83 | 1.000 | 0.970 |
| 823.3168 | 0 | 0.91 | 0.86 | 1.000 | 0.963 |
| 47678.881 | 0 | 0.80 | 0.87 | 1.000 | 1.000 |
| 278064.91 | 0 | 0.91 | 0.88 | 1.000 | 0.984 |
| 410072.533 | 0 | 0.89 | 0.88 | 1.000 | 0.983 |
| 165185.165 | 0 | 0.88 | 0.94 | 1.000 | 0.991 |
| 1898205.22 | 0 | 1.01 | 0.96 | 1.000 | 0.780 |
| 172733.1407 | 0 | 0.99 | 0.99 | 1.000 | 0.832 |
| 679935.3 | 0 | 1.12 | 1.00 | 1.000 | 0.250 |
| 259315.11 | 0 | 1.03 | 1.03 | 1.000 | 0.623 |
| 1628085.84 | 0 | 0.97 | 1.04 | 1.000 | 0.829 |
| 1897002.3 | 0 | 1.10 | 1.07 | 1.000 | 0.144 |
| 2053618.24 | 0 | 1.01 | 1.07 | 1.000 | 0.747 |
| 649756.2503 | 0 | 1.11 | 1.10 | 1.000 | 0.392 |
| 46228.446 | 0 | 1.19 | 1.15 | 1.000 | 0.019 |
| 2787081.3 | 0 | 1.17 | 1.20 | 1.000 | 0.066 |
| 871665.25 | 0 | 1.32 | 1.26 | 1.000 | 0.002 |
| 1679.11 | 0 | 1.36 | 1.37 | 1.000 | 0.001 |
| 1872090.5 | 0 | 1.39 | 1.44 | 1.000 | 0.001 |
| 2292892.3 | 0 | 1.52 | 1.46 | 1.000 | 2.15E−05 |
| 853.266 | 0 | 1.43 | 1.47 | 1.000 | 4.77E−05 |
| 41978.12 | 0 | 1.43 | 1.50 | 1.000 | 3.11E−04 |
| 28116.180 | 0 | 1.65 | 1.60 | 1.000 | 1.78E−06 |
| 2292949.3 | 0 | 1.85 | 1.73 | 1.000 | 1.47E−06 |
| 28026.777 | 0 | 1.71 | 1.76 | 1.000 | 1.54E−05 |
| 1118061.514 | 0 | 1.96 | 1.93 | 1.000 | 9.01E−07 |
| 2580425.3 | 0 | 2.07 | 2.01 | 1.000 | 8.92E−07 |
| 853.7698 | 0 | 1.93 | 2.04 | 1.000 | 1.92E−06 |
| 2293212.3 | 0 | 2.00 | 2.07 | 1.000 | 8.92E−07 |
| 418240.389 | 0 | 2.20 | 2.11 | 1.000 | 8.95E−07 |
| 39491.2479 | 0 | 2.10 | 2.20 | 1.000 | 1.33E−06 |
| 1263095.48 | 0 | 2.29 | 2.23 | 1.000 | 9.00E−07 |
| 1262967.3 | 0 | 2.36 | 2.36 | 1.000 | 9.01E−07 |
| 28116.176 | 0 | 2.48 | 2.45 | 1.000 | 9.01E−07 |
| 853.7674 | 0 | 2.82 | 2.73 | 1.000 | 8.86E−07 |
| 445970.5 | 0 | 2.83 | 2.92 | 1.000 | 8.95E−07 |
| 1737424.64 | 0 | 2.99 | 3.14 | 1.000 | 9.02E−07 |
| 28116.1423 | 0 | 3.52 | 3.69 | 1.000 | 9.05E−07 |
| 2021311.24 | 0 | 4.21 | 4.26 | 1.000 | 9.03E−07 |
| 821.3904 | 1 | 5.59 | 5.65 | 1.000 | 8.98E−07 |
| 46506.122 | 2 | 21.75 | 21.61 | 0.972 | 9.10E−07 |

[symbol]

indicates data missing or illegible when filed

TABLE 44
Summary of Simulation 60-100 ng (average generated mcfDNA length of 60, 100 ng of
cfDNA) using the RpoB1-R1327 primer. Bacterial species, represented by their genome ID,
whose presence and abundance were considered as significant (p-value < 0.05) are
highlighted in grey. Total mcfDNA Fragments per Genome with Conserved Region for Primer
indicates the total number of fragments generated for the 30 trials of the simulation. SPA
Fragments >24 bp long refers to SPA fragments of 25 base pairs or greater; SPA
Fragments >49 bp long refers to SPA fragments of 50 base pairs or greater.

| | | | Average | | | |
|---|---|---|---|---|---|---|
| | Total | Average | mcfDND | | | |
| | mcfDNA | mcfDNA | Fragment | | | |
| | Fragments | Fragments | Length | | Average | Average |
| | per Genome | per Genome | with | Average | Maximum | Count |
| | with Conserved | with Conserved | Conserved | SPA | SPA | of SPA |
| | Region | Region | Region | Fragment | Fragment | Fragments >24 |
| Genome | for Primer | for | for Primer | Length | Length | bp long |
| 328813.45 | 2309 | 77 | 71 | 23 | 68 | 31 |
| 2044939.1074 | 2538 | 85 | 71 | 23 | 73 | 33 |
| 2292961.3 | 2579 | 86 | 71 | 23 | 68 | 33 |
| 166486.952 | 2549 | 85 | 71 | 23 | 70 | 35 |
| 88431.960 | 2845 | 95 | 71 | 22 | 72 | 37 |
| 1898203.1773 | 2949 | 98 | 71 | 23 | 74 | 39 |
| 360807.1171 | 3101 | 103 | 71 | 22 | 72 | 39 |
| 360807.64 | 3050 | 102 | 71 | 22 | 71 | 39 |
| 1971605.56 | 3037 | 101 | 71 | 23 | 69 | 40 |
| 2212467.8 | 3218 | 107 | 71 | 22 | 75 | 41 |
| 47678.882 | 3199 | 107 | 70 | 22 | 69 | 41 |
| 46503.2088 | 3625 | 121 | 71 | 23 | 72 | 47 |
| 823.3168 | 3763 | 125 | 71 | 23 | 72 | 49 |
| 47678.881 | 3720 | 124 | 71 | 22 | 72 | 48 |
| 278064.91 | 3886 | 130 | 71 | 22 | 74 | 49 |
| 410072.533 | 3899 | 130 | 71 | 22 | 72 | 51 |
| 165185.165 | 4118 | 137 | 71 | 22 | 73 | 52 |
| 1898205.22 | 4175 | 139 | 71 | 22 | 72 | 54 |
| 172733.1407 | 4344 | 145 | 70 | 22 | 73 | 57 |
| 679935.3 | 4291 | 143 | 70 | 22 | 73 | 55 |
| 259315.11 | 4611 | 154 | 71 | 23 | 75 | 61 |
| 1628085.84 | 4481 | 149 | 70 | 22 | 73 | 57 |
| 1897002.3 | 4666 | 156 | 71 | 22 | 74 | 60 |
| 2053618.24 | 4657 | 155 | 70 | 22 | 70 | 58 |
| 649756.2503 | 4824 | 161 | 71 | 22 | 75 | 62 |
| 46228.446 | 4969 | 166 | 71 | 22 | 74 | 65 |
| 2787081.3 | 5267 | 176 | 71 | 23 | 75 | 70 |
| 871665.25 | 5428 | 181 | 71 | 22 | 74 | 70 |
| 1679.11 | 5943 | 198 | 71 | 23 | 79 | 78 |
| 1872090.5 | 6187 | 206 | 71 | 23 | 76 | 83 |
| 2292892.3 | 6324 | 211 | 71 | 22 | 75 | 82 |
| 853.266 | 6373 | 212 | 71 | 22 | 76 | 82 |
| 41978.12 | 6452 | 215 | 71 | 22 | 77 | 83 |
| 28116.180 | 6852 | 228 | 71 | 23 | 79 | 91 |
| 2292949.3 | 7573 | 252 | 71 | 22 | 77 | 99 |
| 28026.777 | 7667 | 256 | 71 | 22 | 79 | 100 |
| 1118061.514 | 8281 | 276 | 71 | 22 | 76 | 108 |
| 2580425.3 | 8645 | 288 | 71 | 22 | 79 | 115 |
| 853.7698 | 9082 | 303 | 71 | 22 | 79 | 117 |
| 2293212.3 | 8851 | 295 | 71 | 22 | 79 | 115 |
| 418240.389 | 9082 | 303 | 71 | 22 | 77 | 118 |
| 39491.2479 | 9480 | 316 | 71 | 22 | 77 | 123 |
| 1263095.48 | 9676 | 323 | 71 | 22 | 80 | 126 |
| 1262967.3 | 10322 | 344 | 70 | 22 | 00 | 135 |
| 28116.176 | 10695 | 357 | 71 | 22 | 80 | 140 |
| 853.7674 | 11955 | 399 | 71 | 22 | 00 | 154 |
| 445970.5 | 12718 | 424 | 71 | 22 | 80 | 167 |
| 1737424.64 | 13729 | 458 | 71 | 22 | 82 | 179 |
| 28116.1423 | 16102 | 537 | 71 | 22 | 83 | 210 |
| 2021311.24 | 18586 | 620 | 71 | 22 | 86 | 242 |
| 821.3904 | 24720 | 824 | 71 | 22 | 86 | 320 |
| 46506.122 | 94169 | 3139 | 71 | 22 | 90 | 1225 |

| | | Calculated % | | p-value | p-value |
|---|---|---|---|---|---|
| | | Relative | | Wilcoxon | Wilcoxon |
| | Average | Abundance | | test | test |
| | Count | Based | Theoretical | H₀: Count | H₀: Count |
| | of SPA | on SPA | Relative | of SPA | of SPA |
| | Fragments >49 | Fragments | Abundance | fragments | fragments |
| Genome | bp long | >25 bp long | % Input | longer than | longer than |
| 328813.45 | 5 | 0.54 | 0.54 | 4.02E−05 | 8.90E−07 |
| 2044939.1074 | 5 | 0.58 | 0.58 | 3.39E−05 | 8.98E−07 |
| 2292961.3 | 6 | 0.59 | 0.58 | 5.12E−06 | 8.84E−07 |
| 166486.952 | 6 | 0.61 | 0.59 | 1.31E−05 | 8.85E−07 |
| 88431.960 | 7 | 0.65 | 0.63 | 1.26E−06 | 8.96E−07 |
| 1898203.1773 | 7 | 0.69 | 0.64 | 2.48E−06 | 8.96E−07 |
| 360807.1171 | 7 | 0.70 | 0.71 | 2.41E−06 | 9.05E−07 |
| 360807.64 | 6 | 0.69 | 0.71 | 2.71E−06 | 8.97E−07 |
| 1971605.56 | 7 | 0.71 | 0.72 | 4.19E−06 | 9.05E−07 |
| 2212467.8 | 7 | 0.73 | 0.72 | 3.32E−06 | 8.98E−07 |
| 47678.882 | 7 | 0.73 | 0.73 | 2.78E−06 | 9.01E−07 |
| 46503.2088 | 9 | 0.83 | 0.83 | 8.82E−07 | 9.04E−07 |
| 823.3168 | 9 | 0.87 | 0.86 | 1.30E−06 | 9.00E−07 |
| 47678.881 | 8 | 0.84 | 0.87 | 1.29E−06 | 9.04E−07 |
| 278064.91 | 9 | 0.86 | 0.88 | 1.23E−06 | 9.05E−07 |
| 410072.533 | 9 | 0.90 | 0.88 | 8.28E−07 | 9.01E−07 |
| 165185.165 | 10 | 0.92 | 0.94 | 8.64E−07 | 8.95E−07 |
| 1898205.22 | 9 | 0.96 | 0.96 | 4.31E−06 | 9.03E−07 |
| 172733.1407 | 9 | 1.00 | 0.99 | 1.53E−06 | 9.03E−07 |
| 679935.3 | 10 | 0.98 | 1.00 | 1.29E−06 | 9.05E−07 |
| 259315.11 | 10 | 1.07 | 1.03 | 8.84E−07 | 9.04E−07 |
| 1628085.84 | 10 | 1.01 | 1.04 | 8.73E−07 | 8.97E−07 |
| 1897002.3 | 10 | 1.05 | 1.07 | 1.14E−06 | 9.05E−07 |
| 2053618.24 | 9 | 1.02 | 1.07 | 8.87E−07 | 9.05E−07 |
| 649756.2503 | 10 | 1.10 | 1.10 | 1.26E−06 | 8.98E−07 |
| 46228.446 | 10 | 1.15 | 1.15 | 1.30E−06 | 9.03E−07 |
| 2787081.3 | 13 | 1.23 | 1.20 | 8.85E−07 | 9.05E−07 |
| 871665.25 | 12 | 1.23 | 1.26 | 8.82E−07 | 9.01E−07 |
| 1679.11 | 14 | 1.38 | 1.37 | 8.56E−07 | 9.02E−07 |
| 1872090.5 | 14 | 1.46 | 1.44 | 8.92E−07 | 9.02E−07 |
| 2292892.3 | 13 | 1.46 | 1.46 | 8.72E−07 | 8.93E−07 |
| 853.266 | 15 | 1.45 | 1.47 | 8.35E−07 | 9.01E−07 |
| 41978.12 | 14 | 1.47 | 1.50 | 9.01E−07 | 9.08E−07 |
| 28116.180 | 16 | 1.60 | 1.60 | 8.88E−07 | 8.98E−07 |
| 2292949.3 | 18 | 1.74 | 1.73 | 8.95E−07 | 8.95E−07 |
| 28026.777 | 19 | 1.76 | 1.76 | 8.88E−07 | 9.05E−07 |
| 1118061.514 | 19 | 1.91 | 1.93 | 8.87E−07 | 9.06E−07 |
| 2580425.3 | 20 | 2.02 | 2.01 | 8.98E−07 | 9.06E−07 |
| 853.7698 | 21 | 2.07 | 2.04 | 8.84E−07 | 9.09E−07 |
| 2293212.3 | 19 | 2.02 | 2.07 | 8.91E−07 | 9.06E−07 |
| 418240.389 | 21 | 2.08 | 2.11 | 9.01E−07 | 9.00E−07 |
| 39491.2479 | 20 | 2.18 | 2.20 | 8.93E−07 | 9.08E−07 |
| 1263095.48 | 23 | 2.22 | 2.23 | 9.00E−07 | 9.03E−07 |
| 1262967.3 | 23 | 2.39 | 2.36 | 9.01E−07 | 9.06E−07 |
| 28116.176 | 25 | 2.46 | 2.45 | 9.00E−07 | 9.10E−07 |
| 853.7674 | 27 | 2.71 | 2.73 | 8.93E−07 | 9.09E−07 |
| 445970.5 | 28 | 2.94 | 2.92 | 9.05E−07 | 9.00E−07 |
| 1737424.64 | 31 | 3.17 | 3.14 | 9.00E−07 | 9.05E−07 |
| 28116.1423 | 36 | 3.71 | 3.69 | 9.01E−07 | 9.09E−07 |
| 2021311.24 | 42 | 4.27 | 4.26 | 9.02E−07 | 9.10E−07 |
| 821.3904 | 56 | 5.64 | 5.65 | 9.02E−07 | 9.11E−07 |
| 46506.122 | 215 | 21.64 | 21.61 | 9.12E−07 | 9.11E−07 |

### Example 11: Specificity Analysis of Spa Fragment Sequencing

Several studies have shown that high resolution phylogenetic identification of bacteria is a prerequisite to accurately link bacteria to specific disease phenotypes, including the development of adenomas and early-stage carcinomas in colorectal cancer. Therefore, one of the key requirements for SPA fragment sequencing is high-resolution identification of microbial species in liquid biopsy samples at the species and subspecies level. We therefore tried to answer the following questions:
1. Specificity of SPA fragments—How phylogenetically accurate are the 46 distinct 50 base pair long SPA fragments generated using the RpoB1-R1327 primer (EXAMPLE 11)?
2. How does the sensitivity and specificity of the SPA fragment sequencing method compare to deep metagenome sequencing of cfDNA fragments followed by taxonomic classification using read-based metagenome analysis methods (EXAMPLE 12)?

Description of the community used for the simulations: To understand the specificity of the SPA fragment sequencing method, the same gut community described in EXAMPLE 10 was used for the simulations. The 52-member community, whose composition was obtained with PacBio sequencing, is described in Table 45. The sequences of the SPA fragments obtained for each of the community members are also presented. SPA fragments that were identical between multiple community members are highlighted in grey.

TABLE 45

| | | PacBio | SPA | | |
|---|---|---|---|---|---|
| | PATRIC | Relative | Relative | Rpob_SPA | |
| Genome | Genome | Abundance | Abundance | fragments | SPA Fragment |
| Name | ID | % | % | code | sequence (50 bp) |
| Bacteroides | 46506.122 | 21.61 | 21.64 | rpob_SPA1 | TTGAGATTATCAA |
| stercoris | | | | | GTATCTGATTGAG |
| AM51-2BH | | | | | TTGATAAACTCAA |
| | | | | | AAGCAGATGTG |
| | | | | | (SEQ ID NO: 296) |
| Bacteroides | 821.3904 | 5.65 | 5.64 | rpob_SPA2 | TTGAAATCATTAA |
| vulgatus VPI- | | | | | GTATCTGATTGAG |
| 5710 | | | | | CTGATTAACTCTA |
| | | | | | AAGCGGATGTT |
| | | | | | (SEQ ID NO: 297) |
| Agathobacter | 2021311.24 | 4.26 | 4.27 | rpob_SPA3 | TCGCATCCATCAA |
| sp. COPD130 | | | | | CTATAATATGCAT |
| | | | | | CTGGAGTGGGGC |
| | | | | | ATCGGAACAGAT |
| | | | | | (SEQ ID NO: 298) |
| Bacteroides | 28116.1423 | 3.69 | 3.71 | rpob_SPA4 | TTGAAATCATCAA |
| ovatus | | | | | ATATCTGATTGAG |
| 1001275B_ | | | | | TTGATTAACTCAA |
| 160808_G11 | | | | | AAGCGGATGTG |
| | | | | | (SEQ ID NO: 299) |
| Blautia | 1737424.64 | 3.14 | 3.17 | rpob_SPA5 | TTGCGTCCATTAA |
| massiliensis | | | | | CTACAATATGCAT |
| MSK.13.24 | | | | | CTGGAGTATGGC |
| | | | | | CTTGGTAACGAT |
| | | | | | (SEQ ID NO: 300) |
| Alistipes | 445970.5 | 2.92 | 2.94 | rpob_SPA6 | TCGCCATCATCAA |
| putredinis | | | | | GTACCTGATCCAG |
| DSM 17216 | | | | | CTCATCAACTCGA |
| | | | | | AAGCCGAGGTG |
| | | | | | (SEQ ID NO: 301) |
| Faecali- | 853.7674 | 2.73 | 2.71 | rpob_SPA7 | TGTCCTCCATCAA |
| bacterium | | | | | CTACCTGAACGGT |
| prausnitzii | | | | | CTGGGCCACGGC |
| COPD342 | | | | | GTTGGCACCACC |
| | | | | | (SEQ ID NO: 302) |
| Bacteroides | 28116.176 | 2.45 | 2.46 | rpob_SPA4 | TTGAAATCATCAA |
| ovatus AF26- | | | | | ATATCTGATTGAG |
| 20AA | | | | | TTGATTAACTCAA |
| | | | | | AAGCGGATGTG |
| | | | | | (SEQ ID NO: 299) |
| Ruminococcus | 1262967.3 | 2.36 | 2.39 | rpob_SPA8 | TTGCTTCTATTAA |
| sp. CAG:9 | | | | | CTACAATATGCAT |
| | | | | | CTGGAATATGGC |
| | | | | | CTTGGCAATGCC |
| | | | | | (SEQ ID NO: 303) |
| Paraprevotella | 1263095.48 | 2.23 | 2.22 | rpob_SPA9 | TCGAGATTATCAA |
| clara | | | | | GTATCTGATAGA |
| CAG: 116 | | | | | GCTGATAAACTC |
| MGS: 116 | | | | | AAAGGCACTTGT |
| | | | | | C (SEQ ID NO: 304) |
| [Eubacterium] | 39491.2479 | 2.2 | 2.18 | rpob_SPA10 | TCGCAACTATCAA |
| rectale | | | | | CTACAATATGCAC |
| BIOML-A1 | | | | | TTAGAGTGGGGC |
| | | | | | GCAGGAACAGAT |
| | | | | | (SEQ ID NO: 305) |
| Blautia | 418240.389 | 2.11 | 2.08 | rpob_SPA11 | TCGCTTCCATCAA |
| wexlerae | | | | | CTACAACATGCAT |
| 1001270J_ | | | | | CTGGAATACGGC |
| 160509_E6 | | | | | GCAGGAAATGCC |
| | | | | | (SEQ ID NO: 306) |
| Ruminococcus | 2293212.3 | 2.07 | 2.02 | rpob_SPA12 | TTGCTTCTATTAA |
| sp. AM40- | | | | | CTACAATATGCAT |
| 10AC | | | | | CTGGAATATGGC |
| | | | | | CTTGGTAATGCC |
| | | | | | (SEQ ID NO: 307) |
| Faecali- | 853.7698 | 2.04 | 2.07 | rpob_SPA13 | TGTCTTCCATCAA |
| bacterium | | | | | CTATCTGAACGGC |
| prausnitzii | | | | | CTGGGCCACGGC |
| COPD315 | | | | | ATCGGCACCACC |
| | | | | | (SEQ ID NO: 308) |
| Faecali- | 2580425.3 | 2.01 | 2.02 | rpob_SPA13 | TGTCTTCCATCAA |
| bacterium | | | | | CTATCTGAACGGC |
| sp. | | | | | CTGGGCCACGGC |
| Marseille-P9312 | | | | | ATCGGCACCACC |
| | | | | | (SEQ ID NO: 309) |
| Alistipes obesi | 1118061.514 | 1.93 | 1.91 | rpob_SPA14 | TCGCCATTATCAA |
| MGYG- | | | | | GTACCTCATCCAG |
| HGUT-01415 | | | | | CTCATCAACTCGC |
| | | | | | GCGCCGAGGTG |
| | | | | | (SEQ ID NO: 310) |
| Bifido- | 28026.777 | 1.76 | 1.76 | rpob_SPA15 | AGTTCCCGGGCA |
| bacterium_ | | | | | AGCGTGACGGCC |
| pseudo- | | | | | AGGATGTGGATC |
| catenulatum | | | | | TGCGCGTGGACG |
| LFYP_29 | | | | | TC (SEQ ID NO: |
| | | | | | 311) |
| Bacteroides | 2292949.3 | 1.73 | 1.74 | rpob_SPA16 | TCGAAATTATCAA |
| sp. AM30-16 | | | | | ATATCTCATCGAG |
| | | | | | TTGATTAACTCGA |
| | | | | | AAGCGGATGTG |
| | | | | | (SEQ ID NO: 312) |
| Bacteroides | 28116.180 | 1.6 | 1.60 | rpob_SPA4 | TTGAAATCATCAA |
| ovatus OF01- | | | | | ATATCTGATTGAG |
| 19AC | | | | | TTGATTAACTCAA |
| | | | | | AAGCGGATGTG |
| | | | | | (SEQ ID NO: 299) |
| Ruminococcus | 41978.12 | 1.5 | 1.47 | rpob_SPA17 | TCGCTACGGTTTC |
| sp. | | | | | TTACTTCCTCAAC |
| UBA10663 | | | | | CTTTGCGAGGGC |
| | | | | | GTTGGTACTGTT |
| | | | | | (SEQ ID NO: 313) |
| Faecali- | 853.266 | 1.47 | 1.45 | rpob_SPA18 | TGTCCTCCATCAA |
| bacterium | | | | | CTACCTGAACGGT |
| prausnitzii | | | | | CTGGGCTACGGC |
| APC923/51-1 | | | | | ATCGGCACCACC |
| | | | | | (SEQ ID NO: 314) |
| Firmicutes | 2292892.3 | 1.46 | 1.46 | rpob_SPA19 | TGGCTTCAATTAA |
| bacterium | | | | | CTACAATATGCAT |
| AM31-12AC | | | | | CTGGAATATGGT |
| | | | | | ATGGGTAATGAT |
| | | | | | (SEQ ID NO: 315) |
| Acetatifactor | 1872090.5 | 1.44 | 1.46 | rpob_SPA20 | TGGCTTCCATCAA |
| sp. COPD172 | | | | | CTATAATATGCAT |
| | | | | | CTGGAGTATGGC |
| | | | | | CTGGGCAACGAT |
| | | | | | (SEQ ID NO: 316) |
| Bifido- | 1679.11 | 1.37 | 1.38 | rpob_SPA21 | CCTTCCCGGGCAA |
| bacterium | | | | | GCGCAACGGCGA |
| longum | | | | | AGACGTTGACCT |
| subsp. longum | | | | | GCGCGTGGACGT |
| 9 | | | | | C (SEQ ID NO: 317) |
| Blautia faecis | 871665.25 | 1.26 | 1.23 | rpob_SPA22 | TTGCTTCTATTAA |
| MSK.11.45 | | | | | TTACAATATGCAT |
| | | | | | CTGGAATACGGC |
| | | | | | ATTGGAAATGAC |
| | | | | | (SEQ ID NO: 318) |
| Blautia faecis | 2787081.3 | 1.2 | 1.23 | rpob_SPA22 | TTGCTTCTATTAA |
| D40t1_170626_ | | | | | TTACAATATGCAT |
| H2 | | | | | CTGGAATACGGC |
| | | | | | ATTGGAAATGAC |
| | | | | | (SEQ ID NO: 318) |
| [Ruminococcus] | 46228.446 | 1.15 | 1.15 | rpob_SPA23 | TTGCATCCATCAA |
| lactaris | | | | | TTACAATATGCAT |
| SRR7721875- | | | | | CTTGAGTATGGCA |
| bin.26 | | | | | TGGGTAATGAT |
| | | | | | (SEQ ID NO: 319) |
| Anaerostipes | 649756.2503 | 1.1 | 1.10 | rpob_SPA24 | TAGCATCCATCAA |
| hadrus | | | | | CTACAATATCCAT |
| S01C.meta.bin_ | | | | | TTAGAGTATGGA |
| 9 | | | | | ATTGGACATGAT |
| | | | | | (SEQ ID NO: 320) |
| Eubacterium | 1897002.3 | 1.07 | 1.05 | rpob_SPA25 | TAGCTTCTATTAA |
| sp. 38_16 | | | | | CTACAATATCCAT |
| | | | | | CTGGAATATGGT |
| | | | | | GTTGGTAATGAC |
| | | | | | (SEQ ID NO: 321) |
| Subdoli- | 2053618.24 | 1.07 | 1.02 | rpob_SPA26 | TTGCCTCCGTCAA |
| granulum sp. | | | | | CTACCTGCTGGGC |
| S08B.meta.bin | | | | | CTTGATCACGGCA |
| _8 | | | | | TCGGCACCACC |
| | | | | | (SEQ ID NO: 322) |
| Agathobaculum | 1628085.84 | 1.04 | 1.01 | rpob_SPA27 | TCGCTTCCATCTG |
| butyrici- | | | | | CTATCTGCTCAAC |
| producens | | | | | CTCGGTCACGGC |
| COPD228 | | | | | ATCGGCACGGTT |
| | | | | | (SEQ ID NO: 323) |
| uncultured | 259315.11 | 1.03 | 1.07 | rpob_SPA28 | TGGCTTCCATCAA |
| Faecali- | | | | | CTACCTGAACGGT |
| bacterium sp. | | | | | CTGGGCCACAAC |
| UMGS184 | | | | | ATTGGCACCACC |
| | | | | | (SEQ ID NO: 324) |
| Alistipes | 679935.3 | 1 | 0.98 | rpob_SPA29 | TCGCCATTATCAA |
| finegoldii | | | | | ATACCTGATCCAG |
| DSM 17242 | | | | | CTGATCAACTCCA |
| | | | | | AGGCCGACGTG |
| | | | | | (SEQ ID NO: 325) |
| uncultured | 172733.1407 | 0.99 | 1.00 | rpob_SPA30 | TCGCCTCCATCAA |
| Clostridiales | | | | | CTACATGAACGC |
| bacterium | | | | | GCTGGCGCACGG |
| UMGS84 | | | | | CATCGTCTATAAG |
| | | | | | (SEQ ID NO: 326) |
| Rumino- | 1898205.22 | 0.96 | 0.96 | rpob_SPA31 | TTGCTTCCGTCAA |
| coccaceae | | | | | CTACCTGCTGGGC |
| bacterium | | | | | CTTGACCATGGCA |
| UBA9091 | | | | | TCGGCGTGACC |
| | | | | | (SEQ ID NO: 327) |
| uncultured | 165185.165 | 0.94 | 0.92 | rpob_SPA32 | TTGCTTCTATTAA |
| Eubacterium | | | | | TTATAATATGCAC |
| sp. UMGS39 | | | | | CTTGAATACGGC |
| | | | | | GTTGGTACAAAG |
| | | | | | (SEQ ID NO: 328) |
| uncultured | 278064.91 | 0.88 | 0.86 | rpob_SPA33 | TCGCGGCGGTAG |
| Dialister sp. | | | | | ACTATCTTTTGAA |
| ERR414242- | | | | | TATGATCCAGGG |
| bin.5 | | | | | CTATGGACGCCA |
| | | | | | G (SEQ ID NO: 329) |
| Coprococcus | 410072.533 | 0.88 | 0.90 | rpob_SPA34 | TGGCGTCTATCAA |
| comes | | | | | TTACAATATGCAT |
| MSK.16.14 | | | | | CTTGAATATGGA |
| | | | | | ATCGGTAAAGAT |
| | | | | | (SEQ ID NO: 330) |
| Bacteroides | 47678.881 | 0.87 | 0.84 | rpob_SPA35 | TTGAAATCATTAA |
| caccae | | | | | ATATCTGATTGAG |
| BIOML-A2 | | | | | TTAATTAACTCAA |
| | | | | | AGGCAGATGTG |
| | | | | | (SEQ ID NO: 331) |
| Parabacteroids | 823.3168 | 0.86 | 0.87 | rpob_SPA36 | TCGAGATCATCA |
| distasonis | | | | | AGTACCTGATCG |
| LMAG: 27 | | | | | AGTTGATCAACTC |
| | | | | | GAAGGCTATCGT |
| | | | | | G (SEQ ID NO: 332) |
| Para- | 46503.2088 | 0.83 | 0.83 | rpob_SPA37 | TTGAGATCATCAA |
| bacteroides | | | | | ATATCTGATTGAG |
| merdae | | | | | TTGATCAACTCGA |
| 1001136B_ | | | | | AAGCGATCGTT |
| 160425_B1 | | | | | (SEQ ID NO: 333) |
| Bacteroides | 47678.882 | 0.73 | 0.73 | rpob_SPA35 | TTGAAATCATTAA |
| caccae | | | | | ATATCTGATTGAG |
| BIOML-A1 | | | | | TTAATTAACTCAA |
| | | | | | AGGCAGATGTG |
| | | | | | (SEQ ID NO: 331) |
| Faecali- | 1971605.56 | 0.72 | 0.71 | rpob_SPA38 | TGGCTTCCATCAA |
| bacterium sp. | | | | | CTACCTGAACGGT |
| S04C.meta.bin_ | | | | | CTGGGCCACAAT |
| 2 | | | | | ATTGGCACCACC |
| | | | | | (SEQ ID NO: 334) |
| Bacteroidaceae | 2212467.8 | 0.72 | 0.73 | rpob_SPA39 | TCGAAATTATCAA |
| bacterium | | | | | ATATCTTATCGAG |
| MGYG- | | | | | TTGATTAACTCGA |
| HGUT-00144 | | | | | AGACCGATGTC |
| | | | | | (SEQ ID NO: 335) |
| Roseburia | 360807.1171 | 0.71 | 0.70 | rpob_SPA40 | TCGCATCCATCAA |
| inulinivorans | | | | | TTACAATATGCAT |
| SRR5519173- | | | | | TTAGAGTATGGTA |
| bin.6 | | | | | TTGGTCATGAT |
| | | | | | (SEQ ID NO: 336) |
| Roseburia | 360807.64 | 0.71 | 0.69 | rpob_SPA40 | TCGCATCCATCAA |
| inulinivorans | | | | | TTACAATATGCAT |
| AF28-15 | | | | | TTAGAGTATGGTA |
| | | | | | TTGGTCATGAT |
| | | | | | (SEQ ID NO: 336) |
| Lachno- | 1898203.1773 | 0.64 | 0.69 | rpob_SPA41 | TAGGTTCTATTAA |
| spiraceae | | | | | CTACTGCTTAAAC |
| bacterium | | | | | TTAGAGTATGGC |
| MGYG- | | | | | GTAGGACAGGAT |
| HGUT-00193 | | | | | (SEQ ID NO: 337) |
| Dorea | 88431.960 | 0.63 | 0.65 | rpob_SPA42 | TAGCTTCTATTAA |
| longicatena | | | | | CTACAATATGCAT |
| MSK.11.4 | | | | | CTGGAATATGGC |
| | | | | | ATCGGAACTGAT |
| | | | | | (SEQ ID NO: 338) |
| Roseburia | 166486.952 | 0.59 | 0.61 | rpob_SPA43 | TTGCATCCATCAA |
| intestinalis | | | | | CTACAATATGCAC |
| ERR321618- | | | | | TTAGAGTATGGTA |
| bin. 7 | | | | | TCGGAAATGAT |
| | | | | | (SEQ ID NO: 339) |
| Clostridia | 2044939.1074 | 0.58 | 0.58 | rpob_SPA44 | TTGCGTCTGTAAA |
| bacterium | | | | | CTATTGTCTAAAC |
| COPD107 | | | | | CTTGCTAACGGTA |
| | | | | | TAGGTACTGTT |
| | | | | | (SEQ ID NO: 340) |
| Blautia sp. | 2292961.3 | 0.58 | 0.59 | rpob_SPA45 | TTGCTTCTATCAA |
| AF19-10LB | | | | | CTACAATATGCAT |
| | | | | | CTGGAATATGGC |
| | | | | | ATTGGTAATGAC |
| | | | | | (SEQ ID NO: 341) |
| Alistipes | 328813.45 | 0.54 | 0.54 | rpob_SPA46 | TCGCCATCATCAA |
| onderdonkii | | | | | ATACCTGATCCAG |
| D10-10 | | | | | CTGATCAACTCGA |
| | | | | | AGGCCGACGTC |
| | | | | | (SEQ ID NO: 342) |

Composition (species name and genome ID) and relative species abundances of the gut microbiome community used for the simulations. Long read PacBio sequencing was used to determine the community composition. The community composition based on the rpoB gene-derived SPA fragment sequencing simulation was determined using the parameters described above. The codes and sequences for the unique 50 base pair SPA fragments generated for each species are shown. SPA fragments that are identical between multiple community members are highlighted in in grey.

Specificity analysis of SPA fragments obtained using the RpoB1-R1327 primer: To analyze the phylogenetic specificity of the SPA fragments listed in Table 45, we compared them to a phylogenetic gene database containing over 50,000 unique RpoB gene entries. The results of this comparison are presented in Table 46 and show the following:
The 50 base pair SPA fragments for the 52 community members showed 100% correct phylogenetic identification on the genus level and were also highly specific on the species level when compared to the reference database of 50,000+ non-redundant RpoB gene entries. Three of the SPA fragments identified multiple, closely related species:
   In addition to recognizing Bacteroides ovatus, the SPA2 fragment also recognized the closely related species Bacteroides xylanisolvens; and in addition to recognizing Alistipes onderdonkii, the rpob_SPA46 fragment also recognized the closely related species Alistipes finegoldii and Alistipes shahii.
   The rpob_SPA8 fragment recognized the Blautia_A wexlerae_A, Blautia_A wexlerae and Blautia_A sp003480185, which according to the new classification of the Genome Taxonomy Database (Parks et al, 2018) represent very closely related but distinct species; the same is the case for the rpob_SPA40 fragment, which recognizes the very closely related but distinct species Roseburia inulinivorans and Roseburia sp900552665.
The fragment rpob_SPA21 enabled identification of Bifidobacterium longum at the species level but failed to discriminate on the subspecies level between Bifidobacterium longum subsp. longum and Bifidobacterium longum subsp. infantis; and the rpob_SPA24 fragment enabled identification of Anaerostipes hadrus at the species level but failed to discriminate on the subspecies level between Anaerostipes hadrus and Anaerostipes hadrus_B.
It was also noted that the Faecalibacterium species present in the community could be identified to the species level by their unique SPA fragment, and in several cases to the Faecalibacterium prausnitzii subspecies level. The only exception was the fragment rpob_SPA18, which recognized the two very closely related subspecies Faecalibacterium prausnitzii_J and Faecalibacterium prausnitzii.

Overall, the results shows that SPA fragments generated 3′ of the RpoB1-R1327 primer annealing site have very high phylogenetic specificity to reliably classify bacteria at both the taxonomic genus and species level.

TABLE 46
Simulated composition of the gut microbiome community based on rpoB gene-derived SPA fragment analysis. Each
community member is identified by its GTDB taxonomy and PATRIC genome ID. The genus-level and species-level
identification of each community member, based on its 50 base pair rpoB gene-derived SPA fragment, is presented
based on GTDB taxonomy (Parks et al, 2018). For each community member, the relative abundance and SPA fragment
identifier are listed. SPA fragments, which identified multiple species, are highlighted in grey.

| | | PacBio | SPA | | | |
|---|---|---|---|---|---|---|
| Microbial | | Relative | Relative | Rpob SPA | | |
| community species | PATRIC | Abundance | Abundance | fragments | Rpob SPA | rpob SPA |
| (GTDB taxonomy) | Genome ID | % | % | code | genus level | species level |
| Bacteroides | 46506.122 | 21.61 | 21.64 | rpob_SPA1 | Bacteroides | Bacteroides |
| stercoris | | | | | | stercoris |
| Phocaeicola | 821.3904 | 5.65 | 5.64 | rpob_SPA2 | Phocaeicola | Phocaeicola |
| vulgatus | | | | | | vulgatus |
| Agathobacter | 2021311.24 | 4.26 | 4.27 | rpob_SPA3 | Agathobacter | Agathobacter |
| faecis | | | | | | faecis |
| Bacteroides | 28116.1423 | 3.69 | 3.71 | rpob_SPA4 | Bacteroides | Bacteroides ovatus |
| ovatus | | | | | | Bacteroides |
| | | | | | | xylanisolvens |
| Blautia_A | 1737424.64 | 3.14 | 3.17 | rpob_SPA5 | Blautia_A | Blautia_A |
| massiliensis | | | | | | massiliensis |
| Alistipes | 445970.5 | 2.92 | 2.94 | rpob_SPA6 | Alistipes | Alistipes putredinis |
| putredinis | | | | | | |
| Faecalibacterium | 853.7674 | 2.73 | 2.71 | rpob_SPA7 | Faecalibacterium | Faecalibacterium |
| prausnitzii_C | | | | | | prausnitzii_C |
| Bacteroides | 28116.176 | 2.45 | 2.46 | rpob_SPA4 | Bacteroides | Bacteroides ovatus |
| ovatus | | | | | | Bacteroides |
| | | | | | | xylanisolvens |
| Blautia_A | 1262967.3 | 2.36 | 2.39 | rpob_SPA8 | Blautia_A | Blautia_A |
| wexlerae_A | | | | | | wexlerae_A |
| | | | | | | Blautia_A wexlerae |
| | | | | | | Blautia_A |
| | | | | | | sp003480185 |
| Paraprevotella | 1263095.48 | 2.23 | 2.22 | rpob_SPA9 | Paraprevotella | Paraprevotella clara |
| clara | | | | | | |
| Agathobacter | 39491.2479 | 2.2 | 2.18 | rpob_SPA10 | Agathobacter | Agathobacter rectalis |
| rectalis | | | | | | |
| Fusicatenibacter | 418240.389 | 2.11 | 2.08 | rpob_SPA11 | Fusicatenibacter | Fusicatenibacter |
| saccharivorans | | | | | | saccharivorans |
| Blautia_A | 2293212.3 | 2.07 | 2.02 | rpob_SPA12 | Blautia_A | Blautia_A |
| sp003480185 | | | | | | sp003480185 |
| Faecalibacterium | 853.7698 | 2.04 | 2.07 | rpob_SPA13 | Faecalibacterium | Faecalibacterium |
| prausnitzii_G | | | | | | prausnitzii_G |
| Faecalibacterium | 2580425.3 | 2.01 | 2.02 | rpob_SPA13 | Faecalibacterium | Faecalibacterium |
| prausnitzii_G | | | | | | prausnitzii_G |
| Alistipes | 1118061.514 | 1.93 | 1.91 | rpob_SPA14 | Alistipes | Alistipes communis |
| communis | | | | | | |
| Bifidobacterium | 28026.777 | 1.76 | 1.76 | rpob_SPA15 | Bifidobacterium | Bifidobacterium |
| pseudocatenulatum | | | | | | pseudocatenulatum |
| Bacteroides | 2292949.3 | 1.73 | 1.74 | rpob_SPA16 | Bacteroides | Bacteroides |
| uniformis | | | | | | uniformis |
| Bacteroides | 28116.180 | 1.6 | 1.60 | rpob_SPA4 | Bacteroides | Bacteroides ovatus |
| ovatus | | | | | | Bacteroides |
| | | | | | | xylanisolvens |
| Ruminococcus_D | 41978.12 | 1.5 | 1.47 | rpob_SPA17 | Ruminococcus_D | Ruminococcus_D |
| bicirculans | | | | | | bicirculans |
| Faecalibacterium | 853.266 | 1.47 | 1.45 | rpob_SPA18 | Faecalibacterium | Faecalibacterium |
| prausnitzii_J | | | | | | prausnitzii_J |
| | | | | | | Faecalibacterium |
| | | | | | | prausnitzii |
| Schaedlerella | 2292892.3 | 1.46 | 1.46 | rpob_SPA19 | Schaedlerella | Schaedlerella |
| sp900066545 | | | | | | sp900066545 |
| Acetatifactor | 1872090.5 | 1.44 | 1.46 | rpob_SPA20 | Acetatifactor | Acetatifactor |
| sp900066565 | | | | | | sp900066565 |
| Bifidobacterium | 1679.11 | 1.37 | 1.38 | rpob_SPA21 | Bifidobacterium | Bifidobacterium |
| longum | | | | | | longum subsp. |
| | | | | | | longum |
| | | | | | | Bifidobacterium |
| | | | | | | longum subsp. |
| | | | | | | infantis |
| Blautia_A faecis | 871665.25 | 1.26 | 1.23 | rpob_SPA22 | Blautia_A | Blautia_A faecis |
| Blautia_A faecis | 2787081.3 | 1.2 | 1.23 | rpob_SPA22 | Blautia_A | Blautia_A faecis |
| Mediterraneibacter | 46228.446 | 1.15 | 1.15 | rpob_SPA23 | Mediterraneibacter | Mediterraneibacter |
| lactaris | | | | | | lactaris |
| Anaerostipes | 649756.2503 | 1.1 | 1.10 | rpob_SPA24 | Anaerostipes | Anaerostipes hadrus |
| hadrus | | | | | | Anaerostipes |
| | | | | | | hadrus_B |
| Anaerobutyricum | 1897002.3 | 1.07 | 1.05 | rpob_SPA25 | Anaerobutyricum | Anaerobutyricum |
| soehngenii | | | | | | soehngenii |
| Gemmiger | 2053618.24 | 1.07 | 1.02 | rpob_SPA26 | Gemmiger | Gemmiger formicilis |
| formicilis | | | | | | |
| Agathobaculum | 1628085.84 | 1.04 | 1.01 | rpob_SPA27 | Agathobaculum | Agathobaculum |
| butyriciproducens | | | | | | butyriciproducens |
| Faecalibacterium | 259315.11 | 1.03 | 1.07 | rpob_SPA28 | Faecalibacterium | Faecalibacterium |
| sp900539885 | | | | | | sp900539885 |
| Alistipes | 679935.3 | 1 | 0.98 | rpob_SPA29 | Alistipes | Alistipes finegoldii |
| finegoldii | | | | | | |
| ER4 | 172733.1407 | 0.99 | 1.00 | rpob_SPA30 | ER4 | ER4 sp000765235 |
| sp000765235 | | | | | | |
| Gemmiger | 1898205.22 | 0.96 | 0.96 | rpob_SPA31 | Gemmiger | Gemmiger qucibialis |
| qucibialis | | | | | | |
| Lachnospira | 165185.165 | 0.94 | 0.92 | rpob_SPA32 | Lachnospira | Lachnospira |
| sp000437735 | | | | | | sp000437735 |
| Dialister invisus | 278064.91 | 0.88 | 0.86 | rpob_SPA33 | Dialister | Dialister invisus |
| Bariatricus comes | 410072.533 | 0.88 | 0.90 | rpob_SPA34 | Bariatricus | Bariatricus comes |
| Bacteroides | 47678.881 | 0.87 | 0.84 | rpob_SPA35 | Bacteroides | Bacteroides caccae |
| caccae | | | | | | |
| Parabacteroides | 823.3168 | 0.86 | 0.87 | rpob_SPA36 | Parabacteroides | Parabacteroides |
| distasonis | | | | | | distasonis |
| Parabacteroides | 46503.2088 | 0.83 | 0.83 | rpob_SPA37 | Parabacteroides | Parabacteroides |
| merdae | | | | | | merdae |
| Bacteroides | 47678.882 | 0.73 | 0.73 | rpob_SPA35 | Bacteroides | Bacteroides caccae |
| caccae | | | | | | |
| Faecalibacterium | 1971605.56 | 0.72 | 0.71 | rpob_SPA38 | Faecalibacterium | Faecalibacterium |
| prausnitzii_D | | | | | | prausnitzii_D |
| Barnesiella | 2212467.8 | 0.72 | 0.73 | rpob_SPA39 | Barnesiella | Barnesiella |
| intestinihominis | | | | | | intestinihominis |
| Roseburia | 360807.1171 | 0.71 | 0.70 | rpob_SPA40 | Roseburia | Roseburia |
| sp900552665 | | | | | | inulinivorans |
| | | | | | | Roseburia |
| | | | | | | sp900552665 |
| Roseburia | 360807.64 | 0.71 | 0.69 | rpob_SPA40 | Roseburia | Roseburia |
| inulinivorans | | | | | | inulinivorans |
| | | | | | | Roseburia |
| | | | | | | sp900552665 |
| KLE1615 | 1898203.1773 | 0.64 | 0.69 | rpob_SPA41 | KLE1615 | KLE1615 |
| sp900066985 | | | | | | sp900066985 |
| Dorea_A | 88431.960 | 0.63 | 0.65 | rpob_SPA42 | Dorea_A | Dorea_A longicatena |
| longicatena | | | | | | |
| Roseburia | 166486.952 | 0.59 | 0.61 | rpob_SPA43 | Roseburia | Roseburia intestinalis |
| intestinalis | | | | | | |
| CAG-41 | 2044939.1074 | 0.58 | 0.58 | rpob_SPA44 | CAG-41 | CAG-41 |
| sp900066215 | | | | | | sp900066215 |
| Blautia_A | 2292961.3 | 0.58 | 0.59 | rpob_SPA45 | Blautia_A | Blautia_A |
| sp000436615 | | | | | | sp000436615 |
| Alistipes | 328813.45 | 0.54 | 0.54 | rpob_SPA46 | Alistipes | Alistipes onderdonkii |
| onderdonkii | | | | | | Alistipes megaguti |
| | | | | | | Alistipes shahii |

### Example 12: Simulation of Sensitivity and Specificity Analysis of Deep Next Generation Sequencing

Simulation of sensitivity and specificity analysis of deep NGS sequencing of mcfDNA fragments followed by taxonomic classification using read-based metagenome analysis methods: The current approach to analyze microbial signatures in cfDNA involves deep NGS sequencing. After filtering out the human DNA reads, the mcfDNA reads are analyzed; this is customary done using read-based taxonomic classifiers. To understand the usefulness of read-based taxonomic classifiers for mcfDNA informed community analysis we simulated mcfDNA fragments and classified them with either Kaiju (Menzel et al, 2016) or Kraken 2 (Wood et al, 2019), two commonly used read-based taxonomic classifiers. For this simulation we used the assumption that on a routine basis 100 cfDNA samples were sequenced in parallel on a NovaSeq 6000 NGS sequencer. Since the maximum capacity of the NovaSeq 6000 is approximately 20 billion reads, this would enable sequencing of a maximum of 200 million cfDNA fragments per sample. This is in line with the numbers published by Poore et al (2020). Based on the assumption that 1% of the cfDNA represents mcfDNA fragments, around 2 million mcfDNA fragments sequence reads will be generated per sample.

For each genome in the microbial community of Table 40, the length weighted relative abundance of total sample fragments was determined to account for the larger number of mcfDNA fragments generated from larger genomes. This abundance was subsequently used to determine the number of mcfDNA fragments generated per genome. The mcfDNA fragment sizes were randomly selected from a truncated normal distribution with fragment sizes between 1 and 200 base pairs and an average of 60 base pairs; these represents the same parameters as used for the SPA fragment simulation and matches best with the reported size distribution for mcfDNA fragments (Burnham et al, 2016). The fragment start and end positions were randomly selected from the genomes.

The results of the taxonomic assignment of fragments by Kaiju and Kraken 2 to different phylogenetic levels, ranging from phylum to species, is presented in Table 47. The community compositions determined by PacBio sequencing and the SPA fragment sequencing simulation using the RpoB1-R1327 primer are included for reference. Based on the results presented in Table 47 it can be concluded that Kaiju and Kraken 2 failed to correctly assign short mcfDNA reads to their taxonomic classification or to correctly deconvolute the community composition. This is in contrast to the results obtained for the SPA fragment sequencing simulation, which closely matched the community composition obtained by PacBio sequencing that was used as input for all three simulations. It is also important to remember that for all three simulations, similar mcfDNA fragments with an average length of 60 base pairs and a similar size distribution were used.

TABLE 47
High-level phylogenetic breakdown and assignment of simulated
mcfDNA reads to different phylogenetic levels by Kaiju and Kraken 2.
For comparison, phylogenetic breakdown of the community obtained by
PacBio sequencing and simulated SPA fragment sequencing are
included. The numbers between brackets represent the number
of reads that were assigned by Kaiju and Kraken 2 to a
phylogenetic level; this excludes fragments identified as viruses and
unclassified reads.

| | | SPA | | | | |
|---|---|---|---|---|---|---|
| Phylogenetic | | fragment | | | | |
| level | PacBio | sequencing | Kaiju | | Kraken 2 | |
| Phylum | 4 | 4 | 70 | (1,307,526) | 42 | (856,014) |
| Class | 4 | 4 | 90 | (1,216,360) | 78 | (849,019) |
| Order | 6 | 6 | 177 | (1,212,705) | 174 | (848,572) |
| Family | 11 | 11 | 327 | (930,470) | 384 | (818,206) |
| Genus | 27 | 27 | 735 | (818,814) | 1220 | (771,360) |
| Species | 46 | 46 | 2,436 | (193,935) | 3,605 | (629,023) |

Further details on the phylogenetic assignment of mcfDNA reads to the genus level by Kaiju and Kraken 2 are presented in Table 48 and Table 49, respectively. In the original community, all 52 members are present at a relative abundance ranging from 3.541 to 21.61 (see Table 40). Of the reads, 40.77 and 38.04 could be assigned by Kaiju and Kraken 2, respectively, to the genus level, represented by genera with a relative abundance of 0.01% or above. This number is in line with the results published by Poore et al (2020), with 35.8% of the mcfDNA reads being assigned to the genus level. A further comparison of the genus level taxonomic assignment is provided in Table 50.

TABLE 48
Composition on the genus level of the simulated gut
microbiome community using Kaiju
(version 1.7.2) for taxonomic classification of
in silico generated mcfDNA fragments.

| Genus-level | Percentage of mcfDNA |
|---|---|
| assignment | fragments assigned |
| by Kaiju | (%) |
| Bacteroides | 23.98 |
| Faecalibacterium | 3.51 |
| Alistipes | 2.96 |
| Roseburia | 2.72 |
| Ruminococcus | 1.68 |
| Paraprevotella | 1.45 |
| Bifidobacterium | 1.02 |
| Parabacteroides | 0.61 |
| Blautia | 0.59 |
| Eubacterium | 0.49 |
| Clostridium | 0.35 |
| Coprococcus | 0.33 |
| Subdoligranulum | 0.31 |
| Dorea | 0.25 |
| Dialister | 0.24 |
| Butyricicoccus | 0.09 |
| Gemmiger | 0.06 |
| Prevotella | 0.03 |
| Fusicatenibacter | 0.02 |
| Clostridioides | 0.02 |
| Barnesiella | 0.02 |
| Anaerobutyricum | 0.01 |
| Anaerostipes | 0.01 |
| Oscillibacter | 0.01 |
| Lachnoclostridium | 0.01 |
| Total assigned | 40.77 |

TABLE 49
Composition on the genus level of the
simulated gut microbiome community using
Kraken 2 (version 2.08) for taxonomic classification
of in silico generated mcfDNA fragments.
Kraken 2 (version 2.1.2) was also run with
no significant improvement in the results.

| | Percentage of |
|---|---|
| Genus-level assignment | mcfDNA fragments |
| by Kraken 2 | assigned (%) |
| Bacteroides | 23.41 |
| Alistipes | 2.74 |
| Faecalibacterium | 2.73 |
| Blautia | 2.07 |
| Bifidobacterium | 1.64 |
| Paraprevotella | 1.00 |
| Parabacteroides | 0.98 |
| Ruminococcus | 0.74 |
| Roseburia | 0.61 |
| Anaerobutyricum | 0.60 |
| Anaerostipes | 0.50 |
| Lachnoclostridium | 0.10 |
| Clostridium | 0.08 |
| Eubacterium | 0.07 |
| Prevotella | 0.05 |
| Butyricimonas | 0.05 |
| Clostridioides | 0.05 |
| Mordavella | 0.04 |
| Bacillus | 0.03 |
| Paenibacillus | 0.03 |
| Faecalitalea | 0.03 |
| Muribaculum | 0.02 |
| Barnesiella | 0.02 |
| Butyrivibrio | 0.02 |
| Streptococcus | 0.02 |
| Longibaculum | 0.02 |
| Streptomyces | 0.02 |
| Pseudomonas | 0.02 |
| Alloprevotella | 0.01 |
| Tannerella | 0.01 |
| Odoribacter | 0.01 |
| Duncaniella | 0.01 |
| Porphyromonas | 0.01 |
| Proteiniphilum | 0.01 |
| Chryseobacterium | 0.01 |
| Flavobacterium | 0.01 |
| Capnocytophaga | 0.01 |
| Hymenobacter | 0.01 |
| Mucilaginibacter | 0.01 |
| Sphingobacterium | 0.01 |
| Pedobacter | 0.01 |
| Chitinophaga | 0.01 |
| Pseudobutyrivibrio | 0.01 |
| Ruthenibacterium | 0.01 |
| Flavonifractor | 0.01 |
| Hungatella | 0.01 |
| Flintibacter | 0.01 |
| Dysosmobacter | 0.01 |
| Oscillibacter | 0.01 |
| Staphylococcus | 0.01 |
| Lactobacillus | 0.01 |
| Enterococcus | 0.01 |
| Corynebacterium | 0.01 |
| Citrobacter | 0.01 |
| Acinetobacter | 0.01 |
| Vibrio | 0.01 |
| Burkholderia | 0.01 |
| Campylobacter | 0.01 |
| Total assigned | 38.04 |

TABLE 50
Comparison between the composition on the genus level of the gut
microbiome community between the SPA fragment sequencing
simulation and simulated
NGS sequencing of mcfDNA using Kaiju or Kraken 2 for taxonomic
classification. To facilitate comparison, some of the genera listed in
Table 46 have been combined,
reducing the total number of genera from 27 to 25. N.A.: not applicable;
the genus was either not found or no reads were assigned to it.
The genera Phocaeicola and Mediterraneibacter were not
present in the databases used
for taxonomic classification by Kaiju or Kraken 2, and their
abundances were included in the genera Bacteroides and
Ruminococcus, respectively, to which they previously belonged.

| | | | Relative | Relative |
|---|---|---|---|---|
| | Relative | Relative | Genus | Genus |
| Microbial | Abundance | Abundance | Abundance | Abundance |
| community | % PacBio | % SPA | % Kaiju | % Kraken |
| genus | sequencing | simulation | simulation | simulation |
| Bacteroides | 32.67 | 32.7 | 23.98 | 23.41 |
| Blautia | 10.61 | 10.62 | 0.59 | 2.07 |
| Faecalibacterium | 10 | 10.05 | 3.51 | 2.73 |
| Agathobacter | 6.46 | 6.45 | 0.00005 | N.A. |
| Alistipes | 6.39 | 6.37 | 2.96 | 2.74 |
| Phocaeicola | 5.65 | 5.64 | Bacteroides | Bacteroides |
| Agathobaculum/ | 3.27 | 3.23 | 1.458 | 1.00 |
| Paraprevotella | | | | |
| Bifidobacterium | 3.13 | 3.14 | 1.02 | 1.64 |
| Fusicatenibacter | 2.11 | 2.08 | 0.022 | N.A. |
| Gemmiger | 2.03 | 2.01 | 0.06 | N.A. |
| Roseburia | 2.01 | 2 | 2.72 | 0.61 |
| Parabacteroides | 1.69 | 1.7 | 0.61 | 0.98 |
| Lachnospira | 1.58 | 1.61 | N.A. | 0.90 |
| family bacteria | | | | |
| Ruminococcus | 1.5 | 1.47 | 1.68 | 0.74 |
| Schaedlerella | 1.46 | 1.46 | N.A. | N.A. |
| Acetatifactor | 1.44 | 1.46 | 0.00045 | N.A. |
| Mediterraneibacter | 1.15 | 1.15 | Ruminococcus | Ruminococcus |
| Anaerostipes | 1.1 | 1.1 | 0.012 | 0.50 |
| Anaerobutyricum | 1.07 | 1.02 | 0.014 | 0.60 |
| Oscillospiraceae | 0.99 | 1 | N.A. | 0.03 |
| family bacteria | | | | |
| Bariatricus | 0.88 | 0.86 | 0.0003 | N.A. |
| Dialister | 0.88 | 0.9 | 0.24 | <0.01 |
| Barnesiella | 0.72 | 0.71 | 0.018 | 0.02 |
| Dorea | 0.63 | 0.65 | 0.25 | N.A. |
| CAG-41 | 0.58 | 0.59 | N.A. | N.A. |
| sp900066215 | | | | |
| Total assigned- | 100% | 100% | 39.1448% | 37.98% |
| genus level | | | | |

Based on the results presented in Table 50 it can be concluded that all three simulations identified the most abundant genera, including Bacteroides, Blautia, Faecalibacterium, Alistipes, Phocaeicola, Agathobaculum Paraprevotella, Bifidobacterium and Fusicatenibacter. However, compared to the input data for the simulations, the numbers for their relative abundances are imprecise for Kaiju and Kraken 2. This becomes even more obvious for low abundant species. In addition, the read-based taxonomic classification tools fail to provide any meaningful insights when multiple closely related species are present.

Species and subspecies level insights are required to draw meaningful conclusions between microbial signatures and diseases, including cancer detection and prognostics. The simulated compositions on the species level of the gut microbiome community using Kaiju or Kraken 2 for taxonomic classification of in silico generated mcfDNA fragments were very imprecise. For Bacteroides stercoris, the dominant species present at 21.61% in the community, Kaiju was able to match 2.6% of the mcfDNA fragments to this species, while Kraken 2 failed to link any mcfDNA fragments to this species. This clearly shows that read-based taxonomic classification tools are lacking the sensitivity and specificity required to analyze microbial signatures present in mcfDNA from biopsy samples.

Conclusion: Short DNA fragments with an average length of approximately 60 base pairs are an intrinsic property of mcfDNA. In contrast to the result from the simulation using SPA fragment sequencing-based analysis, where the fragments were generated using the RpoB1-R1327 primer, simulations using deep metagenome sequencing of cfDNA fragments followed by taxonomic classification of mcfDNA using read-based metagenome analysis methods showed that the current read-based tools are unsuitable for taxonomic classification of the short sequencing reads obtained from mcfDNA. As such this approach lacks the sensitivity and specificity to provide meaningful insights for disease detection and progression monitoring. An approach to overcome this limitation would require very deep sequencing and assembly of short reads into larger fragments. In addition to a significantly higher sequencing cost, limitations in the assembly of short sequencing reads makes this approach unsuitable for scalable application to the routine analysis of microbial patterns in biopsy samples.

### Example 13: Cpn60 Gene-Based Spa Fragment Sequencing

As concluded from EXAMPLE 11, SPA fragment sequences obtained with the primer RpoB1-R1327 provided excellent phylogenetic resolution for gut microbiome bacteria at the genus level and in most instances at the species and subspecies level. However, in some instances, it failed to discriminate between very closely related species, such as Bacteroides ovatus and Bacteroides xylanisolvens, and Alistipes onderdonkii, Alistipes finegoldii and Alistipes shahii.

Design of the Cpn60-R571 SPA primer: To further improve the phylogenetic resolution compared to SPA fragment sequencing based on the rpoB gene (using primer RpoB1-R1327) we analyzed the 60 kDa chaperonin protein gene (cpn60 gene, also referred to as the groEL gene) for SPA fragment sequencing. Using the method described herein above and exemplified in Example 2, a conserved region spanning position 571 to 593 (position numbers based on the Escherichia coli cpn60 gene) was identified for SPA fragment sequencing; this primer annealing region is located downstream of a hypervariable DNA region to be used for phylogenetic identification. The degenerate nucleotide sequence of this region is presented in FIG. 7B. The primer Cpn60-R571 was tested for SPA fragment amplification of the region upstream of position 571 of the cpn60 gene as described in this Example. The Cpn60-R571 primer has the sequence listed below, using the following nucleotide codes: A: adenine; G: guanidine; C: cytosine; T: thymine; R: purine (A or G); Y: pyrimidine (T or C); K: amino (T or G); B: not A (T, G or C); N: any nucleotide (A, G, C or T).

Cpn60-R571 primer: 5′ CCN.YKR.TCR.AAB.YGC.ATN.CCY.TC 3′ (SEQ ID NO: 3)

As described herein above, a conserved primer annealing region is located adjacent to at least one of a 25 nucleotide-long or a 50 nucleotide-long variable region with preferably an average sequence variance of <0.1 and <0.075, respectively. As can be seen in Table 51, the 25 nucleotide-long variable region located upstream of the Cpn60-R571 primer annealing site has an average sequence variance of 0.0851.

TABLE 51
Average sequence variance for the Cpn60-R571 primer region and the regions upstream or downstream of the
primer annealing region. For both regions located adjacent to the primer region, the variance is shown
for 25, 50, 75, 100 or 200 nucleotides (nt) upstream (5′) or downstream (3′) of the beginning or
end of the primer annealing sequence. The variance score is calculated as the average of the variance
of the percentage of the nucleotides adenine, guanidine, cytosine and thymine at each position of the
cpn60 gene. A lower number is indicative for more variance, while a higher number is indicative for less
variance and a more conserved DNA sequence. The maximum theoretical variance score for a region is 0.25
(would represent a 100% conserved DNA region). Regions with a variance score <0.1 are highlighted in grey.

| | Average of variance | | | | | | | | | | |
|---|---|---|---|---|---|---|---|---|---|---|---|
| | Region upstream of primer | | | | | | Region downstream of primer | | | | |
| Primer name - | 200 nt | 100 nt | 75 nt | 50 nt | 25 nt | Primer | 25 nt | 50 nt | 75 nt | 100 nt | 200 nt |
| recognized | before | before | before | before | before | Primer | after | after | after | after | after |
| region | primer | primer | primer | primer | primer | region | primer | primer | primer | primer | primer |
| Cpn60-R571 | 0.0879 | 0.1249 | 0.1251 | 0.1115 | 0.0851 | 0.1859 | 0.1319 | 0.1112 | 0.1119 | 0.1136 | 0.118 |

In silico sensitivity analysis for Cpn60-R571-based SPA fragment sequences: Using a similar consortium (see Table 40) and parameters for the simulations as described in EXAMPLE 10, a simulation was performed to determine the sensitivity of SPA fragment sequencing using the Cpn60-R571 primer annealing site. The 52-member community, whose composition was obtained with PacBio sequencing, is described in Table 52. The sequences of the SPA fragments obtained for each of the community members are also presented. The 50 base pair SPA fragments that are identical for multiple closely related community members are highlighted in grey. Based on the results from EXAMPLE 10, mcfDNA fragments with an average sequence length of 60 base pairs were used in this simulation. The results from the simulation using the Cpn60-R571 primer showed that mcfDNA fragments with an average length of 60 base pairs can be reliably used to determine the microbial community composition when the strains are present at approximately 0.5%0 (Table 53). These results are very similar to the results that were obtained for the simulation using the RpoB1-R1327 primer (Table 44).

TABLE 52

| | | PacBio | SPA | Cpn60 | |
|---|---|---|---|---|---|
| | | Relative | Relative | SPA | |
| Genome | PATRIC | Abundance | Abundance | fragments | SPA Fragment |
| Name | Genome ID | % | % | code | sequence (50 bp) |
| Bacteroides stercoris | 46506.122 | 21.61 | 21.64 | cpn60_ | TTATCACTATCGAAGAG |
| strain AM51-2BH | | | | SPA1 | GCTAAGGGTACCGATAC |
| | | | | | CACTATCGGTGTAGTA |
| | | | | | (SEQ ID NO: 343) |
| Bacteroides vulgatus | 821.3904 | 5.65 | 5.68 | cpn60_ | TGATTACTATCGAAGAA |
| strain VPI-5710 | | | | SPA2 | GCTAAAGGAACGGATAC |
| | | | | | TACCATCGGTGTGGTA |
| | | | | | (SEQ ID NO: 344) |
| Agathobacter sp. | 2021311.24 | 4.26 | 4.29 | cpn60_ | TCATCACAATCGAAGAG |
| strain COPD130 | | | | SPA3 | TCCAAAACCATGCAGAC |
| | | | | | AGAGCTTGACCTGGTA |
| | | | | | (SEQ ID NO: 345) |
| Bacteroides ovatus | 28116.1423 | 3.69 | 3.64 | cpn60_ | TGATTACTATCGAAGAA |
| strain | | | | SPA4 | GCAAAAGGAACAGACAC |
| 1001275B_160808_ | | | | | TACTATCGGTGTAGTA |
| G11 | | | | | (SEQ ID NO: 346) |
| Blautia massiliensis | 1737424.64 | 3.14 | 3.13 | cpn60_ | TTATCACTGTTGAGGAGT |
| strain MSK.13.24 | | | | SPA5 | CCAAGACCATGCATACA |
| | | | | | GAGCTTGACCTTGTA |
| | | | | | (SEQ ID NO: 347) |

| | | | | | |
|---|---|---|---|---|---|
| Alistipes putredinis | 445970.5 | 2.92 | 2.98 | cpn60_ | TCATCACCGTCGAGGAG |
| DSM 17216 | | | | SPA6 | GCCAAGGGTACCGAAAC |
| | | | | | CCATGTGGATGTGGTC |
| | | | | | (SEQ ID NO: 348) |
| Faecalibacterium | 853.7274 | 2.73 | 2.77 | cpn60_ | TCACCATCGAGGAGAAC |
| prausnitzii strain | [853.7674] | | | SPA7 | AAGACCACCGCCGAGAC |
| S03C.meta.bin_9 | | | | | CTACAACGAGATCGTG |
| [Faecalibacterium | | | | | (SEQ ID NO: 349) |
| prausnitzii strain | | | | | |
| COPD342] | | | | | |
| Bacteroides ovatus | 28116.176 | 2.45 | 2.48 | cpn60_ | TGATTACTATCGAAGAA |
| strain AF26-20AA | | | | SPA4 | GCAAAAGGAACAGACAC |
| | | | | | TACTATCGGTGTAGTA |
| | | | | | (SEQ ID NO: 346) |
| Blautia wexlerae | 418240.179 | 2.36 | 2.37 | cpn60_ | TTATCACAGTAGAAGAA |
| strain | [1262967.3] | | | SPA8 | TCCAAGACAATGCACAC |
| S09A.meta.bin_3 | | | | | AGAACTTGACCTTGTA |
| [Ruminococcus sp. | | | | | (SEQ ID NO: 350) |
| CAG: 9] | | | | | |
| Paraprevotella clara | 1263095.48 | 2.23 | 2.23 | cpn60_ | TGATTACCATCGAAGAA |
| CAG: 116 strain | | | | SPA9 | GCCAAGGGACGCGACAC |
| MGS: 116 | | | | | TACTATCGGTGTGGTG |
| | | | | | (SEQ ID NO: 351) |
| [Eubacterium] | 39491.2479 | 2.2 | 2.15 | cpn60_ | TTATCACAATTGAAGAG |
| rectale strain | | | | SPA10 | TCAAAGACAATGCAGAC |
| BIOML-A1 | | | | | AGAGCTTGACCTTGTA |
| | | | | | (SEQ ID NO: 352) |
| Blautia wexlerae | 418240.389 | 2.11 | 2.13 | cpn60_ | TTATCACCATCGAGGAG |
| strain | | | | SPA11 | TCCAAGACCATGCAGAA |
| 1001270J_160509_ | | | | | CGAGCTGGAGCTGGTA |
| E6 | | | | | (SEQ ID NO: 353) |
| Ruminococcus sp. | 2293212.3 | 2.07 | 2.09 | cpn60_ | TTATCACAGTAGAAGAA |
| AM40-10AC | | | | SPA8 | TCCAAGACAATGCACAC |
| | | | | | AGAACTTGACCTTGTA |
| | | | | | (SEQ ID NO: 354) |
| Faecalibacterium | 853.7698 | 2.04 | 2.03 | cpn60_ | TCACCATCGAGGAGAAC |
| prausnitzii strain | | | | SPA12 | AAGACCACTGCCGAGAC |
| COPD315 | | | | | CTACAACGAGATCGTC |
| | | | | | (SEQ ID NO: 355) |
| Faecalibacterium sp. | 2580425.3 | 2.01 | 2.00 | cpn60_ | TCACCATCGAGGAGAAC |
| Marseille-P9312 | | | | SPA12 | AAGACCACTGCCGAGAC |
| | | | | | CTACAACGAGATCGTC |
| | | | | | (SEQ ID NO: 356) |
| Alistipes obesi strain | 1118061.514 | 1.93 | 1.95 | cpn60_ | TCATCACGGTCGAGGAG |
| MGYG-HGUT- | | | | SPA13 | GCCAAAGGCACCGACAC |
| 01415 | | | | | CCATGTGGACGTGGTC |
| | | | | | (SEQ ID NO: 357) |
| Bifidobacterium | 28026.777 | 1.76 | 1.69 | cpn60_ | TCGTGACCGTTGAGGAC |
| pseudocatenulatum | | | | SPA14 | AACAACCGCTTCGGCCT |
| LFYP 29 | | | | | GGATCTGGACTTTACC |
| | | | | | (SEQ ID NO: 358) |
| Bacteroides sp. | 2292949.3 | 1.73 | 1.71 | cpn60_ | TTATCACTATCGAAGAG |
| AM30-16 | | | | SPA15 | GCAAAGGGTACTGATAC |
| | | | | | TACTATCGGTGTGGTT |
| | | | | | (SEQ ID NO: 359) |
| Bacteroides ovatus | 28116.180 | 1.6 | 1.63 | cpn60_ | TGATTACTATCGAAGAA |
| strain OF01-19AC | | | | SPA4 | GCAAAAGGAACAGACAC |
| | | | | | TACTATCGGTGTAGTA |
| | | | | | (SEQ ID NO: 346) |
| Ruminococcus sp. | 41978.12 | 1.5 | 1.50 | cpn60_ | TTATCACTCTTGAGGAGT |
| strain UBA10663 | | | | SPA16 | CAAAGACTGCTGAAACT |
| | | | | | TACAGCGAAGTCGTT |
| | | | | | (SEQ ID NO: 360) |
| Faecalibacterium | 853.266 | 1.47 | 1.48 | cpn60_ | TCACCATCGAGGAGAAC |
| prausnitzii strain | | | | SPA17 | AAGACCACTGCCGAGAC |
| APC923/51-1 | | | | | CTACAACGAGATCGTG |
| | | | | | (SEQ ID NO: 361) |
| Firmicutes | 2292892.3 | 1.46 | 1.44 | cpn60_ | TTATTACAATCGAAGAA |
| bacterium AM31- | | | | SPA18 | TCTAAAACAATGCAGAC |
| 12AC | | | | | AGAGCTTGACCTTGTG |
| | | | | | (SEQ ID NO: 362) |
| Acetatifactor sp. | 1872090.5 | 1.44 | 1.41 | cpn60_ | TTATCACCATTGAAGAGT |
| strain COPD172 | | | | SPA19 | CCAAGACCATGCAGACC |
| | | | | | GAACTGGATCTGGTA |
| | | | | | (SEQ ID NO: 363) |
| Bifidobacterium | 1679.11 | 1.37 | 1.38 | cpn60_ | TTGTGACCGTTGAAGAC |
| longum subsp. | | | | SPA20 | AACAACCGCTTCGGCCT |
| longum strain 9 | | | | | GGACCTCGACTTCACC |
| | | | | | (SEQ ID NO: 364) |
| Blautia faecis strain | 871665.25 | 1.26 | 1.25 | cpn60_ | TTATTACTGTAGAAGAGT |
| MSK.11.45 | | | | SPA21 | CCAAGACCATGCACACA |
| | | | | | GAGCTTGACCTTGTA |
| | | | | | (SEQ ID NO: 365) |
| Ruminococcus sp. | 2787081.3 | 1.2 | 1.19 | cpn60_ | TTATTACTGTAGAAGAGT |
| D40tl_170626_H2 | | | | SPA21 | CCAAGACCATGCACACA |
| | | | | | GAGCTTGACCTTGTA |
| | | | | | (SEQ ID NO: 366) |
| [Ruminococcus] | 46228.446 | 1.15 | 1.17 | cpn60_ | TGATTACGATCGAGGAG |
| lactaris strain | | | | SPA22 | TCCAAGACTATGCAGAC |
| SRR7721875-bin.26 | | | | | AGAACTGGATCTTGTA |
| | | | | | (SEQ ID NO: 367) |
| Anaerostipes hadrus | 649756.2503 | 1.1 | 1.10 | cpn60_ | TTATCACGATCGAAGAA |
| strain | | | | SPA23 | TCTAAAACAATGAAAAC |
| S01C.meta.bin_9 | | | | | AGAATTAGATTTAGTA |
| | | | | | (SEQ ID NO: 368) |
| Eubacterium sp. | 1897002.3 | 1.07 | 1.06 | cpn60_ | TTATTACAATCGAAGAG |
| 38_16 | | | | SPA25 | TCTAAGACAATGAAAAC |
| | | | | | AGAGCTTGACCTTGTA |
| | | | | | (SEQ ID NO: 369) |
| Subdoligranulum sp. | 2053618.24 | 1.07 | 1.11 | cpn60_ | TCACCATCGAGGAGAAC |
| strain | | | | SPA24 | AAGACCACTGCCGAGAC |
| S08B.meta.bin_8 | | | | | CTACACCGAGGTCGTC |
| | | | | | (SEQ ID NO: 370) |
| Agathobaculum | 1628085.84 | 1.04 | 1.01 | cpn60_ | TTATCACCGTTGAGGAGT |
| butyriciproducens | | | | SPA26 | CCAAGACCGCTGAGACC |
| strain COPD228 | | | | | TACTCGGAGGTTGTT |
| | | | | | (SEQ ID NO: 371) |
| uncultured | 259315.11 | 1.03 | 1.02 | cpn60_ | TCACCATTGAGGAGAAC |
| Faecalibacterium sp. | | | | SPA27 | AAGACCACTGCTGAGAC |
| strain UMGS184 | | | | | CTACAACGAGATCGTA |
| | | | | | (SEQ ID NO: 372) |
| Alistipes finegoldii | 679935.3 | 1 | 1.01 | cpn60_ | TCATCACCGTCGAGGAG |
| DSM 17242 | | | | SPA28 | GCCAAAGGCACCGAGAC |
| | | | | | CCACGTGGAGGTGGTC |
| | | | | | (SEQ ID NO: 373) |
| uncultured | 172733.1407 | 0.99 | 0.95 | cpn60_ | TCATCACCATCGAGGAG |
| Clostridiales | | | | SPA29 | TCCAAGACCGCCGAGAC |
| bacterium strain | | | | | CTACAGCGAGGTCGTC |
| UMGS84 | | | | | (SEQ ID NO: 374) |
| Ruminococcaceae | 1898205.22 | 0.96 | 0.98 | cpn60_ | TCACCATTGAGGAGAAC |
| bacterium strain | | | | SPA30 | AAGACCACTGCTGAAAC |
| UBA9091 | | | | | CTACACCGAGGTAGTG |
| | | | | | (SEQ ID NO: 375) |
| uncultured | 165185.165 | 0.94 | 0.91 | cpn60_ | TTATCACAATCGAAGAA |
| Eubacterium sp. | | | | SPA31 | TCTAAGACCATGAAGAC |
| strain UMGS39 | | | | | AGAGCTTGACCTTGTA |
| | | | | | (SEQ ID NO: 376) |
| uncultured Dialister | 278064.91 | 0.88 | 0.86 | cpn60_ | TTATTACTGTAGAAGATT |
| sp. strain | | | | SPA32 | CCAAAACTATGGGTACA |
| ERR414242-bin.5 | | | | | AGCCTTAAAGTTGTG |
| | | | | | (SEQ ID NO: 377) |
| Coprococcus comes | 410072.533 | 0.88 | 0.88 | cpn60_ | TTATCACAATTGAAGAG |
| strain MSK.16.14 | | | | SPA33 | TCAAAGACAATGAAGAC |
| | | | | | AGAGCTTGACCTTGTA |
| | | | | | (SEQ ID NO: 378) |
| Bacteroides caccae | 47678.881 | 0.87 | 0.86 | cpn60_ | TTATCACTATCGAAGAA |
| strain BIOML-A2 | | | | SPA34 | GCAAAAGGTACTGACAC |
| | | | | | TACAATCGGTGTAGTA |
| | | | | | (SEQ ID NO: 379) |
| Parabacteroides | 823.3168 | 0.86 | 0.86 | cpn60_ | TTATCACGGTTGAGGAA |
| distasonis strain | | | | SPA35 | GCTAAAGGTACTGAAAC |
| LMAG: 27 | | | | | TACAGTTGACGTAGTT |
| | | | | | (SEQ ID NO: 380) |
| Parabacteroides | 46503.2088 | 0.83 | 0.82 | cpn60_ | TTATCACTGTAGAAGAA |
| merdae strain | | | | SPA36 | GCTAAAGGCACGGAAAC |
| 1001136B_160425_ | | | | | AACAGTAGACGTGGTA |
| B1 | | | | | (SEQ ID NO: 381) |
| Bacteroides caccae | 47678.882 | 0.73 | 0.71 | cpn60_ | TTATCACTATCGAAGAA |
| strain BIOML-A1 | | | | SPA34 | GCAAAAGGTACTGACAC |
| | | | | | TACAATCGGTGTAGTA |
| | | | | | (SEQ ID NO: 379) |
| Faecalibacterium sp. | 1971605.56 | 0.72 | 0.77 | cpn60_ | TCACCATTGAGGAGAAC |
| strain | | | | SPA37 | AAGACCACCGCTGAGAC |
| S04C.meta.bin_2 | | | | | CTACAACGAGATCGTG |
| | | | | | (SEQ ID NO: 382) |
| Bacteroidaceae | 2212467.8 | 0.72 | 0.75 | cpn60_ | TTATCACGGTAGAAGAG |
| bacterium strain | | | | SPA38 | GCCAAAGGTACCGATAC |
| MGYG-HGUT- | | | | | GACTGTCGATATTGTA |
| 00144 | | | | | (SEQ ID NO: 383) |
| Roseburia | 360807.1171 | 0.71 | 0.70 | cpn60_ | TTATCACAATCGAAGAG |
| inulinivorans strain | | | | SPA39 | TCCAAGACGATGCAGAC |
| SRR5519173-bin.6 | | | | | AGAGCTTGATCTTGTA |
| | | | | | (SEQ ID NO: 384) |
| Roseburia | 360807.64 | 0.71 | 0.71 | cpn60_ | TTATCACAATCGAAGAG |
| inulinivorans strain | | | | SPA40 | TCCAAGACGATGCAGAC |
| AF28-15 | | | | | AGAGCTTGACCTTGTA |
| | | | | | (SEQ ID NO: 385) |
| Lachnospiraceae | 1898203.1773 | 0.64 | 0.64 | cpn60_ | TTATTACCATCGAGGAGT |
| bacterium strain | | | | SPA41 | CTAAGACCATGAAGACA |
| MGYG-HGUT- | | | | | GAGCTGGATCTTGTA |
| 00193 | | | | | (SEQ ID NO: 386) |
| Dorea longicatena | 88431.960 | 0.63 | 0.60 | cpn60_ | TCATCACAATTGAAGAA |
| strain MSK.11.4 | | | | SPA42 | TCTAAAACTATGAAGAC |
| | | | | | AGAGCTGGACCTTGTA |
| | | | | | (SEQ ID NO: 387) |
| Roseburia | 166486.952 | 0.59 | 0.60 | cpn60_ | TTATCACGATCGAGGAA |
| intestinalis strain | | | | SPA43 | TCTAAGACAATGCAGAC |
| ERR321618-bin.7 | | | | | AGAGCTTGACTTAGTA |
| | | | | | (SEQ ID NO: 388) |
| Clostridia bacterium | 2044939.1074 | 0.58 | 0.55 | cpn60_ | TTATCACAGTTGAAGAA |
| strain COPD107 | | | | SPA45 | TCAAAGACTGCCGAAAC |
| | | | | | ATATTCTGAAATTGTT |
| | | | | | (SEQ ID NO: 389) |
| Blautia sp. AF19- | 2292961.3 | 0.58 | 0.57 | cpn60_ | TTATCACAGTAGAAGAA |
| 10LB | | | | SPA44 | TCCAAGACCATGCATAC |
| | | | | | AGAACTTGACCTGGTA |
| | | | | | (SEQ ID NO: 390) |
| Alistipes | 328813.45 | 0.54 | 0.53 | cpn60_ | TGATCACCGTCGAGGAG |
| onderdonkii strain | | | | SPA46 | GCCAAGGGTACCGAGAC |
| D10-10 | | | | | CCATGTGGAGGTCGTA |
| | | | | | (SEQ ID NO: 391) |

Composition (species name and genome ID) and relative species abundances of the gut microbiome community used for the simulations. Long read PacBio sequencing was used to determine the community composition. The community composition based on the SPA fragment sequencing simulation was determined using the parameters described above and is also presented in Table 53. The codes and sequences for the unique 50 base pair SPA fragments generated for each species are shown. SPA fragments that are identical for multiple community members are highlighted in in grey. Compared to the strain selection for the RpoB1-R1327 simulation, two strains for which no cpn60 gene could be identified were replaced by closely related strains: Faecalibacterium prausnitzii strain COPD342 and Ruminococcus sp. CAG: 9 were replaced by Faecalibacterium prausnitzii strain S03C.meta.bin_9 and Blautia wexlerae strain S09A.meta.bin_3, respectively.

TABLE 53
Summary of Simulation 60-100 ng (average generated mcfDNA length of 60, 100 ng of cfDNA)
using the Cpn60-R571 primer.

| | | | | | | | | | | p-value | p-value |
|---|---|---|---|---|---|---|---|---|---|---|---|
| | | | | | | | | | | Wilcoxon | Wilcoxon |
| | | | | | | | | Based | | test | test |
| | | | | | | | | on | | H₀: | H₀: |
| | Total | Average | Average | | | | | SPA | | Count | Count |
| | mcfDNA | mcfDNA | mcfDNA | | | Avg | Avg | Fragments | | of | of |
| | Fragments | Fragments | Length | | | Count | Count | >24 bp | | SPA | SPA |
| | with | with | with | | Average | of | of | long | | fragments | fragments |
| | Conserved | Conserved | Conserved | Average | Maximum | SPA | SPA | Calculated | Theoretical | longer | longer |
| | Region | Region | Region | SPA | SPA | Fragments | Fragments | % | Relative | than | than |
| | for | for | for | Fragment | Fragment | >24 bp | >49 bp | Relative | Abundance % | 49 bp | 24 bp |
| Genome | Primer | Primer | Primer | Length | Length | long | long | Abundance | Input | <3 | <10 |
| 328813.45 | 2550 | 85 | 70 | 24 | 73 | 35 | 7 | 0.53 | 0.54 | 1.25E−06 | 8.95E−07 |
| 2044939.1074 | 2676 | 89 | 70 | 23 | 71 | 36 | 00 | 0.55 | 0.58 | 1.87E−06 | 9.06E−07 |
| 2292961.3 | 2724 | 91 | 70 | 24 | 73 | 38 | 00 | 0.57 | 0.58 | 1.27E−06 | 8.93E−07 |
| 88431.960 | 2998 | 100 | 71 | 23 | 77 | 40 | 0 | 0.60 | 0.63 | 8.86E−07 | 9.04E−07 |
| 166486.952 | 2770 | 92 | 71 | 24 | 75 | 40 | 00 | 0.60 | 0.59 | 2.15E−06 | 9.04E−07 |
| 1898203.1773 | 2997 | 100 | 70 | 24 | 74 | 42 | 0 | 0.64 | 0.64 | 1.23E−06 | 8.96E−07 |
| 360807.1171 | 3379 | 113 | 71 | 24 | 75 | 46 | 0 | 0.70 | 0.71 | 8.72E−07 | 8.95E−07 |
| 360807.64 | 3346 | 112 | 70 | 24 | 76 | 47 | 10 | 0.71 | 0.71 | 8.81E−07 | 9.04E−07 |
| 47678.882 | 3468 | 116 | 70 | 23 | 76 | 47 | 10 | 0.71 | 0.73 | 1.30E−06 | 9.01E−07 |
| 2212467.8 | 3488 | 116 | 71 | 24 | 77 | 50 | 10 | 0.75 | 0.72 | 8.48E−07 | 8.90E−07 |
| 1971605.56 | 3539 | 118 | 70 | 24 | 77 | 51 | 10 | 0.77 | 0.72 | 8.69E−07 | 8.98E−07 |
| 46503.2088 | 3909 | 130 | 71 | 24 | 76 | 54 | 11 | 0.82 | 0.83 | 8.77E−07 | 8.97E−07 |
| 47678.881 | 4141 | 138 | 70 | 23 | 79 | U | 12 | 0.86 | 0.87 | 1.02E−06 | 8.92E−07 |
| 823.3168 | 4148 | 138 | 70 | 24 | 76 | 57 | 12 | 0.86 | 0.86 | 8.93E−07 | 9.03E−07 |
| 278064.91 | 4189 | 140 | 70 | 24 | 78 | 57 | 13 | 0.86 | 0.88 | 8.66E−07 | 8.99E−07 |
| 410072.533 | 4095 | 137 | 71 | 24 | 77 | 58 | 12 | 0.88 | 0.88 | 8.86E−07 | 9.05E−07 |
| 165185.165 | 4462 | 149 | 70 | 23 | 79 | 60 | 12 | 0.91 | 0.94 | 8.75E−07 | 9.04E−07 |
| 172733.1407 | 4613 | 154 | 70 | 23 | 78 | 63 | 13 | 0.95 | 0.99 | 8.86E−07 | 8.93E−07 |
| 1898205.22 | 4638 | 155 | 71 | 24 | 77 | 65 | 14 | 0.98 | 0.96 | 8.85E−07 | 9.06E−07 |
| 679935.3 | 4719 | 157 | 70 | 24 | 77 | 67 | 13 | 1.01 | 1 | 8.73E−07 | 9.08E−07 |
| 1628085.84 | 4920 | 164 | 71 | 23 | 76 | 67 | 13 | 1.01 | 1.04 | 8.79E−07 | 8.97E−07 |
| 259315.11 | 4855 | 162 | 70 | 24 | 79 | 67 | 14 | 1.02 | 1.03 | 8.86E−07 | 9.08E−07 |
| 1897002.3 | 5152 | 172 | 70 | 23 | 76 | 71 | 15 | 1.06 | 1.07 | 8.81E−07 | 9.08E−07 |
| 649756.2503 | 5216 | 174 | 71 | 24 | 79 | 73 | 16 | 1.10 | 1.1 | 8.88E−07 | 9.02E−07 |
| 2053618.24 | 5013 | 167 | 71 | 24 | 79 | 73 | 15 | 1.11 | 1.07 | 8.92E−07 | 8.97E−07 |
| 46228.446 | 5605 | 187 | 70 | 24 | 77 | 77 | 17 | 1.17 | 1.15 | 8.89E−07 | 8.99E−07 |
| 2787081.3 | 5699 | 190 | 70 | 24 | 79 | 79 | 16 | 1.19 | 1.2 | 8.96E−07 | 9.04E−07 |
| 871665.25 | 6019 | 201 | 70 | 23 | 78 | 82 | 17 | 1.25 | 1.26 | 8.37E−07 | 9.02E−07 |
| 1679.11 | 6483 | 216 | 70 | 24 | 77 | 91 | 19 | 1.38 | 1.37 | 9.00E−07 | 9.09E−07 |
| 1872090.5 | 6751 | 225 | 70 | 23 | 82 | 93 | 18 | 1.41 | 1.44 | 8.86E−07 | 9.01E−07 |
| 2292892.3 | 6804 | 227 | 70 | 24 | 00 | 95 | 0 | 1.44 | 1.46 | 8.82E−07 | 8.97E−07 |
| 853.266 | 6941 | 23 | 70 | 24 | 79 | 98 | 20 | 1.48 | 1.47 | 8.91E−07 | 9.04E−07 |
| 41978.12 | 7065 | 236 | 70 | 24 | 79 | 99 | 20 | 1.50 | 1.5 | 8.99E−07 | 9.01E−07 |
| 28116.180 | 7645 | 255 | 70 | 24 | 00 | 108 | 22 | 1.63 | 1.6 | 8.92E−07 | 9.04E−07 |
| 28026.777 | 8190 | 273 | 70 | 23 | 79 | 112 | 23 | 1.69 | 1.76 | 8.95E−07 | 9.09E−07 |
| 2292949.3 | 8221 | 274 | 70 | 24 | 81 | 113 | 24 | 1.71 | 1.73 | 8.95E−07 | 9.06E−07 |
| 1118061.514 | 9206 | 307 | 70 | 24 | 82 | 129 | 26 | 1.95 | 1.93 | 9.05E−07 | 9.05E−07 |
| 2580425.3 | 9488 | 316 | 70 | 24 | 84 | 132 | 27 | 2.00 | 2.01 | 8.93E−07 | 9.08E−07 |
| 853.7698 | 9613 | 320 | 71 | 24 | 83 | 135 | 27 | 2.03 | 2.04 | 9.02E−07 | 9.04E−07 |
| 2293212.3 | 9917 | 331 | 71 | 24 | 83 | 139 | 31 | 2.09 | 2.07 | 9.01E−07 | 9.05E−07 |
| 418240.389 | 10164 | 339 | 70 | 24 | 83 | 141 | 29 | 2.13 | 2.11 | 9.00E−07 | 9.09E−07 |
| 39491.2479 | 10371 | 346 | 70 | 23 | 83 | 143 | 28 | 2.15 | 2.2 | 8.97E−07 | 9.04E−07 |
| 1263095.48 | 10648 | 355 | 70 | 23 | 82 | 148 | 28 | 2.23 | 2.23 | 9.06E−07 | 9.08E−07 |
| 418240.179 | 11275 | 376 | 70 | 24 | 82 | 157 | 32 | 2.37 | 2.36 | 8.95E−07 | 8.97E−07 |
| 28116.176 | 11785 | 393 | 70 | 24 | 85 | 164 | 33 | 2.48 | 2.45 | 8.98E−07 | 9.10E−07 |
| 853.7274 | 13065 | 436 | 71 | 24 | 83 | 183 | 38 | 2.77 | 2.73 | 9.01E−07 | 9.08E−07 |
| 445970.5 | 14063 | 469 | 70 | 24 | 85 | 198 | 42 | 2.98 | 2.92 | 9.06E−07 | 9.05E−07 |
| 1737424.64 | 15042 | 50 | 70 | 24 | 86 | 207 | 44 | 3.13 | 3.14 | 8.97E−07 | 9.10E−07 |
| 28116.1423 | 17520 | 584 | 70 | 24 | 84 | 241 | 49 | 3.64 | 3.69 | 8.98E−07 | 9.09E−07 |
| 2021311.24 | 20417 | 681 | 71 | 24 | 88 | 284 | 63 | 4.29 | 4.26 | 8.99E−07 | 9.10E−07 |
| 821.3904 | 26954 | 898 | 70 | 24 | 90 | 376 | 76 | 5.68 | 5.65 | 9.06E−07 | 9.12E−07 |
| 46506.122 | 102927 | 3431 | 70 | 24 | 93 | 1432 | 296 | 21.64 | 21.61 | 9.10E−07 | \|9.12E−07 |

Bacterial species, represented by their genome ID, whose presence and abundance were considered as significant (p-value< 0.05) are highlighted in grey.
Total mcfDNA Fragments per Genome with Conserved Region for Primer indicates the total number of fragments generated for the 30 trials of the simulation.
SPA Fragments >24 bp long refers to SPA fragments of 25 base pairs or greater; SPA Fragments >49 bp long refers to SPA fragments of 50 base pairs or greater.

Specificity analysis of SPA fragments obtained using the Cpn60-R571 primer: To analyze the phylogenetic specificity of the Cpn60-SPA fragments listed in Table 52, we compared them to the reference phylogenetic gene database which contains over 40,000 unique cpn60 gene entries. We also compared the results with those obtained using the RpoB1-R1327 primer. The results of this comparison are presented in Table 54 and show the following:
As shown in EXAMPLE 11, the rpoB gene-derived SPA fragments rpob_SPA4, rpob_SPA8, rpob_SPA40 and rpob_SPA46 were unable to distinguish between closely related Bacteroides, Blautia_A, Roseburia and Alistipes species, respectively; and SPA fragments rpob_SPA18, rpob_SPA21 and rpoB_SPA24 failed to discriminate on the subspecies level between Faecalibacterium prausnitzii and Faecalibacterium prausnitzii_j, Bifidobacterium longum subsp. longum and Bifidobacterium longum subsp. infantis, and Anaerostipes hadrus and Anaerostipes hadrus_B, respectively.
The cpn60 gene-derived SPA fragments cpn60_SPA5 and cpn60_SPA8, cpn60_SPA7 and cpn60_SPA37, cpn60_SPA19, cpn60_SPA34 and cpn60_SPA40 were unable to distinguish between closely related Blautia_A, Faecalibacterium, Acetatifactor, Bacteroides and Roseburia species, respectively; and SPA fragment cpn60_SPA24 and SPA fragment cpn60_SPA34 failed to discriminate on the subspecies level between Anaerostipes hadrus and Anaerostipes hadrus_B, and Bifidobacterium longum subsp. longum, Bifidobacterium longum subsp. infantis and Bifidobacterium longum subsp. imperatoris, respectively.
It should also be noted that the simulated community compositions using rpoB gene-derived SPA fragments and cpn60 gene-derived SPA fragments are very similar.

Unexpectedly, the phylogenetic resolution on the species level was gene dependent and, therefore, combining the results from multiple phylogenetic genes will result in better phylogenetic deconvolution of the community. As shown in Table 54, in several cases where SPA fragments derived from a single phylogenetic identifier gene failed to provide species level resolution, the combination of rpoB and cpn60 gene-derived SPA fragments from the same species allowed for improved phylogenetic resolution at the species level. Improved phylogenetic identification on the species level by rpoB gene-derived SPA fragments (compared to cpn60 gene-derived SPA fragments) was observed for Blautia_A massiliensis (rpoB_SPA5 fragment), Faecalibacterium prausnitzii_C (rpoB_SPA7 fragment), Blautia_A sp003480185 (rpoB_SPA12 fragment), Acetatifactor sp900066565 (rpoB_SPA20 fragment), Bacteroides caccae (rpoB_SPA35 fragment), and Faecalibacterium prausnitzii_D (rpoB_SPA38 fragment); and improved phylogenetic identification on the species level by cpn60 gene-derived SPA fragments (compared to rpoB gene-derived SPA fragments) was observed for Bacteroides ovatus (cpn60_SPA4 fragment), Roseburia sp900552665 (cpn60_SPA39 fragment) and Alistipes onderdonkii (cpn60_SPA46 fragment); and on the subspecies level for Faecalibacterium prausnitzii_J (cpn60_SPA17 fragment). Thus, using the combination of rpoB and cpn60 gene-derived SPA fragments, species-level taxonomic classification ambiguities were solved for Faecalibacterium, Acetatifactor and Bacteroides, and remained for Blautia_A species (rpob_SPA8 and cpn60_SPA8 fragments) and Roseburia species (rpob_SPA40 and cpn60_SPA40 fragments); and subspecies-level taxonomic classification ambiguities were solved for Faecalibacterium prausnitzii and remained for Bifidobacterium longum (rpob_SPA21 and cpn60_SPA20 fragments) and Anaerostipes hadrus (rpob_SPA24 and cpn60_SPA23 fragments).

Based on this result a new method is provided, referred to as multi loci SPA fragment sequencing, which combines SPA fragments from multiple phylogenetic identifier genes to analyze the composition of microbial communities as is described in EXAMPLE 14

TABLE 54
Simulated composition of the gut microbiome community based on rpoB and cpn60
gene-derived SPA fragment analysis.

| | Cpn60 | Cpn60 | | | RpoB | Rpob | | |
|---|---|---|---|---|---|---|---|---|
| SPA | SPA | SPA | | Cpn60 SPA | SPA | SPA | | |
| GTDB | Rel. | frag. | Cpn60 SPA | species | Rel. | fragments | Rpob SPA | RpoB SPA |
| Species | Ab. % | code | genus level | level | Ab. % | code | genus level | species level |
| Bacteroides | 21.64 | cpn60_ | Bacteroides | Bacteroides | 21.64 | rpob_SPA1 | Bacteroides | Bacteroides |
| stercoris | | SPA1 | | stercoris | | | Phocaeicola | stercoris |
| Phocaeicola | 5.68 | cpn60_ | Phocaeicola | Phocaeicola | 5.64 | rpob_SPA2 | | Phocaeicola |
| vulgatus | | SPA2 | | vulgatus | | | | vulgatus |
| Agathobacter | 4.29 | cpn60_ | Agathobacter | Agathobacter | 4.27 | rpob_SPA3 | Agathobacter | Agathobacter |
| faecis | | SPA3 | | faecis | | | | faecis |
| Bacteroides | 3.64 | cpn60_ | Bacteroides | Bacteroides | 3.71 | rpob_SPA4 | Bacteroides | Bacteroides |
| ovatus | | SPA4 | | ovatus | | | | ovatus |
| | | | | | | | | Bacteroides |
| | | | | | | | | xylanis |
| | | | | | | | | olvens |
| Blautia_A | 3.13 | cpn60_ | Blautia_A | Blautia_A | 3.17 | rpob_SPA5 | Blautia_A | Blautia_A |
| massiliensis | | SPA5 | | massiliensis | | | | massiliensis |
| | | | | Blautia_A | | | | |
| | | | | sp900066335 | | | | |
| | | | | Blautia_A | | | | |
| | | | | sp900066205 | | | | |
| Alistipes | 2.98 | cpn60_ | Alistipes | Alistipes | 2.94 | rpob_SPA6 | Alistipes | Alistipes |
| putredinis | | SPA6 | | putredinis | | | | putredinis |
| Faecali- | 2.77 | cpn60_ | Faecali- | Faecali- | 2.71 | rpob_SPA7 | Faecali- | Faecali- |
| bacterium | | SPA7 | bacterium | bacterium | | | bacterium | bacterium |
| prausnitzii_C | | | | prausnitzii_C | | | | prausnitzii_C |
| | | | | Faecali- | | | | |
| | | | | bacterium | | | | |
| | | | | prausnitzii | | | | |
| | | | | Faecali- | | | | |
| | | | | bacterium | | | | |
| | | | | sp003449675 | | | | |
| | | | | Faecali- | | | | |
| | | | | bacterium | | | | |
| | | | | prausnitzii_A | | | | |
| Bacteroides | 2.48 | cpn60_ | Bacteroides | Bacteroides | 2.46 | rpob_SPA4 | Bacteroides | Bacteroides |
| ovatus | | SPA4 | | ovatus | | | | ovatus |
| | | | | | | | | Bacteroides |
| | | | | | | | | xylanis |
| | | | | | | | | olvens |
| Blautia_A | 2.37 | cpn60_ | Blautia_A | Blautia_A | 2.39 | rpob_SPA8 | Blautia_A | Blautia_A |
| wexlerae_A | | SPA8 | | wexlerae | | | | wexlerae_A |
| | | | | Blautia_A | | | | Blautia_A |
| | | | | wexlerae_A | | | | wexlerae |
| | | | | Blautia_A | | | | Blautia_A |
| | | | | wexlerae_B | | | | sp003480185 |
| | | | | Blautia_A | | | | |
| | | | | sp000285855 | | | | |
| | | | | Blautia_A | | | | |
| | | | | sp003480185 | | | | |
| | | | | Blautia_A | | | | |
| | | | | sp003477525 | | | | |
| Paraprevotella | 2.23 | cpn60__ | Parapre | Paraprevotella | 2.22 | rpob_SPA9 | Paraprevotella | Paraprevotella |
| clara | | SPA9 | votella | clara | | | | clara |
| Agathobacter | 2.15 | cpn60_ | Agathobacter | Agathobacter | 2.18 | rpob_ | Agathobacter | Agathobacter |
| rectalis | | SPA10 | | rectalis | | SPA10 | | rectalis |
| Fusicateni- | 2.13 | cpn60_ | Fusicateni- | Fusicateni- | 2.08 | rpob_ | Fusicateni- | Fusicateni- |
| bacter | | SPA11 | bacter | bacter | | SPA11 | bacter | bacter |
| saccharivorans | | | | saccharivorans | | | | saccharivorans |
| Blautia_A | 2.09 | cpn60_ | Blautia_A | Blautia_A | 2.02 | rpob_ | Blautia_A | Blautia_A |
| sp003480185 | | SPA8 | | wexlerae | | SPA12 | | sp003480185 |
| | | | | Blautia_A | | | | |
| | | | | wexlerae_A | | | | |
| | | | | Blautia_A | | | | |
| | | | | wexlerae_B | | | | |
| | | | | Blautia_A | | | | |
| | | | | sp000285855 | | | | |
| | | | | Blautia_A | | | | |
| | | | | sp003480185 | | | | |
| | | | | Blautia_A | | | | |
| | | | | sp003477525 | | | | |
| Faecali- | 2.03 | cpn60_ | Faecali- | Faecali- | 2.07 | rpob_ | Faecali- | Faecali- |
| bacterium | | SPA12 | bacterium | bacterium | | SPA13 | bacterium | bacterium |
| prausnitzii_G | | | | prausnitzii_G | | | | prausnitzii_G |
| Faecali- | 2.00 | cpn60_ | Faecali- | Faecali- | 2.02 | rpob_ | Faecali- | Faecali- |
| bacterium | | SPA12 | bacterium | bacterium | | SPA13 | bacterium | bacterium |
| prausnitzii_G | | | | prausnitzii_G | | | | prausnitzii_G |
| Alistipes | 1.95 | cpn60_ | Alistipes | Alistipes | 1.91 | rpob_ | Alistipes | Alistipes |
| communis | | SPA13 | | communis | | SPA14 | | communis |
| Bifido- | 1.69 | cpn60_ | Bifido- | Bifido- | 1.76 | rpob_ | Bifido- | Bifido- |
| bacterium | | SPA14 | bacterium | bacterium | | SPA15 | bacterium | bacterium |
| pseudo | | | | pseudo | | | | pseudo |
| catenulatum | | | | catenulatum | | | | catenulatum |
| Bacteroides | 1.71 | cpn60_ | Bacteroides | Bacteroides | 1.74 | rpob_ | Bacteroides | Bacteroides |
| uniformis | | SPA15 | | uniformis | | SPA16 | | uniformis |
| Bacteroides | 1.63 | cpn60_ | Bacteroides | Bacteroides | 1.60 | rpob_ | Bacteroides | Bacteroides |
| ovatus | | SPA4 | | ovatus | | SPA4 | | ovatus |
| | | | | | | | | Bacteroides |
| | | | | | | | | xylanis |
| | | | | | | | | olvens |
| Ruminococcus_D | 1.50 | cpn60_ | Ruminococcus_D | Ruminococcus_D | 1.47 | rpob_ | Ruminococcus_D | Ruminococcus_D |
| bicirculans | | SPA16 | | bicirculans | | SPA17 | | bicirculans |
| Faecali- | 1.48 | cpn60_ | Faecali- | Faecali- | 1.45 | rpob_ | Faecali- | Faecali- |
| bacterium | | SPA17 | bacterium | bacterium | | SPA18 | bacterium | bacterium |
| prausnitzii_J | | | | prausnitzii_J | | | | prausnitzii_J |
| | | | | | | | | Faecali- |
| | | | | | | | | bacterium |
| | | | | | | | | prausnitzii |
| Schaedlerella | 1.44 | cpn60_ | Schaedlerella | Schaedlerella | 1.46 | rpob_ | Schaedlerella | Schaedlerella |
| sp900066545 | | SPA18 | | sp900066545 | | SPA19 | | sp900066545 |
| Acetatifactor | 1.41 | cpn60_ | Acetatifactor | Acetatifactor | 1.46 | rpob_ | Acetatifactor | Acetatifactor |
| sp900066565 | | SPA19 | | sp900066565 | | SPA20 | | sp900066565 |
| | | | | Acetatifactor | | | | |
| | | | | sp900066365 | | | | |
| Bifido- | 1.38 | cpn60_ | Bifido- | Bifido- | 1.38 | rpob_ | Bifido- | Bifido- |
| bacterium | | SPA20 | bacterium | bacterium | | SPA21 | bacterium | bacterium |
| longum | | | | longum | | | | longum |
| | | | | Bifido- | | | | Bifido- |
| | | | | bacterium | | | | bacterium |
| | | | | infantis | | | | infantis |
| | | | | Bifido- | | | | |
| | | | | bacterium | | | | |
| | | | | imperatoris | | | | |
| Blautia_A | 1.25 | cpn60_ | Blautia_A | Blautia_A | 1.23 | rpob_ | Blautia_A | Blautia_A |
| faecis | | SPA21 | | faecis | | SPA22 | | faecis |
| Blautia_A | 1.19 | cpn60_ | Blautia_A | Blautia_A | 1.23 | rpob_ | Blautia_A | Blautia_A |
| faecis | | SPA21 | | faecis | | SPA22 | | faecis |
| Mediterranei- | 1.17 | cpn60_ | Mediterranei- | Mediterranei- | 1.15 | rpob_ | Mediterranei- | Mediterranei- |
| bacter | | SPA22 | bacter | bacter | | SPA23 | bacter | bacter |
| lactaris | | | | lactaris | | | | lactaris |
| Anaerostipes | 1.10 | cpn60_ | Anaerostipes | Anaerostipes | 1.10 | rpob_ | Anaerostipes | Anaerostipes |
| hadrus | | SPA23 | | hadrus | | SPA24 | | hadrus |
| | | | | Anaerostipes | | | | Anaerostipes |
| | | | | hadrus_B | | | | hadrus_B |
| Anaero- | 1.06 | cpn60_ | Anaero- | Anaero- | 1.05 | rpob_ | Anaero- | Anaero- |
| butyricum | | SPA25 | butyricum | butyricum | | SPA25 | butyricum | butyricum |
| soehngenii | | | | soehngenii | | | | soehngenii |
| Gemmiger | 1.11 | cpn60_ | Gemmiger | Gemmiger | 1.02 | rpob_ | Gemmiger | Gemmiger |
| formicilis | | SPA24 | | formicilis | | SPA26 | | formicilis |
| Agatho- | 1.01 | cpn60_ | Agatho- | Agatho- | 1.01 | rpob_ | Agatho- | Agatho- |
| baculum | | SPA26 | baculum | baculum | | SPA27 | baculum | baculum |
| butyrici- | | | | butyrici- | | | | butyrici- |
| producens | | | | producens | | | | producens |
| Faecali- | 1.02 | cpn60_ | Faecali- | Faecali- | 1.07 | rpob_ | Faecali- | Faecali- |
| bacterium | | SPA27 | bacterium | bacterium | | SPA28 | bacterium | bacterium |
| sp900539885 | | | | sp900539885 | | | | sp900539885 |
| Alistipes | 1.01 | cpn60_ | Alistipes | Alistipes | 0.98 | rpob_ | Alistipes | Alistipes |
| finegoldii | | SPA28 | | finegoldii | | SPA29 | | finegoldii |
| ER4 | 0.95 | cpn60_ | ER4 | ER4 | 1.00 | rpob_ | ER4 | ER4 |
| sp000765235 | | SPA29 | | sp000765235 | | SPA30 | | sp000765235 |
| Gemmiger | 0.98 | cpn60_ | Gemmiger | Gemmiger | 0.96 | rpob_ | Gemmiger | Gemmiger |
| qucibialis | | SPA30 | | qucibialis | | SPA31 | | qucibialis |
| Lachnospira | 0.91 | cpn60_ | Lachnospira | Lachnospira | 0.92 | rpob_ | Lachnospira | Lachnospira |
| sp000437735 | | SPA31 | | sp000437735 | | SPA32 | | sp000437735 |
| Dialister | 0.86 | cpn60_ | Dialister | Dialister | 0.86 | rpob_ | Dialister | Dialister |
| invisus | | SPA32 | | invisus | | SPA33 | | invisus |
| Bariatricus | 0.88 | cpn60_ | Bariatricus | Bariatricus | 0.90 | rpob_ | Bariatricus | Bariatricus |
| comes | | SPA33 | | comes | | SPA34 | | comes |
| Bacteroides | 0.86 | cpn60_ | Bacteroides | Bacteroides | 0.84 | rpob_ | Bacteroides | Bacteroides |
| caccae | | SPA34 | | caccae | | SPA35 | | caccae |
| | | | | Bacteroides | | | | |
| | | | | sp900556215 | | | | |
| Para- | 0.86 | cpn60_ | Para- | Para- | 0.87 | rpob_ | Para- | Para- |
| bacteroides | | SPA35 | bacteroides | bacteroides | | SPA36 | bacteroides | bacteroides |
| distasonis | | | | distasonis | | | | distasonis |
| Para- | 0.82 | cpn60_ | Para- | Para- | 0.83 | rpob_ | Parabac | Para- |
| bacteroides | | SPA36 | bacteroides | bacteroides | | SPA37 | teroides | bacteroides |
| merdae | | | | merdae | | | | merdae |
| Bacteroides | 0.71 | cpn60_ | Bacteroides | Bacteroides | 0.73 | rpob_ | Bacteroides | Bacteroides |
| caccae | | SPA34 | | caccae | | SPA35 | | caccae |
| | | | | Bacteroides | | | | |
| | | | | sp900556215 | | | | |
| Faecali- | 0.77 | cpn60_ | Faecali | Faecali- | 0.71 | rpob_ | Faecali- | Faecali- |
| bacterium | | SPA37 | bacterium | bacterium | | SPA38 | bacterium | bacterium |
| prausnitzii_D | | | | prausnitzii_D | | | | prausnitzii_D |
| | | | | Faecali- | | | | |
| | | | | bacterium | | | | |
| | | | | sp900539945 | | | | |
| Barnesiella | 0.75 | cpn60_ | Barnesiella | Barnesiella | 0.73 | rpob_ | Barnesiella | Barnesiella |
| intestini- | | SPA38 | | intestini- | | SPA39 | | intestini- |
| hominis | | | | hominis | | | | hominis |
| Roseburia | 0.70 | cpn60_ | Roseburia | Roseburia | 0.70 | rpob_ | Roseburia | Roseburia |
| sp900552665 | | SPA39 | | sp900552665 | | SPA40 | | inulini- |
| | | | | | | | | vorans |
| | | | | | | | | Roseburia |
| | | | | | | | | sp900552665 |
| Roseburia | 0.71 | cpn60_ | Roseburia | Roseburia | 0.69 | rpob_ | Roseburia | Roseburia |
| inulini- | | SPA40 | | inulini- | | SPA40 | | inulini- |
| vorans | | | | vorans | | | | vorans |
| | | | | Roseburia | | | | Roseburia |
| | | | | sp900552665 | | | | sp900552665 |
| KLE1615 | 0.64 | cpn60_ | KLE1615 | KLE1 | 0.69 | rpob_ | KLE1615 | KLE16 |
| sp900066985 | | SPA41 | | 615 | | SPA41 | | 15 |
| | | | | sp9000 | | | | sp9000 |
| | | | | 66985 | | | | 66985 |
| Dorea_A | 0.60 | cpn60_ | Dorea_A | Dorea_A | 0.65 | rpob_ | Dorea_A | Dorea_A |
| longicatena | | SPA42 | | longicatena | | SPA42 | | longicatena |
| Roseburia | 0.60 | cpn60_ | Roseburia | Roseburia | 0.61 | rpob_ | Roseburia | Roseburia |
| intestinalis | | SPA43 | | | | SPA43 | | intestinalis |
| CAG-41 | 0.55 | cpn60_ | CAG-41 | CAG-41 | 0.58 | rpob_ | CAG-41 | CAG-41 |
| sp900066215 | | SPA45 | | sp900066215 | | SPA44 | | sp900066215 |
| Blautia_A | 0.57 | cpn60_ | Blautia_A | Blautia_A | 0.59 | rpob_ | Blautia_A | Blautia_A |
| sp000436615 | | SPA44 | | sp000436615 | | SPA45 | | sp000436615 |
| Alistipes | 0.53 | cpn60_ | Alistipes | Alistipes | 0.54 | rpob_ | Alistipes | Alistipes |
| onderdonkii | | SPA46 | | onderdonkii | | SPA46 | | onderdonkii |
| | | | | | | | | Alistipes |
| | | | | | | | | megaguti |
| | | | | | | | | Alistipes |
| | | | | | | | | shahii |

Each community member is identified by its GTDB taxonomy (Parks et al, 2018).
The genus-level and species-level identification of each community member, based on 50 base pair long rpoB and cpn60 gene-derived SPA fragments, is also presented based on their GTDB taxonomy.
For each community member the relative abundances and SPA fragment identifiers are listed.
SPA fragments, which identified multiple community members, are highlighted in grey.
In case the rpoB and cpn60 gene-derived SPA fragments provided different levels of phylogenetic resolution, the SPA fragment identifier that provided the best phylogenetic resolution and its corresponding species are highlighted in bold.

### Example 14: Multi Loci Spa Fragment Sequencing Further Improves Specificity

As concluded from EXAMPLE 11 and EXAMPLE 13, SPA fragment sequences obtained with the primers RpoB1-R1327 and Cpn60-R571 provided excellent phylogenetic resolution for gut microbiome bacteria at the genus level and in many instances at the species and subspecies level. However, in some instances, these SPA fragments failed to discriminate between very closely related species and subspecies. To further improve the phylogenetic resolution of SPA fragment sequencing we provide a new approach, referred to as “Multi Loci SPA Fragment Sequencing”; In this approach two or more phylogenetic identifier genes are targeted using different gene-specific SPA primers in the same amplification reaction via multiplexing PCR. One example of a protocol is as follows:
Isolation of cfDNA using standard protocols.
End repair and 5′-phosphorylation of cfDNA fragments followed by the 3′ addition of a deoxy-adenine to create a 3′-sticky end formed by a single adenine nucleotide using standard protocols.
Ligation of an adaptor, which in this embodiment is an asymmetric linker cassette created by annealing the primers SPA-cas1 and SPA-cas2, using T4 DNA ligase.
Single point linker cassette repair. To generate multi loci SPA fragments, multiplexing PCR is performed on the ligation product using three primers: (a) the SPA1-amp primer that recognizes the repaired 5′ asymmetrical end of the linker cassette; (b) a primer that recognizes the primer annealing site specific for the conserved region of the first phylogenetic marker gene, such as the RpoB1-R1327 primer; and (c) a primer that recognizes the primer annealing site specific for the conserved region of the second phylogenetic marker gene, such as the Cpn60-R571 primer. All primer sequences are provided in Table 1.
Once the asymmetric linker cassette has been repaired, the primer (SPAT-amp primer) that recognizes the repaired 5′ asymmetrical end of the linker cassette can anneal and PCR amplification is initiated. In the case of the reverse RpoB1-R1327 and Cpn60-R571 primers, this will result in the amplification of DNA sequences located upstream of position 1327 of the rpoB gene and upstream of position 571 of the cpn60 gene, respectively.
In a follow up PCR step, adapter sequences are added to the amplified SPA fragments using the primers RpoB1-SPA-seq-R1327, Cpn60-SPA-seq-R571 and SPA1-seq-F (see Table 1). Alternatively, these primers can be directly used in STEP 4. Subsequently, multiplexing indices and sequencing adapters, such as Illumina sequencing adapters, can be attached using the Nextera XT Index Kit, after which fragments are paired-end sequenced using NGS Illumina sequencing, e.g. on the Illumina NextSeq 2000 (Illumina, Inc., San Diego, CA). This approach will result in sequenced fragments that share the sequence of either the RpoB1-R1327 primer or the Cpn60-R571 primer, followed by sequences that vary in length and nucleotide composition. Sequences derived from the same microorganisms and extended from the same primer will be identical except for the length of the sequenced fragment, which will vary as a function of the distance between the respective primer annealing site and the end of the mcfDNA fragment.

The processing and analysis of the SPA fragment sequences can include the following steps:
1. Similar to single loci SPA fragment sequencing, the reads are filtered based on read quality. Error correction can be done using software such as DADA2 (Callahan et al, 2016), which makes use of a parametric error model. The remaining error-corrected reads of different lengths can be deduplicated while recording the number of duplicates by sequence for calculating community composition.
2. Multi loci SPA fragment sequencing can include a step to deconvolute the reads on the phylogenetic gene level. Unique SPA fragments are aligned on the sequences of the RpoB1-R1327 primer or the Cpn60-R571 primer and sorted in gene specific “buckets”. This is schematically shown in Step 1 of FIG. 3B. Subsequently, the sequences of each bucket are sorted into bins of matching sequences representative for the same species. In a next step, the rpoB and cpn60 gene databases are searched for the longest read in each bin of matching sequences for species identification. If a fragment does not match exactly to the database entries, the closest match species is assigned, noting the likelihood of a false match.
3. For each phylogenetic gene, the community composition is calculated based on the percent of reads assigned to each species, taking into consideration the number of duplicate reads identified in step 1.
4. To reconciliate the outcomes obtained for the SPA fragments obtained from different phylogenetic identifier genes, their results are compared and consolidated into a consensus community description (species and their relative abundances), as is schematically shown in Step 2 of FIG. 3B.

### REFERENCES

Abadio A. K. R., et al. (2011). Comparative genomics allowed the identification of drug targets against human fungal pathogens. BMC Genomics 12:75.
Abdulamir A. S., Hafidh R. R., Bakar F. A. (2011). The association of Streptococcus bovis/gallolyticus with colorectal tumors: The nature and the underlying mechanisms of its etiological role. Journal of Experimental & Clinical Cancer Research 30: 11.
Altschul, S. F., Gish, W., Miller, W., Myers, E. W., Lipman, D. J. (1990), Basic local alignment search tool. J. Mol. Biol. 215:403-410.
Ananthakrishnan A. N., et al. (2017). Gut Microbiome Function Predicts Response to Antiintegrin Biologic Therapy in Inflammatory Bowel Diseases. Cell Host & Microbe 21: 603-610.
Anttila T., et al (2001). Serotypes of Chlamydia trachomatis and risk for development of cervical squamous cell carcinoma. JAMA 285:47-51.
Arahal D. R. (2014). Chapter 6—Whole-Genome Analyses: Average Nucleotide Identity. Methods in Microbiology 41: 103-122.
Banerjee S., et al. (2021). Prognostic correlations with the microbiome of breast cancer subtypes. Cell Death & Disease 12: Article number 831.
Baxter N. T., et al. (2016). Microbiota-based model improves the sensitivity of fecal immunochemical test for detecting colonic lesions. Genome Med. 8: Article number 37.
Bhattacharjee S., Lukiw W. J. (2013). Alzheimer's disease and the microbiome. Frontiers in Cellular Neuroscience 7: Article 153.
Boleij A., et al. (2011). Novel clues on the specific association of Streptococcus gallolyticus subsp gallolyticus with colorectal cancer. Journal of Infectious Diseases 203: 1101-1109.
Boulange C. L., et al. (2016). Impact of the gut microbiota on inflammation, obesity, and metabolic disease. Genome Medicine 8:42
Bullman S., et al. (2017). Analysis of Fusobacterium persistence and antibiotic response in colorectal cancer. Science 358: 1443-1448.
Bumham P., Kim M. S., Agbor-Enoh S., Luikart H., Valantine H. A., Khush K. K., et al. (2016). Single-stranded DNA library preparation uncovers the origin and diversity of ultrashort cell-free DNA in plasma. Sci Rep-Uk. 6.
Callahan, B. J., et al. (2016). DADA2: High-resolution sample inference from Illumina amplicon data. Nature methods 13: 581-583.
Castellarin M., et al. (2012). Fusobacterium nucleatum infection is prevalent in human colorectal carcinoma. Genome Res. 22: 299-306.
Chandra V. and McAllister F. (2021). Therapeutic potential of microbial modulation in pancreatic cancer. Gut 70: 1419-1425.
Chen C., et al. (2018). Oral microbiota of periodontal health and disease and their changes after nonsurgical periodontal therapy. The ISME Journal 12:1210-1224.
Cohen R. J., Shannon B. A., McNeal J. E., Shannon T., Garrett K. L. (2005). Propionibacterium acnes associated with inflammation in radical prostatectomy specimens: A possible link to cancer evolution?J. Urol. 173: 1969-1974.
Coutinho H. D. M., Falcao-Silva V. S., Gonçalves G. F. (2008). Pulmonary bacterial pathogens in cystic fibrosis patients and antibiotic therapy: a tool for the health workers. Int Arch Med. 1: 24.
De Abreu A. L. P., et al. (2016). Association of human papillomavirus, Neisseria gonorrhoeae and Chlamydia trachomatis co-infections on the risk of high-grade squamous intraepithelial cervical lesion. Am J Cancer Res 6: 1371-1383.
Decker B., Sholl L. M. (2020). Cell-Free DNA Testing. In: Genomic Medicine. Springer: 41-54.
Depoorter E., et al. (2016). Burkholderia: an update on taxonomy and biotechnological potential as antibiotic producers. Appl Microbiol Biotechnol 100: 5215-5229.
Dominy S. S., et al. (2019). Porphyromonas gingivalis in Alzheimer's disease brains: Evidence for disease causation and treatment with small-molecule inhibitors. Sci. Adv. 5: eaau3333
Fang X., Monk J. M., MihN., Du B., Sastry A. V., Kavvas E., Seif Y., Smarr L., and Palsson B. O. (2018). Escherichia coli B2 strains prevalent in inflammatory bowel disease patients have distinct metabolic capabilities that enable colonization of intestinal mucosa. BMC Systems Biology 12: 66.
Fernández-Carballo B. L., Broger T., Wyss R., Banaei N., Denkinger C. M. (2019). Toward the development of a circulating free DNA-based in vitro diagnostic test for infectious diseases: a review of evidence for tuberculosis. J Clin Microbiol. 57: e01234-18.
Fernández-Garcia, D., Hills, A., Page, K. et al. (2019). Plasma cell-free DNA (cfDNA) as a predictive and prognostic marker in patients with metastatic breast cancer. Breast Cancer Res 21: 149. https://doi.org/10.1186/s13058-019-1235-8
Geller L. T., et al. (2017). Potential role of intratumor bacteria in mediating tumor resistance to the chemotherapeutic drug gemcitabine. Science 357: 1156-1160.
Ghebremedhin B., Layer F., Konig W., Konig B. (2008). Genetic classification and distinguishing of Staphylococcus species based on different partial gap, 16S rRNA, hsp60, rpoB, sodA, and tuf gene sequences. J Clin Microbiol. 46: 1019-1025.
Gopalakrishnan V., Weiner B., Ford C. B., Sellman B. R., Hammond S. A., Freeman D. J., Dennis P., Soria J-C., Wortman J. R., Henn M. R. (2020). Intervention strategies for microbial therapeutics in cancer immunotherapy. Immuno-Oncology Technology 6: 9-17.
Gorelick J. I., Senterfit L. B., Vaughan E. D. Jr. (1988). Quantitative bacterial tissue cultures from 209 prostatectomy specimens: Findings and implications. J. Urol. 139: 57-60.
Gupta R. S., Lo B, Son J. (2018). Phylogenomics and Comparative Genomic Studies Robustly Support Division of the Genus Mycobacterium into an Emended Genus Mycobacterium and Four Novel Genera. Frontiers in Microbiology 9: article 67.
Gupta S., et al. (2019). Amplicon sequencing provides more accurate microbiome information in healthy children compared to culturing. Communications Biology 2: 291.
Gyles C., Boerlin P. (2014). Horizontally transferred genetic elements and their role in pathogenesis of bacterial disease. Veterinary Pathology 51: 328-340.
Han D., Li R., Shi J., Tan P., Zhang R., Li J. (2020). Liquid biopsy for infectious diseases: a focus on microbial cell-free DNA sequencing. Theranostics 10: 5501-5513.
Head S. R., et al. (2014). Library construction for next-generation sequencing: overviews and challenges. BioTechniques 56, 61-64, 66, 68, passim.
Jahr S., Hentze H., Englisch S., Hardt D., Fackelmayer F. O., Hesch R. D., et al. (2001). DNA fragments in the blood plasma of cancer patients: quantitations and evidence for their origin from apoptotic and necrotic cells. Cancer Res. 61: 1659-1665.
Jin C., et al. (2019). Commensal microbiota promote lung cancer development via γδT cells. Cell 176: 998-1013.
Kanagasingam S., Chukkapalli S. S, Richard Welbury and Sim K. Singhrao. (2020). Porphyromonas gingivalis is a Strong Risk Factor for Alzheimer's Disease. Journal of Alzheimer's Disease Reports 4: 501-511.
Kordahi M. C., et al. (2021). Genomic and functional characterization of a mucosal symbiont involved in early-stage colorectal cancer. Cell Host & Microbe 29: 1-10.
Lancefield R. C. (1933). A serological differentiation of human and other groups of hemolytic streptococci. J Exp Med. 57: 571-595.
Liang W., Zhao Y., Huang W., Gao Y., Xu W., Tao J., et al. (2019). Non-invasive diagnosis of early-stage lung cancer using high-throughput targeted DNA methylation sequencing of circulating tumor DNA (ctDNA). Theranostics 9: 2056-2070.
Links M. G., Dumonceaux T. J., Hemmingsen S. M., Hill J. E. (2012). The chaperonin-60 universal target is a barcode for bacteria that enables de novo assembly of metagenomic sequence data. PLoS One 7: e49755.
Malinowski B., et al. (2019). The role of Tannerella forsythia and Porphyromonas gingivalis in pathogenesis of esophageal cancer. Infectious Agents and Cancer 14:3.
Menzel P., et al. (2016). Fast and sensitive taxonomic classification for metagenomics with Kaiju. Nat. Commun. 7: 11257.
Mui U. N., Haley C. T., Tyring S. K. (2017). Viral Oncology: Molecular Biology and Pathogenesis. J. Clin. Med. 6 (111). doi:10.3390/jcm6120111.
Narunsky-Haziza L., et al. (2022). Pan-cancer analyses reveal cancer-type-specific fungal ecologies and bacteriome interactions. Cell 185: 3789-3806.
Nejman D., et al. (2020). The human tumor microbiome is composed of tumor type-specific intracellular bacteria. Science 368: 973-980.
Nomura J., Rieg G., Bluestone G., Tsai T., Lai A., Terashita D., et al. (2019). Rapid detection of invasive Mycobacterium chimaera disease via a novel plasma-based next-generation sequencing test. BMC Infect Dis. 19: 371.
Ogier J.-C., Pages S., Galan M., Barret M., and Gaudriault S. (2019). rpoB, a promising marker for analyzing the diversity of bacterial communities by amplicon sequencing. BMC Microbiology 19: 171.
Parks D. H., Imelfort M., Skennerton C. T., Hugenholtz P., Tyson G. W. (2015). CheckM: assessing the quality of microbial genomes recovered from isolates, single cells, and metagenomes. Genome Research 25: 1043-1055.
Parks D. H., et al. (2018). A standardized bacterial taxonomy based on genome phylogeny substantially revises the tree of life. Nature Biotechnology 36: 996-1004.
Parks D. H., Chuvochina M., Reeves P. R., Beatson S. A., Hugenholtz P. (2021). Reclassification of Shigella species as later heterotypic synonyms of Escherichia coli in the Genome Taxonomy Database. BioRxiv: https://doi.org/10.1101/2021.09.22.461432
Pasquereau-Kotula E., Martins M., Aymeric L., Dramsi S. (2018). Significance of Streptococcus gallolyticus subsp. gallolyticus association with colorectal cancer. Frontiers in Microbiology 9: article 614.
Peters B. A., Hayes R. B., Goparaju C., Reid C., Pass H. I., Ahn J. (2019). The microbiome in lung cancer tissue and recurrence-free survival. Cancer Epidemiol Biomarkers Prev. 28: 731-740.
Pleguezuelos-Manzano C., et al. (2020). Mutational signature in colorectal cancer caused by genotoxic pks⁺ E. coli. Nature 580: 269-273.
Poirier S., et al. (2018). Deciphering intra-species bacterial diversity of meat and seafood spoilage microbiota using gyrB amplicon sequencing. A comparative analysis with 16S rDNA V3-V4 amplicon sequencing. PLoS One. 13: e0204629.
Poore G. D., et al. (2020). Microbiome analyses of blood and tissues suggest cancer diagnostic approach. Nature 579: 567-574.
Rassoulian Barrett S. L., Holmes E. A., Long D. R., Shean R. C., Bautista G. E., Ravishankar S., Peddu V., Cookson B. T., Singh P. K., Greninger A. L., Salipante S. J. (2020). Cell free DNA from respiratory pathogens is detectable in the blood plasma of Cystic Fibrosis patients. Scientific Reports 10, Article number: 6903.
Riggo M. P. and Lennon A. (2007). Development of a novel PCR assay for detection of Prevotella oris in clinical specimens. FEMS Microbiol Lett. 276: 123 128.
Riquelme E., et al. (2019). Tumor microbiome diversity and composition influence pancreatic cancer outcomes. Cell 178: 795-806.
Savi D., et al. (2019). Impact of clonally-related Burkholderia contaminans strains in two patients attending an Italian cystic fibrosis centre: a case report. BMC Pulmonary Medicine 19: 164.
Shahir N. M., et al. (2020). Crohn's Disease Differentially Affects Region-Specific Composition and Aerotolerance Profiles of Mucosally Adherent Bacteria. Inflammatory Bowel Disease 26: 1843-1855.
Sepich-Poore G. D., Zitvogel L., Straussman R., Hasty J., Wargo J. A., Knight R. (2021). The microbiome and human cancer. Science 371: 1331-1343.
Shahir N. M., et al. (2020). Crohn's disease differentially affects region-specific composition and aerotolerance profiles of mucosally adherent bacteria. Inflammatory Bowel Disease 26: 1843-1855.
Socransky S. S., Haffajee A. D., Cugini M. A., Smith C., Kent Jr. R. L. (1998). Microbial complexes in subgingival plaque. J Clin Periodontol 25: 134-144.
Sun Y., An K., Yang C. (2019). Circulating cell-free DNA. In: Liquid Biopsy. Intech Open.
Thomas A. M., et al. (2019). Metagenomic analysis of colorectal cancer datasets identifies cross-cohort microbial diagnostic signatures and a link with choline degradation. Nat. Med. 25: 667-678.
Urbaniak C., et al. (2016). The microbiota of breast tissue and its association with breast cancer. Appl. Environ. Microbiol. 82: 5039-5048
Venkataramani V., et al. (2019). Glutamatergic synaptic input to glioma cells drives brain tumour progression. Nature 573: 532-538.
Vos M., Quince C., Pijl A. S., de Hollander M., Kowalchuk G. A. (2012). A comparison of rpoB and 16S rRNA as markers in pyrosequencing studies of bacterial diversity. PLoS One 7:e30600.
Vudatha V., Ranson M., Blair L., Ahmed A. A. (2019). Rapid detection of bacilli Calmette-Guerin-associated mycotic aortic aneurysm using novel cell-free DNA assay. J Vasc Surg Cases Innov Tech. 5: 143-148.
Wattam A. R., et al. (2014). PATRIC, the bacterial bioinformatics database and analysis resource. Nucleic Acids Res 42, D581-591, doi:10.1093/nar/gkt1099.
Witt R. G., Blair L., Frascoli M., Rosen M. J., Nguyen Q.-H., Bercovici S., et al. (2020). Detection of microbial cell-free DNA in maternal and umbilical cord plasma in patients with chorioamnionitis using next generation sequencing. PLoS ONE 15 (4): e0231239.
Wood D. E., Salzberg S. L. (2014). Kraken: ultrafast metagenomic sequence classification using exact alignments. Genome Biology 15:R46.
Wood D. E, Jennifer Lu J., Langmead B. (2019). Improved metagenomic analysis with Kraken 2. Genome Biology 20, Article number: 257 Wu J., Hu S., Zhang L., Xin J., Sun C., Wang L., et al. (2020). Tumor circulome in the liquid biopsies for cancer diagnosis and prognosis. Theranostics 10: 4544-4556.
Zeller G., et al. (2014). Potential of fecal microbiota for early stage detection of colorectal cancer. Mol. Syst. Biol. 10: 766
Zeng Q., et al. (2019). Synaptic proximity enables NMDAR signalling to promote brain metastasis. Nature 573: 526-531.
Zhou Y., Hemmige V., Dalai S. C., Hong D. K., Muldrew K., Mohajer M. A. (2019). Utility of whole-genome next-generation sequencing of plasma in identifying opportunistic infections in HIV/AIDS. The Open AIDS Journal 13: 7-11.

1. A method of amplifying microbial cell free DNA (mcfDNA), comprising:
performing, on a sample comprising microbial cell-free DNA (mcfDNA), an amplification reaction using (i) one or more degenerate primers comprising complementarity to one or more conserved regions, wherein the one or more conserved regions span at least 18 nucleotides of one or more phylogenetic marker genes designated for a set of reference microbes and (ii) a second primer comprising complementarity to (i) a repaired version of an adaptor ligated to ends of the mcfDNA or (ii) an end of the mcfDNA,
wherein at least 25 adjacent nucleotides upstream or downstream of an end of the one or more conserved regions comprise a hypervariable region, and the one or more degenerate primers are oriented to prime polymerase extension of the hypervariable region to generate amplified mcfDNA fragments.

2. (canceled)

3. The method of claim 1, further comprising sequencing the amplified mcfDNA fragments.

4. The method of claim 3, further comprising, using a computer:
a. aligning the mcfDNA fragment sequences on a sequence of the one or more degenerate primers and assigning matching sequences from the hypervariable region as representative of the same microbial species;
b. for each microbial species in part (a), searching a database of the one or more phylogenetic marker genes against the mcfDNA fragment sequences and assigning the microbial species based on the closest match; and
c. for the one or more phylogenetic marker genes, calculating a microbial community composition based on the relative abundance of the mcfDNA fragment sequences assigned to each microbial species.

5. (canceled)

6. The method of claim 4, wherein there are two or more phylogenetic marker genes, and further comprising determining the microbial community composition by calculating a mathematical mean of the relative abundance of each species for each of the two or more phylogenetic marker genes.

7. The method of claim 4, wherein the microbial community composition comprises one or more members of Eukaryotes, bacteria, or fungi.

8.-12. (canceled)

13. The method of claim 1, wherein the ends of the mcfDNA comprise an adaptor and the second primer comprises complementarity to a repaired version of the adaptor.

14. The method of claim 1, wherein the adaptor is a double stranded asymmetric linker cassette comprising a 5′ asymmetrical end and a 3′ end where the two strands are complementary.

15. (canceled)

16. The method of claim 14, wherein the second primer is complementary to a repaired 5′ end of the asymmetric linker cassette, and wherein in the amplification reaction polymerase extension from the one or more degenerate primers results in repair of the asymmetric linker cassette.

17.-19. (canceled)

20. The method of claim 1, wherein the one or more phylogenetic marker genes comprises rpoB.

21. The method of claim 1, wherein the one or more phylogenetic marker genes comprises cpn60.

22. The method of claim 1, wherein the one or more phylogenetic marker genes comprises 16S rRNA.

23. The method of claim 1, wherein the one or more phylogenetic marker genes comprises a combination of two or more of rpoB, cpn60, or 16S rRNA.

24.-35. (canceled)

36. The method of claim 1, wherein the one or more phylogenetic marker genes comprises DNA gyrase subunit B (gyrB), heat shock protein 60 (hsp60), superoxide dismutase A protein (sodA), TU elongation factor (tuf), DNA recombinase proteins (including recA, recE), trr1 gene that encodes for thioredoxin reductase; rim8 gene that encodes for a protein involved in the proteolytic activation of a transcriptional factor in response to alkaline pH; kre2 gene that encodes for α-1,2-mannosyltransferase; or erg6 gene that encodes for Δ(24)-sterol C-methyltransferase.

37. The method of claim 1, wherein the set of reference microbes comprises fungal microbes, wherein the one or more phylogenetic marker genes comprises a human fungal phylogenetic marker gene designated for the set of reference fungal microbes, and wherein the one or more degenerate primers comprises complementarity to a conserved region of the human fungal phylogenetic marker gene.

38. The method of claim 37, wherein the human fungal phylogenetic marker gene comprises nuclear ribosomal internal transcribed spacer region 1 (ITS1) or nuclear ribosomal internal transcribed spacer region 2 (ITS2).

39. The method of claim 37, wherein the amplified mcfDNA fragments comprise mcfDNA from one or a combination of members of the Ascomycota, Basidiomycota and Mucoromycota, including Alternaria species, Aspergillus species, Blastomyces species, Candida species, Capnodiales species, Cladosporium species, Malassezia species, Phaeosphaeria species, Pseudozyma species, Saccharomyces species, Sporobolomyces species, Vishniacozyma species, and Yarrowia species.

40. The method of claim 1, further comprising including in the amplification reaction a functional gene primer to determine the presence of a functional gene designated for the set of reference microbes, wherein the functional gene primer comprises complementarity to a conserved region of the functional gene.

41. The method of claim 40, where the functional gene is a pathogenicity factor, a PKS gene cluster essential for colibactin synthesis, or a choline trimethylaminelyase gene.

42. The method of claim 1, further comprising including in the amplification reaction a viral gene primer to determine the presence of a viral gene, wherein the viral gene primer comprises complementarity to a conserved region of the viral gene.

43. The method of claim 42, wherein the viral gene comprises a human DNA- or RNA-based oncovirus gene.

44. The method of claim 43, wherein the oncovirus is one or a combination of Epstein-Barr Virus (EBV), Human Papillomavirus (HPV), Hepatitis B virus (HBV), Human Herpesvirus-8 (HHV-8), or Merkel Cell Polyomavirus (MCPyV).

45. The method of claim 1, wherein the sample comprises a bodily fluid, a tissue, or an extracellular bodily substance.

46. The method of claim 45, wherein the bodily fluid comprises whole blood, a blood fraction, serum, plasma, or combinations thereof.

47. The method of claim 45, wherein the sample comprises a biopsy sample from a solid tumor, a skin graft, a liquid biopsy sample other than blood, or combinations thereof.

48. The method of claim 45, wherein the sample comprises a stool sample.

49.-55. (canceled)

56. The method of claim 4, wherein the calculated microbial community composition is a screening for one or more of: tuberculosis and other diseases caused by Mycobacterium species; pulmonary infection risks and causes in cystic fibrosis patients; the risk and onset of sepsis in patients with compromised immune systems; detection of opportunistic bacterial pathogens originating from the oral cavity that have been linked to Alzheimer's disease, pancreatic cancer and other conditions such as endocarditis; women's health issues including Chlamydia linked to mucopurulent cervicitis, pelvic inflammatory disease, tubal factor infertility, ectopic pregnancy and cervical cancer; detection and monitoring of progression in cancer; monitoring of minimal residual disease after oncology treatments; detection and monitoring of progression and minimal residual disease of breast cancer including triple negative breast cancer; detection of esophageal cancer, precancerous colonic polyps and early stage colorectal cancer, and detection and monitoring of progression and minimal residual disease of gastrointestinal cancers in general; detection and monitoring of progression and minimal residual disease in lung cancer; non-invasive analysis of the microbiome in pancreatic cancer patients to propose treatment protocols and prognostics for long-term survival; detection of Clostridium difficile infections; post-transplant bloodstream infections and Graft versus Host Disease (GvHD); detection of hospital acquired infections by emerging pathogens of clinical concern; detection of an infection in an immune compromised person; or detection of infection or inflammation of the gastrointestinal track in Irritable Bowel Disease (Crohn's disease, Ulcerative colitis).

57.-86. (canceled)


SEQUENCE LISTING (391 sequences)

SEQ ID NO: 1 - DNA, 26 bp, synthetic construct
  source 1..26: mol_type = other DNA
  misc_difference 9: note = a, g, c or t
cgrttdccnarrtgrtcratrtcrtc

SEQ ID NO: 2 - DNA, 23 bp, synthetic construct
  source 1..23: mol_type = other DNA
  misc_difference 12: note = a, g, c or t
tghacrtcdcgnacytcrwadcc

SEQ ID NO: 3 - DNA, 23 bp, synthetic construct
  source 1..23: mol_type = other DNA
  misc_difference 3: note = a, g, c or t
  misc_difference 18: note = a, g, c or t
ccnykrtcraabygcatnccytc

SEQ ID NO: 4 - DNA, 26 bp, synthetic construct
  source 1..26: mol_type = other DNA
  misc_difference 18: note = a, g, c or t
gaygayatygaycayytngghaaycg

SEQ ID NO: 5 - DNA, 23 bp, synthetic construct
  source 1..23: mol_type = other DNA
  misc_difference 6: note = a, g, c or t
  misc_difference 21: note = a, g, c or t
garggnatgcrvttygaymrngg

SEQ ID NO: 6 - DNA, 23 bp, synthetic construct
  source 1..23: mol_type = other DNA
  misc_difference 6: note = a, g, c or t
  misc_difference 9: note = a, g, c or t
carytntcncarttyatggayca

SEQ ID NO: 7 - DNA, 20 bp, synthetic construct
  source 1..20: mol_type = other DNA
  misc_difference 9: note = a, g, c or t
  misc_difference 12: note = a, g, c or t
  misc_difference 18: note = a, g, c or t
ccratrttnggnccytcngg

SEQ ID NO: 8 - DNA, 26 bp, synthetic construct
  source 1..26: mol_type = other DNA
  misc_difference 12: note = a, g, c or t
gcdcgrttdccnarrtgrtcratrtc

SEQ ID NO: 9 - DNA, 60 bp, synthetic construct
  source 1..60: mol_type = other DNA
  misc_difference 43: note = a, g, c or t
gtctcgtgggctcggagatgtgtataagagacagcgrttdccnarrtgrtcratrtcrtc

SEQ ID NO: 10 - DNA, 23 bp, synthetic construct
  source 1..23: mol_type = other DNA
  modified_base 12: mod_base = OTHER; note = Inosine
gghtwygargtncghgaygtdca

SEQ ID NO: 11 - DNA, 23 bp, synthetic construct
  source 1..23: mol_type = other DNA
  modified_base 3: mod_base = OTHER; note = Inosine
  modified_base 15: mod_base = OTHER; note = Inosine
gcngghtwygargtncghgaygt

SEQ ID NO: 12 - DNA, 57 bp, synthetic construct
  source 1..57: mol_type = other DNA
  modified_base 46: mod_base = OTHER; note = Inosine
gtctcgtgggctcggagatgtgtataagagacaggghtwygargtncghgaygtdca

SEQ ID NO: 13 - DNA, 57 bp, synthetic construct
  source 1..57: mol_type = other DNA
  misc_difference 46: note = a, g, c or t
gtctcgtgggctcggagatgtgtataagagacagtghacrtcdcgnacytcrwadcc

SEQ ID NO: 14 - DNA, 59 bp, synthetic construct
  source 1..59: mol_type = other DNA
gtctcgtgggctcggagatgtgtataagagacagtgacgytgcatgttbgmrcccatma

SEQ ID NO: 15 - DNA, 17 bp, synthetic construct
  source 1..17: mol_type = other DNA
  misc_difference 9: note = a, g, c or t
cctacgggnggcwgcag

SEQ ID NO: 16 - DNA, 51 bp, synthetic construct
  source 1..51: mol_type = other DNA
  misc_difference 43: note = a, g, c or t
gtctcgtgggctcggagatgtgtataagagacagcctacgggnggcwgcag

SEQ ID NO: 17 - DNA, 21 bp, synthetic construct
  source 1..21: mol_type = other DNA
gactachvgggtatctaatcc

SEQ ID NO: 18 - DNA, 55 bp, synthetic construct
  source 1..55: mol_type = other DNA
gtctcgtgggctcggagatgtgtataagagacaggactachvgggtatctaatcc

SEQ ID NO: 19 - DNA, 57 bp, synthetic construct
  source 1..57: mol_type = other DNA
  misc_difference 37: note = a, g, c or t
  misc_difference 52: note = a, g, c or t
gtctcgtgggctcggagatgtgtataagagacagccnykrtcraabygcatnccytc

SEQ ID NO: 20 - DNA, 50 bp, synthetic construct
  source 1..50: mol_type = other DNA
  misc_difference 20: note = a, g, c or t
  misc_difference 21: note = a, g, c or t
  misc_difference 22: note = a, g, c or t
  misc_difference 23: note = a, g, c or t
  misc_difference 24: note = a, g, c or t
  misc_difference 25: note = a, g, c or t
  modified_base 50: mod_base = OTHER; note = Phosphorothioated DNA nucleotide
gacagggatttgctggtcgnnnnnnaattcaactaggcttaatccgacgt

SEQ ID NO: 21 - DNA, 24 bp, synthetic construct
  source 1..24: mol_type = other DNA
  modified_base 1: mod_base = OTHER; note = 5' phosphate modified nucleotide
cgtcggattaagcctagttgagca

SEQ ID NO: 22 - DNA, 19 bp, synthetic construct
  source 1..19: mol_type = other DNA
gacagggatttgctggtcg

SEQ ID NO: 23 - DNA, 47 bp, synthetic construct
  source 1..47: mol_type = other DNA
tcgtcggcagcgtcagatgtgtataagagacagggatttgctggtcg

SEQ ID NO: 24 - DNA, 49 bp, synthetic construct
  source 1..49: mol_type = other DNA
  misc_difference 25: note = a, g, c or t
  misc_difference 26: note = a, g, c or t
  misc_difference 27: note = a, g, c or t
  misc_difference 28: note = a, g, c or t
  misc_difference 29: note = a, g, c or t
  misc_difference 30: note = a, g, c or t
  misc_difference 31: note = a, g, c or t
  misc_difference 32: note = a, g, c or t
  misc_difference 33: note = a, g, c or t
  misc_difference 34: note = a, g, c or t
caagcagaagacggcatacgagatnnnnnnnnnngtctcgtgggctcgg

SEQ ID NO: 25 - DNA, 53 bp, synthetic construct
  source 1..53: mol_type = other DNA
  misc_difference 30: note = a, g, c or t
  misc_difference 31: note = a, g, c or t
  misc_difference 32: note = a, g, c or t
  misc_difference 33: note = a, g, c or t
  misc_difference 34: note = a, g, c or t
  misc_difference 35: note = a, g, c or t
  misc_difference 36: note = a, g, c or t
  misc_difference 37: note = a, g, c or t
  misc_difference 38: note = a, g, c or t
  misc_difference 39: note = a, g, c or t
aatgatacggcgaccaccgagatctacacnnnnnnnnnntcgtcggcagcgtc

SEQ ID NO: 26 - DNA, 50 bp, Staphylococcus auricularis
  source 1..50: mol_type = genomic DNA
ctactctcactatggtcgtatgtgtccaatcgaaacaccagagggtccaa

SEQ ID NO: 27 - DNA, 98 bp, Flavobacterium sp.
  source 1..98: mol_type = genomic DNA
ctatactcactacggacgtttatgtccaattgaaactcctgagggaccaaacattggtttgatttcatctcttggggtgtatgctaaagtgaatggta

SEQ ID NO: 28 - DNA, 128 bp, Pseudomonas toyotomiensis
  source 1..128: mol_type = genomic DNA
cccgactcactatggtcgcgtgtgcccgatcgaaacgccggaaggtccgaacatcggtctgatcaactcgctggctgcctacgcccgcaccaaccagtacggcttcctggaaagcccgtaccgcgtgg

SEQ ID NO: 29 - DNA, 86 bp, Finegoldia magna
  source 1..86: mol_type = genomic DNA
cgactctcactacggtagaatctgtccgatagaaacaccagaaggaccaaacatcggtcttataacttccatgacaacttattcta

SEQ ID NO: 30 - DNA, 54 bp, Parvularcula sp.
  source 1..54: mol_type = genomic DNA
cccgacccactatggccgcatctgcccgatcgagacgccggaaggcccgaatat

SEQ ID NO: 31 - DNA, 45 bp, Pseudomonas stutzeri
  source 1..45: mol_type = genomic DNA
cccgacccattacggtcgtgtgtgcccgatcgagacgccgaaagg

SEQ ID NO: 32 - DNA, 128 bp, Pseudomonas sp.
  source 1..128: mol_type = genomic DNA
cccgacgcattacggtcgtgtatgcccgatcgaaacgccggaaggtccgaacatcggtctgatcaactccctggctgcctatgcgcgcaccaaccagtacggcttcctcgaaagcccataccgtgtgg

SEQ ID NO: 33 - DNA, 46 bp, Peptoniphilus harei
  source 1..46: mol_type = genomic DNA
taactcacattacggaagaatgtgtcctattgagacaccagaaggt

SEQ ID NO: 34 - DNA, 47 bp, unidentified
  source 1..47: mol_type = unassigned DNA
tcccacgcactacggccgcgtctgcccgatcgagacgcctgaaggcc

SEQ ID NO: 35 - DNA, 47 bp, Azoarcus sp.; identical to SEQ ID NO: 34
  source 1..47: mol_type = genomic DNA

SEQ ID NO: 36 - DNA, 75 bp, Sphingopyxis terrae
  source 1..75: mol_type = genomic DNA
tccgacgcactatggccgtatctgcccgatcgaaacgccggaaggcccgaacatcggtctgatcaacagactcgc

SEQ ID NO: 37 - DNA, 127 bp, unidentified
  source 1..127: mol_type = unassigned DNA
ttgaaagtgccgcatggtgagagcggtatcgtcgtagacgtaaagaaatattcgcgtgccaatggcgacgatctggcaccgggtcttaacgaagtcgttcgcgtttatatcgcgacaaagcgcaaga

SEQ ID NO: 38 - DNA, 130 bp, Staphylococcus sp.
  source 1..130: mol_type = genomic DNA
tgtttgatccccacgctttcgcacatcagcgtcagttacagaccagaaagtcgccttcgccactggtgttcctccatatctctgcgcatttcaccgctacacatggaattccactttcctcttctgcact

SEQ ID NO: 39 - DNA, 51 bp, Flavobacterium sp.
  source 1..51: mol_type = genomic DNA
tgttcgctacccacgctttcgtccatcagcgtcaatccattagtagtaacc

SEQ ID NO: 40 - DNA, 112 bp, Rheinheimera sp.
  source 1..112: mol_type = genomic DNA
tgtttgctccccacgctttcgcacctgagcgtcagtgttgtgccagggggccgccttcgccactggtattcctccaaatctctacgcatttcaccgctacacttggaattct

SEQ ID NO: 41 - DNA, 53 bp, Rhodococcus sp.
  source 1..53: mol_type = genomic DNA
tgttcgctacccacgctttcgctcctcagcgtcagttactgcccagagacccg

SEQ ID NO: 42 - DNA, 82 bp, Dietzia sp.
  source 1..82: mol_type = genomic DNA
tgttcgctacccatgctttcgctcctcagcgtcagttactacccagagacccgccttcgccaccggtgttcctcctgatatc

SEQ ID NO: 43 - DNA, 130 bp, unidentified
  source 1..130: mol_type = unassigned DNA
tgttcgctccccacgctttcgctcctcagcgtcaggaaaggcccagagaaccgccttcgccactggtgttcctcctgatatctgcgcattccaccgctccaccaggaattccattctcccctaccttcct

SEQ ID NO: 44 - DNA, 53 bp, Angustibacter sp.
  source 1..53: mol_type = genomic DNA
tgttcgctccccatgctttcgctcctcagcgtcagttacggcccagagatccg

SEQ ID NO: 45 - DNA, 130 bp, Aerococcus urinae
  source 1..130: mol_type = genomic DNA
tgtttgctacccacgctttcgggcctcagcgtcagtgacagaccagaaagtcgccttcgccactggtgttcttccatatatctacgcattccaccgctacacatggagttccactttcctcttctgtact

SEQ ID NO: 46 - DNA, 91 bp, Pseudomonas sp.
  source 1..91: mol_type = genomic DNA
tgtttgctccccacgctttcgcacctcagtgtcagtatcagtccaggtggtcgccttcgccactggtgttccttcctatatctacgcattt

SEQ ID NO: 47 - DNA, 91 bp, Pseudomonas saponiphila
  source 1..91: mol_type = genomic DNA
tgtttgctccccacgctttcgcacctcagtgtcagtatcagtccaggtggtcgccttcgccactggtgttccttcctatatctacgtattt

SEQ ID NO: 48 - DNA, 47 bp, Klebsiella sp.
  source 1..47: mol_type = genomic DNA
tgtttgctccccacgctttcgcacctgagcgtcagtctttgtccagg

SEQ ID NO: 49 - DNA, 40 bp, Comamonas sp.
  source 1..40: mol_type = genomic DNA
tgtttgctccccacgctttcgtgcatgagcgtcagtgcag

SEQ ID NO: 50 - DNA, 50 bp, Mycobacterium sp.
  source 1..50: mol_type = genomic DNA
gtcagaccacgatgaccgttccgggcggcgtcgaggtgccggtggaaacc

SEQ ID NO: 51 - DNA, 50 bp, Mycobacterium sp.
  source 1..50: mol_type = genomic DNA
gtcagaccacgatgatcgttccgggcggcgtcgaggtgccggtggaaacc

SEQ ID NO: 52 - DNA, 50 bp, Mycobacterium abscessus
  source 1..50: mol_type = genomic DNA
gccagaccacgatgaccgcccccggtggcgtcgaggtgccggtggatgtg

SEQ ID NO: 53 - DNA, 50 bp, Mycobacterium sp.
  source 1..50: mol_type = genomic DNA
gccagaccacgatgaccgcccccggcggcgtcgaggtgccggtggacgtg

SEQ ID NO: 54 - DNA, 50 bp, Mycobacterium abscessus
  source 1..50: mol_type = genomic DNA
gccagaccacgatgaccgcccccggcggcgtcgaggtgccggtggatgtg

SEQ ID NO: 55 - DNA, 50 bp, Mycobacterium abscessus
  source 1..50: mol_type = genomic DNA
gccagaccacgatgaccgcccccgggggcgtcgaggtgccggtggatgtt

SEQ ID NO: 56 - DNA, 50 bp, Mycobacterium abscessus
  source 1..50: mol_type = genomic DNA
gccagaccacgatgaccgcccccgggggcgtcgaggtgccggtggatgtg

SEQ ID NO: 57 - DNA, 50 bp, Mycobacterium avium
  source 1..50: mol_type = genomic DNA
gtcagcccacgatgaccgtcccgggcggcatcgaggtgccggtggagacc

SEQ ID NO: 58 - DNA, 50 bp, Mycobacterium sp.
  source 1..50: mol_type = genomic DNA
gtcagcccacgatgaccgtccccggcggcatcgaggtgccggtggagacc

SEQ ID NO: 59 - DNA, 50 bp, Mycobacterium chimaera
  source 1..50: mol_type = genomic DNA
agcccgctgtcatgactgtccccggcggcatcgaggtgccggtggagacc

SEQ ID NO: 60 - DNA, 50 bp, Mycobacterium leprae
  source 1..50: mol_type = genomic DNA
gtcagtcgacaatgactgtcccaggtggggtagaagtgccagtggaaact

SEQ ID NO: 61 - DNA, 50 bp, Mycobacterium xenopi
  source 1..50: mol_type = genomic DNA
ggcacgccacgatgaaggtccccggtggcgtcgaggtgccggtggagacc

SEQ ID NO: 62 - DNA, 50 bp, Mycobacterium sp.
  source 1..50: mol_type = genomic DNA
gccagcccacgatgaccgtccccggcggcatcgaggtgccggtggagacc

SEQ ID NO: 63 - DNA, 50 bp, Mycobacterium kansasii
  source 1..50: mol_type = genomic DNA
gccaggccacgatgaccgtgccgggcggggtcgaggtgccggtggaaacc

SEQ ID NO: 64 - DNA, 50 bp, Mycobacterium sp.
  source 1..50: mol_type = genomic DNA
agcccgccgtcatgactgtgcccggcggggtcgaggtcccggtggaaacc

SEQ ID NO: 65 - DNA, 50 bp, Mycobacterium gilvum
  source 1..50: mol_type = genomic DNA
gtgaccagacgatgaccgcgcccggcggctccgaggtgcccgtcgaggtc

SEQ ID NO: 66 - DNA, 50 bp, Mycobacterium sp.
  source 1..50: mol_type = genomic DNA
gccagaccacgatgaccgtccccggcggcgtcgaggtcccggtcgaggtg

SEQ ID NO: 67 - DNA, 50 bp, Mycobacterium sp.
  source 1..50: mol_type = genomic DNA
gccagaccgcgatgaccgctccgggcggtgtcgaggtgccggtcgagacc

SEQ ID NO: 68 - DNA, 50 bp, Mycobacterium sp.
  source 1..50: mol_type = genomic DNA
gccagacctcgatgacggtgcccggcggtgtcgaggtgccggtcgaggtg

SEQ ID NO: 69 - DNA, 50 bp, Staphylococcus aureus
  source 1..50: mol_type = genomic DNA
ttgcttcaatgagttacttctttaacttattaagcggtattggatataca

SEQ ID NO: 70 - DNA, 50 bp, Staphylococcus sp.
  source 1..50: mol_type = genomic DNA
tcgcttcaatgagttacttctttaacttattaagtggtattggatataca

SEQ ID NO: 71 - DNA, 50 bp, Staphylococcus sp.
  source 1..50: mol_type = genomic DNA
tcgcttcaatgagttatttctttaacttattaagtggtattggatataca

SEQ ID NO: 72 - DNA, 50 bp, Staphylococcus sp.
  source 1..50: mol_type = genomic DNA
ttgcttcaatgagttatttctttaacttattaagtggtattggatataca

SEQ ID NO: 73 - DNA, 50 bp, Staphylococcus aureus
  source 1..50: mol_type = genomic DNA
tcgcttcaatgagttacttctttaacttattaagcggtattggatataca

SEQ ID NO: 74 - DNA, 50 bp, Staphylococcus aureus
  source 1..50: mol_type = genomic DNA
tcgcttcaatgagttacttctttaatttattaagtggtattggatataca

SEQ ID NO: 75 - DNA, 50 bp, Staphylococcus aureus
  source 1..50: mol_type = genomic DNA
gttgaaacttgcgcacatggttgatgataaattacatgcgcgttcaacag

SEQ ID NO: 76 - DNA, 50 bp, Pseudomonas sp.
  source 1..50: mol_type = genomic DNA
tcgatgtgctcaagaccctcgtcgacatccgtaacggcaagggcatcgtc

SEQ ID NO: 77 - DNA, 50 bp, Pseudomonas sp.
  source 1..50: mol_type = genomic DNA
tcgatgtgctcaagaccctggtcgacatccgtaacggcaagggcatcgtc

SEQ ID NO: 78 - DNA, 50 bp, Pseudomonas aeruginosa
  source 1..50: mol_type = genomic DNA
tcgatgtgctcaagaccctcgtcgatatccgtaacggcaagggcatcgtc

SEQ ID NO: 79 - DNA, 50 bp, Pseudomonas sp.
  source 1..50: mol_type = genomic DNA
tcgaggtccttaagaccctggtcgatatccgtaacggcaaaggcattgtc

SEQ ID NO: 80 - DNA, 50 bp, unidentified
  source 1..50: mol_type = unassigned DNA
tcgcgacgatcaagatcctcgtcgagctgcgcaacggcaagggcgaagtg

SEQ ID NO: 81 - DNA, 50 bp, unidentified
  source 1..50: mol_type = unassigned DNA
tcgcgacgatcaagatcctcgtcgaactgcgcaacggcaagggcgaagtg

SEQ ID NO: 82 - DNA, 50 bp, Burkholderia sp.
  source 1..50: mol_type = genomic DNA
tcgcgacgatcaagatcctcgtcgagttgcgcaacggcaagggcgaagtg

SEQ ID NO: 83 - DNA, 50 bp, Burkholderia sp.
  source 1..50: mol_type = genomic DNA
tcgcgacgatcaagatcctcgtcgagctgcgcaacggcaagggcgaagta

SEQ ID NO: 84 - DNA, 50 bp, unidentified
  source 1..50: mol_type = unassigned DNA
tcgcgacgatcaagatcctcgtcgagctgcgcaatggcaagggcgaagtg

SEQ ID NO: 85 - DNA, 50 bp, Burkholderia sp.
  source 1..50: mol_type = genomic DNA
tcgcgacgatcaagatcctggtcgagctgcgcaacggcaagggcgaagtg

SEQ ID NO: 86 - DNA, 50 bp, Burkholderia sp.
  source 1..50: mol_type = genomic DNA
tcgcgacgatcaagattctcgtcgagctgcgcaacggcaagggcgaagtg

SEQ ID NO: 87 - DNA, 100 bp, Burkholderia sp.
  source 1..100: mol_type = genomic DNA
cagccgtgacgagatcaccggcccgatgacgctgcaggacgacgacatcctcgcgacgatcaagatcctcgtcgagctgcgcaacggcaagggcgaagtg

SEQ ID NO: 88 - DNA, 100 bp, Burkholderia ubonensis
  source 1..100: mol_type = genomic DNA
cggccgcgacgagatcaccggcccgatgacgctgcaggacgacgacatcctcgcgacgatcaagatcctcgtcgagctgcgcaacggcaagggcgaagtg

SEQ ID NO: 89 - DNA, 100 bp, Burkholderia sp.
  source 1..100: mol_type = genomic DNA
cggccgtgacgaaatcaccggcccgatgacgctgcaggacgacgacatcctcgcgacgatcaagatcctcgtcgagctgcgcaacggcaagggcgaagtg

SEQ ID NO: 90 - DNA, 100 bp, unidentified
  source 1..100: mol_type = unassigned DNA
cggccgtgacgaaatcacgggcccgatgacgctgcaggacgacgacatcctcgcgacgatcaagatcctcgtcgagctgcgcaacggcaagggcgaagtg

SEQ ID NO: 91 - DNA, 100 bp, unidentified
  source 1..100: mol_type = unassigned DNA
cggccgtgacgaaatcgtcggcccgatgacgctgcaggacgacgacatcctcgcgacgatcaagatcctcgtcgagctgcgcaacggcaagggcgaagtg

SEQ ID NO: 92 - DNA, 100 bp, Burkholderia sp.
  source 1..100: mol_type = genomic DNA
cggccgtgacgagatcaccggcccgatgacgctgcaggacgacgacatcctcgcgacgatcaagatcctcgtcgagctgcgcaacggcaagggcgaagtg

SEQ ID NO: 93 - DNA, 100 bp, Burkholderia sp.
  source 1..100: mol_type = genomic DNA
cggccgtgacgagatcatcggcccgatgacgctgcaggacgacgacatcctcgcgacgatcaagatcctcgtcgagctgcgcaacggcaagggcgaagtg

SEQ ID NO: 94 - DNA, 100 bp, Burkholderia sp.
  source 1..100: mol_type = genomic DNA
cggccgtgacgagatcgtcggcccgatgacgctgcaggacgacgacatcctcgcgacgatcaagatcctcgtcgagctgcgcaacggcaagggcgaagtg

SEQ ID NO: 95 - DNA, 100 bp, unidentified
  source 1..100: mol_type = unassigned DNA
cggccgtgatgaaatcgtcggtccgatgacgctgcaggacgacgacattctcgcgacgatcaagatcctcgtcgagctgcgcaacggcaagggcgaagtg

SEQ ID NO: 96 - DNA, 100 bp, Burkholderia ubonensis
  source 1..100: mol_type = genomic DNA
cggtcgcgacgagatcgtcggcccgatgacgctgcaggacgacgacatcctcgcgacgatcaagatcctcgtcgagctgcgcaacggcaagggcgaagtg

SEQ ID NO: 97 - DNA, 100 bp, Burkholderia sp.
  source 1..100: mol_type = genomic DNA
cggtcgtgacgaaatcgtcggcccgatgacgctgcaggacgacgacatcctcgcgacgatcaagatcctcgtcgagctgcgcaacggcaagggcgaagtg

SEQ ID NO: 98 - DNA, 100 bp, Burkholderia sp.
  source 1..100: mol_type = genomic DNA
gggccgtgacgaaatcaccggcccgatgacgctgcaggacgacgacatcctcgcgacgatcaagatcctcgtcgagctgcgcaacggcaagggcgaagtg

SEQ ID NO: 99 - DNA, 100 bp, unidentified
  source 1..100: mol_type = unassigned DNA
gggccgtgacgaaatcgtcggcccgatgacgctgcaggacgacgacatcctcgcgacgatcaagatcctcgtcgagctgcgcaacggcaagggcgaagtg

SEQ ID NO: 100 - DNA, 100 bp, Burkholderia sp.
  source 1..100: mol_type = genomic DNA
gggtcgtgacgaaatcaccggcccgatgacgctgcaggacgacgacatcctcgcgacgatcaagatcctcgtcgagctgcgcaacggcaagggcgaagtg

SEQ ID NO: 101 - DNA, 100 bp, Burkholderia sp.
  source 1..100: mol_type = genomic DNA
cggccgcgacgagatcgtcggcccgatgacgctgcaggacgacgacatcctcgcgacgatcaagatcctcgtcgagctgcgcaacggcaagggcgaagtg

SEQ ID NO: 102 - DNA, 100 bp, unidentified
  source 1..100: mol_type = unassigned DNA
cggccgcgatgaaatcgtcggcccgatgacgctgcaggacgacgacatcctcgcgacgatcaagatcctcgtcgagctgcgcaacggcaagggcgaagtg

SEQ ID NO: 103 - DNA, 50 bp, unidentified
  source 1..50: mol_type = unassigned DNA
tcgcgacgatcaagatcctcgtcgagctgcgcaacggcaagggcgaagtc

SEQ ID NO: 104 - DNA, 50 bp, Burkholderia sp.
  source 1..50: mol_type = genomic DNA
tcgcgacgatcaagatcctcgtcgagttgcgcaacggcaagggcgaagtc

SEQ ID NO: 105 - DNA, 50 bp, Burkholderia sp.
  source 1..50: mol_type = genomic DNA
tcgcgacgatcaagattctcgtcgagctgcgcaacggcaagggcgaagtc

SEQ ID NO: 106 - DNA, 50 bp, Haemophilus sp.
  source 1..50: mol_type = genomic DNA
ttgcggtaatgcgtaaattgatcgacatccgtaatggtcgtggcgaagta

SEQ ID NO: 107 - DNA, 50 bp, unidentified
  source 1..50: mol_type = unassigned DNA
ttcgtgtgatgaaaaaactcatcgatatccgtaatggtcgtggtgaagtg

SEQ ID NO: 108 - DNA, 50 bp, Haemophilus influenzae
  source 1..50: mol_type = genomic DNA
ttgcggtaatgcgtaaattgattgacatccgtaatggtcgtggcgaagta

SEQ ID NO: 109 - DNA, 50 bp, Haemophilus sp.
  source 1..50: mol_type = genomic DNA
ttcgtgtgatgaaaaaactcatcgacatccgtaatggtcgtggtgaagtg

SEQ ID NO: 110 - DNA, 50 bp, Haemophilus influenzae
  source 1..50: mol_type = genomic DNA
ttgcggtaatgcgtaaattgattgacatccgtaatggtcgcggcgaagta

SEQ ID NO: 111 - DNA, 50 bp, Haemophilus sp.
  source 1..50: mol_type = genomic DNA
ttcgtgtgatgaaaaaactcatcgacatccgtaatggtcgtggtgaagta

SEQ ID NO: 112 - DNA, 50 bp, Haemophilus sp.
  source 1..50: mol_type = genomic DNA
ttgcggtaatgcgtaaattaatcgacatccgtaatggtcgtggcgaagta

SEQ ID NO: 113 - DNA, 50 bp, Haemophilus influenzae
  source 1..50: mol_type = genomic DNA
tcgcggtaatgcgtaaattgattgacatccgtaatggtcgtggcgaagta

SEQ ID NO: 114 - DNA, 50 bp, Haemophilus influenzae
  source 1..50: mol_type = genomic DNA
ttgcggtaatgcgtaaattaattgacatccgtaatggtcgtggcgaagta

SEQ ID NO: 115 - DNA, 50 bp, Streptococcus sp.
  source 1..50: mol_type = genomic DNA
ttgctgagatgagctacttcctcaacttggctgaaggacttggccgtgta

SEQ ID NO: 116 - DNA, 50 bp, Streptococcus sp.
  source 1..50: mol_type = genomic DNA
tggcagaaatgtcttacttcttgaaccttgctgaaggtcttggaaaagtt

SEQ ID NO: 117 - DNA, 50 bp, Streptococcus pyogenes
  source 1..50: mol_type = genomic DNA
tggcagaaatgtcttacttcttgaaccttgcagaaggtcttggaaaagtt

SEQ ID NO: 118 - DNA, 50 bp, Streptococcus pyogenes
  source 1..50: mol_type = genomic DNA
tggcagaaatgtcatacttcttgaaccttgctgaaggtcttggaaaagtt

SEQ ID NO: 119 - DNA, 50 bp, Streptococcus mutans
  source 1..50: mol_type = genomic DNA
tagctgaaatgtcttatttccttaacttggctgagggtctaggtaaagtt

SEQ ID NO: 120 - DNA, 50 bp, Streptococcus suis
  source 1..50: mol_type = genomic DNA
tggctgaaatgagctacttcctcaacttggctgagggtcttggtcgtgta

SEQ ID NO: 121 - DNA, 50 bp, Streptococcus suis
  source 1..50: mol_type = genomic DNA
tggctgaaatgagctacttcctcaacttggctgaaggacttggtcgcgta

SEQ ID NO: 122 - DNA, 50 bp, Streptococcus sp.
  source 1..50: mol_type = genomic DNA
ttgccgagatgagctacttcctcaacttggctgaaggacttggccgtgta

SEQ ID NO: 123 - DNA, 50 bp, Streptococcus sp.
  source 1..50: mol_type = genomic DNA
ttgctgagatgagctacttcctcaacttggctgaaggccttggccgtgta

SEQ ID NO: 124 - DNA, 50 bp, Streptococcus sp.
  source 1..50: mol_type = genomic DNA
ttgctgagatgagttacttcctcaacttggctgaaggacttggccgtgta

SEQ ID NO: 125 - DNA, 50 bp, Streptococcus sp.
  source 1..50: mol_type = genomic DNA
ttgctgaaatgagctacttcctcaacttggctgaaggacttggccgtgta

SEQ ID NO: 126 - DNA, 50 bp, Streptococcus sp.
  source 1..50: mol_type = genomic DNA
ttgctgagatgagctacttcctcaacttggctgaagggcttggccgtgta

SEQ ID NO: 127 - DNA, 50 bp, Streptococcus agalactiae
  source 1..50: mol_type = genomic DNA
tagcagagatgtcatacttcttaaaccttgcagagggtatcggtaaggta

SEQ ID NO: 128 - DNA, 50 bp, Streptococcus sp.
  source 1..50: mol_type = genomic DNA
tggctgagatgagctacttcctcaacttagcagaaggcatcggccgtgtg

SEQ ID NO: 129 - DNA, 50 bp, Streptococcus intermedius
  source 1..50: mol_type = genomic DNA
tggctgagatgagctacttcctcaacttagcagagggcatcggccgtgtg

SEQ ID NO: 130 - DNA, 50 bp, Streptococcus intermedius
  source 1..50: mol_type = genomic DNA
tggctgagatgagctacttcctcaacttagcagaaggcatcggccgtgta

SEQ ID NO: 131 - DNA, 50 bp, Streptococcus sp.
  source 1..50: mol_type = genomic DNA
tggctgagatgaattacttcttgaacctcgctgaaggacttggtcgtgtg

SEQ ID NO: 132 - DNA, 50 bp, Streptococcus equi
  source 1..50: mol_type = genomic DNA
tggctgagatgtcttatttccttaaccttgctgaaggtcttggaaaggtc

SEQ ID NO: 133 - DNA, 50 bp, Streptococcus sp.
  source 1..50: mol_type = genomic DNA
ttgcagagatgagctacttccttaacttggcagaaggtatcggacgtgtg

SEQ ID NO: 134 - DNA, 50 bp, Streptococcus sp.
  source 1..50: mol_type = genomic DNA
ttgcagagatgagctacttcctcaacttggctgaaggtatcggacgtgtg

SEQ ID NO: 135 - DNA, 50 bp, Streptococcus sp.
  source 1..50: mol_type = genomic DNA
tggctgagatgagctacttcctcaacttggcagaaggtatcggtcgtgtg

SEQ ID NO: 136 - DNA, 50 bp, Streptococcus sp.
  source 1..50: mol_type = genomic DNA
ttgcagagatgagctacttcctcaacttggcggaaggtatcggacgtgtg

SEQ ID NO: 137 - DNA, 50 bp, Streptococcus uberis
  source 1..50: mol_type = genomic DNA
tggctgaaatgtcatacttcttaaatctttctgaagggattggaaaagtt

SEQ ID NO: 138 - DNA, 50 bp, Streptococcus sp.
  source 1..50: mol_type = genomic DNA
tggcagaaatgagctatttcttgaaccttgcagaaggtattggccgcgtg

SEQ ID NO: 139 - DNA, 50 bp, Streptococcus parasanguinis
  source 1..50: mol_type = genomic DNA
tggcagaaatgagctatttcttgaaccttgcagaaggccttggccgtgta

SEQ ID NO: 140 - DNA, 50 bp, Streptococcus sanguinis
  source 1..50: mol_type = genomic DNA
tggctgagatgagctacttcctcaacttggctgaaggcattggtcgcgtg

SEQ ID NO: 141 - DNA, 50 bp, Streptococcus parauberis
  source 1..50: mol_type = genomic DNA
ttgcagaaatgtcttatttcttaaacctttctgaaggtattggtaaagta

SEQ ID NO: 142 - DNA, 50 bp, Streptococcus sp.
  source 1..50: mol_type = genomic DNA
tggctgaaatgtcatacttccttaaccttgctgaaggtctaggtaaagtt

SEQ ID NO: 143 - DNA, 50 bp, Streptococcus iniae
  source 1..50: mol_type = genomic DNA
ttgcagaaatgtcatatttcttgaaccttgcagagggtcttggaaaagtt

SEQ ID NO: 144 - DNA, 50 bp, Streptococcus sp.
  source 1..50: mol_type = genomic DNA
tggctgaaatgagctacttcctcaaccttgctgaaggtatcggtaaagta

SEQ ID NO: 145 - DNA, 50 bp, Streptococcus sp.
  source 1..50: mol_type = genomic DNA
tggctgaaatgagttacttcctcaaccttgctgaaggtatcggtaaagta

SEQ ID NO: 146 - DNA, 50 bp, Streptococcus sp.
  source 1..50: mol_type = genomic DNA
tggctgaaatgagctacttcctcaaccttgctgaaggtatcggtaaagtt

SEQ ID NO: 147 - DNA, 50 bp, Streptococcus sp.
  source 1..50: mol_type = genomic DNA
tggctgaaatgtcatacttccttaatcttgctgaaggtcttggtaaagtt

SEQ ID NO: 148 - DNA, 50 bp, Streptococcus sp.
  source 1..50: mol_type = genomic DNA
tggcagaaatgtcttacttccttaaccttgctgaaggtctaggtaaagtt

SEQ ID NO: 149 - DNA, 50 bp, Streptococcus sp.
  source 1..50: mol_type = genomic DNA
tggctgaaatgtcatacttccttaaccttgctgaaggtcttggtaaagtt

SEQ ID NO: 150 - DNA, 50 bp, Enterococcus faecalis
  source 1..50: mol_type = genomic DNA
ttgcttcaatgagctacttcttcaacttaatggaagatatcggcaatgtc

SEQ ID NO: 151 - DNA, 50 bp, Enterococcus sp.
  source 1..50: mol_type = genomic DNA
ttgcttcaatgagctacttcttcaacttaatggaagatatcggtaatgtc

SEQ ID NO: 152 - DNA, 50 bp, Enterococcus sp.
  source 1..50: mol_type = genomic DNA
ttgcttcaatgagctatttcttgaacttgatggaaggtatcggcaatgtc

SEQ ID NO: 153 - DNA, 50 bp, Enterococcus sp.
  source 1..50: mol_type = genomic DNA
ttgcttcaatgagctatttcttgaacttgatggaaggtatcggcaatgtt

SEQ ID NO: 154 - DNA, 50 bp, Porphyromonas gingivalis
  source 1..50: mol_type = genomic DNA
ttgagatcatcaagtatcttattgagttagtaaattccaaggcatcagta

SEQ ID NO: 155 - DNA, 50 bp, Porphyromonas cangingivalis
  source 1..50: mol_type = genomic DNA
ctgcgatcattgctcatctcgtagagttgaagaacagcaagcaggtcgtc

SEQ ID NO: 156 - DNA, 50 bp, unidentified
  source 1..50: mol_type = unassigned DNA
tggccatcatcaagtacctcatcgggcttgtcaactctaaggaggtcgtc

SEQ ID NO: 157 - DNA, 50 bp, unidentified
  source 1..50: mol_type = unassigned DNA
tggccatcatcaagtacctcatcgggcttgtcaactctaaggaagtcgtc

SEQ ID NO: 158 - DNA, 50 bp, Porphyromonas uenonis
  source 1..50: mol_type = genomic DNA
ttgctatcatacgccacctgatcaagctcgtcaatggtaaggcacctgtc

SEQ ID NO: 159 - DNA, 50 bp, unidentified
  source 1..50: mol_type = unassigned DNA
ttgcgatcatacgtcatctgatcaagctcgtcaatggtaaggctcctgtc

SEQ ID NO: 160 - DNA, 50 bp, unidentified
  source 1..50: mol_type = unassigned DNA
tttccattgttaaccaccttctattgttagcaacaacgggtgctaacgtt

SEQ ID NO: 161 - DNA, 50 bp, Porphyromonas asaccharolytica
  source 1..50: mol_type = genomic DNA
ttgcgatcatacgtcacttgatcaagctcgtcaatggtaaggctccagtc

SEQ ID NO: 162 - DNA, 50 bp, unidentified
  source 1..50: mol_type = unassigned DNA
tggccatcatcaagtacctcatcggtcttgtcaactctaaggaggtcgtc

SEQ ID NO: 163 - DNA, 50 bp, Porphyromonas macacae
  source 1..50: mol_type = genomic DNA
ttgaaattattaaatatctgattcaattagttaactccaaagcggtggtg

SEQ ID NO: 164 - DNA, 50 bp, Prevotella pallens
  source 1..50: mol_type = genomic DNA
tcgctatcattaagtatttgataaatcttgtaaattcaaatgcaacagtt

SEQ ID NO: 165 - DNA, 50 bp, Prevotella histicola
  source 1..50: mol_type = genomic DNA
ttgaaattatcaagtaccttataagtcttgtaaattcaaatgctacagtc

SEQ ID NO: 166 - DNA, 50 bp, Prevotella melaninogenica
  source 1..50: mol_type = genomic DNA
ttgagattattaagtatcttatcagccttatcaattcaaatgctacggtt

SEQ ID NO: 167 - DNA, 50 bp, Prevotella copri
  source 1..50: mol_type = genomic DNA
ttgagatcattaaatatcttattcagctgatcaactctagtgcaacagtt

SEQ ID NO: 168 - DNA, 50 bp, Prevotella copri
  source 1..50: mol_type = genomic DNA
ttgagattattaaatatcttattcagctgattaactctagtgcaacagtt

SEQ ID NO: 169 - DNA, 50 bp, Prevotella intermedia
  source 1..50: mol_type = genomic DNA
tcgagattatcaagtatttgataaacctcgtaaattcgaatgcaacagtt

SEQ ID NO: 170 - DNA, 50 bp, Prevotella intermedia
  source 1..50: mol_type = genomic DNA
tcgagattatcaagtatttgattaacctcgtaaattcgaatgcaacagtt

SEQ ID NO: 171 - DNA, 50 bp, Prevotella sp.
  source 1..50: mol_type = genomic DNA
ttgagattatcaagtacctcattagcttagtcaattcaaatgcaaccgtt

SEQ ID NO: 172 - DNA, 50 bp, Prevotella nanceiensis
  source 1..50: mol_type = genomic DNA
tcgcaattatacgatacttgattcagcttatcaattcgaatgcaacagtc

SEQ ID NO: 173 - DNA, 50 bp, Prevotella salivae
  source 1..50: mol_type = genomic DNA
ttgcgattatcaaatatctcattcagcttgtcaattctaatgttacagtt

SEQ ID NO: 174 - DNA, 50 bp, Prevotella salivae
  source 1..50: mol_type = genomic DNA
ttgcgattatcaaataccttattcagcttgtcaattctaatgttacagtt

SEQ ID NO: 175 - DNA, 50 bp, Prevotella sp.
  source 1..50: mol_type = genomic DNA
tcgcgattataaaatatttgataaaccttgtgaattcaaatgccactgtt

SEQ ID NO: 176 - DNA, 50 bp, Prevotella denticola
  source 1..50: mol_type = genomic DNA
ttgaaatcatcaaatatctcatcagcctgatcaactcaaatgccacggtt

SEQ ID NO: 177 - DNA, 50 bp, Prevotella buccae
  source 1..50: mol_type = genomic DNA
ttgagattatcaaatatctgattcagctgattaactccaatgctactgta

SEQ ID NO: 178 - DNA, 50 bp, Prevotella stercorea
  source 1..50: mol_type = genomic DNA
ttgccatcatccgctatctcatccagttggttaactctaacgcaactgtt

SEQ ID NO: 179 - DNA, 50 bp, Prevotella oris
  source 1..50: mol_type = genomic DNA
ttgaaatcataaaatatctcatccagttggttaattccaatgccactgtt

SEQ ID NO: 180 - DNA, 50 bp, Prevotella disiens
  source 1..50: mol_type = genomic DNA
ttgagattatcaaatatttgataaacctcatcaattctaacgcaactgtt

SEQ ID NO: 181 - DNA, 50 bp, Prevotella bryantii
  source 1..50: mol_type = genomic DNA
ttgctattatcaagtacttgattaagcttgttaattctcaggctactgtt

SEQ ID NO: 182 - DNA, 50 bp, Prevotella shahii
  source 1..50: mol_type = genomic DNA
ttgaaattatcaaatatctcattcagctggttaactctaatgcaaccgtg

SEQ ID NO: 183 - DNA, 50 bp, Tannerella forsythia
  source 1..50: mol_type = genomic DNA
ttgagattatcaaatatctgattgaattgatcaactcgaaggcggtggta

SEQ ID NO: 184 - DNA, 50 bp, Tannerella forsythia
  source 1..50: mol_type = genomic DNA
ttgagattatcaaatatctgattgaactgattaattcgaaggcagttgta

SEQ ID NO: 185 - DNA, 50 bp, Tannerella sp.
  source 1..50: mol_type = genomic DNA
tcgaaatcatcaaatacctcatcgagctgatcaactccaaggcggttgtt

SEQ ID NO: 186 - DNA, 50 bp, Bacteroides sp.
  source 1..50: mol_type = genomic DNA
ttgagatcattaaatatctgattgagttgattaactctaaagcagatgtg

SEQ ID NO: 187 - DNA, 50 bp, Bacteroides fragilis
  source 1..50: mol_type = genomic DNA
tcgagatcatcaaatatctgattgagctgattaattcaaaagcagatgta

SEQ ID NO: 188 - DNA, 50 bp, Bacteroides sp.
  source 1..50: mol_type = genomic DNA
tcgagatcatcaaatatctgattgagctgattaactcaaaagcagatgta

SEQ ID NO: 189 - DNA, 50 bp, Helicobacter pylori
  source 1..50: mol_type = genomic DNA
tcaccaccgttaaatacctcatgaaaatcaaaaacaatcagggcaagatt

SEQ ID NO: 190 - DNA, 50 bp, Helicobacter pylori
  source 1..50: mol_type = genomic DNA
tcaccaccgttaaatacctcatgaagatcaaaaacaatcaaggcaagatt

SEQ ID NO: 191 - DNA, 50 bp, Helicobacter pylori
  source 1..50: mol_type = genomic DNA
tcaccaccgttaaatacctcatgaagatcaaaaacaatcagggcaagatt

SEQ ID NO: 192 - DNA, 50 bp, Chlamydia trachomatis
  source 1..50: mol_type = genomic DNA
gacaaaccctgtcgcagaattgacgcacaagcgtcgtctgtcagcattag

SEQ ID NO: 193 - DNA, 50 bp, Chlamydia trachomatis
  source 1..50: mol_type = genomic DNA
taagatccacgctcgttctataggaccttactctctcgttacgcagcaac

SEQ ID NO: 194 - DNA, 50 bp, Neisseria sp.
  source 1..50: mol_type = genomic DNA
tcgcctcgattgcgactttggtcgagttgcgtaacggccatggcgaagtg

SEQ ID NO: 195 - DNA, 50 bp, Neisseria meningitidis
  source 1..50: mol_type = genomic DNA
tcgcctcgattgcgactttggtcgagttgcgtaacggccatggtgaagtt

SEQ ID NO: 196 - DNA, 50 bp, Neisseria lactamica
  source 1..50: mol_type = genomic DNA
tcgcctcgattgcgactttggtcgagctgcgtaacggtcacggcgaagtg

SEQ ID NO: 197 - DNA, 50 bp, Neisseria sp.
  source 1..50: mol_type = genomic DNA
ttgcttctattgcgacattggttgaactgcgtaacggtcatggcgaagta

SEQ ID NO: 198 - DNA, 50 bp, Neisseria gonorrhoeae
  source 1..50: mol_type = genomic DNA
tcgcctcgattgcgactttggtcgagttgcgtaactaccatggcgaagtg

SEQ ID NO: 199 - DNA, 50 bp, Neisseria sp.
  source 1..50: mol_type = genomic DNA
ttgtttcaattgctaccttagttgaattacgtaatcataatgatggtgtt

SEQ ID NO: 200 - DNA, 50 bp, Neisseria mucosa
  source 1..50: mol_type = genomic DNA
ttgcatcaattgctactttagttgaattgcgaaacggtcatggcgaagtg

SEQ ID NO: 201 - DNA, 50 bp, Neisseria sp.
  source 1..50: mol_type = genomic DNA
tggcttcgattgcaacgttggttgagttgcgtaacggtcacggtgaagtg

SEQ ID NO: 202 - DNA, 50 bp, Neisseria shayeganii
  source 1..50: mol_type = genomic DNA
tggcttccatcgccactttggtggagttgcgcaacgggcatggcgaagtg

SEQ ID NO: 203 - DNA, 50 bp, Neisseria sp.
  source 1..50: mol_type = genomic DNA
tcgcttcgattgccactttggttgaattgcgtaacggtcacggcgaagtg

SEQ ID NO: 204 - DNA, 50 bp, Neisseria sp.
  source 1..50: mol_type = genomic DNA
ttgtttctattgccactttagttgagctgcgtaatggacatggtgaagta

SEQ ID NO: 205 - DNA, 50 bp, Neisseria meningitidis
  source 1..50: mol_type = genomic DNA
aacgccgcgtatccgcattgggtccgggcggtttgacccgcgaacgtgca

SEQ ID NO: 206 - DNA, 50 bp, Neisseria gonorrhoeae
  source 1..50: mol_type = genomic DNA
aacgccgtgtatctgcattgggcccgggcggtttgacccgcgaacgtgcc

SEQ ID NO: 207 - DNA, 50 bp, Neisseria meningitidis
  source 1..50: mol_type = genomic DNA
aacgccgcgtatctgcattgggtccgggcggtttgacccgcgaacgtgcc

SEQ ID NO: 208 - DNA, 50 bp, Neisseria meningitidis
  source 1..50: mol_type = genomic DNA
aacgccgcgtatccgcattgggtccgggcggtttgacccgcgaacgtgcc

SEQ ID NO: 209 - DNA, 50 bp, Neisseria meningitidis
  source 1..50: mol_type = genomic DNA
aacgccgtgtatctgcattgggtccgggcggtttgacccgcgaacgtgca

SEQ ID NO: 210 - DNA, 50 bp, Neisseria sp.
  source 1..50: mol_type = genomic DNA
aacgccgtgtatctgcattgggcccgggcggtttgactcgcgaacgtgca

SEQ ID NO: 211 - DNA, 50 bp, Neisseria gonorrhoeae
  source 1..50: mol_type = genomic DNA
aacgccgtgtatctgcattgggtccgggcggtttgacccgcgaacgtgcc

SEQ ID NO: 212 - DNA, 50 bp, Neisseria lactamica
  source 1..50: mol_type = genomic DNA
aacgccgtgtatctgcgttgggtccgggcggtttgacccgcgaacgtgca

SEQ ID NO: 213 - DNA, 50 bp, Neisseria sp.
  source 1..50: mol_type = genomic DNA
agcgtcgtgtgtctgctttaggtccaggtggtttgacacgtgaacgtgca

SEQ ID NO: 214 - DNA, 50 bp, Neisseria zoodegmatis
  source 1..50: mol_type = genomic DNA
agcgtcgtgtgtctgctttaggtccgggtggtttgacacgtgaacgtgca

SEQ ID NO: 215 - DNA, 50 bp, Neisseria meningitidis
  source 1..50: mol_type = genomic DNA
aacgtcgtgtatctgcattgggtccgggcggtttgacccgcgaacgtgca

SEQ ID NO: 216 - DNA, 50 bp, Neisseria sp.
  source 1..50: mol_type = genomic DNA
aacgtcgtgtttctgccttgggcccgggtggtttgacccgtgagcgtgcc

SEQ ID NO: 217 - DNA, 50 bp, Neisseria sp.
  source 1..50: mol_type = genomic DNA
aacgtcgtgtttctgctttgggtccaggcggtttgacccgtgaacgtgct

SEQ ID NO: 218 - DNA, 50 bp, Neisseria sp.
  source 1..50: mol_type = genomic DNA
aacgccgtgtatccgcattgggtccgggcggcttgacccgcgaacgtgca

SEQ ID NO: 219 - DNA, 50 bp, Neisseria sp.
  source 1..50: mol_type = genomic DNA
aacgccgtgtatctgcattgggccctggtggtttgactcgcgaacgtgca

SEQ ID NO: 220 - DNA, 50 bp, Neisseria sp.
  source 1..50: mol_type = genomic DNA
agcgtcgtgtgtctgctttaggtccgggcggtttgacacgtgaacgtgcg

SEQ ID NO: 221 - DNA, 50 bp, Aggregatibacter actinomycetemcomitans
  source 1..50: mol_type = genomic DNA
tcagtgtgatgaaaaaattgattgatatccgtaatggccgtggtgaagtg

SEQ ID NO: 222 - DNA, 50 bp, Aggregatibacter aphrophilus
  source 1..50: mol_type = genomic DNA
tcagtgtgatgaagaaactgattgatattcgtaatggtcgcggtgaagtg

SEQ ID NO: 223 - DNA, 50 bp, Aggregatibacter actinomycetemcomitans
  source 1..50: mol_type = genomic DNA
tcagtgtgatgaagaaattgattgatatccgtaatggccgtggtgaagtg

SEQ ID NO: 224 - DNA, 50 bp, Aggregatibacter aphrophilus
  source 1..50: mol_type = genomic DNA
tcagtgtgatgaaaaaactgattgatattcgtaatggtcgcggagaagtg

SEQ ID NO: 225 - DNA, 50 bp, Aggregatibacter segnis
  source 1..50: mol_type = genomic DNA
taagtgtcatgaagaaattgatcgaaattcgtaacggtcgtggtgaagtg

SEQ ID NO: 226 - DNA, 50 bp, Bacillus sp.
  source 1..50: mol_type = genomic DNA
tcgcttccatcagctatttcttcaacttgctgcatggtgtcggcgataca

SEQ ID NO: 227 - DNA, 50 bp, Bacillus clausii
  source 1..50: mol_type = genomic DNA
tcgcttccatcagctatttcttcaacttgttgcatggtgtcggcgataca

SEQ ID NO: 228 - DNA, 50 bp, Fusobacterium sp.
  source 1..50: mol_type = genomic DNA
ctattaaatatgttatagagcttaataatggtgatcaaaatgttcatact

SEQ ID NO: 229 - DNA, 50 bp, Fusobacterium sp.
  source 1..50: mol_type = genomic DNA
ctattaaatatgttatagatcttaataatggcgatcaaaatgttcatact

SEQ ID NO: 230 - DNA, 50 bp, Fusobacterium periodonticum
  source 1..50: mol_type = genomic DNA
caatgaaatatgttactgacctttataatggtgaccaaaatgttcataca

SEQ ID NO: 231 - DNA, 50 bp, Fusobacterium necrophorum
  source 1..50: mol_type = genomic DNA
cgatacaatatgtcattgatttaaataatggggaatctcatgtccatacc

SEQ ID NO: 232 - DNA, 50 bp, Fusobacterium periodonticum
  source 1..50: mol_type = genomic DNA
caatgaaatatgttactgacctttataatggtgatcaaaatgttcataca

SEQ ID NO: 233 - DNA, 50 bp, Fusobacterium sp.
  source 1..50: mol_type = genomic DNA
tagctacaatgaagtatgtaattaacttaaataatggaaatggacatact

SEQ ID NO: 234 - DNA, 50 bp, Fusobacterium sp.
  source 1..50: mol_type = genomic DNA
ctattaagtatgttatagagctaaataatggtgaccaaaatgttcatact

SEQ ID NO: 235 - DNA, 50 bp, Fusobacterium nucleatum
  source 1..50: mol_type = genomic DNA
ctattagatatgttatagatcttaataatggcgatcaaaatgttcatact

SEQ ID NO: 236 - DNA, 50 bp, Fusobacterium sp.
  source 1..50: mol_type = genomic DNA
taggaacaatgaaatatgtaattaatctaaataatggaaatggacacact

SEQ ID NO: 237 - DNA, 50 bp, Fusobacterium nucleatum
  source 1..50: mol_type = genomic DNA
ctattaagtatgttatagaacttaataatggtgaacaaaatgttcatact

SEQ ID NO: 238 - DNA, 50 bp, Fusobacterium ulcerans
  source 1..50: mol_type = genomic DNA
ttggaacaatgaaatatgtaattaatctaaataatggaaatggacatact

SEQ ID NO: 239 - DNA, 50 bp, Fusobacterium nucleatum
  source 1..50: mol_type = genomic DNA
ctattaaatatgttatagaacttaataatggtgatcaaaatgttcatact

SEQ ID NO: 240 - DNA, 50 bp, Fusobacterium sp.
  source 1..50: mol_type = genomic DNA
ctattaaatatgttatagatcttaataatggtgatcaaaatgttcatact

SEQ ID NO: 241 - DNA, 50 bp, Fusobacterium nucleatum
  source 1..50: mol_type = genomic DNA
ctattaaatatgtaatagagcttaataatggtgatcaaaatgttcatact

SEQ ID NO: 242 - DNA, 50 bp, Fusobacterium sp.
  source 1..50: mol_type = genomic DNA
cgattcaatatgtcattgatttaaataatggagaatcccatgtacataca

SEQ ID NO: 243 - DNA, 50 bp, Fusobacterium varium
  source 1..50: mol_type = genomic DNA
ttggaacaatgaaatatgtaattaatttgaataatggaaatgggcatact

SEQ ID NO: 244 - DNA, 50 bp, Fusobacterium necrogenes
  source 1..50: mol_type = genomic DNA
ttgcaactatgaagtatgtaattaatttaaacaatggaaatggacatact

SEQ ID NO: 245 - DNA, 50 bp, Fusobacterium naviforme
  source 1..50: mol_type = genomic DNA
tcgcctccatcaattacaacatgcatatcgaggagggcatcggcagcaac

SEQ ID NO: 246 - DNA, 50 bp, Clostridium sp.
  source 1..50: mol_type = genomic DNA
tagcttcaataagttatgagttcaatatattctataatataggaaatatt

SEQ ID NO: 247 - DNA, 50 bp, unidentified
  source 1..50: mol_type = unassigned DNA
tcgatgtattacgtacattggttgaaatccgtaacggtaaaggtgaagtc

SEQ ID NO: 248 - DNA, 50 bp, Acinetobacter sp.
  source 1..50: mol_type = genomic DNA
ttgatgtattacgtacattagttgaaatccgtaacggtaaaggtgaagtc

SEQ ID NO: 249 - DNA, 50 bp, Acinetobacter sp.
  source 1..50: mol_type = genomic DNA
tcgatgtattacgtacgttggttgaaatccgtaacggtaaaggcgaagta

SEQ ID NO: 250 - DNA, 50 bp, Acinetobacter sp.
  source 1..50: mol_type = genomic DNA
tcgatgtattacgtacattagttgaaatccgtaacggtaaaggtgaagtc

SEQ ID NO: 251 - DNA, 50 bp, Acinetobacter sp.
  source 1..50: mol_type = genomic DNA
tcgatgtattacgtactttagttgaaattcgtaacggtaagggtgaggtc

SEQ ID NO: 252 - DNA, 50 bp, Acinetobacter sp.
  source 1..50: mol_type = genomic DNA
ttgatgtattacgtacattggttgaaatccgtaacggtaaaggtgaagtc

SEQ ID NO: 253 - DNA, 50 bp, Acinetobacter sp.
  source 1..50: mol_type = genomic DNA
tcgatgtgttacgtactttagttgaaattcgtaacggtaagggtgaggtc

SEQ ID NO: 254 - DNA, 50 bp, Acinetobacter sp.
  source 1..50: mol_type = genomic DNA
tagatgtattacgtacgttggttgaaatccgtaacggtaaaggcgaagta

SEQ ID NO: 255 - DNA, 50 bp, Acinetobacter sp.
  source 1..50: mol_type = genomic DNA
ctgatgtattaaaaacattagtagaaatccgtaacggtaaaggtgaagtc

SEQ ID NO: 256 - DNA, 50 bp, Acinetobacter sp.
  source 1..50: mol_type = genomic DNA
ttgatgtactgcgtacattggtagaaatccgtaacggtaaaggtgaagtc

SEQ ID NO: 257 - DNA, 50 bp, Acinetobacter sp.
  source 1..50: mol_type = genomic DNA
ttgatgtactgcgtacattggttgaaatccgtaacggtaaaggtgaagtc

SEQ ID NO: 258 - DNA, 50 bp, Acinetobacter sp.
  source 1..50: mol_type = genomic DNA
tcgatgtattacgtacattggttgaaatccgtaatggtaaaggtgaagtc

SEQ ID NO: 259 - DNA, 50 bp, Acinetobacter sp.
  source 1..50: mol_type = genomic DNA
ctgatgtactacgtacattggttgagattcgtaacggtaaaggtgaagtt

SEQ ID NO: 260 - DNA, 50 bp, unidentified
  source 1..50: mol_type = unassigned DNA
ttgatgttatgaaaaagctcatcgatatccgtaacggtaaaggcgaagtc

SEQ ID NO: 261 - DNA, 50 bp, unidentified
  source 1..50: mol_type = unassigned DNA
tcgaagtgatgaagaagctcatcgatatccgtaacggtaaaggcgaagtg

SEQ ID NO: 262 - DNA, 50 bp, unidentified
  source 1..50: mol_type = unassigned DNA
aacgtcgtatctccgcactcggcccaggcggtctgacccgtgaacgtgca

SEQ ID NO: 263 - DNA, 50 bp, unidentified
  source 1..50: mol_type = unassigned DNA
aacgtcgtatctcggcactcggcccaggcggtctgacccgtgaacgtgca

SEQ ID NO: 264 - DNA, 50 bp, unidentified
  source 1..50: mol_type = unassigned DNA
aacgtcgtatctccgcactcggcccgggtggtctgacccgtgaacgtgca

SEQ ID NO: 265 - DNA, 50 bp, Escherichia coli
  source 1..50: mol_type = genomic DNA
aacgtcgtatctccgcactcggcccaggtggtctgacccgtgaacgtgca

SEQ ID NO: 266 - DNA, 50 bp, Escherichia sp.
  source 1..50: mol_type = genomic DNA
agcgtcgtatctccgcactcggcccaggcggtctgacccgtgaacgtgca

SEQ ID NO: 267 - DNA, 50 bp, Escherichia coli
  source 1..50: mol_type = genomic DNA
aacgtcgtatctcggcacttggcccaggcggtctgacccgtgaacgtgca

SEQ ID NO: 268 - DNA, 50 bp, Escherichia albertii
  source 1..50: mol_type = genomic DNA
aacgtcgtatctccgcactcggccctggcggtctgactcgtgaacgcgcg

SEQ ID NO: 269 - DNA, 50 bp, Escherichia coli
  source 1..50: mol_type = genomic DNA
aacgtcgtatctcggcccttggcccaggcggtctgacccgtgaacgtgca

SEQ ID NO: 270 - DNA, 50 bp, Escherichia coli
  source 1..50: mol_type = genomic DNA
aacgtcgtatctcagcactcggcccaggtggtctgacccgtgaacgtgca

SEQ ID NO: 271 - DNA, 50 bp, Escherichia coli
  source 1..50: mol_type = genomic DNA
aacgtcgtatctccgcactcggcccgggcggtctgacccgtgaacgtgca

SEQ ID NO: 272 - DNA, 50 bp, Escherichia coli
  source 1..50: mol_type = genomic DNA
aacgtcgtatttccgcactcggcccaggcggtctgacccgtgaacgtgca

SEQ ID NO: 273 - DNA, 50 bp, Escherichia sp.
  source 1..50: mol_type = genomic DNA
aacgtcgtatctctgcactcggcccaggcggtctgacccgtgaacgtgca

SEQ ID NO: 274 - DNA, 50 bp, Escherichia coli
  source 1..50: mol_type = genomic DNA
aacgtcgtatcttcgcactcggcccaggcggtctgacccgtgaacgtgca

SEQ ID NO: 275 - DNA, 50 bp, unidentified
  source 1..50: mol_type = unassigned DNA
aacgtcgtatctccgcactcggtccaggcggtctgacccgtgaacgtgca

SEQ ID NO: 276 - DNA, 50 bp, Escherichia sp.
  source 1..50: mol_type = genomic DNA
aacgtcgtatctctgcactcggtccaggcggtctgacccgtgaacgtgca

SEQ ID NO: 277 - DNA, 50 bp, unidentified
  source 1..50: mol_type = unassigned DNA
aacgtcgtatctccgcactcggcccaggcggtctgacccgtgagcgcgca

SEQ ID NO: 278 - DNA, 50 bp, unidentified; identical to SEQ ID NO: 273
  source 1..50: mol_type = unassigned DNA

SEQ ID NO: 279 - DNA, 50 bp, unidentified
  source 1..50: mol_type = unassigned DNA
aacgtcgtatctctgcactcggcccaggcggtctgacccgtgaacgcgca

SEQ ID NO: 280 - DNA, 50 bp, unidentified
  source 1..50: mol_type = unassigned DNA
agcgtcgtatctctgcactcggcccaggcggtctgacccgtgagcgcgca

SEQ ID NO: 281 - DNA, 50 bp, Serratia sp.
  source 1..50: mol_type = genomic DNA
aacgtcgtatctctgcattgggcccaggcggtctgacccgtgaacgtgcc

SEQ ID NO: 282 - DNA, 50 bp, Enterobacter sp.
  source 1..50: mol_type = genomic DNA
aacgtcgtatctctgcactcggcccaggcggtctgactcgtgaacgcgca

SEQ ID NO: 283 - DNA, 50 bp, Enterobacter sp.
  source 1..50: mol_type = genomic DNA
agcgtcgtatctctgcactcggcccaggcggtctgacccgtgaacgcgca

SEQ ID NO: 284 - DNA, 50 bp, unidentified
  source 1..50: mol_type = unassigned DNA
aacgtcgtatctctgcactcggcccaggcggtctgacccgtgagcgcgca

SEQ ID NO: 285 - DNA, 50 bp, Enterobacter sp.
  source 1..50: mol_type = genomic DNA
aacgtcgtatttctgcactcggcccaggcggtctgacccgtgaacgtgca

SEQ ID NO: 286 - DNA, 50 bp, unidentified
  source 1..50: mol_type = unassigned DNA
agcgtcgtatctctgcactcggcccaggcggtctgacccgtgaacgtgca

SEQ ID NO: 287 - DNA, 50 bp, unidentified
  source 1..50: mol_type = unassigned DNA
aacgtcgtatctctgcactcggcccgggcggtctgacccgtgagcgcgca

SEQ ID NO: 288 - DNA, 50 bp, unidentified
  source 1..50: mol_type = unassigned DNA
agcgtcgtatctctgcactcggcccgggcggtctgacccgtgagcgcgca

SEQ ID NO: 289 - DNA, 50 bp, unidentified; identical to SEQ ID NO: 271
  source 1..50: mol_type = unassigned DNA

SEQ ID NO: 290 - DNA, 50 bp, Serratia fonticola
  source 1..50: mol_type = genomic DNA
agcgtcgtatctctgcattgggcccaggcggtctgacccgtgaacgtgcc

SEQ ID NO: 291 - DNA, 25 bp, unidentified
  source 1..25: mol_type = unassigned DNA
ttgaaatcatcaaatatctgattga

SEQ ID NO: 292 - DNA, 25 bp, unidentified
  source 1..25: mol_type = unassigned DNA
ttgcttctattaattacaatatgca

SEQ ID NO: 293 - DNA, 25 bp, unidentified
  source 1..25: mol_type = unassigned DNA
tcgcatccatcaattacaatatgca

SEQ ID NO: 294 - DNA, 25 bp, unidentified
  source 1..25: mol_type = unassigned DNA
ttgaaatcattaaatatctgattga

SEQ ID NO: 295 - DNA, 25 bp, unidentified
  source 1..25: mol_type = unassigned DNA
tgtcttccatcaactatctgaacgg

SEQ ID NO: 296 - DNA, 50 bp, Bacteroides stercoris
  source 1..50: mol_type = genomic DNA
ttgagattatcaagtatctgattgagttgataaactcaaaagcagatgtg

SEQ ID NO: 297 - DNA, 50 bp, Bacteroides vulgatus
  source 1..50: mol_type = genomic DNA
ttgaaatcattaagtatctgattgagctgattaactctaaagcggatgtt

SEQ ID NO: 298 - DNA, 50 bp, unidentified
  source 1..50: mol_type = unassigned DNA
tcgcatccatcaactataatatgcatctggagtggggcatcggaacagat

SEQ ID NO: 299 - DNA, 50 bp, Bacteroides ovatus
  source 1..50: mol_type = genomic DNA
ttgaaatcatcaaatatctgattgagttgattaactcaaaagcggatgtg

SEQ ID NO: 300 - DNA, 50 bp, Blautia sp.
  source 1..50: mol_type = genomic DNA
ttgcgtccattaactacaatatgcatctggagtatggccttggtaacgat

SEQ ID NO: 301 - DNA, 50 bp, Alistipes putredinis
  source 1..50: mol_type = genomic DNA
tcgccatcatcaagtacctgatccagctcatcaactcgaaagccgaggtg

SEQ ID NO: 302 - DNA, 50 bp, Faecalibacterium prausnitzii
  source 1..50: mol_type = genomic DNA
tgtcctccatcaactacctgaacggtctgggccacggcgttggcaccacc

SEQ ID NO: 303 - DNA, 50 bp, Ruminococcus sp.
  source 1..50: mol_type = genomic DNA
ttgcttctattaactacaatatgcatctggaatatggccttggcaatgcc

SEQ ID NO: 304 - DNA, 50 bp, Paraprevotella clara
  source 1..50: mol_type = genomic DNA
tcgagattatcaagtatctgatagagctgataaactcaaaggcacttgtc

SEQ ID NO: 305 - DNA, 50 bp, Eubacterium rectale
  source 1..50: mol_type = genomic DNA
tcgcaactatcaactacaatatgcacttagagtggggcgcaggaacagat

SEQ ID NO: 306 - DNA, 50 bp, Blautia wexlerae
  source 1..50: mol_type = genomic DNA
tcgcttccatcaactacaacatgcatctggaatacggcgcaggaaatgcc

SEQ ID NO: 307 - DNA, 50 bp, Ruminococcus sp.
  source 1..50: mol_type = genomic DNA
ttgcttctattaactacaatatgcatctggaatatggccttggtaatgcc

SEQ ID NO: 308 - DNA, 50 bp, Faecalibacterium prausnitzii
  source 1..50: mol_type = genomic DNA
tgtcttccatcaactatctgaacggcctgggccacggcatcggcaccacc

SEQ ID NO: 309 - DNA, 50 bp, Faecalibacterium sp.; identical to SEQ ID NO: 308
  source 1..50: mol_type = genomic DNA

SEQ ID NO: 310 - DNA, 50 bp, Alistipes sp.
  source 1..50: mol_type = genomic DNA
tcgccattatcaagtacctcatccagctcatcaactcgcgcgccgaggtg

SEQ ID NO: 311 - DNA, 50 bp, Bifidobacterium pseudocatenulatum
  source 1..50: mol_type = genomic DNA
agttcccgggcaagcgtgacggccaggatgtggatctgcgcgtggacgtc

SEQ ID NO: 312 - DNA, 50 bp, Bacteroides sp.
  source 1..50: mol_type = genomic DNA
tcgaaattatcaaatatctcatcgagttgattaactcgaaagcggatgtg

SEQ ID NO: 313 - DNA, 50 bp, Ruminococcus sp.
  source 1..50: mol_type = genomic DNA
tcgctacggtttcttacttcctcaacctttgcgagggcgttggtactgtt

SEQ ID NO: 314 - DNA, 50 bp, Faecalibacterium prausnitzii
  source 1..50: mol_type = genomic DNA
tgtcctccatcaactacctgaacggtctgggctacggcatcggcaccacc

SEQ ID NO: 315 - DNA, 50 bp, unidentified
  source 1..50: mol_type = unassigned DNA
tggcttcaattaactacaatatgcatctggaatatggtatgggtaatgat

SEQ ID NO: 316 - DNA, 50 bp, unidentified
  source 1..50: mol_type = unassigned DNA
tggcttccatcaactataatatgcatctggagtatggcctgggcaacgat

SEQ ID NO: 317 - DNA, 50 bp, Bifidobacterium longum
  source 1..50: mol_type = genomic DNA
ccttcccgggcaagcgcaacggcgaagacgttgacctgcgcgtggacgtc

SEQ ID NO: 318 - DNA, 50 bp, Blautia sp.
  source 1..50: mol_type = genomic DNA
ttgcttctattaattacaatatgcatctggaatacggcattggaaatgac

SEQ ID NO: 319 - DNA, 50 bp, Ruminococcus lactaris
  source 1..50: mol_type = genomic DNA
ttgcatccatcaattacaatatgcatcttgagtatggcatgggtaatgat

SEQ ID NO: 320 - DNA, 50 bp, Anaerostipes sp.
  source 1..50: mol_type = genomic DNA
tagcatccatcaactacaatatccatttagagtatggaattggacatgat

SEQ ID NO: 321 - DNA, 50 bp, Eubacterium sp.
  source 1..50: mol_type = genomic DNA
tagcttctattaactacaatatccatctggaatatggtgttggtaatgac

SEQ ID NO: 322 - DNA, 50 bp, Subdoligranulum sp.
  source 1..50: mol_type = genomic DNA
ttgcctccgtcaactacctgctgggccttgatcacggcatcggcaccacc

SEQ ID NO: 323 - DNA, 50 bp, unidentified
  source 1..50: mol_type = unassigned DNA
tcgcttccatctgctatctgctcaacctcggtcacggcatcggcacggtt

SEQ ID NO: 324 - DNA, 50 bp, Faecalibacterium sp.
  source 1..50: mol_type = genomic DNA
tggcttccatcaactacctgaacggtctgggccacaacattggcaccacc

SEQ ID NO: 325 - DNA, 50 bp, Alistipes finegoldii
  source 1..50: mol_type = genomic DNA
tcgccattatcaaatacctgatccagctgatcaactccaaggccgacgtg

SEQ ID NO: 326 - DNA, 50 bp, unidentified
  source 1..50: mol_type = unassigned DNA
tcgcctccatcaactacatgaacgcgctggcgcacggcatcgtctataag

SEQ ID NO: 327 - DNA, 50 bp, unidentified
  source 1..50: mol_type = unassigned DNA
ttgcttccgtcaactacctgctgggccttgaccatggcatcggcgtgacc

SEQ ID NO: 328 - DNA, 50 bp, Eubacterium sp.
  source 1..50: mol_type = genomic DNA
ttgcttctattaattataatatgcaccttgaatacggcgttggtacaaag

SEQ ID NO: 329 - DNA, 50 bp, Dialister sp.
  source 1..50: mol_type = genomic DNA
tcgcggcggtagactatcttttgaatatgatccagggctatggacgccag

SEQ ID NO: 330 - DNA, 50 bp, Coprococcus comes
  source 1..50: mol_type = genomic DNA
tggcgtctatcaattacaatatgcatcttgaatatggaatcggtaaagat

SEQ ID NO: 331 - DNA, 50 bp, Bacteroides caccae
  source 1..50: mol_type = genomic DNA
ttgaaatcattaaatatctgattgagttaattaactcaaaggcagatgtg

SEQ ID NO: 332 - DNA, 50 bp, Parabacteroides distasonis
  source 1..50: mol_type = genomic DNA
tcgagatcatcaagtacctgatcgagttgatcaactcgaaggctatcgtg

SEQ ID NO: 333 - DNA, 50 bp, Parabacteroides merdae
  source 1..50: mol_type = genomic DNA
ttgagatcatcaaatatctgattgagttgatcaactcgaaagcgatcgtt

SEQ ID NO: 334 - DNA, 50 bp, Faecalibacterium sp.
  source 1..50: mol_type = genomic DNA
tggcttccatcaactacctgaacggtctgggccacaatattggcaccacc

SEQ ID NO: 335 - DNA, 50 bp, unidentified
  source 1..50: mol_type = unassigned DNA
tcgaaattatcaaatatcttatcgagttgattaactcgaagaccgatgtc

SEQ ID NO: 336 - DNA, 50 bp, Roseburia inulinivorans
  source 1..50: mol_type = genomic DNA
tcgcatccatcaattacaatatgcatttagagtatggtattggtcatgat

SEQ ID NO: 337 - DNA, 50 bp, unidentified
  source 1..50: mol_type = unassigned DNA
taggttctattaactactgcttaaacttagagtatggcgtaggacaggat

SEQ ID NO: 338 - DNA, 50 bp, Dorea longicatena
  source 1..50: mol_type = genomic DNA
tagcttctattaactacaatatgcatctggaatatggcatcggaactgat

SEQ ID NO: 339 - DNA, 50 bp, Roseburia intestinalis
  source 1..50: mol_type = genomic DNA
ttgcatccatcaactacaatatgcacttagagtatggtatcggaaatgat

SEQ ID NO: 340 - DNA, 50 bp, unidentified
  source 1..50: mol_type = unassigned DNA
ttgcgtctgtaaactattgtctaaaccttgctaacggtataggtactgtt

SEQ ID NO: 341 - DNA, 50 bp, Blautia sp.
  source 1..50: mol_type = genomic DNA
ttgcttctatcaactacaatatgcatctggaatatggcattggtaatgac

SEQ ID NO: 342 - DNA, 50 bp, Alistipes onderdonkii
  source 1..50: mol_type = genomic DNA
tcgccatcatcaaatacctgatccagctgatcaactcgaaggccgacgtc

SEQ ID NO: 343 - DNA, 50 bp, Bacteroides stercoris
  source 1..50: mol_type = genomic DNA
ttatcactatcgaagaggctaagggtaccgataccactatcggtgtagta

SEQ ID NO: 344 - DNA, 50 bp, Bacteroides vulgatus
  source 1..50: mol_type = genomic DNA
tgattactatcgaagaagctaaaggaacggatactaccatcggtgtggta

SEQ ID NO: 345 - DNA, 50 bp, unidentified
  source 1..50: mol_type = unassigned DNA
tcatcacaatcgaagagtccaaaaccatgcagacagagcttgacctggta

SEQ ID NO: 346 - DNA, 50 bp, Bacteroides ovatus
  source 1..50: mol_type = genomic DNA
tgattactatcgaagaagcaaaaggaacagacactactatcggtgtagta

SEQ ID NO: 347 - DNA, 50 bp, Blautia sp.
  source 1..50: mol_type = genomic DNA
ttatcactgttgaggagtccaagaccatgcatacagagcttgaccttgta

SEQ ID NO: 348 - DNA, 50 bp, Alistipes putredinis
  source 1..50: mol_type = genomic DNA
tcatcaccgtcgaggaggccaagggtaccgaaacccatgtggatgtggtc

SEQ ID NO: 349 - DNA, 50 bp, Faecalibacterium prausnitzii
  source 1..50: mol_type = genomic DNA
tcaccatcgaggagaacaagaccaccgccgagacctacaacgagatcgtg

SEQ ID NO: 350 - DNA, 50 bp, Blautia wexlerae
  source 1..50: mol_type = genomic DNA
ttatcacagtagaagaatccaagacaatgcacacagaacttgaccttgta

SEQ ID NO: 351 - DNA, 50 bp, Paraprevotella clara
  source 1..50: mol_type = genomic DNA
tgattaccatcgaagaagccaagggacgcgacactactatcggtgtggtg

SEQ ID NO: 352 - DNA, 50 bp, Eubacterium rectale
  source 1..50: mol_type = genomic DNA
ttatcacaattgaagagtcaaagacaatgcagacagagcttgaccttgta

SEQ ID NO: 353 - DNA, 50 bp, Blautia wexlerae
  source 1..50: mol_type = genomic DNA
ttatcaccatcgaggagtccaagaccatgcagaacgagctggagctggta

SEQ ID NO: 354 - DNA, 50 bp, Ruminococcus sp.; identical to SEQ ID NO: 350
  source 1..50: mol_type = genomic DNA

SEQ ID NO: 355 - DNA, 50 bp, Faecalibacterium prausnitzii
  source 1..50: mol_type = genomic DNA
tcaccatcgaggagaacaagaccactgccgagacctacaacgagatcgtc

SEQ ID NO: 356 - DNA, 50 bp, Faecalibacterium sp.; identical to SEQ ID NO: 355
  source 1..50: mol_type = genomic DNA

SEQ ID NO: 357 - DNA, 50 bp, Alistipes sp.
  source 1..50: mol_type = genomic DNA
tcatcacggtcgaggaggccaaaggcaccgacacccatgtggacgtggtc

SEQ ID NO: 358 - DNA, 50 bp, Bifidobacterium pseudocatenulatum
  source 1..50: mol_type = genomic DNA
tcgtgaccgttgaggacaacaaccgcttcggcctggatctggactttacc

SEQ ID NO: 359 - DNA, 50 bp, Bacteroides sp.
  source 1..50: mol_type = genomic DNA
ttatcactatcgaagaggcaaagggtactgatactactatcggtgtggtt

SEQ ID NO: 360 - DNA, 50 bp, Ruminococcus sp.
  source 1..50: mol_type = genomic DNA
ttatcactcttgaggagtcaaagactgctgaaacttacagcgaagtcgtt

SEQ ID NO: 361 - DNA, 50 bp, Faecalibacterium prausnitzii
  source 1..50: mol_type = genomic DNA
tcaccatcgaggagaacaagaccactgccgagacctacaacgagatcgtg

SEQ ID NO: 362 - DNA, 50 bp, unidentified
  source 1..50: mol_type = unassigned DNA
ttattacaatcgaagaatctaaaacaatgcagacagagcttgaccttgtg

SEQ ID NO: 363 - DNA, 50 bp, unidentified
  source 1..50: mol_type = unassigned DNA
ttatcaccattgaagagtccaagaccatgcagaccgaactggatctggta

SEQ ID NO: 364 - DNA, 50 bp, Bifidobacterium longum
  source 1..50: mol_type = genomic DNA
ttgtgaccgttgaagacaacaaccgcttcggcctggacctcgacttcacc

SEQ ID NO: 365 - DNA, 50 bp, Blautia sp.
  source 1..50: mol_type = genomic DNA
ttattactgtagaagagtccaagaccatgcacacagagcttgaccttgta

SEQ ID NO: 366 - DNA, 50 bp, Ruminococcus sp.; identical to SEQ ID NO: 365
  source 1..50: mol_type = genomic DNA

SEQ ID NO: 367 - DNA, 50 bp, Ruminococcus lactaris
  source 1..50: mol_type = genomic DNA
tgattacgatcgaggagtccaagactatgcagacagaactggatcttgta

SEQ ID NO: 368 - DNA, 50 bp, Anaerostipes sp.
  source 1..50: mol_type = genomic DNA
ttatcacgatcgaagaatctaaaacaatgaaaacagaattagatttagta

SEQ ID NO: 369 - DNA, 50 bp, Eubacterium sp.
  source 1..50: mol_type = genomic DNA
ttattacaatcgaagagtctaagacaatgaaaacagagcttgaccttgta

SEQ ID NO: 370 - DNA, 50 bp, Subdoligranulum sp.
  source 1..50: mol_type = genomic DNA
tcaccatcgaggagaacaagaccactgccgagacctacaccgaggtcgtc

SEQ ID NO: 371 - DNA, 50 bp, unidentified
  source 1..50: mol_type = unassigned DNA
ttatcaccgttgaggagtccaagaccgctgagacctactcggaggttgtt

SEQ ID NO: 372 - DNA, 50 bp, Faecalibacterium sp.
  source 1..50: mol_type = genomic DNA
tcaccattgaggagaacaagaccactgctgagacctacaacgagatcgta

SEQ ID NO: 373 - DNA, 50 bp, Alistipes finegoldii
  source 1..50: mol_type = genomic DNA
tcatcaccgtcgaggaggccaaaggcaccgagacccacgtggaggtggtc

SEQ ID NO: 374 - DNA, 50 bp, unidentified
  source 1..50: mol_type = unassigned DNA
tcatcaccatcgaggagtccaagaccgccgagacctacagcgaggtcgtc

SEQ ID NO: 375 - DNA, 50 bp, unidentified
  source 1..50: mol_type = unassigned DNA
tcaccattgaggagaacaagaccactgctgaaacctacaccgaggtagtg

SEQ ID NO: 376 - DNA, 50 bp, Eubacterium sp.
  source 1..50: mol_type = genomic DNA
ttatcacaatcgaagaatctaagaccatgaagacagagcttgaccttgta

SEQ ID NO: 377 - DNA, 50 bp, Dialister sp.
  source 1..50: mol_type = genomic DNA
ttattactgtagaagattccaaaactatgggtacaagccttaaagttgtg

SEQ ID NO: 378 - DNA, 50 bp, Coprococcus comes
  source 1..50: mol_type = genomic DNA
ttatcacaattgaagagtcaaagacaatgaagacagagcttgaccttgta

SEQ ID NO: 379 - DNA, 50 bp, Bacteroides caccae
  source 1..50: mol_type = genomic DNA
ttatcactatcgaagaagcaaaaggtactgacactacaatcggtgtagta

SEQ ID NO: 380 - DNA, 50 bp, Parabacteroides distasonis
  source 1..50: mol_type = genomic DNA
ttatcacggttgaggaagctaaaggtactgaaactacagttgacgtagtt

SEQ ID NO: 381 - DNA, 50 bp, Parabacteroides merdae
  source 1..50: mol_type = genomic DNA
ttatcactgtagaagaagctaaaggcacggaaacaacagtagacgtggta

SEQ ID NO: 382 - DNA, 50 bp, Faecalibacterium sp.
  source 1..50: mol_type = genomic DNA
tcaccattgaggagaacaagaccaccgctgagacctacaacgagatcgtg

SEQ ID NO: 383 - DNA, 50 bp, unidentified
  source 1..50: mol_type = unassigned DNA
ttatcacggtagaagaggccaaaggtaccgatacgactgtcgatattgta

SEQ ID NO: 384 - DNA, 50 bp, Roseburia inulinivorans
  source 1..50: mol_type = genomic DNA
ttatcacaatcgaagagtccaagacgatgcagacagagcttgatcttgta

SEQ ID NO: 385 - DNA, 50 bp, Roseburia inulinivorans
  source 1..50: mol_type = genomic DNA
ttatcacaatcgaagagtccaagacgatgcagacagagcttgaccttgta

SEQ ID NO: 386 - DNA, 50 bp, unidentified
  source 1..50: mol_type = unassigned DNA
ttattaccatcgaggagtctaagaccatgaagacagagctggatcttgta

SEQ ID NO: 387 - DNA, 50 bp, Dorea longicatena
  source 1..50: mol_type = genomic DNA
tcatcacaattgaagaatctaaaactatgaagacagagctggaccttgta

SEQ ID NO: 388 - DNA, 50 bp, Roseburia intestinalis
  source 1..50: mol_type = genomic DNA
ttatcacgatcgaggaatctaagacaatgcagacagagcttgacttagta

SEQ ID NO: 389 - DNA, 50 bp, unidentified
  source 1..50: mol_type = unassigned DNA
ttatcacagttgaagaatcaaagactgccgaaacatattctgaaattgtt

SEQ ID NO: 390 - DNA, 50 bp, Blautia sp.
  source 1..50: mol_type = genomic DNA
ttatcacagtagaagaatccaagaccatgcatacagaacttgacctggta

SEQ ID NO: 391 - DNA, 50 bp, Alistipes onderdonkii
  source 1..50: mol_type = genomic DNA
tgatcaccgtcgaggaggccaagggtaccgagacccatgtggaggtcgta